(19) United States
(12) Patent Application Publication
(10) Pub. No.: US 2021/0379099 A1
(43) Pub. Date: Dec. 9, 2021

(54) CUCURBITURIL MOLECULAR CONTAINERS AND METHODS OF USING SAME

(71) Applicant: University of Maryland, College Park, College Park, MD (US)

(72) Inventors: Lyle David ISAACS, Silver Spring, MD (US); Steven MURKLI, College Park, MD (US)

(21) Appl. No.: 17/335,542

(22) Filed: Jun. 1, 2021

Related U.S. Application Data
(60) Provisional application No. 63/032,381, filed on May 29, 2020.

(51) Int. Cl. A61K 31/787

(52) U.S. Cl. CPC A61K 31/787

(57) ABSTRACT

Disclosed herein are water-soluble, cyclic cucurbit[n]uril, compositions containing the same, methods of preparation thereof, and uses thereof. These compounds are useful, for example, as sequestering agents for various agents, such as, for example, drugs of abuse.

### CROSS REFERENCE TO RELATED APPLICATIONS

This application claims priority to U.S. Provisional Application No. 63/032, 381, filed on May 29, 2020, the disclosure of which is incorporated herein by reference.

### STATEMENT REGARDING FEDERALLY SPONSORED RESEARCH

This invention was made with government support under grant nos. CA168365 and GM132345 awarded by the National Institutes of Health and CHE1404911 awarded by the National Science Foundation. The government has certain rights in the invention.

### BACKGROUND OF THE DISCLOSURE

Drug abuse is a major societal problem in the United States and deaths due to overdose are common. Estimates of the costs associated with decreased work productivity and emergency room visits due to illicit drug use exceed $200 billion per year. Illicit drugs are used by an estimated 10.2% of the US population aged 12 and older each month.

Commonly abused illicit drugs include methamphetamine, fentanyl, cocaine, heroin, hallucinogens (phencyclidine and ketamine), and marijuana along with abuse of prescription medicines. Accordingly, the development of therapeutics to treat drug overdose is a pressing societal need. Currently, naloxone—which acts by a pharmacodynamic effect at the opioid receptor—is available to treat overdose with opioids but is ineffective at treating the effects of non-opioids like methamphetamine, cocaine, phencyclidine (PCP), and ketamine. As a powerful alternative, researchers are exploring the use of pharmacokinetic approaches to decrease the freely circulating drug concentration by catalytic destruction or sequestering them in the bloodstream. Human butyrylcholine esterase, for example, which hydrolyzes cocaine to ecgonine methyl ester is being explored as a therapeutic for cocaine. Similarly, antibody-based therapeutics that bind to and sequester methamphetamine, cocaine, and fentanyl in the bloodstream and thereby prevent their passage through the blood brain barrier have been investigated.

Macrocycles have long occupied a central role in the field of supramolecular chemistry. Macrocycles enjoy this privileged status because the preorganization inherent to macrocycles leads to higher binding constants and often highly selective interactions with their target guests. Among the most popular macrocyclic host families are the cyclodextrins, calixarenes, cyclophanes, cucurbit[n]urils (CB[n]), and most recently pillararenes (FIG. 1). Molecular container compounds which also include systems self-assembled by H-bonds, metal-ligand interactions and the hydrophobic effect bind to and sequester guests compounds within their cavities and thereby change their chemical and physical properties. Popular in vitro applications of these molecular containers include their use to prepare sensing ensembles, supramolecular catalysts, supramolecular materials, chiral separations phases, household deodorizers, and molecular machines. For molecular containers with excellent biocompatibility and sufficient affinity, in vivo applications become feasible. For example, the sulfonated calix[4]arene derivative SC4A exhibits excellent biocompatibility and has been investigated as an in vivo (mice) reversal agent for the toxic effects of paraquat (methyl viologen). Squaraine rotaxanes have been used for in vivo imaging and theranostic applications. Most significantly, the cyclodextrin derivatives HP-β-CD and SBE-β-CD (FIG. 1) are widely used as solubilizing excipients for insoluble drugs for parenteral administration to humans whereas Sugammadex is used as an in vivo reversal agent for the post-operative side effects of the neuromuscular blocking agents rocuronium and vecuronium. Recently, water soluble pillararenes have been investigated as in vitro hosts and in vivo reversal agents for neuromuscular blockers and as solubilizing excipients for insoluble drugs.

FIG. 1 shows the structure of CB[n] which features n glycoluril rings connected by 2n methylene bridges which define a hydrophobic cavity rimmed by two symmetry equivalent ureidyl carbonyl portals. Within the field of molecular containers, CB[n] (n=5, 6, 7, 8, 10; FIG. 1) have distinguished themselves because of their remarkably tight binding toward hydrophobic (di)cations in water with Kₐ values that regularly exceed 10⁶ M⁻¹, often exceed 10⁹ M⁻¹, and even reach 10¹⁷ M⁻¹ in special cases. The remarkable binding affinity of CB[n] toward their guests has been traced to their highly electrostatically negative C═O portals which constitute cation binding regions juxtaposed with a hydrophobic cavity that contains high energy water molecules that provide an enthalpic driving force upon complexation. Although unfunctionalized macrocyclic CB[n] exhibit excellent biocompatibility, only CB[7] exhibits both good solubility (>5 mM) and a cavity large enough to encapsulate biologically relevant guests. Accordingly, others have demonstrated the use of CB[7] as an in vivo sequestration agent in several applications including to counteract the toxic effects of paraquat, to alleviate blood coagulation induced by hexadimethrine bromide (mice), to reverse paralysis induced by succinyl choline (mice), to reverse general anesthesia in zebrafish, and to mask the bitter taste and toxicity of various species. Over the past decade, the acyclic CB[n]-type receptors (e.g. M1 and M2, FIG. 1) which have high water solubility and binding affinity have been synthesized and their molecular recognition properties toward specific classes of biologically active guests have been studied. It was previously demonstrated that M1 and M2 can act as solubilizing excipients for insoluble drugs and as in vivo sequestration agents for neuromuscular blockers, the general anesthetics etomidate and ketamine, and drugs of abuse (methamphetamine and fentanyl).

### SUMMARY OF THE DISCLOSURE

The present disclosure provides compounds, compositions, methods of using the compounds or compositions, and articles comprising the compounds or compositions.

In an aspect, the present disclosure provides water-soluble, cyclic cucurbit[n]urils, which may be referred to as CB[n]'s, where n corresponds to the number glycoluril groups of the cucurbit[n]uril present in the macrocycle. There may be 6, 7, or 8 glycoluril groups in a cyclic cucurbit[n]uril of the present disclosure. The cyclic cucurbit[n]urils may have various substituents.

In various examples, a compound of the present disclosure has the following structure:

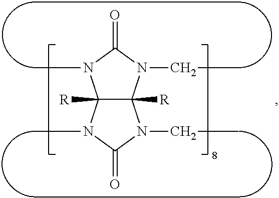

where each R is independently chosen from hydrogen, alkyl groups (e.g., methyl, ethyl, propyl, or isopropyl), hydroxyl groups, alkoxy groups, aryl groups, heteroaryl groups, amino groups, amide groups (e.g., N-and C-linked, primary secondary tertiary), carboxylic acid/carboxylate groups, ester groups (e.g., O— and C═O linked ester groups), imide groups, thiol groups, ether groups, polyether groups, phosphate groups, phosphonate groups, sulfonate groups (e.g., protonated and deprotonated sulfonate groups, where the counterion for the deprotonated sulfonated group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfonate groups (e.g., methyl sulfonate, ethyl sulfonate, propyl sulfonate, butyl sulfonate, and the like) (e.g., protonated and deprotonated alkyl sulfonate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), sulfate groups ((e.g., protonated and deprotonated sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfate groups (e.g., protonated and deprotonated alkyl sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), and the like, and combinations thereof. In various examples, at least one R group is not H. In various examples, the two R groups of a glycoluril group are connected such that they form a heterocycle or carbocycle, such as, for example, a cyclohexyl group, cyclopentyl group, or cyclobutyl group.

In an aspect, the present disclosure provides compositions comprising one or more compound(s). Non-limiting examples of compositions are described herein.

A composition may comprise one or more compound(s) and one or more pharmaceutical agent(s). In various examples, a pharmaceutical agent comprises one or more positively charged nitrogen atom(s) (e.g., ammonium ions, primary ammonium ions, secondary ammonium ions, tertiary ammonium ions, quaternary ammonium ions, or a combination thereof, where the non-hydrogen group(s) on the ammonium are chosen from aliphatic groups, alkyl groups, aryl groups, and combinations thereof).

In an aspect, the present disclosure provides uses of cyclic cucurbit[n]urils. Non-limiting examples of uses of cyclic cucurbit[n]urils are provided herein, for example, non-limiting examples of uses of cyclic cucurbit[n]urils are described in the Statements and Example.

Cyclic cucurbit[n]urils can be used to sequester various materials, which may be chemical compounds. In various non-limiting examples, one or more cyclic cucurbit[n]urils(s) is/are used to sequester one or more neuromuscular blocking agent(s) (such as, for example, rocuronium, tubocurarine, atracurium, (cis)atracurium besylate, mivacurium, gallamine, pancuronium, vecuronium, and rapacuronium, and the like); one or more anesthesia agent(s) (such as, for example, N-methyl D-aspartate (NMDA) receptor antagonists (e.g., ketamine and the like), short-acting anesthetic agents (e.g., etomidate and the like), and the like); one or more pharmaceutical agent(s) (such as, for example, a drug (e.g., anticoagulants, such as, for example, hexadimethrine and the like)); drugs of abuse (e.g., methamphetamine, cocaine, fentanyl, carfentanil, PCP, MDMA, heroin, and the like); one or more pesticide(s) (such as, for example, paraquat, diquat, organochlorines (e.g., DDT, aldrin, and the like), neonicotinoids (e.g., permethrin and the like), organophosphates (e.g., malathion, glyphosate, and the like), pyrethroids, triazines (e.g., atrazine and the like), and the like); one or more dyestuff(s) (such as, for example, methylene blue, nile red, crystal violet, thioflavin T, thiazole orange, proflavin, acridine orange, methylene violet, azure A, neutral red, cyanines, Direct orange 26, disperse dyes (e.g., disperse yellow 3, disperse blue 27, and the like), coumarins, congo red, and the like); one or more malodorous compound(s) (such as, for example, low molecular weight thiols (e.g., C₁-C₄ thiols), low molecular weight amines (e.g., triethylamine, putrescine, cadaverine, and the like), and the like); or one or more chemical warfare agent(s) (such as for example, nitrogen and sulfur mustards (e.g., bis(2-chloroethyl)ethylamine, bis(2-chloroethyl)methylamine, tris(2-chloroethyl)amine, bis(2-chloroethyl) sulfide, bis(2-chloroethylthioethyl) ether, and the like), nerve agents (such as, for example, those from the G, GV, and V series of nerve agents (e.g., tabun, sarin, soman, cyclosarin, 2-(dimethylamino)ethyl N,N-dimethylphosphoramidofluoridate (GV), novichok agents, VE, VG, VM, VX, and the like), and the like); one or more hallucinogen(s) (e.g., ergolines, lysergic acid diethylamide (LSD), psilocybin, tryptamines, dimethyltryptamine (DMT), phenethylamines, mescaline, ayahuasca, dextromethorphan, and the like); one or more toxin(s) (e.g., dioxins, perfluoralkylsulfonates (PFAS), perfluorooctanoic acid (PFOA), decabromobiphenyl ether (DECA), heavy metals (e.g., mercury), muscarine, tyramine, strychnine, tetrodotoxin, saxitoxin and the like, cholesterol, deoxycholic acid, N-Methyl-4-phenyl-1,2,3,6-tetrahydropyridine, phenylalanine, tyrosine, arginine, histamine); one or more metabolite(s) (e.g., toxic metabolites, such as, for example, N-methyl-4-phenylpyridine, spermine, spermidine, N-nitroso compounds e.g., 4-(methylnitrosoamino)-1-(3-pyridyl)-1-butanone); or the like, or a combination thereof.

In an aspect, the present disclosure provides articles comprising compounds of the present disclosure.

The articles may be articles of manufacture. Non-limiting examples of articles include wipes impregnated with one or more compounds of the present disclosure. For example, such a wipe is used to decontaminate a surface from any material capable of being sequestered by a compound (e.g., a cyclic cucurbit[n]uril of the present disclosure). For example, the wipe is used to decontaminate a surface that has or was previously exposed to a toxin, drug of abuse, or the like, or a combination thereof.

### BRIEF DESCRIPTION OF THE FIGURES

For a fuller understanding of the nature and objects of the disclosure, reference should be made to the following detailed description taken in conjunction with the accompanying figures.

FIG. 1 shows structure of CB[n] and acyclic CB[n]-type receptors M1 and M2.

FIG. 2 shows structure of the water soluble CB[8] derivative (Me₄CB[8]) that was used herein.

FIG. 3 shows chemical structures of competitive guests and drugs of abuse used in this study.

FIG. 4 ¹H NMR spectra recorded (600 MHz, RT, D₂O) for a) PCP 8 (0.4 mM), b) an equimolar mixture of Me₄CB[8] and 8 (0.2 mM), c) a mixture of 8 (0.4 mM) and Me₄CB[8] (0.2 mM), d) fentanyl 4 (0.4 mM), e) a equimolar mixture of 4 (0.2 mM) and Me₄CB[8] (0.2 mM), f) a mixture of 4 (0.4 mM) and Me₄CB[8] (0.2 mM), g) a mixture of 4 (0.8 mM) and Me₄CB[8] (0.2 mM).

FIG. 5 shows a cross-eyed stereoview of an MMFF minimized geometry of the Me₄CB[8]•8 complex.

FIG. 6 shows (a) a plot of DP vs time from the titration of Me₄CB[8] (104 μM) and 11 (200 μM) in the cell with 8 (1.0 mM) in the syringe in 20 mM NaH₂PO₄ buffer (pH=7.4); (b) plot of ΔH as a function of molar ratio of Me₄CB[8] to 8. The solid line represents the best non-linear fit of the data to the competition binding model (Kₐ=(5.35±0.19)×10⁸ M⁻¹ and ΔH=(−8.39±0.01) kcal·mol⁻¹.

FIG. 7 shows in vitro cytotoxicity experiments performed for Me₄CB[8]: a) HEPG2 cell viability assay after incubating the cells with Me₄CB[8] container for 24 h (UT=Untreated). This figure is the average SEM values representative of two replicate experiments. Statistical analysis is one-way ANOVA with Dunnett's multiple comparisons test. **P=0.001−0.01; ****P<0.0001. b) HEK293 cell viability assay performed after incubation with Me₄CB[8] container for 24 h (UT=Untreated). This figure is the average SEM values representative of two replicate experiments. Statistical analysis is one-way ANOVA with Dunnett's multiple comparisons test. *P=0.01−0.05; ****P<0.0001. c) HEPG2 cell death after incubation with Me₄CB[8]. AK assay was performed using the supernatant from cells seeded for MTS assay (UT=untreated). This figure is the average and SEM values representative of two replicate experiments. Statistical analysis is one-way ANOVA with Dunnett's multiple comparisons test. ****P<0.001, d). HEK293 cell death after incubation with Me₄CB[8]. AK assay was performed using the supernatant from cells seeded for MTS assay (UT=untreated). This figure is the average and SEM values representative of two replicate experiments. Statistical analysis is one-way ANOVA with Dunnett's multiple comparisons test. ****P<0.0001.

FIG. 8 shows an MTD study performed for Me₄CB[8]. Female Swiss Webster mice (n=5 per group) were dosed via tail vein injection (0.150 mL) on days 0 and 2 (denoted by *) with different concentrations of Me₄CB[8] or phosphate buffered saline (PBS). The normalized average weight change per study group is indicated. Error bars represent SEM.

FIG. 9 shows in vivo reversal of PCP-induced hyperlocomotion by Me₄CB[8]. a) Average locomotion counts for male Swiss Webster mice (N=8) (black bars) treated with nothing (REF), saline (SAL), Me₄CB[8] alone (CON), PCP (PCP, 8) or a premixed solution of PCP and Me₄CB[8] (PCPC). Error bars represent SEM. Star signifies significant increase in locomotion counts (p's<0.05) for PCP compared to REF, SAL, CON and PCPC. b) Average locomotion counts for male Swiss Webster mice (n=9) (grey bars) treated with nothing (REF), saline (SAL), sequential administration of Me₄CB[8] followed 30 s later by PCP (CON+PCP), sequential administration of PCP followed 30 s later by Me₄CB[8] (PCP+CON), or PCP alone (PCP). Error bars represent SEM. Star signifies significant increase in locomotion counts (p's<0.05) for PCP compared to REF, SAL, CPCP and PCPC.

FIG. 10 shows a cartoon of water soluble analogue Me₄CB[8] binding PCP.

FIG. 11 shows chemical structures of the host and drugs of abuse used herein.

FIG. 12 shows a) a plot of change in DP vs time from the titration of CB[8] (162 μM) in the cell with guest 3 (1.87 mM) in the syringe in 20 mM NaH₂PO₄ buffer (pH=7.4); b) plot of ΔH as a function of molar ratio of 3 to CB[8]. The solid line represents the best non-linear fit of the data to the single set of sites model (Kₐ=(1.47±0.09)×10⁵ M⁻¹) and ΔH=(−7.84±0.1)).

FIG. 13 shows a) a plot of change in DP vs time from the titration of CB[8] (50 μM) in the cell with guest 4 (850 μM) in the syringe in 20 mM NaH₂PO₄ buffer (pH=7.4); b) plot of ΔH as a function of molar ratio of 4 to CB[8]. The solid line represents the best non-linear fit of the data to the sequential binding sites model (Kₐ₁=(1.9±0.09)×10⁷ M⁻¹), (Kₐ₂=(3.7±0.04)×10⁵ M⁻¹) and ΔH₁=(−10.8±0.06), ΔH₂=(−7.67±0.04)).

FIG. 14 shows a) a plot of change in DP vs time from the titration of CB[8] (36.6 μM) in the cell with guest 10 (500 μM) in the syringe in 20 mM NaH₂PO₄ buffer (pH=7.4); b) plot of ΔH as a function of molar ratio of 10 to CB[8]. The solid line represents the best non-linear fit of the data to the single set of sites model (Kₐ=(5.34±0.24)×10⁶ M⁻¹) and ΔH=(−7.62±0.03)).

FIG. 15 shows a) a plot of change in DP vs time from the titration of CB[8] (44.3 μM) and 10 (75 μM) in the cell with guest 5 (500 μM) in the syringe in 20 mM NaH₂PO₄ buffer (pH=7.4); b) plot of ΔH as a function of molar ratio of 5 to CB[8]. The solid line represents the best non-linear fit of the data to the single set of sites competition model (Kₐ=(3.41±0.15)×10⁸ M⁻¹) and ΔH=(−13.6±0.04)).

FIG. 16 shows a) a plot of change in DP vs time from the titration of CB[8] (39.8 μM) and 10 (75 μM) in the cell with guest 6 (500 μM) in the syringe in 20 mM NaH₂PO₄ buffer (pH=7.4); b) plot of ΔH as a function of molar ratio of 6 to CB[8]. The solid line represents the best non-linear fit of the data to the single set of sites competition model (Kₐ=(1.7±0.11)×10⁸ M⁻¹) and ΔH=(−15.8±0.1)).

FIG. 17 shows a) plot of change in DP vs time from the titration of CB[8] (20.7 μM) in the cell with guest 11 (250 μM) in the syringe in 20 mM NaH₂PO₄ buffer (pH=7.4); b) plot of ΔH as a function of molar ratio of 11 to CB[8]. The solid line represents the best non-linear fit of the data to the single set of sites model (Kₐ=(3.18±0.3)×10⁷ M⁻¹) and ΔH=(−8.26±0.04)).

FIG. 18 shows a) a plot of change in DP vs time from the titration of CB[8] (50 μM) and 11 (200 μM) in the cell with guest 7 (500 μM) in the syringe in 20 mM NaH₂PO₄ buffer (pH=7.4); b) plot of ΔH as a function of molar ratio of 7 to CB[8]. The solid line represents the best non-linear fit of the data to the single set of sites competition model (Kₐ=(1.09±0.1)×10⁹ M⁻¹) and ΔH=(−17.3±0.16)).

FIG. 19 shows a) a plot of change in DP vs time from the titration of CB[8] (38.8 μM) and 11 (200 μM) in the cell with guest 8 (500 μM) in the syringe in 20 mM NaH₂PO₄ buffer (pH=7.4); b) plot of ΔH as a function of molar ratio of 8 to CB[8]. The solid line represents the best non-linear fit of the data to the single set of sites competition model (Kₐ=(2.1±0.2)×10¹⁰ M⁻¹) and ΔH=(−14.9±0.04)).

FIG. 20 shows a) a plot of change in DP vs time from the titration of CB[8] (100 μM) in the cell and with guest 9 (795 μM) in the syringe in 20 mM NaH₂PO₄ buffer (pH=7.4); b) plot of ΔH as a function of molar ratio of 9 to CB[8]. The solid line represents the best non-linear fit of the data to the single set of sites model (Kₐ=(6.45±0.43)×10⁵ M⁻¹) and ΔH=(−8.26±0.15)).

FIG. 21 shows a) a plot of change in DP vs time from the titration of Me₄CB[8] (116 μM) in the cell with guest 3 (2.50 mM) in the syringe in 20 mM NaH₂PO₄ buffer (pH=7.4); b) plot of ΔH as a function of molar ratio of 3 to Me₄CB[8]. The solid line represents the best non-linear fit of the data to the single set of sites model (Kₐ=(2.98±0.47)×10⁴ M⁻¹ and ΔH=(−4.61±0.04)).

FIG. 22 shows a) a plot of change in DP vs time from the titration of Me₄CB[8] (100 μM) in the cell with guest 4 (1.0 mM) in the syringe in 20 mM NaH₂PO₄ buffer (pH=7.4); b) plot of ΔH as a function of molar ratio of 4 to Me₄CB[8]. The solid line represents the best non-linear fit of the data to the sequential binding sites model (Kₐ₁=(1.98±0.02)×10⁶ M⁻¹) , Kₐ₂=(9.52±0.03)×10⁴) M⁻¹ and ΔH₁=(−3.58±0.01), ΔH₂=−1.94±0.01)).

FIG. 23 shows a) a plot of change in DP vs time from the titration of Me₄CB[8] (102 μM) in the cell with guest 10 (1.0 mM) in the syringe in 20 mM NaH₂PO₄ buffer (pH=7.4); b) plot of ΔH as a function of molar ratio of 10 to Me₄CB[8]. The solid line represents the best non-linear fit of the data to the single set of sites model (Kₐ=(1.04±0.02)×10⁶ M⁻¹ and ΔH=(−5.35±0.01)).

FIG. 24 shows a) a plot of change in DP vs time from the titration of Me₄CB[8] (100 μM) and 10 (200 μM) in the cell with guest 5 (1.0 mM) in the syringe in 20 mM NaH₂PO₄ buffer (pH=7.4); b) plot of ΔH as a function of molar ratio of 5 to Me₄CB[8]. The solid line represents the best non-linear fit of the data to the single set of sites competition model (Kₐ=(4.67±0.17)×10⁷ M⁻¹) and ΔH=(−8.24±0.02)).

FIG. 25 shows a) a plot of change in DP vs time from the titration of Me₄CB[8] (103 μM) and 10 (200 μM) in the cell with guest 6 (1.0 mM) in the syringe in 20 mM NaH₂PO₄ buffer (pH=7.4); b) plot of ΔH as a function of molar ratio of 6 to Me₄CB[8]. The solid line represents the best non-linear fit of the data to the single set of sites competition model (Kₐ=(2.14±0.05)×10⁷ M⁻¹ and ΔH=(−8.43±0.02)).

FIG. 26 shows a) a plot of change in DP vs time from the titration of Me₄CB[8] (100 μM) in the cell with guest 11 (1.0 mM) in the syringe in 20 mM NaH₂PO₄ buffer (pH=7.4); b) plot of ΔH as a function of molar ratio of 11 to Me₄CB[8]. The solid line represents the best non-linear fit of the data to the single set of sites model (Kₐ=(2.79±0.17)×10⁶ M⁻¹ and ΔH=(−5.63±0.03)).

FIG. 27 shows a) a plot of change in DP vs time from the titration of Me₄CB[8] (115 μM) and 11 (200 μM) in the cell with guest 7 (1.5 mM) in the syringe in 20 mM NaH₂PO₄ buffer (pH=7.4); b) plot of ΔH as a function of molar ratio of 7 to Me₄CB[8]. The solid line represents the best non-linear fit of the data to the single set of sites competition model (Kₐ=(2.81±0.67)×10⁷ M⁻¹) and ΔH=(−9.37±0.02)).

FIG. 28 shows a) a plot of change in DP vs time from the titration of Me₄CB[8] (104 μM) and 11 (200 μM) in the cell with guest 8 (1.0 mM) in the syringe in 20 mM NaH₂PO₄ buffer (pH=7.4); b) plot of ΔH as a function of molar ratio of 8 to Me₄CB[8]. The solid line represents the best non-linear fit of the data to the single set of sites competition model (Kₐ=(5.35±0.19)×10⁸M⁻¹) and ΔH=(−8.39±0.01)).

FIG. 29 shows a) a plot of change in DP vs time from the titration of Me₄CB[8] (91.2 μM) in the cell and with guest 9 (1.0 mM) in the syringe in 20 mM NaH₂PO₄ buffer (pH=7.4); b) plot of ΔH as a function of molar ratio of 9 to Me₄CB[8]. The solid line represents the best non-linear fit of the data to the single set of sites model (Kₐ=(2.77±0.15)×10⁵ M⁻¹) and ΔH=(−6.90±0.07)).

FIG. 30 shows a) a plot of change in DP vs time from the titration of Me₄CB[8] (68 μM) in the cell with guest 12 (1.0 mM) in the syringe in 20 mM NaH₂PO₄ buffer (pH=7.4); b) plot of ΔH as a function of molar ratio of 12 to Me₄CB[8]. The solid line represents the best non-linear fit of the data to the single set of sites model (Kₐ=(3.13±0.32)×10⁴ M⁻¹ and ΔH=(−13.8±0.77)).

FIG. 31 shows a) a plot of change in DP vs time from the titration of Me₄CB[8] (105 μM) in the cell with guest 13 (1.0 mM) in the syringe in 20 mM NaH₂PO₄ buffer (pH=7.4); b) plot of ΔH as a function of molar ratio of 13 to Me₄CB[8]. The solid line represents the best non-linear fit of the data to the single set of sites model (Kₐ=(3.07±0.39)×10⁵ M⁻¹ and ΔH=(−9.89±0.29)).

FIG. 32 shows a) a plot of change in DP vs time from the titration of Me₄CB[8] (96 μM) in the cell with guest 14 (1.0 mM) in the syringe in 20 mM NaH₂PO₄ buffer (pH=7.4); b) plot of ΔH as a function of molar ratio of 14 to Me₄CB[8]. The solid line represents the best non-linear fit of the data to the single set of sites model (Kₐ=(7.94±0.07)×10⁴ M⁻¹ and ΔH=(−15.1±0.10)).

FIG. 33 shows ¹H NMR spectra recorded (600 MHz, RT, D₂O) for a) 3 (0.25 mM), b) CB[8] (0.125 mM), c) an equimolar mixture of 3 and CB[8] (0.125 mM), and d) a mixture of 3 (0.250 mM) and CB[8] (0.125 mM).

FIG. 34 shows ¹H NMR spectra recorded (600 MHz, RT, D₂O) for a) 4 (0.25 mM), b) CB[8] (0.125 mM), c) an equimolar mixture of 4 and CB[8] (0.125 mM), d) a mixture of 4 (0.250 mM) and CB[8] (0.125 mM), and e) a mixture of 4 (0.5 mM) and CB[8] (0.125 mM).

FIG. 35 shows ¹H NMR spectra recorded (600 MHz, RT, D₂O) for a) 5 (0.25 mM), b) CB[8] (0.125mM), c) an equimolar mixture of 5 and CB[8] (0.125 mM), and d) a mixture of 5 (0.250 mM) and CB[8] (0.125 mM).

FIG. 36 shows ¹H NMR spectra recorded (600 MHz, RT, D₂O) for a) 6 (0.25 mM), b) CB[8] (0.125 mM), c) an equimolar mixture of 6 and CB[8] (0.125 mM), and d) a mixture of 6 (0.250 mM) and CB[8] (0.125 mM).

FIG. 37 shows ¹H NMR spectra recorded (600 MHz, RT, D₂O) for a) 7 (0.25 mM), b) CB[8] (0.125 mM), c) an equimolar mixture of 7 and CB[8] (0.125 mM), and d) a mixture of 7 (0.250 mM) and CB[8] (0.125 mM).

FIG. 38 shows ¹H NMR spectra recorded (600 MHz, RT, D₂O) for a) 8 (0.25 mM), b) CB[8] (0.125 mM), c) an equimolar mixture of 8 and CB[8] (0.125 mM), and d) a mixture of 8 (0.250 mM) and CB[8] (0.125 mM).

FIG. 39 shows ¹H NMR spectra recorded (600 MHz, RT, D₂O) for a) 9 (0.25 mM), b) CB[8] (0.125 mM), c) an equimolar mixture of 9 and CB[8] (0.125 mM), and d) a mixture of 9 (0.250 mM) and CB[8] (0.125 mM).

FIG. 40 shows ¹H NMR spectra recorded (600 MHz, RT, D₂O) for a) 3 (0.25 mM), b) Me₄CB[8] (0.125 mM), c) an equimolar mixture of 3 and Me₄CB[8] (0.125 mM), and d) a mixture of 3 (0.250 mM) and Me₄CB[8] (0.125 mM).

FIG. 41 shows ¹H NMR spectra recorded (600 MHz, RT, D₂O) for a) 5 (0.25 mM), b) Me₄CB[8] (0.125mM), c) an equimolar mixture of 5 and Me₄CB[8] (0.125 mM), and d) a mixture of 5 (0.250 mM) and Me₄CB[8] (0.125 mM).

FIG. 42 shows ¹H NMR spectra recorded (600 MHz, RT, D₂O) for a) 6 (0.25 mM), b) Me₄CB[8] (0.125 mM), c) an equimolar mixture of 6 and Me₄CB[8] (0.125 mM), and d) a mixture of 6 (0.250 mM) and Me₄CB[8] (0.125 mM).

FIG. 43 shows ¹H NMR spectra recorded (600 MHz, RT, D₂O) for a) 7 (0.25 mM), b) Me₄CB[8] (0.125 mM), c) an equimolar mixture of 7 and Me₄CB[8] (0.125 mM), and d) a mixture of 7 (0.250 mM) and Me₄CB[8] (0.125 mM).

FIG. 44 shows ¹H NMR spectra recorded (600 MHz, RT, D₂O) for a) 9 (0.25 mM), b) Me₄CB[8] (0.125 mM), c) an equimolar mixture of 9 and Me₄CB[8] (0.125 mM), and d) a mixture of 9 (0.250 mM) and Me₄CB[8] (0.125 mM).

FIG. 45 shows ¹H NMR spectra recorded (600 MHz, RT, D₂O) for a) 12 (0.25 mM), b) Me₄CB[8] (0.125 mM), c) an equimolar mixture of 12 and Me₄CB[8] (0.125 mM), and d) a mixture of 12 (0.250 mM) and Me₄CB[8] (0.125 mM).

FIG. 46 shows ¹H NMR spectra recorded (600 MHz, RT, D₂O) for a) 13 (0.25 mM), b) Me₄CB[8] (0.125 mM), c) an equimolar mixture of 13 and Me₄CB[8] (0.125 mM), and d) a mixture of 13 (0.250 mM) and Me₄CB[8] (0.125 mM).

FIG. 47 shows ¹H NMR spectra recorded (600 MHz, RT, D₂O) for a) 14 (0.25 mM), b) Me₄CB[8] (0.125 mM), c) an equimolar mixture of 14 and Me₄CB[8] (0.125 mM), and d) a mixture of 14 (0.250 mM) and Me₄CB[8] (0.125 mM).

FIG. 48 shows HEK293 cell death after incubation with Me₄CB[8]. AK assay was performed using supernatant from cells seeded for MTS assay (UT=untreated). This figure is the average and SEM values representative of two replicate experiments. Statistical analysis is one-way ANOVA with Dunnett's multiple comparisons test. ****P<0.0001.

FIG. 49 shows HEPG2 cell death after incubation with Me₄CB[8]. AK assay was performed using supernatant from cells seeded for MTS assay (UT=untreated). This figure is the average and SEM values representative of two replicate experiments. Statistical analysis is one-way ANOVA with Dunnett's multiple comparisons test. ****P<0.001.

FIG. 50 shows HEK293 cell viability after incubation with Me₄CB[8]. MTS assay was performed after the cells were incubated with Me₄CB[8] for 24 h (UT=untreated). This figure is the average and SEM values representative of two replicate experiments. Statistical analysis is one-way ANOVA with Dunnett's multiple comparisons test. *P=0.01-0.05;****P<0.0001.

FIG. 51 shows HEPG2 cell viability after incubation with Me₄CB[8]. MTS assay was performed after the cells were incubated with Me₄CB[8] for 24 h (UT=untreated). This figure is the average and SEM values representative of two replicate experiments. Statistical analysis is one-way ANOVA with Dunnett's multiple comparisons test. **P=0.001-0.01;****P<0.0001.

FIG. 52 shows MTD study performed for Me₄CB[8]. Female Swiss Webster mice (n=5 per group) were dosed via tail vein on days 0 and 2 (denoted by *) with different concentrations of Me₄CB[8] or phosphate buffered saline (PBS). The normalized average weight change per study group is indicated. Error bars represent SEM.

FIG. 53 shows in vivo reversal of PCP-induced hyperlocomotion by Me₄CB[8]. Average locomotion counts for male Swiss Webster mice (n=8) (grey bars) treated with nothing (REF), saline (SAL), Me₄CB[8] alone (CON), PCP (PCP) or a premixed solution of PCP plus Me₄CB[8] (PCPC). Error bars represent SEM. Color lines represent individual locomotion counts for each mouse. Star signifies significant increase in locomotion counts (p's<0.05) for PCP compared to REF, SAL, CON and PCPC.

FIG. 54 shows in vivo reversal of PCP-Induced Hyperlocomotion by Me₄CB[8]. Average locomotion counts for male Swiss Webster mice (n=9) (great bars) treated with nothing (REF), saline (SAL), sequential administration of Me₄CB[8] followed 30 s later by PCP (CPCP), sequential administration of PCP followed 30 s later by Me₄CB[8], or PCP alone (PCP). Error bars represent SEM. Color lines represent individual locomotion counts for each mouse. Star signifies significant increase in locomotion counts (p's<0.05) for PCP compared to REF, SAL, CPCP and PCPC.

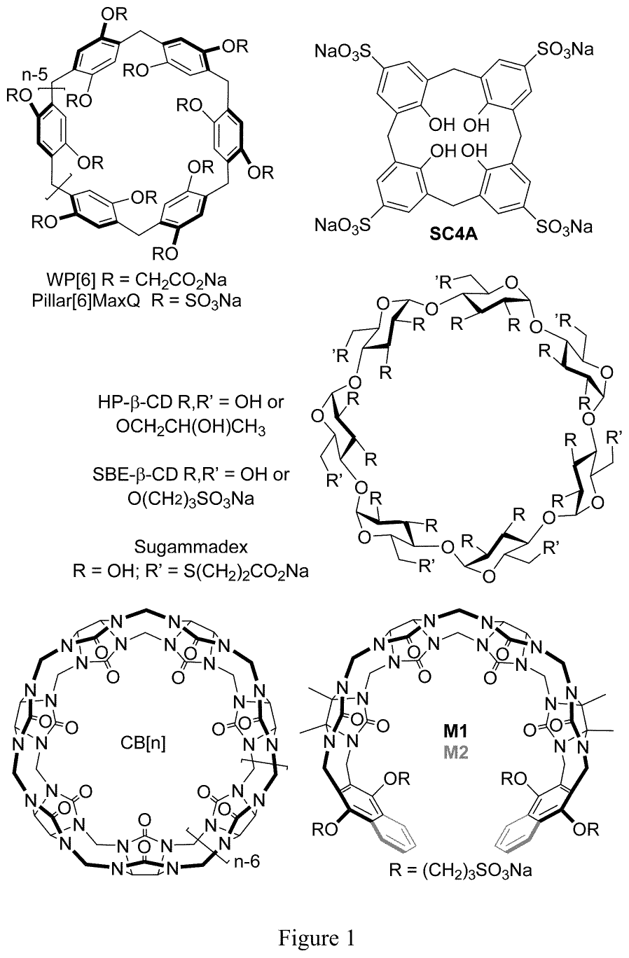

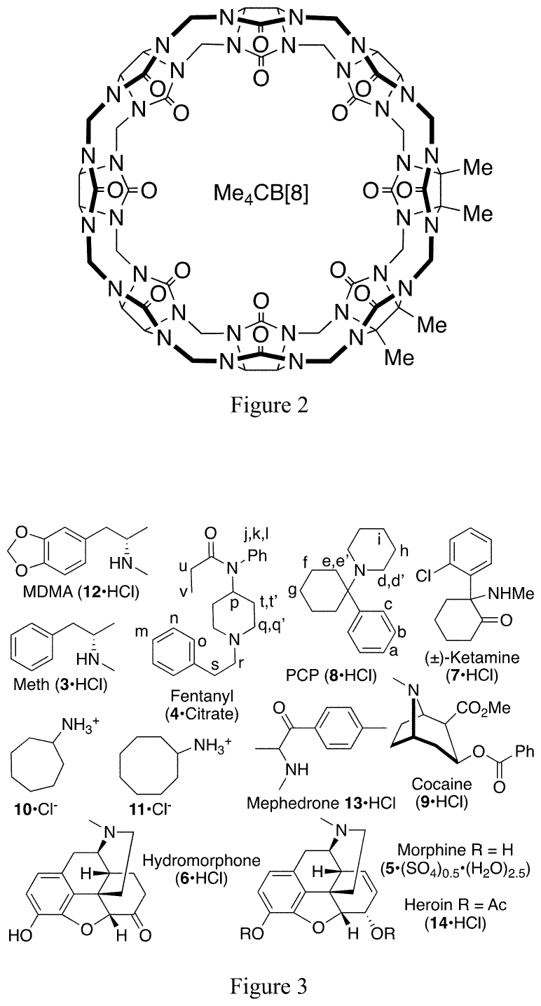

### DETAILED DESCRIPTION OF THE DISCLOSURE

Although claimed subject matter will be described in terms of certain embodiments and examples, other embodiments and examples, including embodiments and examples that do not provide all of the benefits and features set forth herein, are also within the scope of this disclosure. Various structural, logical, and process step changes may be made without departing from the scope of the disclosure.

All ranges provided herein include all values that fall within the ranges to the tenth decimal place, unless indicated otherwise.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

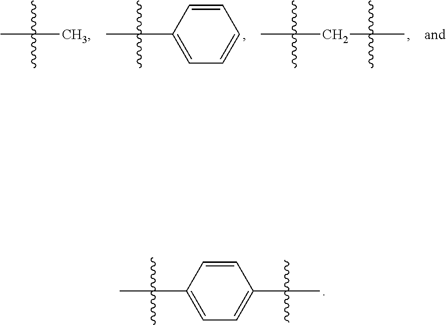

As used herein, unless otherwise indicated, the term “aryl group” refers to C₅ to C₁₈, including all integer numbers of carbons and ranges of numbers of carbons therebetween, aromatic or partially aromatic carbocyclic groups (e.g., C₁, C₂, C₃, C₄, C₅, C₆, C₇, C₈, C₉, C₁₀, C₁₁, C₁₂, C₁₃, C₁₄, C₁₅, C₁₆, C₁₇, and C₁₈). An aryl group may also be referred to as an aromatic group. The aryl groups can comprise polyaryl groups such as, for example, fused ring or biaryl groups. The aryl group can be unsubstituted or substituted with one or more substituent(s). Examples of substituents include, but are not limited to, various sub stituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof. A substituent may be or further comprise a sulfonate group or a sulfate group. Examples of aryl groups include, but are not limited to, phenyl groups, biaryl groups (e.g., biphenyl groups and the like), and fused ring groups (e.g., naphthyl groups, anthracene groups, pyrenyl groups, and the like), which may be unsubstituted or substituted.

As used herein, unless otherwise indicated, the term “heteroaryl group” refers to a C₁ to C₁₈ monocyclic, polycyclic, or bicyclic ring groups (e.g., aryl groups) comprising one or two aromatic rings containing at least one heteroatom (e.g., nitrogen, oxygen, sulfur, and the like) in the aromatic ring(s), including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, and C₁₈). The heteroaryl groups may be substituted or unsubstituted. Examples of heteroaryl groups include, but are not limited to, benzofuranyl groups, thienyl groups, furyl groups, pyridyl groups, pyrimidyl groups, oxazolyl groups, quinolyl groups, thiophenyl groups, isoquinolyl groups, indolyl groups, triazinyl groups, triazolyl groups, isothiazolyl groups, isoxazolyl groups, imidazolyl groups, benzothiazolyl groups, pyrazinyl groups, pyrimidinyl groups, thiazolyl groups, and thiadiazolyl groups, and the like. Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof.

As used herein, unless otherwise indicated, the term “aliphatic” refers to branched or unbranched hydrocarbon groups that, optionally, contain one or more degree(s) of unsaturation. Degrees of unsaturation can arise from, but are not limited to, cyclic aliphatic groups. For example, the aliphatic groups/moieties are a C₁ to C₄₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). Aliphatic groups include, but are not limited to, alkyl groups, alkenyl groups, and alkynyl groups. The aliphatic group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof.

As used herein, “carbocyclic group” refers to a cyclic compound having a ring or multiple rings in which all of the atoms forming the ring(s) are carbon atoms. The rings of the carbocyclic group can be aromatic or nonaromatic, and include compounds that are saturated and partially unsaturated, and fully unsaturated. Examples of such groups include benzene, naphthalene, 1,2-dihydronaphthalene, cyclohexane, cyclopentene, and the like. For example, the carbocyclic group can be a C₃ to C₂₀ carbocyclic group, including all integer numbers of carbons and ranges of numbers of carbons therebetween (e.g., C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). Aliphatic groups may be carbocyclic groups. Carbocyclic groups may be substituted. Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof.

As used herein, “heterocyclic group” refers to a cyclic compound having a ring or multiple rings where at least one of the atoms forming the ring(s) is a heteroatom (e.g., oxygen, nitrogen, sulfur, etc.). The rings of the heterocyclic group can be aromatic or nonaromatic, and include compounds that are saturated, partially unsaturated, and fully unsaturated. Examples of such groups include imidazolidin-2-one, pyridine, quinoline, decahydroquinoline, tetrahydrofuran, pyrrolidine, pyrrolidone, and the like. For example, the heterocyclic group can be a C₁ to C₂₀heterocyclic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). Heterocyclic groups may be substituted. Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof.

As used herein, “carbocyclic ring system” refers to a cyclic compound having a ring or multiple rings in which all of the atoms forming the ring(s) are carbon atoms. Examples of such groups include benzene, naphthalene, 1,2-dihydronaphthalene, cyclohexane, cyclopentene, and the like. The rings of the carbocyclic ring system or heterocyclic ring system can be aromatic or nonaromatic, and include compounds that are saturated, partially unsaturated, and fully unsaturated. For example, the carbocyclic ring system can be a C₃ to C₂₀ carbocyclic group, including all integer numbers of carbons and ranges of numbers of carbons therebetween (e.g., C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). In another example, the carbocyclic ring system can be a phenyl group or naphthyl group. The phenyl group or naphthyl group is attached to the compound via adjacent carbons of the phenyl group or naphthyl group. A carbocyclic ring system may be substituted. Examples of substituents include, but are not limited to, various sub stituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof.

As used herein, “heterocyclic ring system” refers to a cyclic compound having a ring or multiple rings in which at least one of the atoms forming the ring(s) is a heteroatom (e.g., oxygen, nitrogen, sulfur, etc.). The rings of the carbocyclic ring system or heterocyclic ring system can be aromatic or nonaromatic, and include compounds that are saturated, and fully unsaturated. Examples of the heterocyclic ring system include imidazolidin-2-one, pyridine, quinoline, decahydroquinoline, tetrahydrofuran, pyrrolidine, pyrrolidone, and the like. For example, the heterocyclic ring system can be a C₁ to C₂₀heterocyclic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). A heterocyclic ring system may be substituted. Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof.

As used herein, unless otherwise indicated, the term “alkyl group” refers to branched or unbranched saturated hydrocarbon groups. Examples of alkyl groups include, but are not limited to, methyl groups, ethyl groups, n- and isopropyl groups, n-, iso-, sec-, and tert-butyl groups, and the like. For example, the alkyl group can be a C₁ to C₁₂, including all integer numbers of carbons and ranges of numbers of carbons therebetween (e.g., C₁, C₂, C₃, C₄, C₅, C₆, C₇, C₈, C₉, C₁₀, C₁₁, and C₁₂). The alkyl group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof.

The present disclosure provides compounds, compositions, methods of using the compounds or compositions, and articles comprising the compounds or compositions.

In an aspect, the present disclosure provides water-soluble, cyclic cucurbit[n]urils, which may be referred to as CB[n]'s, where n corresponds to the number glycoluril groups of the cucurbit[n]uril present in the macrocycle. There may be 6, 7, or 8 glycoluril groups in a cyclic cucurbit[n]uril of the present disclosure. The cyclic cucurbit[n]urils may have various substituents.

In various examples, a compound of the present disclosure has the following structure:

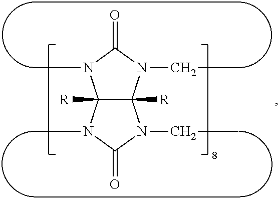

where each R is independently chosen from hydrogen, alkyl groups (e.g., methyl, ethyl, propyl, or isopropyl), hydroxyl groups, alkoxy groups, aryl groups, heteroaryl groups, amino groups, amide groups (e.g., N-and C-linked, primary secondary tertiary), carboxylic acid/carboxylate groups, ester groups (e.g., O— and C═O linked ester groups), imide groups, thiol groups, ether groups, polyether groups, phosphate groups, phosphonate groups, sulfonate groups (e.g., protonated and deprotonated sulfonate groups, where the counterion for the deprotonated sulfonated group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfonate groups (e.g., methyl sulfonate, ethyl sulfonate, propyl sulfonate, butyl sulfonate, and the like) (e.g., protonated and deprotonated alkyl sulfonate groups, where the counterion for the deprotonated sulfate group is M⁺, where M⁺ is chosen from Na⁺, K⁺,Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), sulfate groups ((e.g., protonated and deprotonated sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfate groups (e.g., protonated and deprotonated alkyl sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), and the like, and combinations thereof. Each of the R groups may be further substituted. In various examples, at least one R group is not H. In various examples, the two R groups of a glycoluril group are connected such that they form a heterocycle or carbocycle, such as, for example, a cyclohexyl group, cyclopentyl group, or cyclobutyl group. As an illustrative example, a glycoluril group where two R groups form a cyclohexyl group has the following structure:

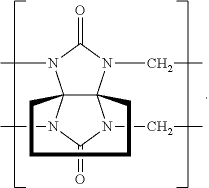

In various examples, R groups may be functional groups upon which additional chemistry may be performed. For example, an R group may terminate with an alkynyl group upon which click chemistry may be performed.

In various examples, 16 of the R groups are substituents other than H, 15 of the R groups are substituents other than H, 14 of the R groups are substituents other than H, 13 of the R groups are substituents other than H, 12 of the R groups are substituents other than H, 11 of the R groups are substituents other than H, 10 of the R groups are substituents other than H, 9 of the R groups are substituents other than H, 8 of the R groups are sub stituents other than H, 7 of the R groups are substituents other than H, 6 of the R groups are substituents other than H, 5 of the R groups are substituents other than H, 4 of the R groups are substituents other than H, 3 of the R groups are substituents other than H, 2 of the R groups are substituents other than H, or 1 of the R groups is a substituent other than H. In various examples all the R groups are the same. In various examples, one or more of the R groups that are not H are the same. In various examples, one or more of the R groups that are not H are different.

In various non-limiting examples, a compound of the present disclosure has the following structure:

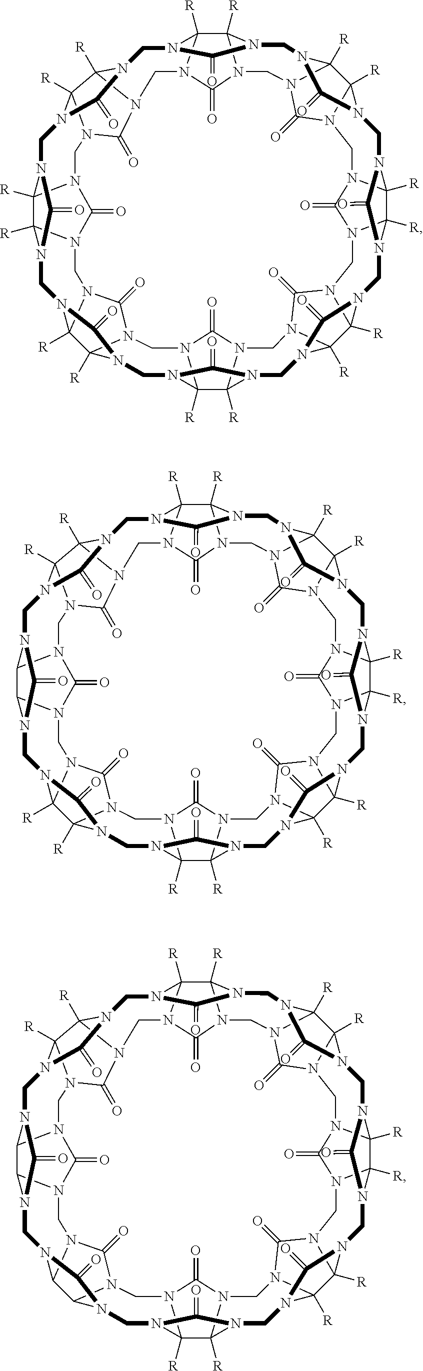

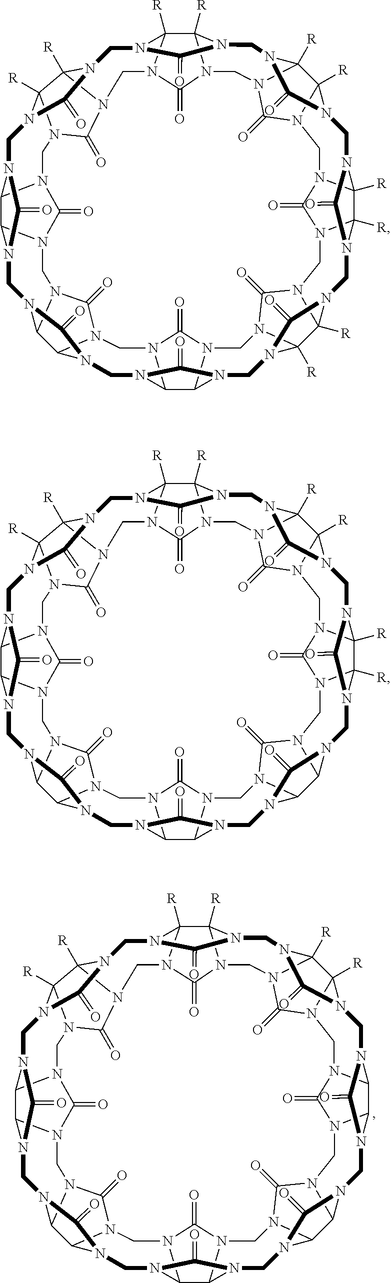

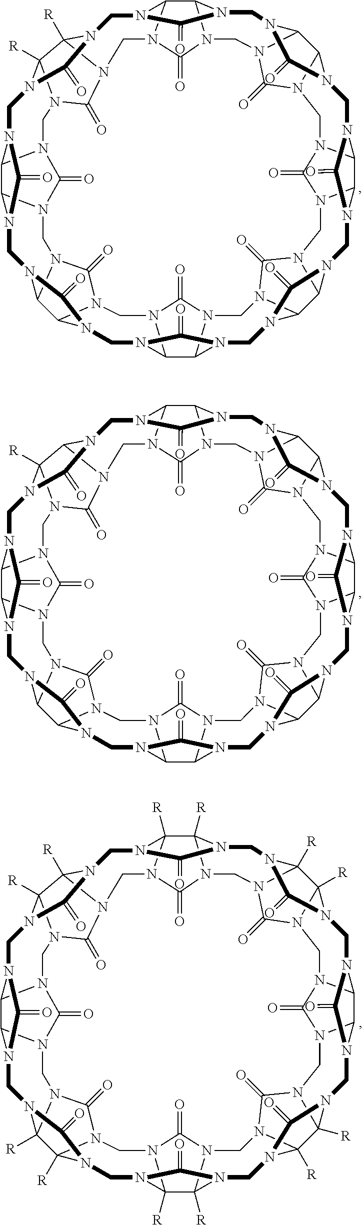

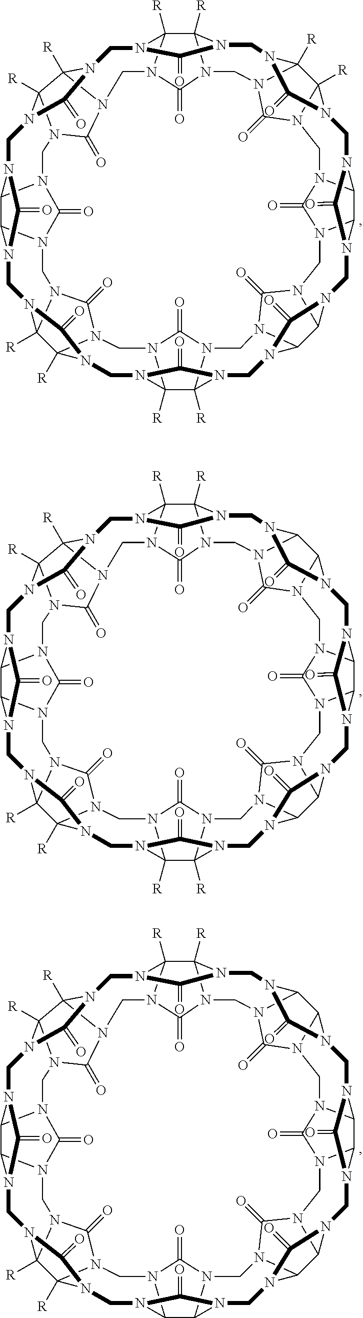

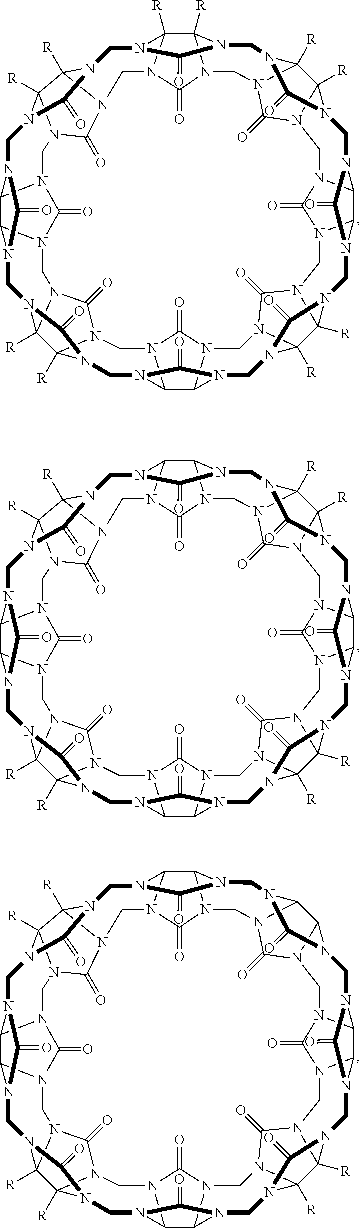

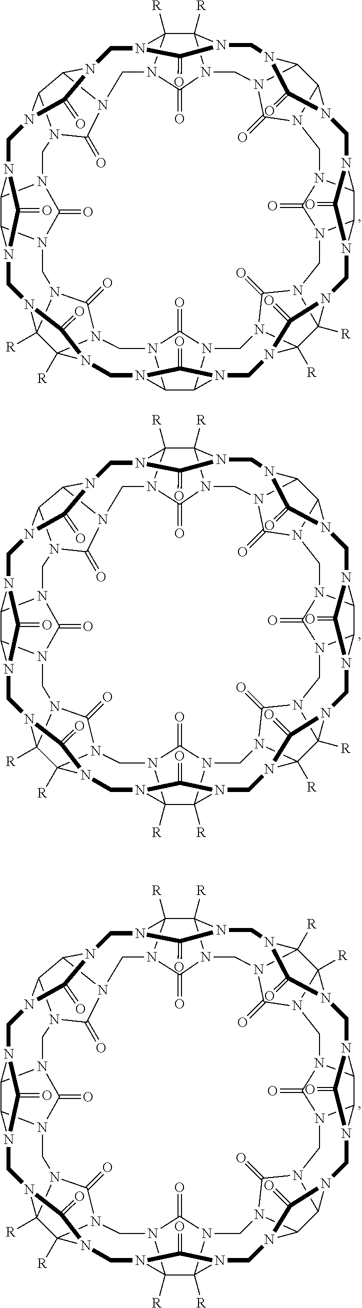

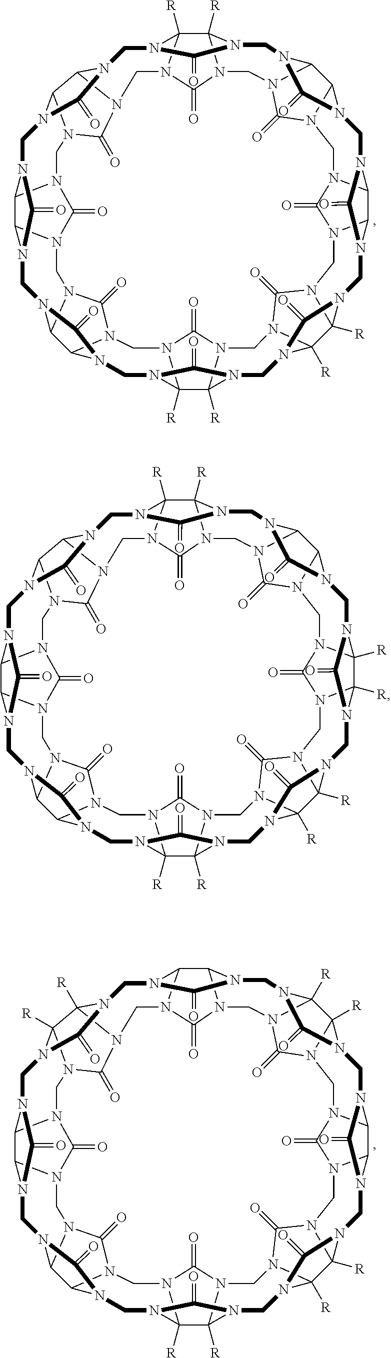

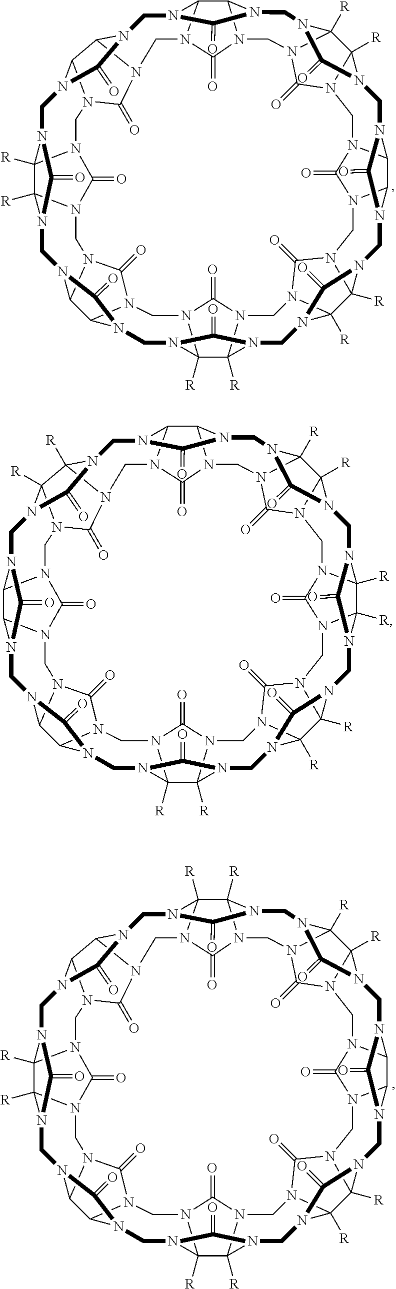

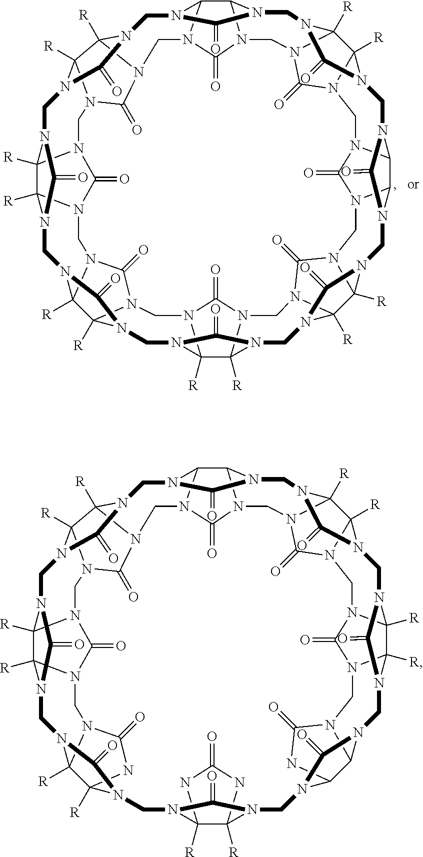

where each R is independently chosen from alkyl groups (e.g., methyl, ethyl, propyl, or isopropyl), hydroxyl groups, alkoxy groups, aryl groups, heteroaryl groups, amino groups, amide groups (e.g., N-and C-linked, primary secondary tertiary), carboxylic acid/carboxylate groups, ester groups (e.g., O— and C═O linked ester groups), imide groups, thiol groups, ether groups, polyether groups, phosphate groups, phosphonate groups, sulfonate groups (e.g., protonated and deprotonated sulfonate groups, where the counterion for the deprotonated sulfonated group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfonate groups (e.g., methyl sulfonate, ethyl sulfonate, propyl sulfonate, butyl sulfonate, and the like) (e.g., protonated and deprotonated alkyl sulfonate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), sulfate groups ((e.g., protonated and deprotonated sulfate groups, where the counterion for the deprotonated sulfate group is M⁺, where M⁺ is chosen from Na⁺, K⁺, Ca²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfate groups (e.g., protonated and deprotonated alkyl sulfate groups, where the counterion for the deprotonated sulfate group is M⁺, where M⁺ is chosen from Na⁺, K⁺, Ca²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), and the like, and combinations thereof.

In various examples, the R groups on a glycoluril group are the same, but different than the R groups on a different glycoluril group. In various examples, at least 4 adjacent R groups that are not hydrogen are the same. A compound of the present disclosure may have the following structure:

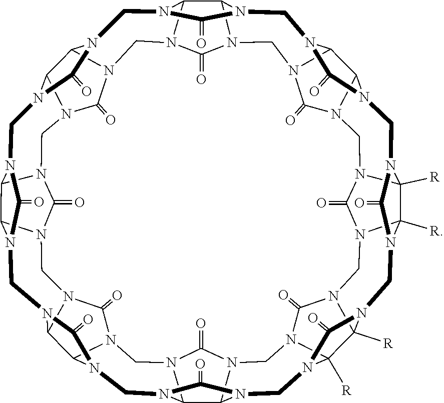

In various examples, the at least four adjacent R groups are all methyl groups or two adjacent glycoluril groups have their respective R groups linked to form a carbocyclic (e.g., two R groups on the same glycoluril are linked such that a six membered ring is formed), all hydroxyl groups, or all alkoxy groups (e.g., methoxy or allyloxy groups). For example, a compound of the present disclosure has the following structure:

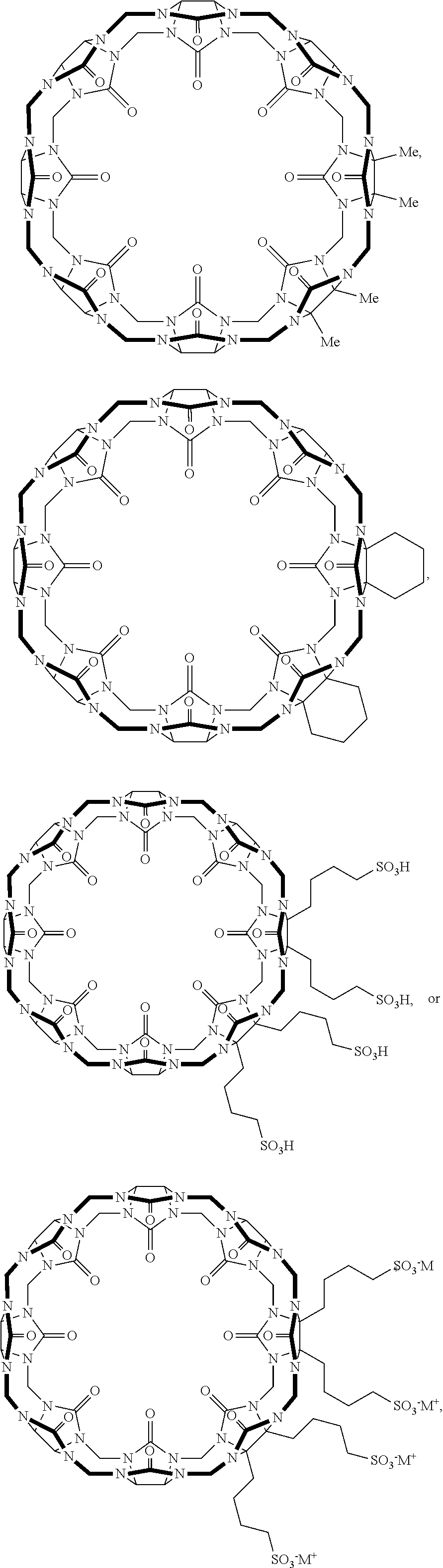

where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS).

In various examples, a compound of the present disclosure has the following structure:

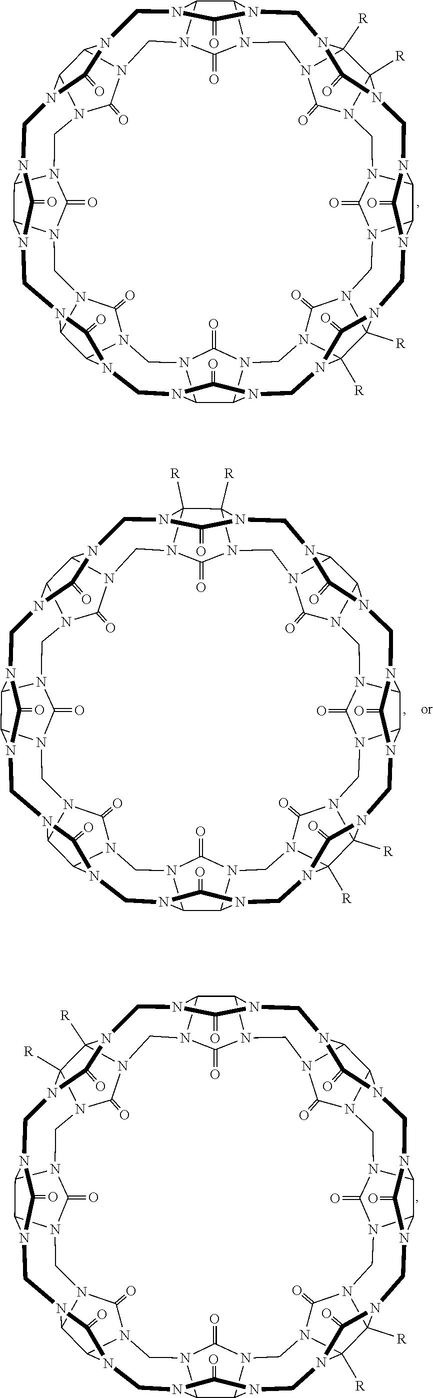

where each R is independently chosen from alkyl groups (e.g., methyl, ethyl, propyl, or isopropyl), hydroxyl groups, alkoxy groups, aryl groups, heteroaryl groups, amino groups, amide groups (e.g., N-and C-linked, primary secondary tertiary), carboxylic acid/carboxylate groups, ester groups (e.g., O— and C═O linked ester groups), imide groups, thiol groups, ether groups, polyether groups, phosphate groups, phosphonate groups, sulfonate groups (e.g., protonated and deprotonated sulfonate groups, where the counterion for the deprotonated sulfonated group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfonate groups (e.g., methyl sulfonate, ethyl sulfonate, propyl sulfonate, butyl sulfonate, and the like) (e.g., protonated and deprotonated alkyl sulfonate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), sulfate groups ((e.g., protonated and deprotonated sulfate groups, where the counterion for the deprotonated sulfate group is M⁺, where M⁺ is chosen from Na⁺, K⁺, Ca²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfate groups (e.g., protonated and deprotonated alkyl sulfate groups, where the counterion for the deprotonated sulfate group is M⁺, where M⁺ is chosen from Na⁺, K⁺, Ca²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), and the like, and combinations thereof. In such examples, a compound of the present disclosure has the following structure:

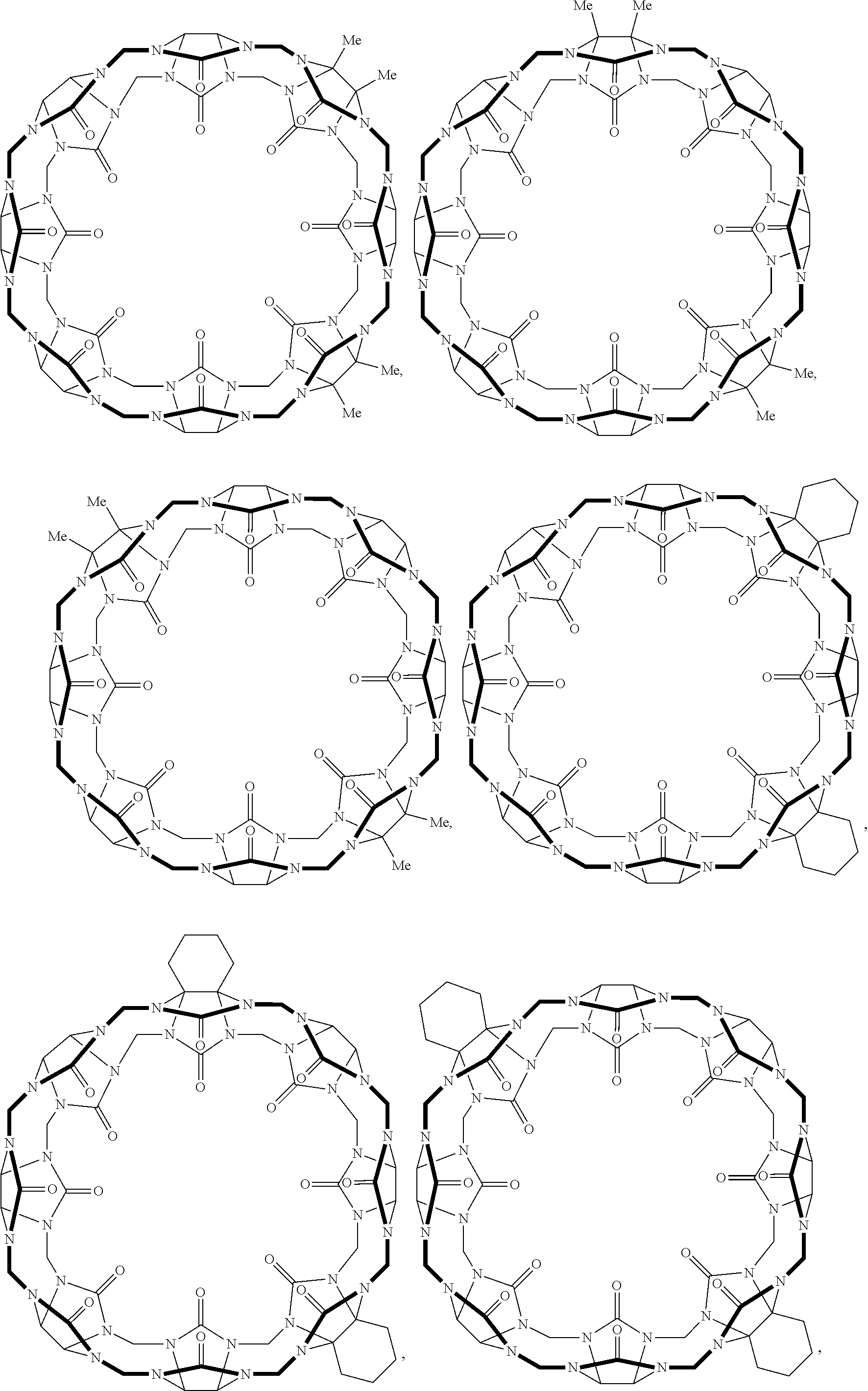

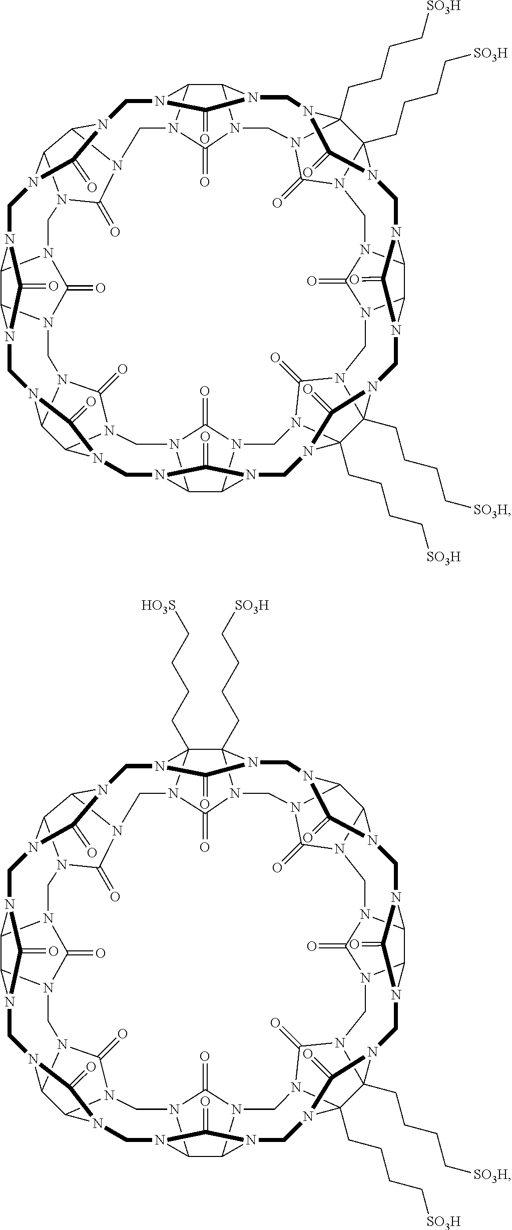

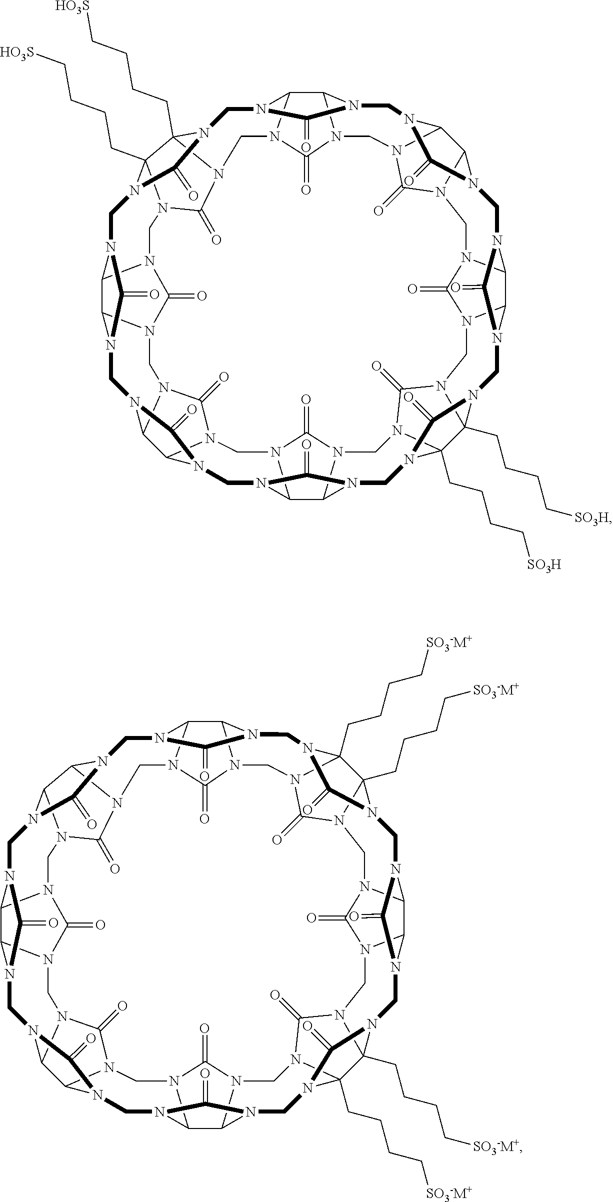

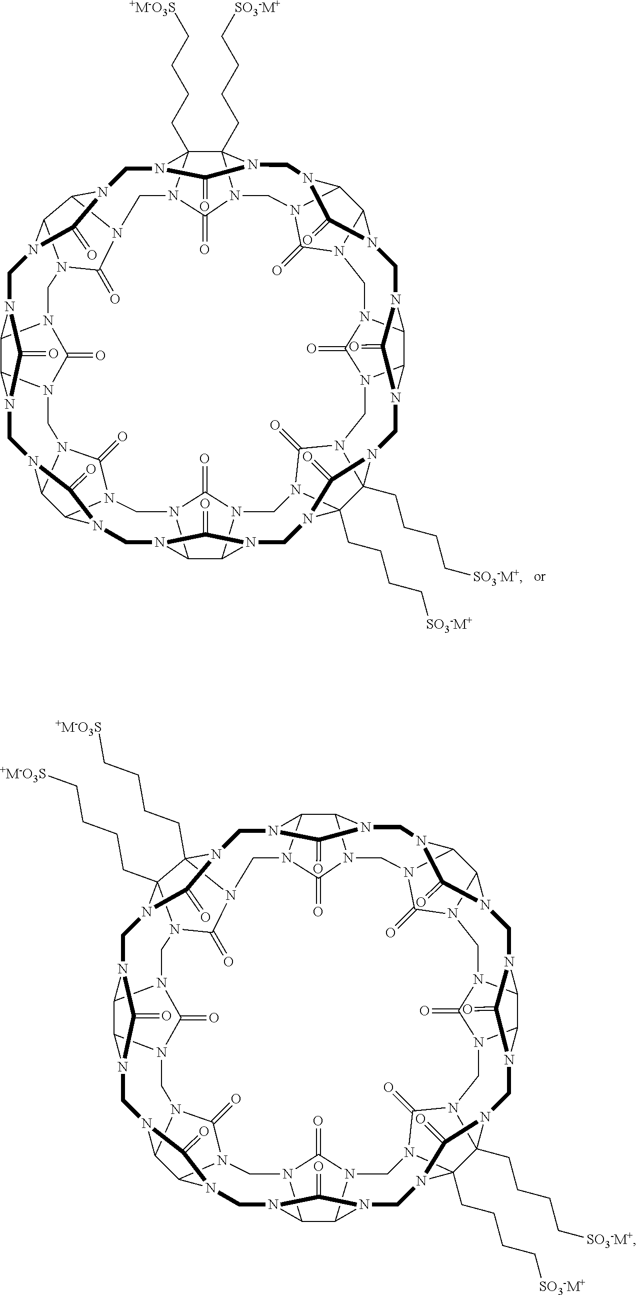

where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS).

In various examples, a compound of the present disclosure has two R groups that are not hydrogens. In such examples, a compound may have the following structure:

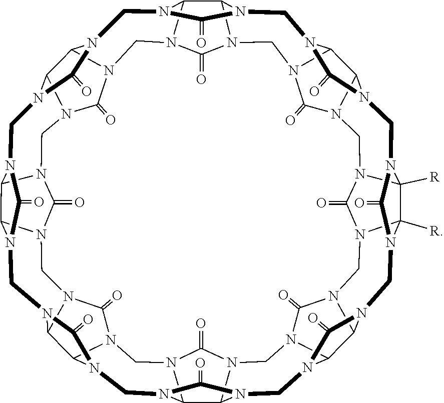

In such examples, the R groups may be the same or different. In examples where the R groups are the same, the R groups may be chosen from carboxylic acid groups, carboxylate groups, ester groups, aryl groups, heteroaryl groups, amide groups (e.g., —CONRR′) groups, phosphate groups, phosphonate groups, alkyl sulfonate groups, and alkyl sulfonic acid groups, or the two R groups may be joined such that they form a cyclic structure such as a cyclobutyl group, a cyclopentyl group, a cyclohexyl group, or a succinimide group. Additional examples of R groups are disclosed herein.

In various examples, the CB may be randomly substituted at various R group positions with a substituent other than hydrogen. For an example, such a compound may have the following structure:

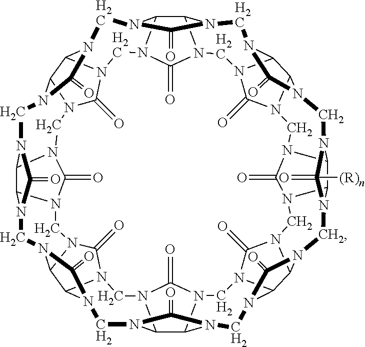

where n is 1, 2, 3, 4, 5, 6, 7, 8, 9, 10, 11, 12, 13, 14, 15, or 16. For example, some or all of the R groups are hydroxyl groups and/or alkoxy groups and the R groups that are not hydroxyl groups and/or alkoxy groups are hydrogen. For example, 1-16 of the R groups are hydroxyl groups or alkoxy groups. These non-hydrogen R groups may be at any R position on the CB. In various examples, the R group are chosen from alkyl groups, aryl groups, heteroaryl groups, carboxylic acid groups, carboxylate groups, phosphate groups, phosphonate groups, sulfonate groups, alkyl sulfonate groups, sulfate groups, alkyl sulfate groups, ester groups (e.g., —O(CO)R or —(CO)OR), amide groups (e.g., —(CO)NHR, —(CO)NRR′, —NH(CO)R, and —NR′(CO)R), ether groups, and polyethylene glycol groups, where the R and R′ groups of the substituents are substituents as defined herein (e.g., halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof). Other examples of R groups are provided herein.

In various examples, a compound of the present disclosure only has one R group that is not hydrogen. For example, the compound has the following structure:

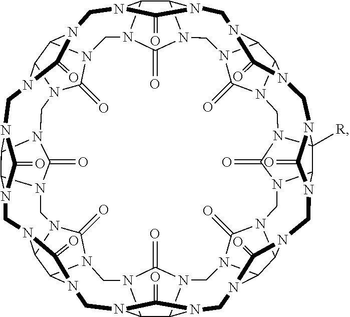

where R group is chosen from alkyl groups, aryl groups, heteroaryl groups, carboxylic acid groups, carboxylate groups, ester groups (e.g., —O(CO)R or —(CO)OR), amide groups (e.g., —(CO)NHR, —(CO)NRR', —NH(CO)R, and —NR′(CO)R), hydroxyl groups, alkoxy groups, ether groups, polyether groups, phosphate groups, phosphonate groups, sulfonate groups, alkyl sulfonate groups, sulfate groups, and alkyl sulfate groups, and polyethylene glycol groups, where the R and R′ groups of the sub stituents are substituents as defined herein (e.g., halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof). Other examples of R groups are provided herein. For example, a compound may have the following structure:

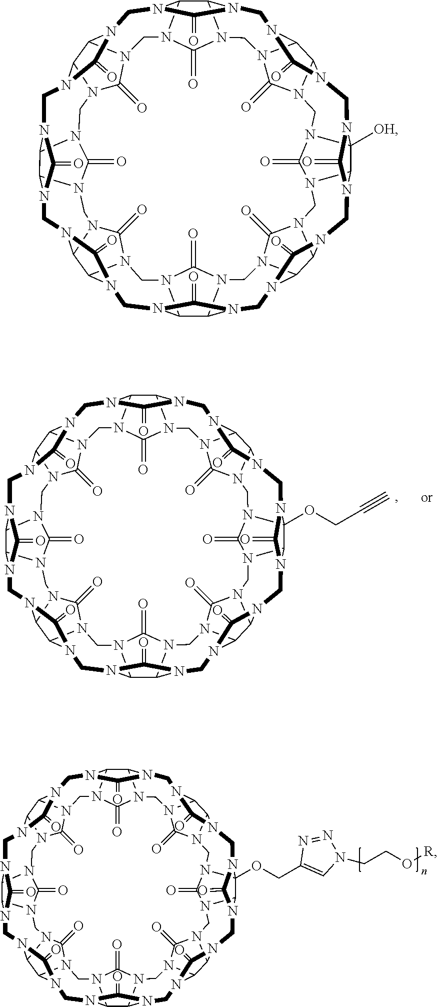

where R is chosen from alkyl groups, aryl groups, heteroaryl groups, carboxylic acid groups, carboxylate groups, ester groups (e.g., —O(CO)R or —(CO)OR), amide groups (e.g., —(CO)NHR, —(CO)NRR', —NH(CO)R, and —NR′(CO)R), hydroxyl groups, alkoxy groups, ether groups, polyether groups, phosphate groups, phosphonate groups, sulfonate groups, alkyl sulfonate groups, sulfate groups, and alkyl sulfate groups and n is 3 to 1000, including all values and ranges therebetween. In various examples, the R group of a PEGylated CB is methyl.

In various examples, a compound of the present disclosure has the following structure:

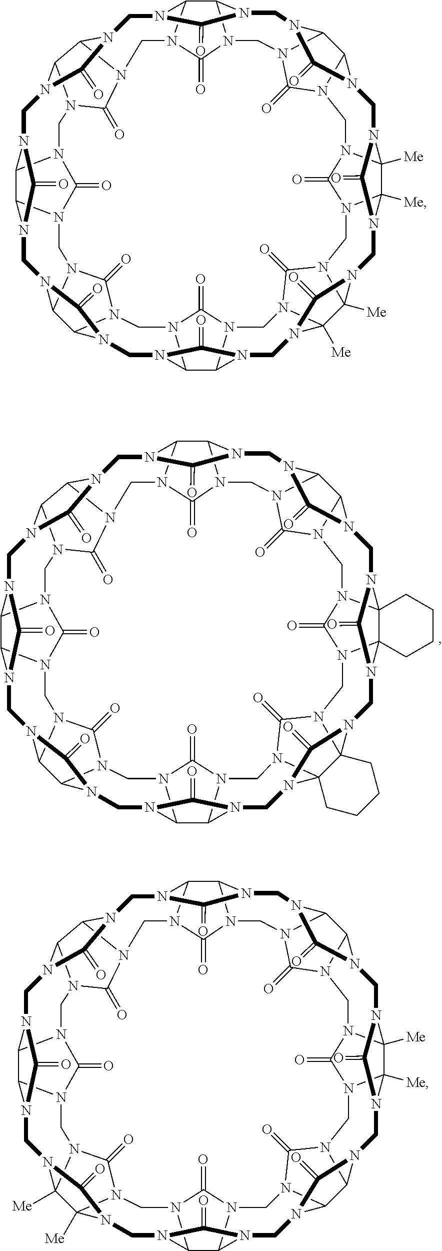

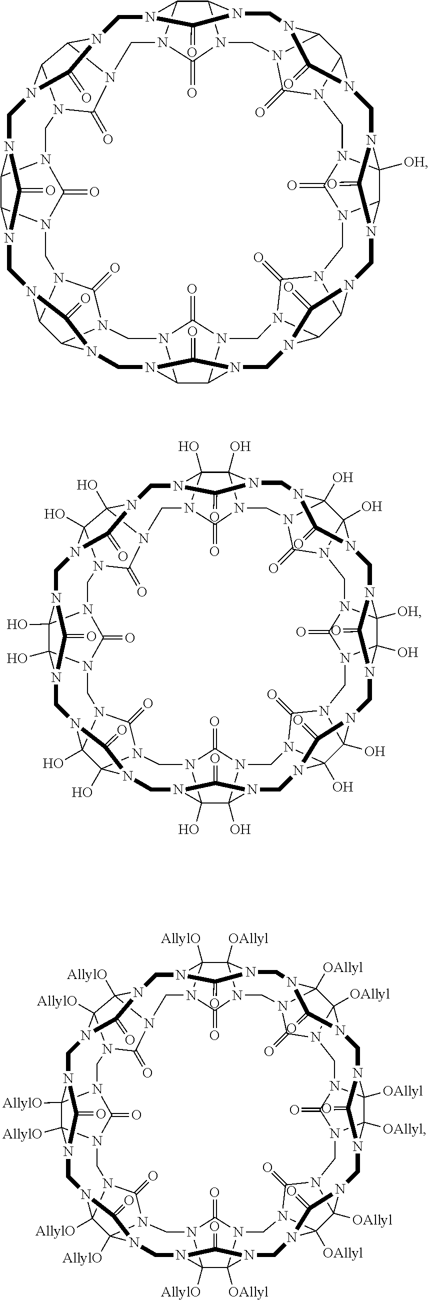

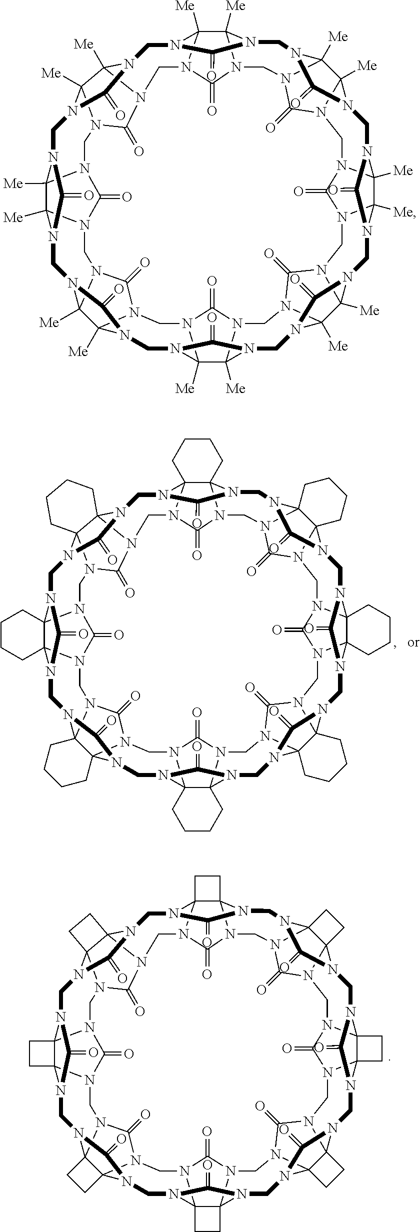

In an aspect, the present disclosure provides compositions comprising one or more compound(s). Non-limiting examples of compositions are described herein.

A composition may comprise one or more compound(s) and one or more pharmaceutical agent(s). In various examples, a pharmaceutical agent comprises one or more positively charged nitrogen atom(s) (e.g., ammonium ions, primary ammonium ions, secondary ammonium ions, tertiary ammonium ions, quaternary ammonium ions, or a combination thereof, where the non-hydrogen group(s) on the ammonium are chosen from aliphatic groups, alkyl groups, aryl groups, and combinations thereof).

A composition may comprise one or more compound (s), one or more pharmaceutical carrier(s), and, optionally, one or more pharmaceutical agent(s). The compositions described herein can be with one or more pharmaceutically acceptable carrier(s). Suitable pharmaceutically acceptable carriers are known in the art. Some non-limiting examples of pharmaceutically acceptable carriers can be found in: Remington: The Science and Practice of Pharmacy (2005) 21st Edition, Philadelphia, Pa. Lippincott Williams & Wilkins. In various examples, the pharmaceutical carrier is pure water or a buffer, such as PBS buffer or the like.

Compositions comprising one or more compound (s) combined with one or more pharmaceutical agent(s), which may form guest-host complexes, can be prepared at any point prior to use of the composition using any suitable technique. The compound-pharmaceutical agent complexes can be formed, for example, by mixing the compound and the pharmaceutical agent in a suitable solvent. It is desirable that the compound and pharmaceutical agent be soluble in the solvent such that the compound and agent form a non-covalent complex. Any suitable solvent can be used. In certain examples, the solvent is an aqueous solution, which includes, but is not necessarily limited to, water and various buffers (e.g., PBS buffer and the like). Non-aqueous solvents could also be used (e.g., MeOH, EtOH, DMSO, and other organic solvents, and combinations thereof), and then removed and the compositions if desired can be re-dissolved in an aqueous solution for administration. In general, a solution of a compound(s) can be provided at a known concentration, examples of which include but are not limited to from 0.1 to 90 mM, inclusive and including all integers to the tenth decimal place there between, and a pharmaceutical agent for which enhanced solubility is desired is added to the solution. The agent(s) can be provided, for example, in a solid form. The combination can be shaken or stirred for a period of time and the amount of pharmaceutical agent that is dissolved is monitored. If all added agent goes into solution, more agent can be added until some detectable portion of it remains undissolved (e.g., a solid). The soluble compound-agent complex can then be isolated and analyzed by any suitable technique, such as by recovering a centrifuged portion and analyzing it by NMR, to determine the concentration of pharmaceutical agent in solution. In various examples, a compound is provided in a composition comprising the drug at a ratio of at least 1 to 1 as pertains to the compound-agent stoichiometry (e.g., compound to drug ratio). In various examples, the compound (e.g., cyclic cucurbit[n]urils) to drug ratio is 100:1 to 1:5, including all ratio values and ranges therebetween (e.g., 100:1, 5:1, 1:2, 1:3, 1:4, or 1:5).

Compositions may be prepared at a patient's bedside or by a pharmaceutical manufacture. In the latter case, the compositions can be provided in any suitable container, such as, for example, a sealed sterile vial, ampoule, or the like, and may be further packaged (the combination of which may be referred to as a kit) to include instruction documents for use by a pharmacist, physician, other health care provider, or the like. The compositions can be provided as a liquid, or as a lyophilized or powder form that can be reconstituted if necessary when ready for use. In particular, the compositions can be provided in combination with any suitable delivery form or vehicle, examples of which include, but are not limited to, liquids, caplets, capsules, tablets, inhalants or aerosol, and the like. The delivery devices may comprise components that facilitate release of the pharmaceutical agents over certain time periods and/or intervals, and can include compositions that enhance delivery of the pharmaceuticals, such as nanoparticle, microsphere or liposome formulations, a variety of which are known in the art and are commercially available. Further, each composition described herein can comprise one or more pharmaceutical agent(s).

Compositions of the present disclsoure may comprise more than one pharmaceutical agent. Likewise, the compositions can comprise distinct host-guest complexes. For example, a first composition comprising one or more cyclic cucurbit[n]urils and a first phamaceutical agent can be separately prepared from a composition which comprises the same compound and a second pharmaceutical agent, and such preparations can be mixed to provide a two-pronged (or more) approach to achieving the desired prophylaxis or therapy in an individual. Further, compositions can be prepared using mixed preparations of any of the cyclic cucurbit[n]uril compounds disclosed herein.

A solid substrate may comprise one or more cyclic cucurbit[n]uril(s) disposed on (e.g., chemically bonded to) at least a portion of a surface of the substrate. At least a portion or all of the cyclic cucurbit[n]uril(s) may be chemically bonded to at least a portion of a surface by covalent bonds, non-covalent bonds, or a combination thereof. Methods of conjugating cyclic cucurbit[n]uril(s) to solid surfaces are known in the art. In various examples, cyclic cucurbit[n]uril(s) are conjugated to a surface by covalent bond- and/or non-covalent bond forming reactions including, but not limited to, amide bond formation, azide alkyne cycloaddition, gold thiol interactions, silicon alcohol condensations, and the like, and combinations thereof.

A solid substrate may comprise (or be) various materials. In various non-limiting examples, a solid substrate comprises or is silica (such as, for example, silica particles), polymer beads, polymer resins (such as, for example, polystyrene, poly NIPAM, polyacrylic acid), metal nanoparticles (e.g., gold nanoparticles, silver nanoparticles, magnetic nanoparticles), a metal (such as, for example, gold and the like), or the like, or a combination thereof.

In an aspect, the present disclosure provides uses of cyclic cucurbit[n]urils. Non-limiting examples of uses of cyclic cucurbit[n]urils are provided herein, for example, non-limiting examples of uses of cyclic cucurbit[n]urils are described in the Statement and Examples.

Cyclic cucurbit[n]urils can be used to sequester various materials, which may be chemical compounds. In various non-limiting examples, one or more cyclic cucurbit[n]urils(s) is/are used to sequester one or more neuromuscular blocking agent(s) (such as, for example, rocuronium, tubocurarine, atracurium, (cis)atracurium besylate, mivacurium, gallamine, pancuronium, vecuronium, and rapacuronium, and the like); one or more anesthesia agent(s) (such as, for example, N-methyl D-aspartate (NMDA) receptor antagonists (e.g., ketamine and the like), short-acting anesthetic agents (e.g., etomidate and the like), and the like); one or more pharmaceutical agent(s) (such as, for example, a drug (e.g., anticoagulants, such as, for example, hexadimethrine and the like)); drugs of abuse (e.g., methamphetamine, cocaine, fentanyl, carfentanil, PCP, MDMA, heroin, and the like); one or more pesticide(s) (such as, for example, paraquat, diquat, organochlorines (e.g., DDT, aldrin, and the like), neonicotinoids (e.g., permethrin and the like), organophosphates (e.g., malathion, glyphosate, and the like), pyrethroids, triazines (e.g., atrazine and the like), and the like); one or more dyestuff(s) (such as, for example, methylene blue, nile red, crystal violet, thioflavin T, thiazole orange, proflavin, acridine orange, methylene violet, azure A, neutral red, cyanines, Direct orange 26, disperse dyes (e.g., disperse yellow 3, disperse blue 27, and the like), coumarins, congo red, and the like); one or more malodorous compound(s) (such as, for example, low molecular weight thiols (e.g., C₁-C₄ thiols), low molecular weight amines (e.g., triethylamine, putrescine, cadaverine, and the like), and the like); or one or more chemical warfare agent(s) (such as for example, nitrogen and sulfur mustards (e.g., bis(2-chloroethyl)ethylamine, bis(2-chloroethyl)methylamine, tris(2-chloroethyl)amine, bis(2-chloroethyl) sulfide, bis(2-chloroethylthioethyl) ether, and the like), nerve agents (such as, for example, those from the G, GV, and V series of nerve agents (e.g., tabun, sarin, soman, cyclosarin, 2-(dimethylamino)ethyl N, N-dimethylphosphoramidofluoridate (GV), novichok agents, VE, VG, VM, VX, and the like), and the like); one or more hallucinogen(s) (e.g., ergolines, lysergic acid diethylamide (LSD), psilocybin, tryptamines, dimethyltryptamine (DMT), phenethylamines, mescaline, ayahuasca, dextromethorphan, and the like); one or more toxin(s) (e.g., dioxins, perfluoralkylsulfonates (PFAS), perfluorooctanoic acid (PFOA), decabromobiphenyl ether (DECA), heavy metals (e.g., mercury), muscarine, tyramine, strychnine, tetrodotoxin, saxitoxin and the like, cholesterol, deoxycholic acid, N-Methyl-4-phenyl-1,2,3,6-tetrahydropyridine, phenylalanine, tyrosine, arginine, histamine); one or more metabolite(s) (e.g., toxic metabolites, such as, for example, N-methyl-4-phenylpyridine, spermine, spermidine, N-nitroso compounds e.g., 4-(methylnitrosoamino)-1-(3-pyridyl)-1-butanone); or the like, or a combination thereof.

A material, which may be a chemical compound, may comprise one or more cationic group. In various examples, a material, which may be a chemical compound, comprises one or more positively charged nitrogen atom(s) (e.g., ammonium ions, primary ammonium ions, secondary ammonium ions, tertiary ammonium ions, quaternary ammonium ions, or a combination thereof, where the non-hydrogen group(s) on the ammonium are chosen from aliphatic groups, alkyl groups, aryl groups, and combinations thereof).

In various examples, a method for sequestering one or more neuromuscular blocking agent(s), one or more anesthesia agent(s), one or more pharmaceutical agent(s), one or more drugs of abuse, one or more pesticide(s), one or more dyestuff(s), one or more malodorous compound(s), one or more chemical warfare agent(s), one or more hallucinogen(s), one or more toxin(s), one or more metabolite(s)or the like, or a combination thereof comprises contacting the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof with one or more cyclic cucurbit[n]uril(s) and/or one or more composition(s), where the neuromuscular blocking agent(s), the anesthesia agent(s), the pharmaceutical agent(s), the pesticide(s), the dyestuff(s), the malodorous compound(s), the chemical warfare agent(s), or a combination thereof are sequestered by the one or more cyclic cucurbit[n]uril(s) and/or one or more composition(s).

The neuromuscular blocking agent(s), the anesthesia agent(s), the pharmaceutical agent(s), drug(s) of abuse, the pesticide(s), the dyestuff(s), the malodorous compound(s), the chemical warfare agent(s), the hallucinogen(s), the toxin(s), the metabolite(s), or a combination thereof may be present in an aqueous sample, in a solid sample (such as, for example, a soil sample), in a gas sample, or the like. An aqueous sample may be derived (e.g., via extraction or other methods to isolate the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof from the solid sample). The aqueous sample may be a wastewater sample (e.g., a municipal wastewater sample, industrial wastewater sample, and the like), an industrial water sample (e.g., water used to make a commercial product, such as, for example, a reagent, a solvent, or the like), a municipal water sample, or the like.

A composition may comprise one or more pharmaceutically active agent(s). In various non-limiting examples, at least a portion (or all) of the one or more compound(s) have a pharmaceutically active agent(s) disposed in the cavity of the one or more compound(s). Without intending to be bound by any particular theory, it is considered that a complex (which may be referred to as a guest-host complex) is formed from (e.g., one or more interaction(s) between (e.g., one or more non-covalent interactions, such as, for example, one or more non-covalent bond(s), is formed between) the compound(s), which may be referred to as hosts, and the neuromuscular blocking agent(s), the anesthesia agent(s), the pharmaceutical agent(s), which may be pharmaceutical agent(s) with undesirable (e.g., low) water solubility, the drugs of abuse, the pesticide(s), the dyestuff(s), the malodorous compound(s), the chemical warfare agent(s), the hallucinogen(s), the toxin(s), the metabolite(s), or a combination thereof, which may be referred to a guest or guests. A guest-host complex can therefore be considered to be an organized chemical entity resulting from the association of the pharmaceutical agent(s) (guest(s)) and the host held together, for example, by non-covalent intermolecular forces.

A composition can comprise various pharmaceutically active agents. Non-limiting examples of pharmaceutical agents include drugs. The pharmaceutically active agent(s) may have various aqueous solubility. A pharmaceutically active agent may have hydrophobic, hydrophilic, or amphiphilic character.

The complexes may be removed from the aqueous sample, the solid sample, the gas sample, or the like. In various examples, the neuromuscular blocking agent(s), the anesthesia agent(s), the pharmaceutical agent(s), drug(s) of abuse, the pesticide(s), the dyestuff(s), the malodorous compound(s), the chemical warfare agent(s), the hallucinogen(s), the toxin(s), the metabolite(s), or a combination thereof are removed from the aqueous sample, the solid sample, the gas sample, or the like using a solid surface with one or more cyclic cucurbit[n]uril(s) disposed thereon.

Cyclic cucurbit[n]urils can be used to sequester various materials in an individual. In various non-limiting examples, the neuromuscular blocking agent(s), the anesthesia agent(s), the pharmaceutical agent(s), the drugs of abuse, the pesticide(s), the dyestuff(s), the malodorous compound(s), the chemical warfare agent(s), one or more hallucinogen(s), one or more toxin(s), one or more metabolite(s), or a combination thereof is present in an individual and the contacting comprises administration of the one or more compound(s) and/or one or more composition(s) to the individual.

Cyclic cucurbit[n]urils can be used to reverse drug-induced neuromuscular block and/or anesthesia and/or the effects of one or more drug(s), which may be drugs of abuse in an individual.

In various non-limiting examples, a method for reversing drug-induced neuromuscular block and/or anesthesia and/or the effects of one or more pharmaceutical agent(s) and/or the effects of one or more drug(s) of abuse in or on an individual comprising administering to an individual in need of reversal of neuromuscular block and/or reversal of anesthesia and/or reversal of the effects of the one or more pharmaceutical agent(s) and/or reversal of one or more drug(s) of abuse), one or more cyclic cucurbit[n]urils, and/or one or more composition(s). The individual may be in need of reversal of drug-induced neuromuscular block. The individual may be in need of reversal of anesthesia. The individual may be in need of reversal of drug-induced neuromuscular block and anesthesia. The individual may be in need of reversal of the effects of one or more pharmaceutical agent(s). The individual may be in need of reversal of the effects of one or more drug(s) of abuse. The individual may have been exposed to the drug(s) of abuse (e.g., carfentanil and the like) in a terrorist attack.

The cyclic cucurbit[n]uril compounds may be used as containers to solubilize chemical compounds. Improvement of solubility for compounds in, for example, aqueous solutions, is desirable for studying drug compounds and for improvement of drug bioavailability for purposes such as, for example, therapeutic and/or prophylactic purposes. For example, the cyclic cucurbit[n]urils are be used to enhance the stability (e.g., decrease degradation, increase shelf life, and the like) of drugs in water, the solid state, or both. Examples of insoluble chemical compounds include, but are not limited to, amiodarone, tamoxifen, estradiol, albendazole, and the like.

In certain examples, the cyclic cucurbit[n]uril compounds can be used to rescue promising drug candidates, which have undesirable solubility and bioavailablity, and thus alleviate the attrition in the drug development process for anti-cancer agents and agents intended to treat other diseases. The containers may be used for targeted delivery of drugs to particular cell types, such as, for example, tumor cells and the like, to increase the effectiveness of existing drugs, reduce their toxic side effect(s), or both.

In various examples, a composition comprises one or more cyclic cucurbit[n]uril(s) and one or more pharmaceutical agent(s). Such compositions may be provided as pharmaceutical preparations as described herein.

It is important to emphasize that the pharmaceutical agent(s) that can be included in compositions comprising one or more cyclic cucurbit[n]uril(s) and one or more pharmaceutical agent(s) is not particularly limited. In certain examples, the pharmaceutical agent(s) combined with one or more cyclic cucurbit[n]uril(s) is/are a pharmaceutical agent or agents that is/are poorly water-soluble. In certain other examples, the pharmaceutical agent(s) combined with one or more cyclic cucurbit[n]uril(s) is/are a pharmaceutical agent or agents that is/are water soluble.

Solubility of any particular pharmaceutical agent can be determined, if desired, using any of a variety of techniques that are well known to those skilled in the art. Solubility can be ascertained if desired at any pH, such as a physiological pH, and/or at any desired temperature. Suitable temperatures include, but are not necessarily limited to, from 4° C. to 70° C., inclusive, and including all integer ° C. values therebetween.

In connection with poorly soluble or low solubility pharmaceutical agents suitable for use in the present disclosure, in various examples, such agents are considered to be those which have a solubility of less than 100 uM in water or an aqueous buffer.

In various other examples, poorly soluble pharmaceutical agents are considered to include compounds, which are Biopharmaceutics Classification System (BCS) class 2 or class 4 drugs. The BCS is well known to those skilled in the art and is based on the aqueous solubility of drugs reported in readily available reference literature, and for drugs that are administered orally it includes a correlation of human intestinal membrane permeability. (See, for example, Takagi et al., (2006) Molecular Pharmaceutics, Vol. 3, No. 6, pp. 631-643.) The skilled artisan will therefore readily be able to recognize a drug as a member of BCS class 2 or class 4 from published literature, or can test a drug with an unknown BCS or other solubility value to determine whether it has properties consistent with either of those classifications, or for otherwise being suitable for use in the present disclosure. In an example, solubility is determined according to the parameters set forth in this matrix:

| | Parts of solvent required for | Solubility Range |
|---|---|---|
| Solubility | 1 part of solute | (mg/mL) |
| very soluble | <1 | ≥1000 |
| freely soluble | from 1 to 10 | 100-1000 |
| soluble | from 10 to 30 | 33-100 |
| sparingly soluble | from 30 to 100 | 10-33 |
| slightly soluble | from 100 to 1000 | 1-10 |
| very slightly soluble | form 1000 to 10000 | 0.1-1 |
| practically insoluble | ≥10000 | <0.1 |

Thus, for the purposes of the present disclosure, a poorly soluble pharmaceutical agent that can be combined with one or more cyclic cucurbit[n]uril(s) can be any pharmaceutical agent that falls into the categories sparingly soluble, slightly soluble, very slightly soluble, and practically insoluble as set forth in the above matrix.

Again, it should be emphasized that other than being characterized as having low solubility in aqueous solution, the pharmaceutical agent with which one or more cyclic cucurbit[n]uril(s), which a compound can be combined is not limited. In this regard, at least one utility of the present disclosure is combination of one or more of a wide variety of distinct pharmaceutical agents with one or more cyclic cucurbit[n]uril(s), and as a consequence of combining these compounds with the pharmaceutical agent(s), solubility of the agent(s) is/are increased. In various examples, types of pharmaceutical agents suitable for solubilization include, but are not limited to, mitotic inhibitors (e.g., taxol, a mitotic inhibitor used in cancer chemotherapy, and the like); nitrogen mustard alkylating agents (e.g., Melphalan, trade name Alkeran used for chemotherapy, and the like); benzimidazoles (e.g., Albendazole, marketed as Albenza, Eskazole, Zentel and Andazol, for treatment of a variety of worm infestations, and the like); antagonists of the estrogen receptor in breast tissue which is used to treat breast cancers (e.g., Tamoxifen, which is an estrogen receptor antagonist when metabolized to its active form of hydroxytamoxifen, and the like); antihistamines (e.g., Cinnarizine, marketed as Stugeron and Stunarone for control of symptoms of motion sickness, and the like); thienopyridine class antiplatelet agents (e.g., Clopidogrel, marketed as Plavix for inhibiting blood clots in coronary artery disease and for other conditions, and the like); and antiarrhythmic agents (e.g., Amiodarone, used for treatment of tachyarrhythmias, and the like). Other pharmaceutical agents not expressly listed here are also included within the scope of the disclosure. Some examples of such agents include, but are not limited to, adjuvants for use in enhancing immunological responses, analgesic agents, detectably labeled agents used for diagnostic imaging, and the like. Combinations of any of these example pharmaceutical agents may be used. Cyclic cucurbit[n]urils may be combined with and improve solubility of pharmaceutical agents that are members of vastly different classes of compounds which are characterized by disparate chemical structures and biological activities.

Compositions of the present disclosure can be administered to any human or non-human animal in need of therapy or prophylaxis for one or more condition(s) for which the pharmaceutical agent is intended to provide a prophylactic of therapeutic benefit. Thus, the individual can be diagnosed with, suspected of having, or be at risk for developing any of a variety of conditions for which a reduction in severity would be desirable. Non-limiting examples of such conditions include cancer, including solid tumors, blood cancers (e.g., leukemia, lymphoma, myeloma, and the like). Specific examples of cancers include, but are not limited to, fibrosarcoma, myxosarcoma, liposarcoma, chondrosarcoma, osteogenic sarcoma, chordoma, angiosarcoma, endotheliosarcoma, lymphangiosarcoma, pseudomyxoma peritonei, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, head and neck cancer, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, thymoma, Waldenstrom's macroglobulinemia, heavy chain disease, and the like.

In addition to various malignancies, compounds of the present disclosure are also suitable for providing a benefit for cardiovascular related disorders, examples of which include, but are not limited to, angina, arrhythmia, atherosclerosis, cardiomyopathy, congestive heart failure, coronary artery disease, carotid artery disease, endocarditis, coronary thrombosis, myocardial infarction, hypertension, hypercholesterolemia/hyperlipidemia, mitral valve prolapse, peripheral artery disease, stroke, thrombosis, embolism, other forms of ischemic damage, and the like.

In addition, the compositions of the present disclosure can be used in connection with treating a variety of infectious diseases. It is expected that a variety of agents used to treat and/or inhibit infectious diseases caused by, for example, bacterial, protozoal, helminthic, fungal origins, viral origins, or the like can be aided by use of compositions of the present disclosure.

Various methods known to those skilled in the art can be used to introduce the compounds and/or compositions of the present disclosure to an individual. These methods include, but are not limited to, intravenous, intramuscular, intracranial, intrathecal, intradermal, subcutaneous, oral routes, and the like, and combinations thereof. The dose of the composition comprising a compound and a pharmaceutical agent will necessarily be dependent upon the needs of the individual to whom the composition is to be administered. These factors include, but are not necessarily limited to, the weight, age, sex, medical history, and nature and stage of the disease for which a therapeutic or prophylactic effect is desired. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using ordinary skill in the art, and given the benefit of the present disclosure.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like.

The steps of the method described in the various examples disclosed herein are sufficient to carry out the methods of the present disclosure. Thus, in an example, the method consists essentially of a combination of the steps of the methods disclosed herein. In another example, the method consists of such steps.

In an aspect, the present disclosure provides articles comprising compounds of the present disclosure.

The articles may be articles of manufacture. Non-limiting examples of articles include wipes impregnated with one or more compounds of the present disclosure. For example, such a wipe is used to decontaminate a surface from any material capable of being sequestered by a compound (e.g., a cyclic cucurbit[n]uril of the present disclosure). For example, the wipe is used to decontaminate a surface that has or was previously exposed to a toxin, drug of abuse, or the like, or a combination thereof.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

The following Statements provide various embodiments of the present disclosure.
Statement 1. A method for sequestering one or more neuromuscular blocking agent(s), one or more anesthesia agent(s), one or more pharmaceutical agent(s), one or more drugs of abuse, one or more pesticide(s), one or more dyestuff(s), one or more malodorous compound(s), one or more chemical warfare agent(s), one or more hallucinogen(s), one or more toxin(s), one or more metabolite(s), or a combination thereof comprising: contacting the neuromuscular blocking agent(s), the anesthesia agent(s), the pharmaceutical agent(s), the drugs of abuse, the pesticide(s), the dyestuff(s), the malodorous compound(s), the chemical warfare agent(s), one or more hallucinogen(s), one or more toxin(s), one or more metabolite(s), or a combination thereof with one or more compound(s) having the following structure:

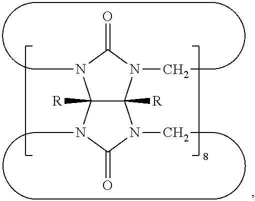

where each R is independently chosen from hydrogen, alkyl groups (e.g., methyl, ethyl, propyl, or isopropyl), hydroxyl groups, alkoxy groups, aryl groups, heteroaryl groups, amino groups, amide groups (e.g., N-and C-linked, primary secondary tertiary), carboxylic acid/carboxylate groups, ester groups (e.g., O— and C═O linked ester groups), imide groups, thiol groups, ether groups, polyether groups, phosphate groups, phosphonate groups, sulfonate groups (e.g., protonated and deprotonated sulfonate groups, where the counterion for the deprotonated sulfonated group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfonate groups (e.g., methyl sulfonate, ethyl sulfonate, propyl sulfonate, butyl sulfonate, and the like) (e.g., protonated and deprotonated alkyl sulfonate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), sulfate groups ((e.g., protonated and deprotonated sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfate groups (e.g., protonated and deprotonated alkyl sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), and the like, and combinations thereof, and the neuromuscular blocking agent(s), the anesthesia agent(s), the pharmaceutical agent(s), the drug(s) of abuse, the pesticide(s), the dyestuff(s), the malodorous compound(s), the chemical warfare agent(s), one or more hallucinogen(s), one or more toxin(s), one or more metabolite(s), or a combination thereof are sequestered by the one or more compound(s). In various examples, at least one R group is not hydrogen
Statement 2. A method according to Statement 1, where the one or more compounds have the following structure:

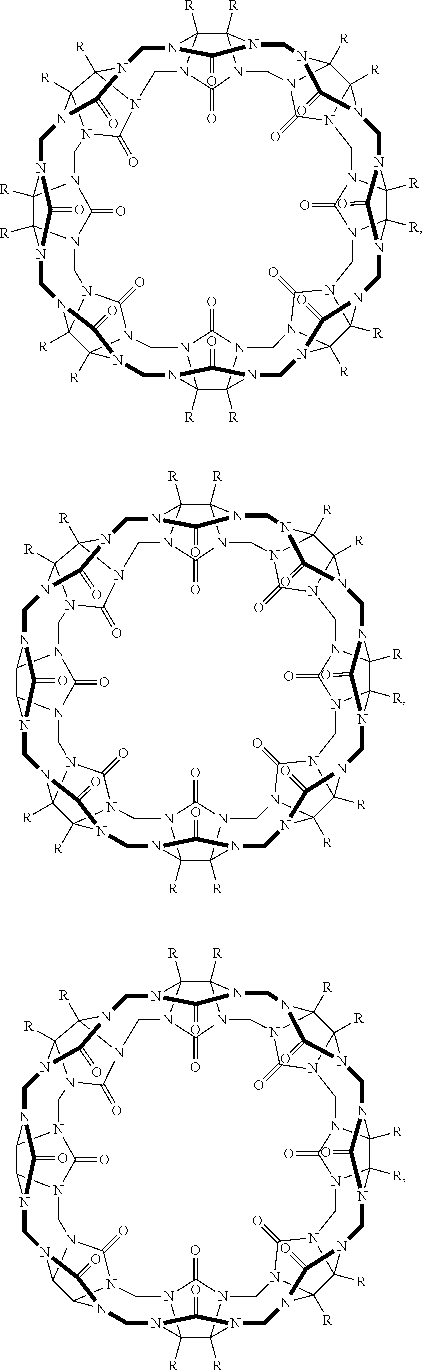

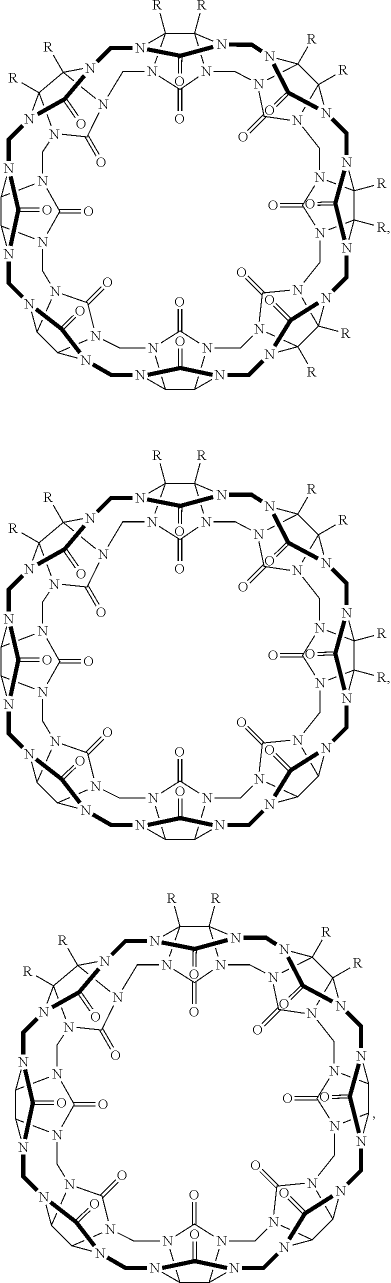

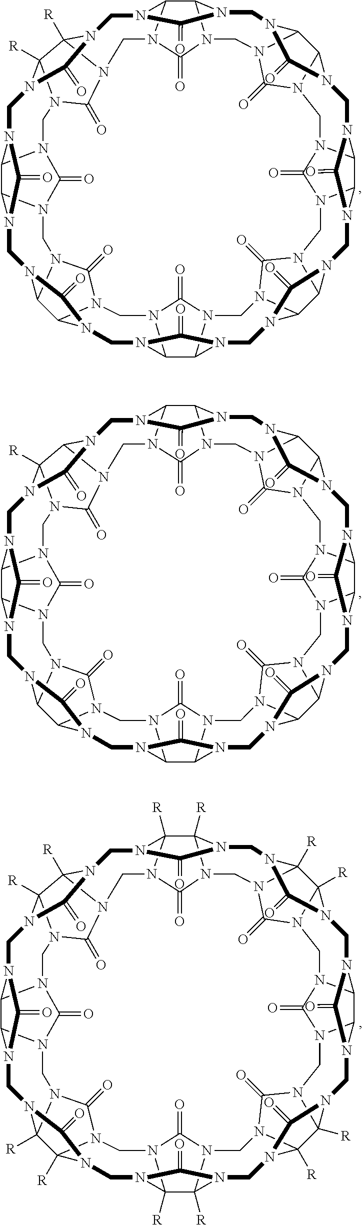

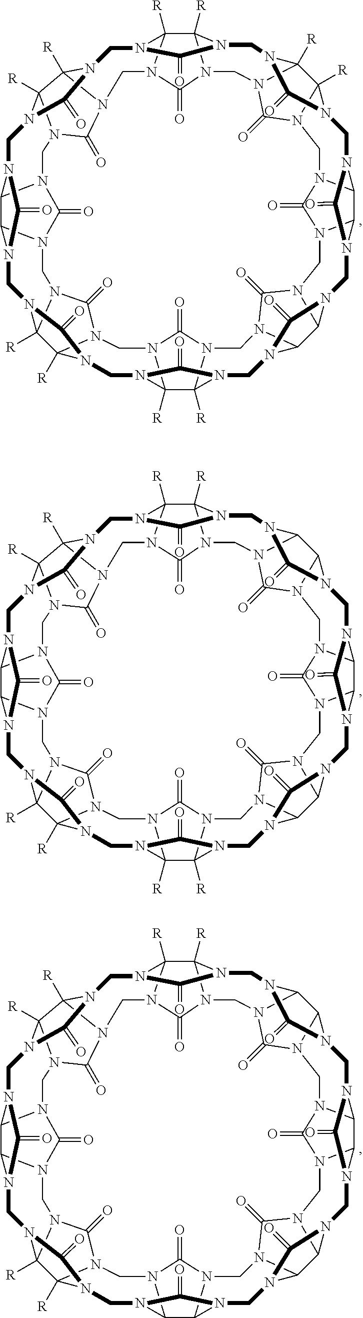

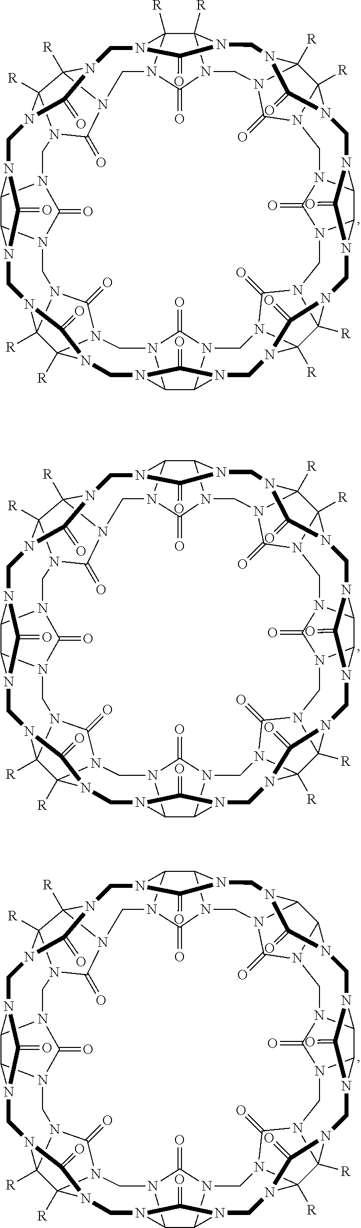

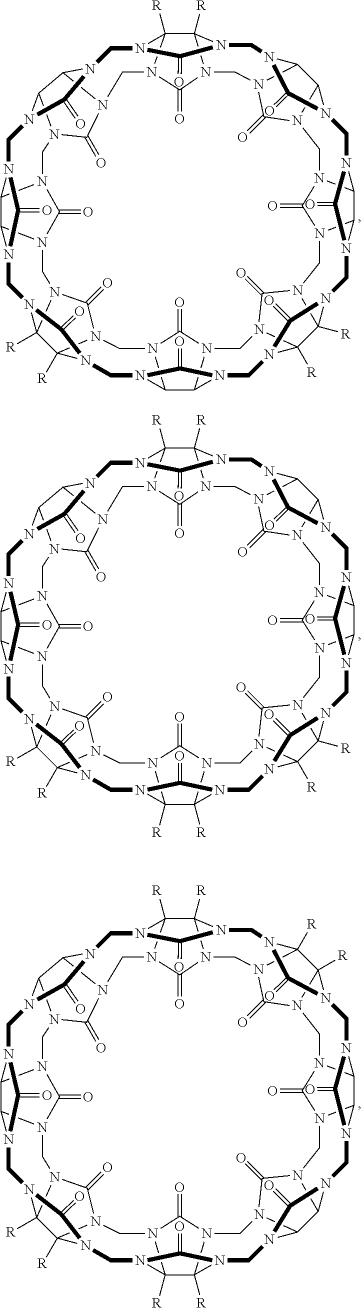

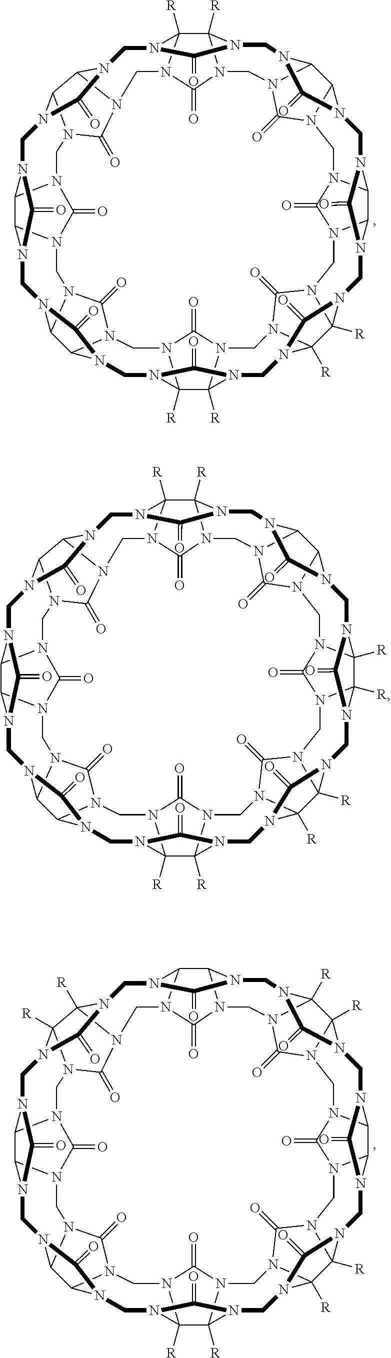

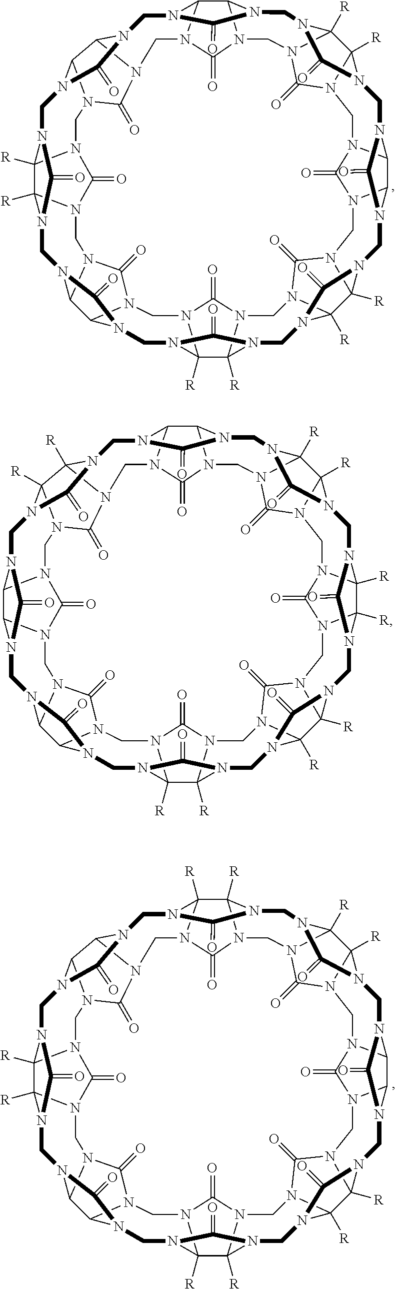

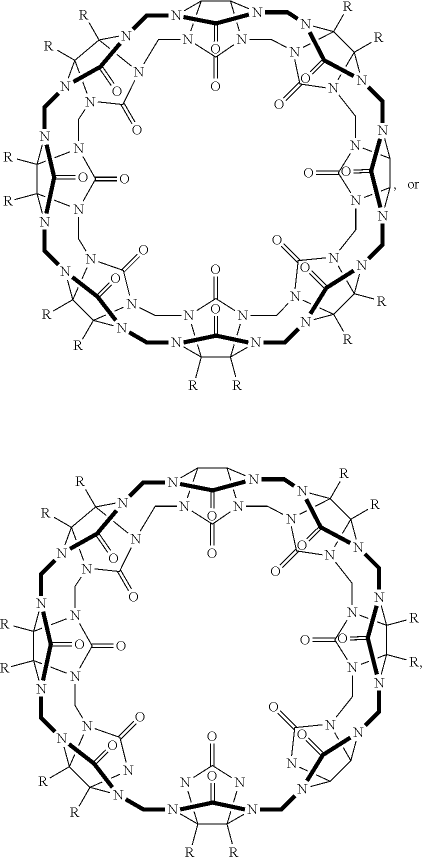

Statement 3. A method according to Statements 1 or 2, where the one or more compounds have the following structure:

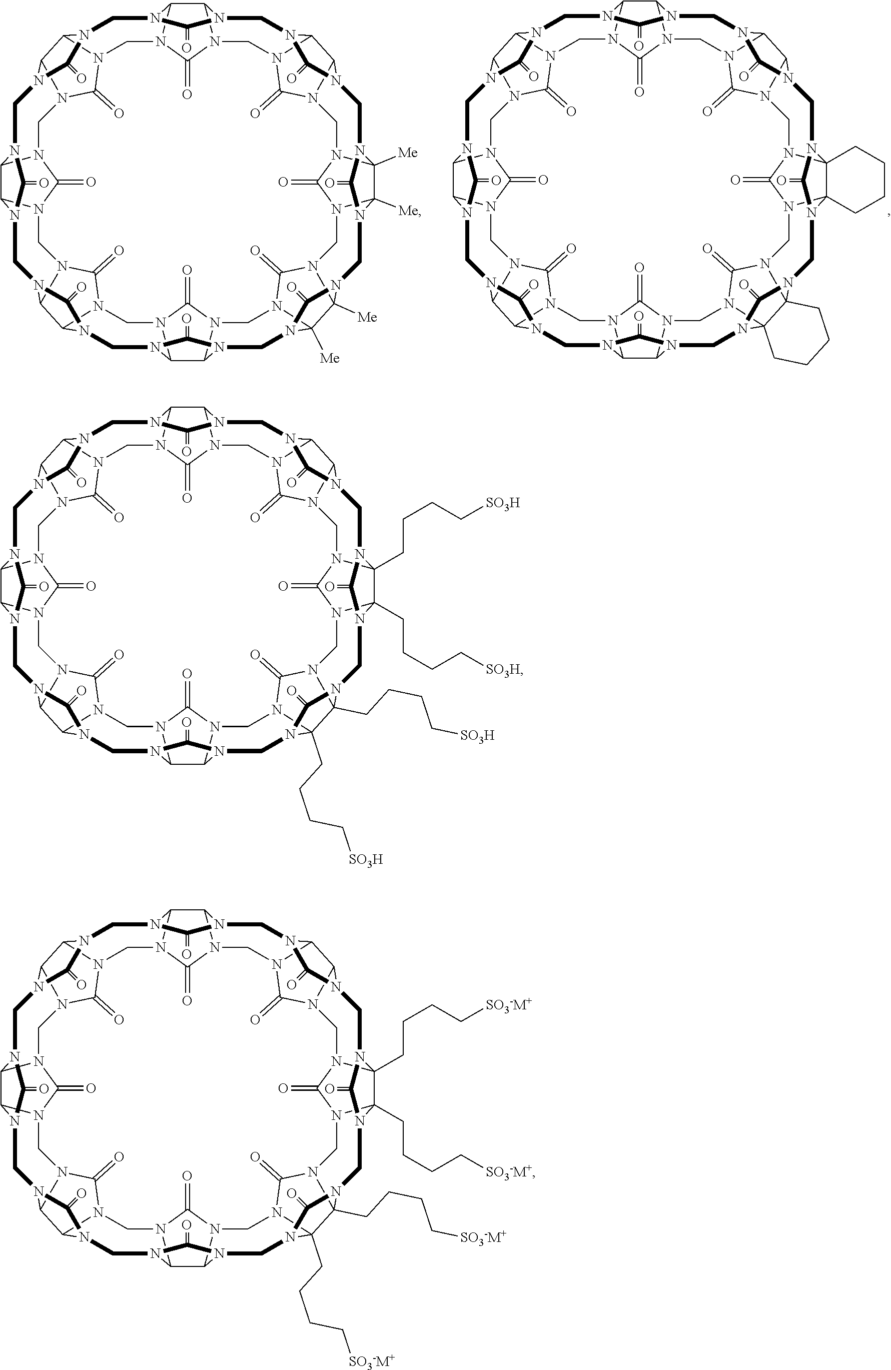

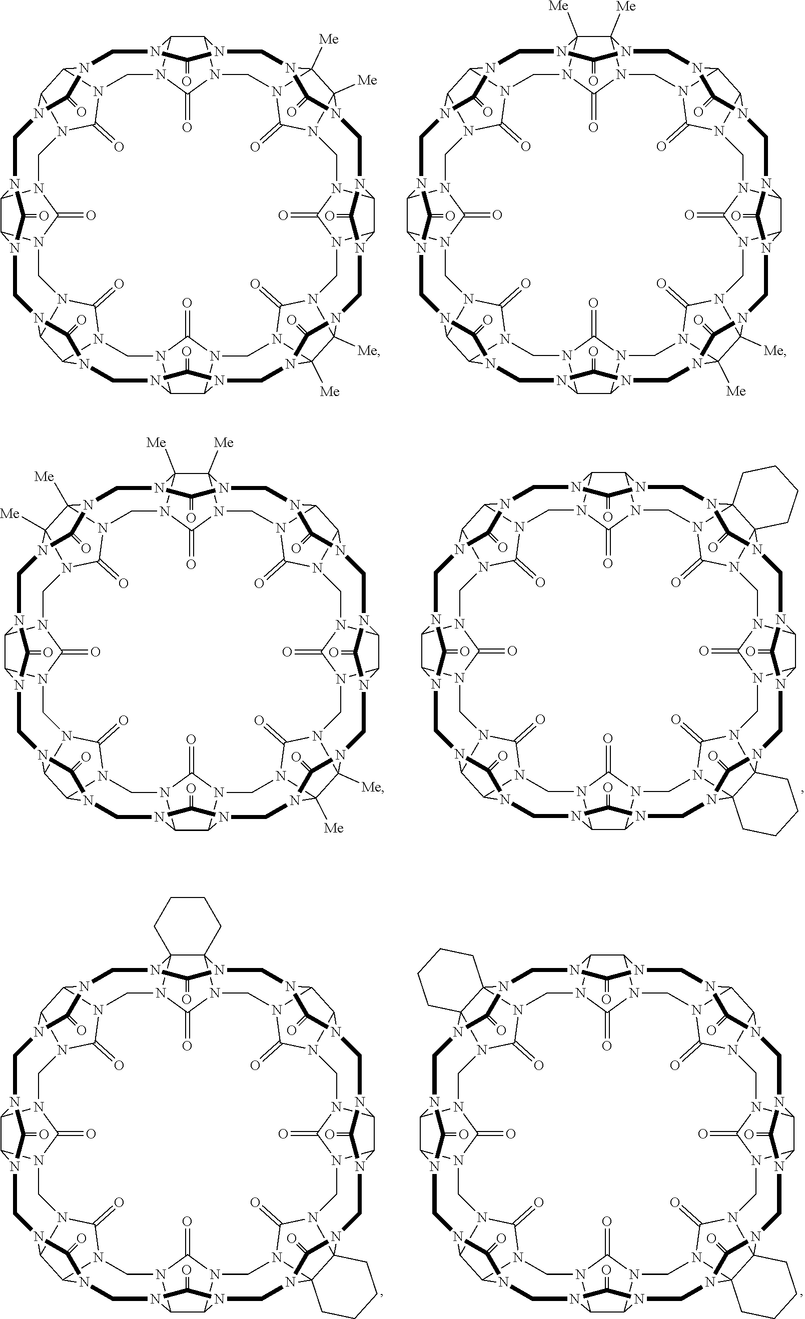

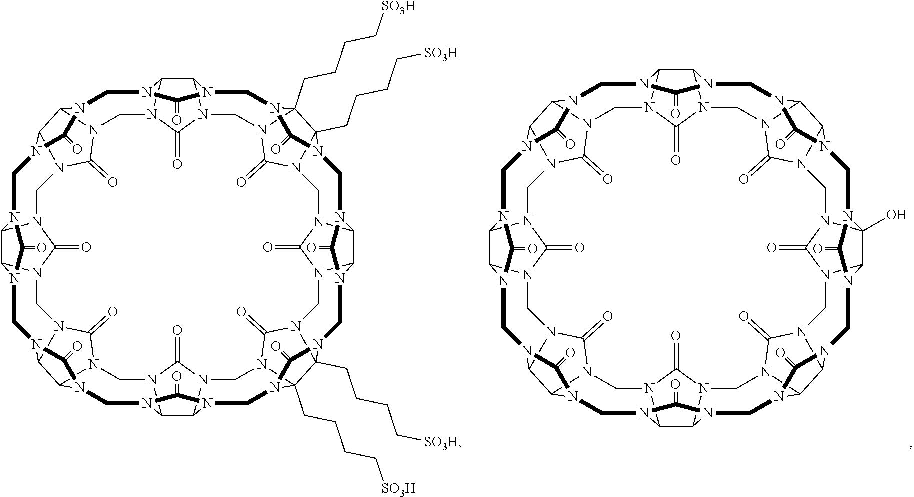

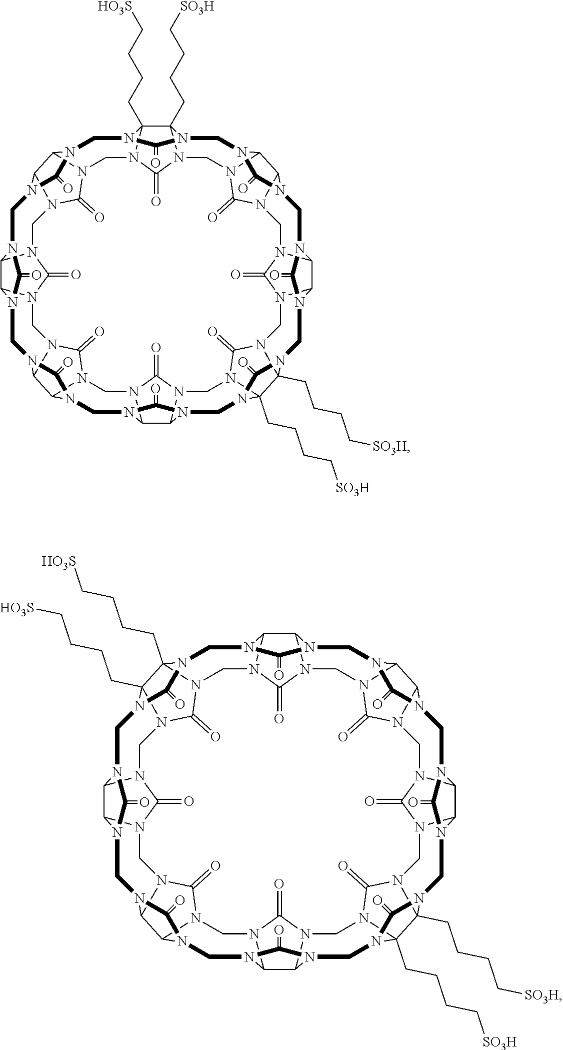

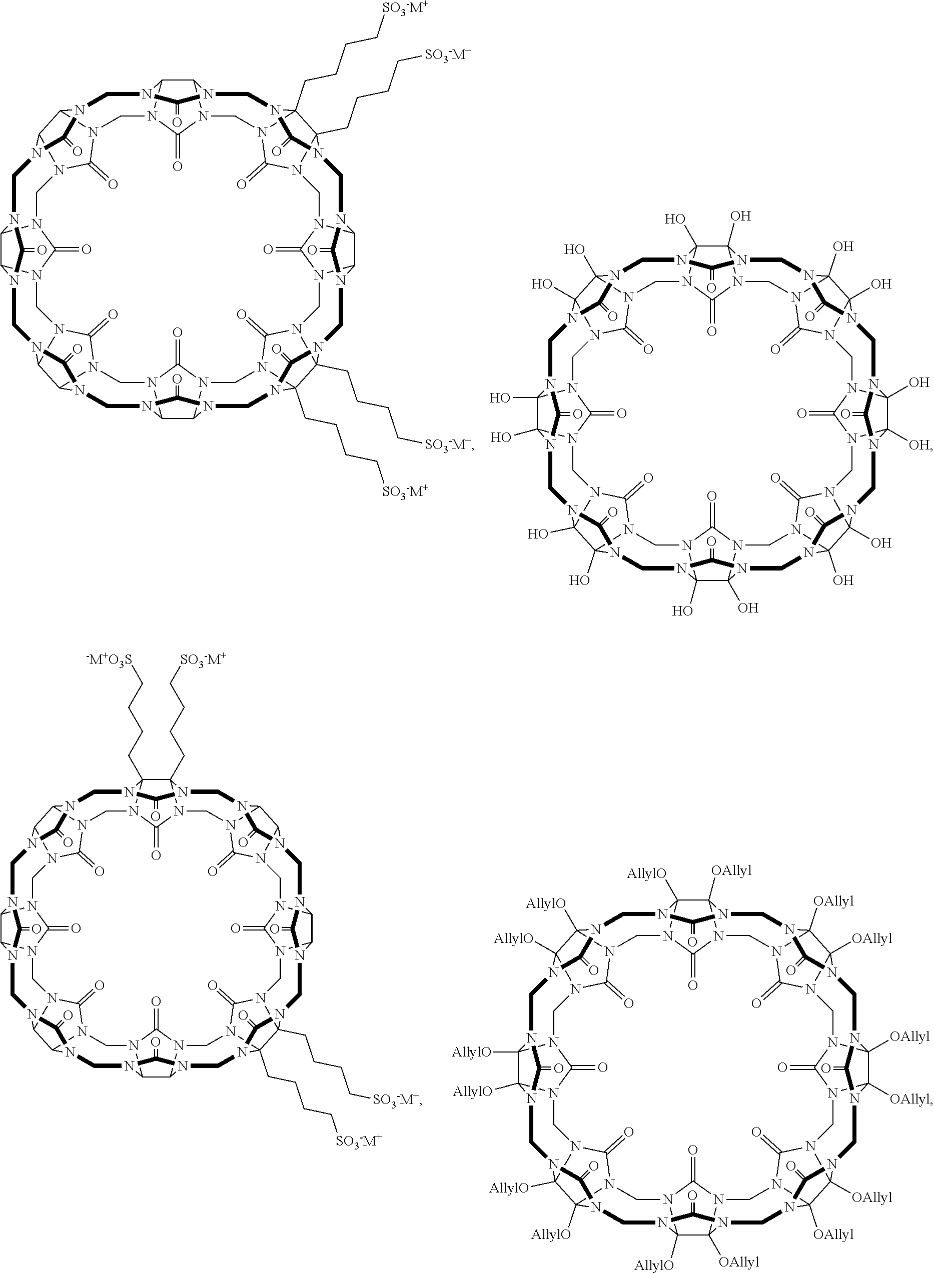

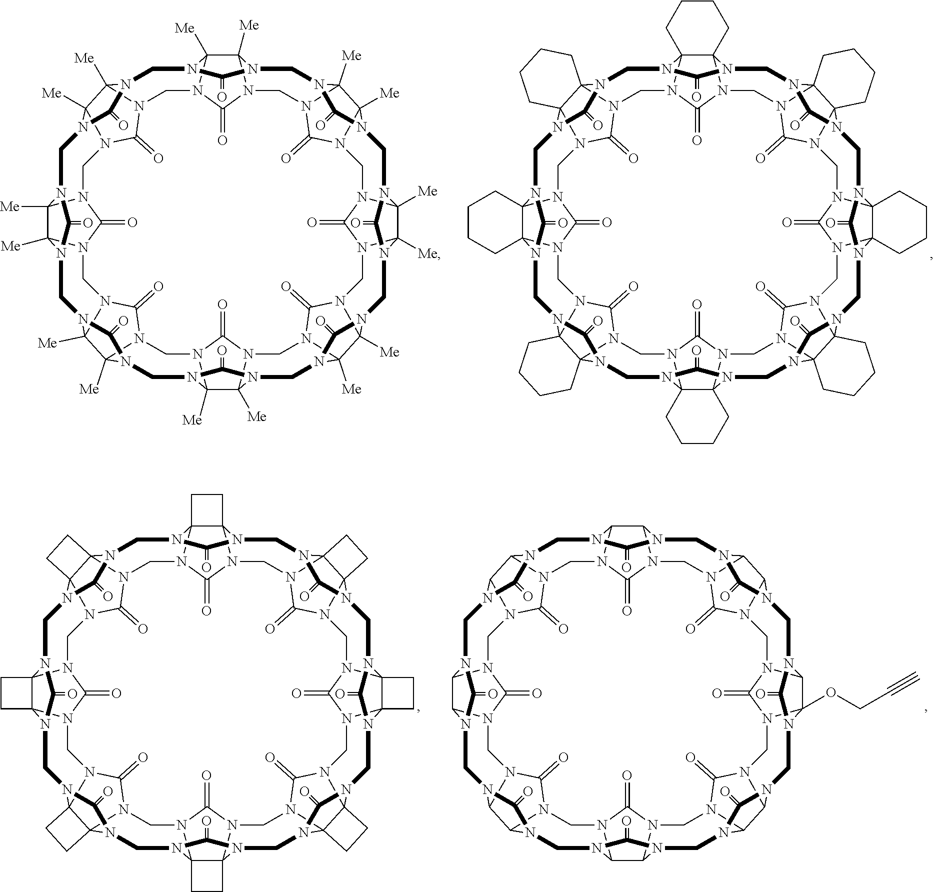

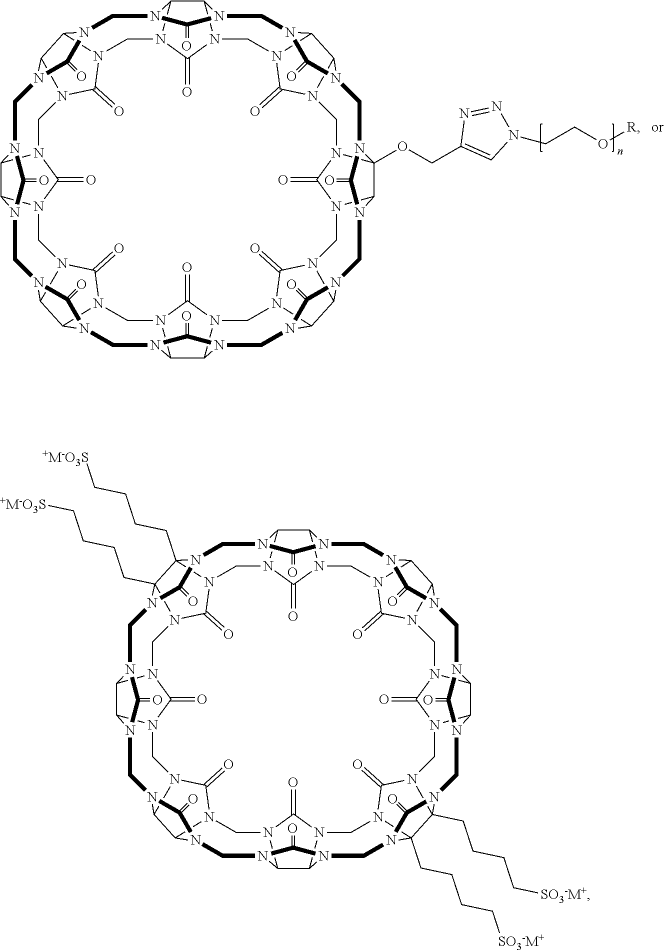

where n is 3 to 1000, including all values and ranges therebetween.
Statement 4. A method according to any one of the preceding Statements, where the one or more compounds have the following structure:

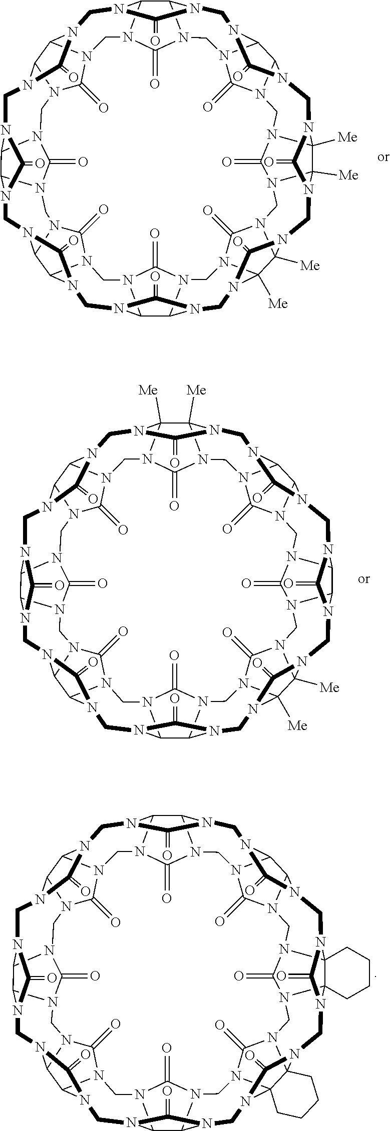

Statement 5. A method according to any one of the preceding Statements, where the neuromuscular blocking agent(s), the anesthesia agent(s), the pharmaceutical agent(s), the drug(s) of abuse, the pesticide(s), the dyestuff(s), the malodorous compound(s), the chemical warfare agent(s), one or more hallucinogen(s), one or more toxin(s), one or more metabolite(s), or a combination thereof is present in an aqueous sample, in a solid sample, in a gas sample, or on a solid surface.
Statement 6. A method according to any one of the preceding Statements, where a complex is formed from the one or more compound(s) and the neuromuscular blocking agent(s), the anesthesia agent(s), the pharmaceutical agent(s), the drugs of abuse, the pesticide(s), the dyestuff(s), the malodorous compound(s), the chemical warfare agent(s), one or more hallucinogen(s), one or more toxin(s), one or more metabolite(s), or a combination thereof.
Statement 7. A method according to any one of the preceding Statements, where the complex is removed from the aqueous sample, the solid sample, or the gas sample.
Statement 8. A method according to any one of the preceding Statements, where the neuromuscular blocking agent(s), the anesthesia agent(s), the pharmaceutical agent(s), the drugs of abuse, the pesticide(s), the dyestuff(s), the malodorous compound(s), the chemical warfare agent(s), one or more hallucinogen(s), one or more toxin(s), one or more metabolite(s), or a combination thereof is present in and/or on an individual and the contacting comprises administration of the one or more compound(s).
Statement 9. A method for reversing drug-induced neuromuscular block and/or anesthesia and/or the effects of one or more pharmaceutical agent(s) and/or effects of one or more drug(s) of abuse in an individual comprising administering to an individual in need of reversal of neuromuscular block and/or reversal of anesthesia and/or reversal of the effects of one or more pharmaceutical agent(s) and/or reversal of the effects of one or more drug(s) of abuse and/or one or more chemical warfare agents one or more compound(s) having the following structure:

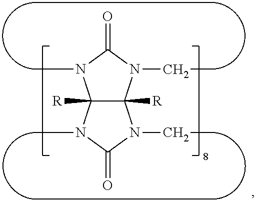

where each R is independently chosen from hydrogen, alkyl groups (e.g., methyl, ethyl, propyl, or isopropyl), hydroxyl groups, alkoxy groups, aryl groups, heteroaryl groups, amino groups, amide groups (e.g., N-and C-linked, primary secondary tertiary), carboxylic acid/carboxylate groups, ester groups (e.g., O— and C═O linked ester groups), imide groups, thiol groups, ether groups, polyether groups, phosphate groups, phosphonate groups, sulfonate groups (e.g., protonated and deprotonated sulfonate groups, where the counterion for the deprotonated sulfonated group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfonate groups (e.g., methyl sulfonate, ethyl sulfonate, propyl sulfonate, butyl sulfonate, and the like) (e.g., protonated and deprotonated alkyl sulfonate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), sulfate groups ((e.g., protonated and deprotonated sulfate groups, where the counterion for the deprotonated sulfate group is M⁺, where M⁺ is chosen from Na⁺, K⁺, Ca²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfate groups (e.g., protonated and deprotonated alkyl sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), and the like, and combinations thereof.
Statement 10. A method according to Statement 9, where the one or more compounds have the following structure:

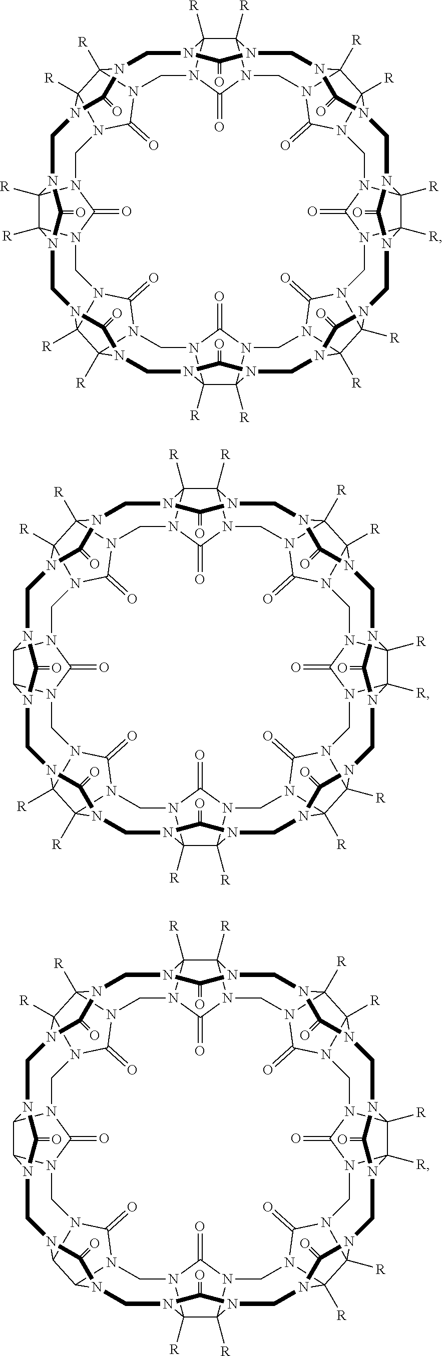

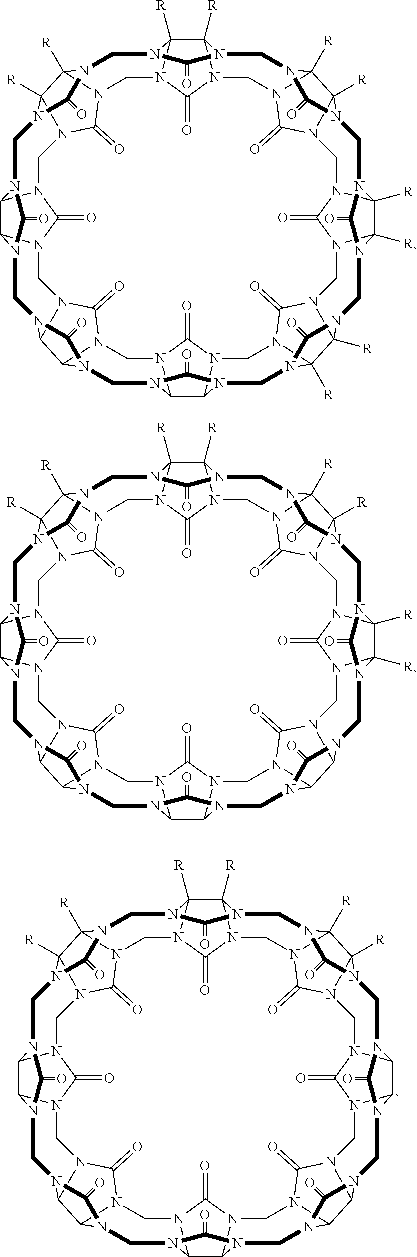

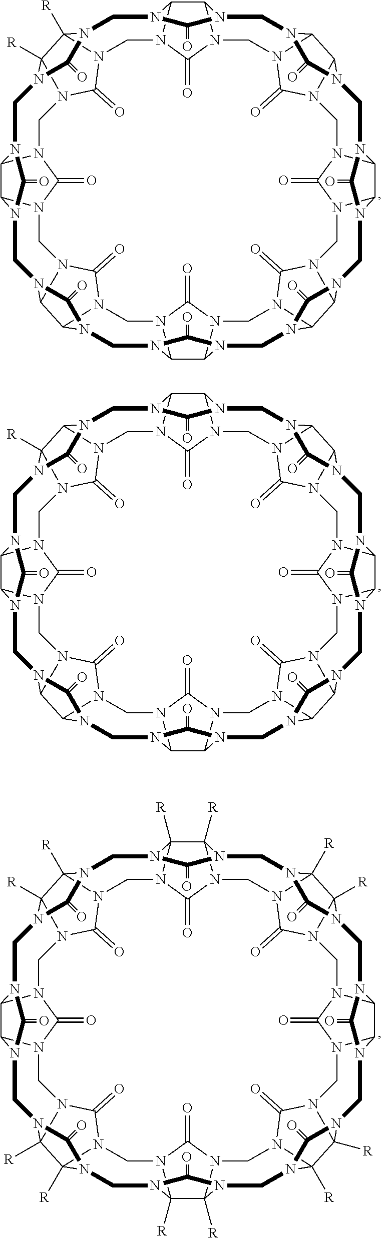

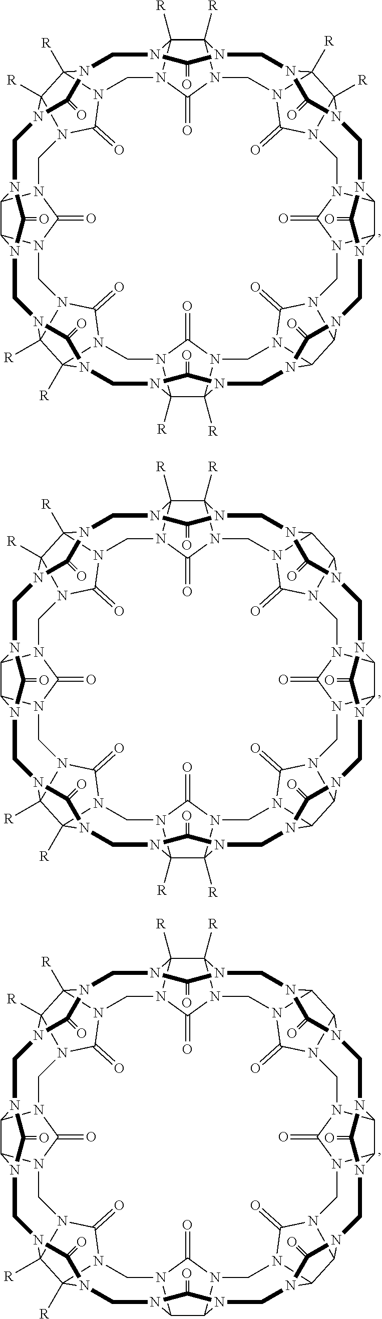

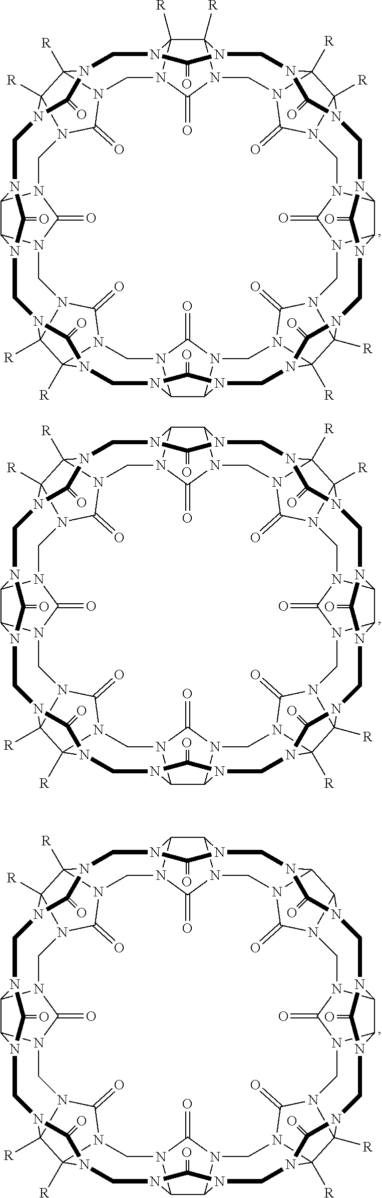

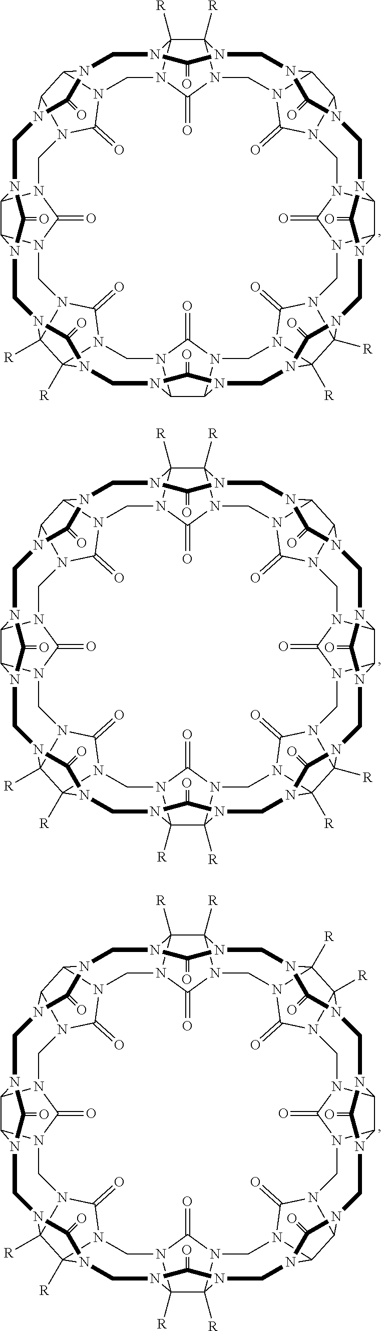

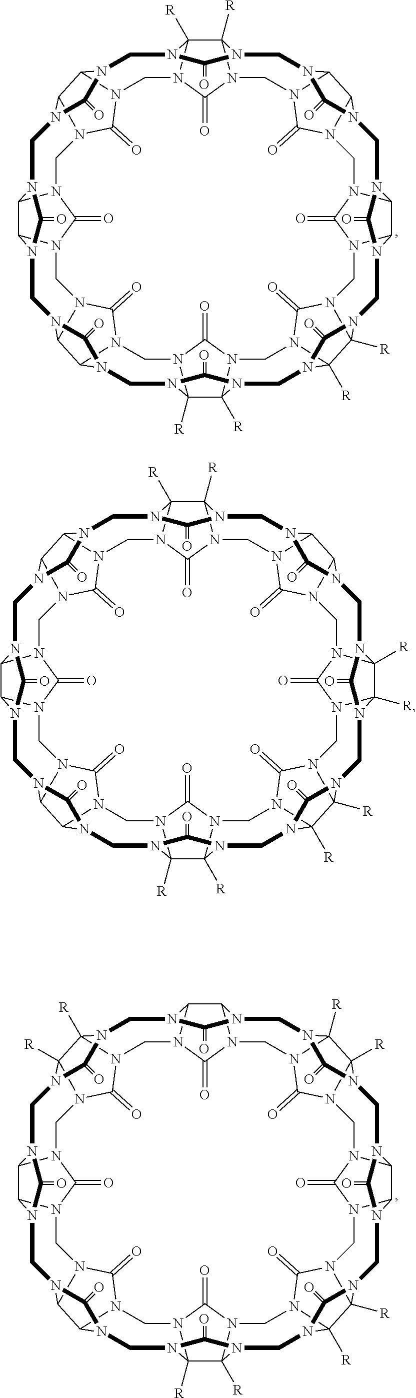

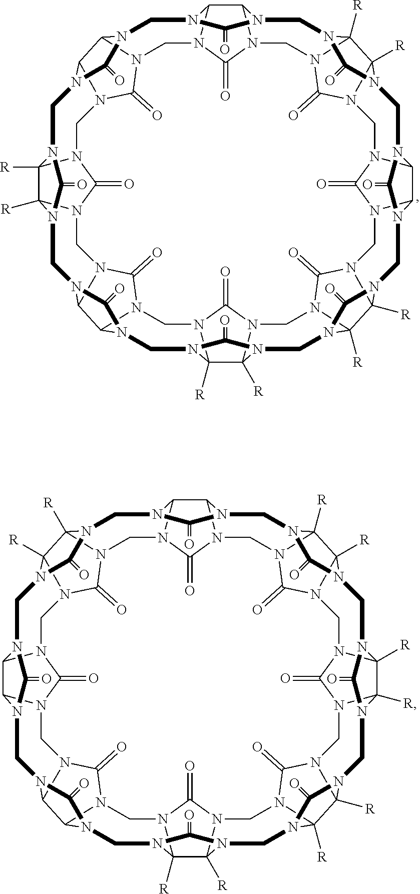

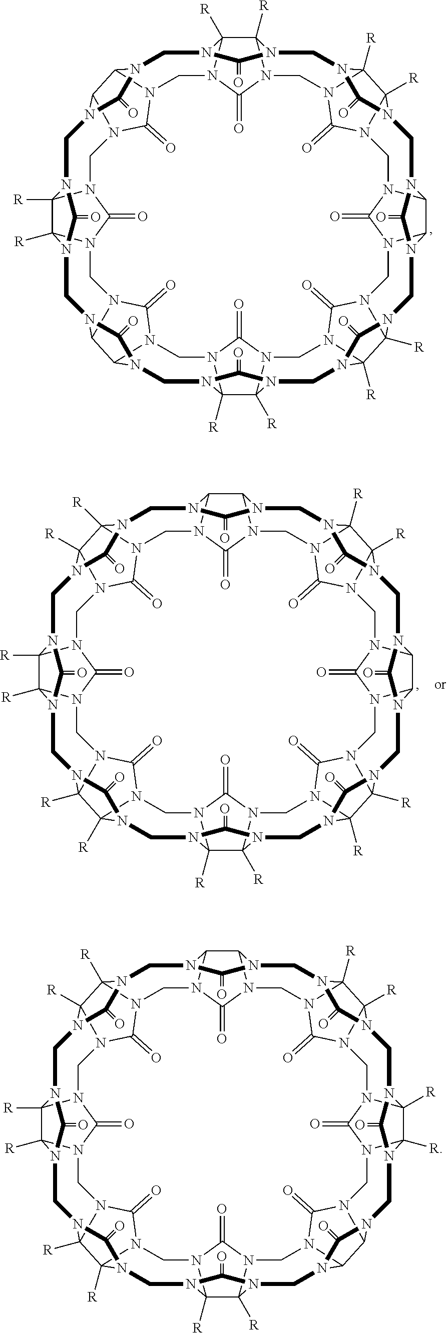

Statement 11. A method according to Statements 9 or 10, where the compound has the following structure:

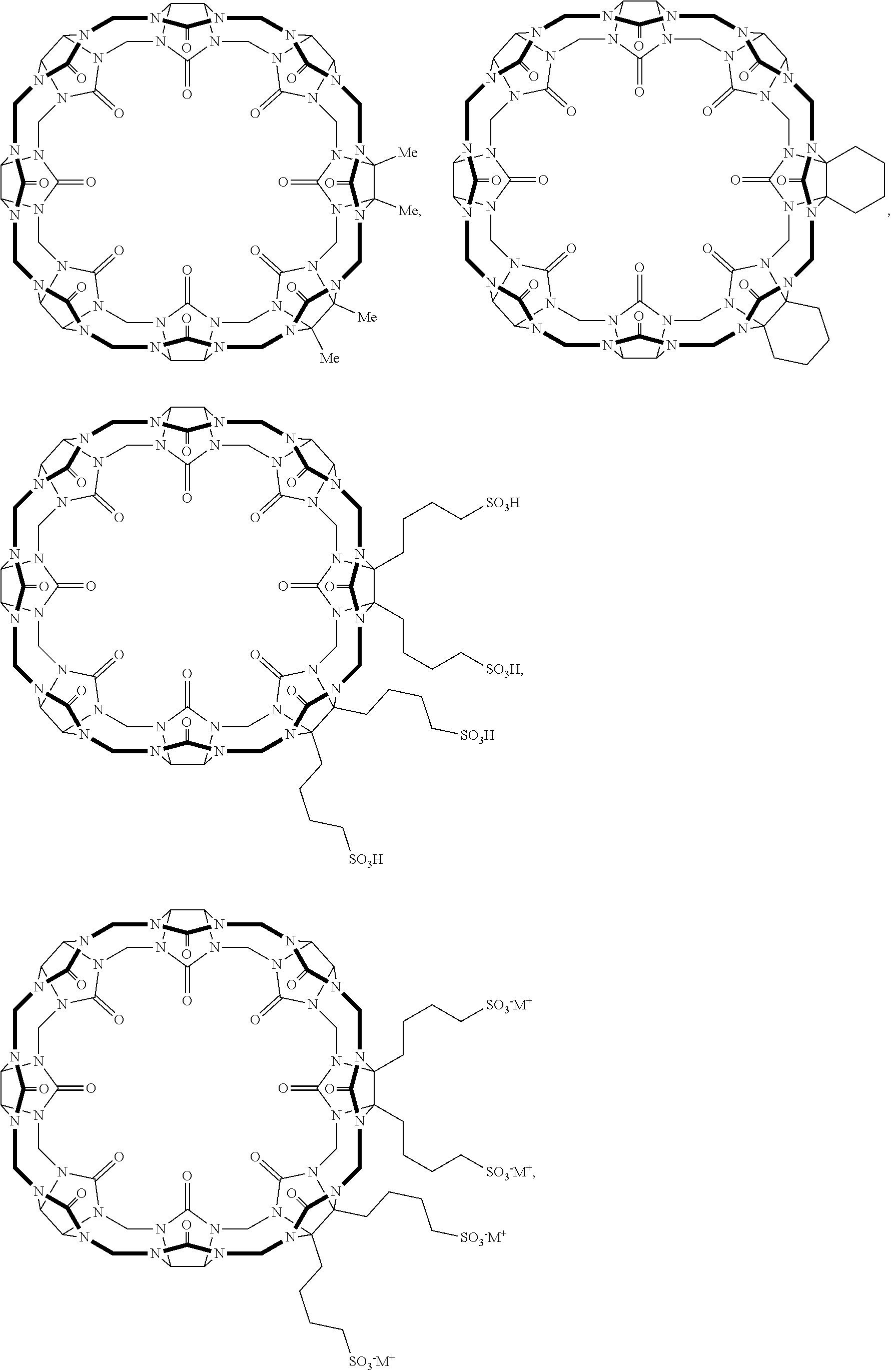

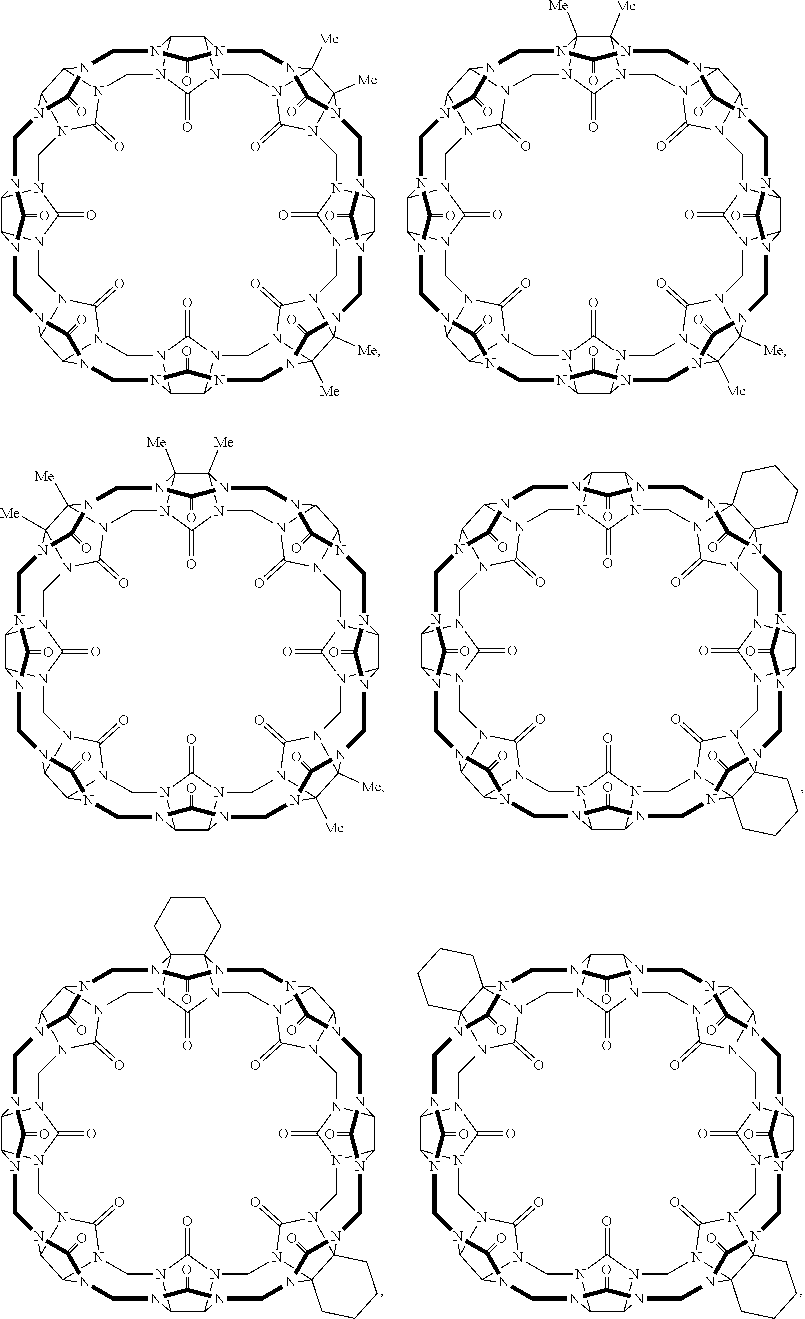

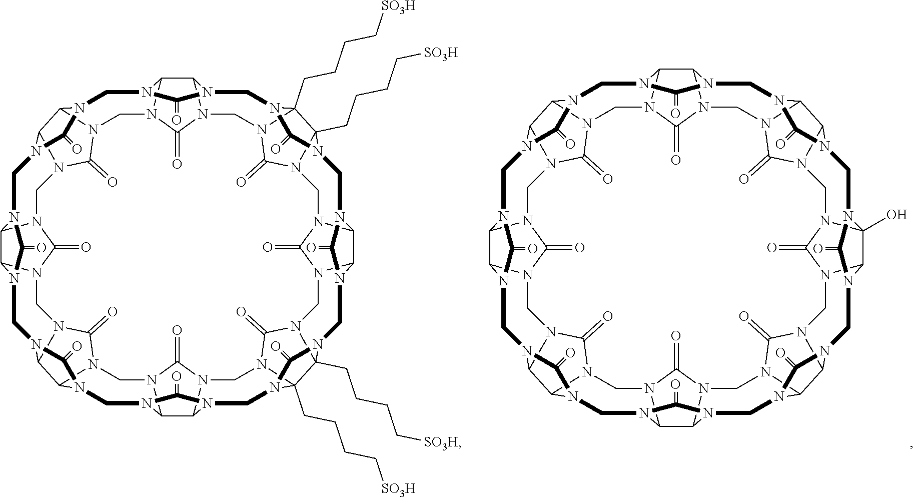

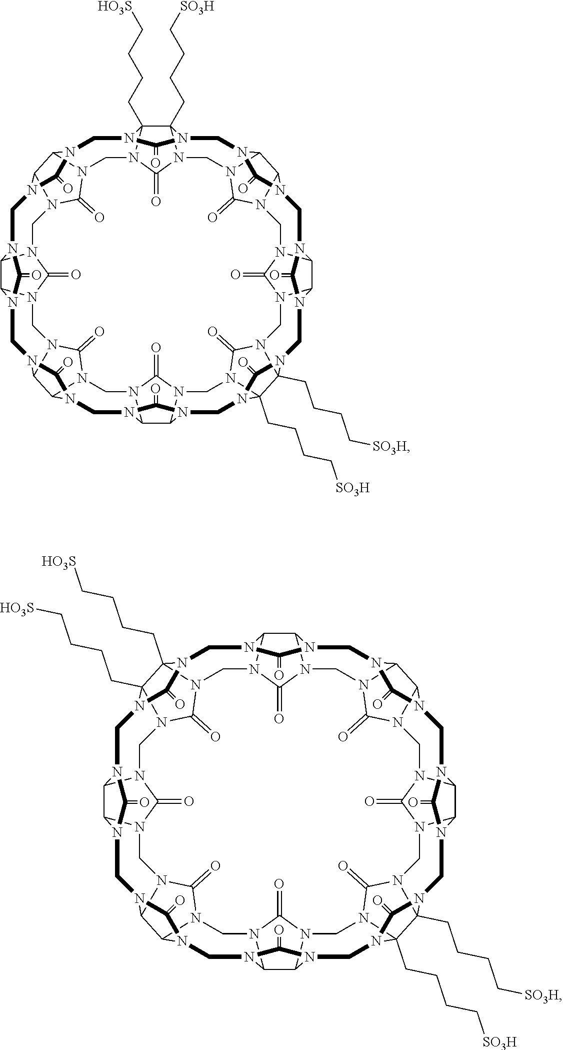

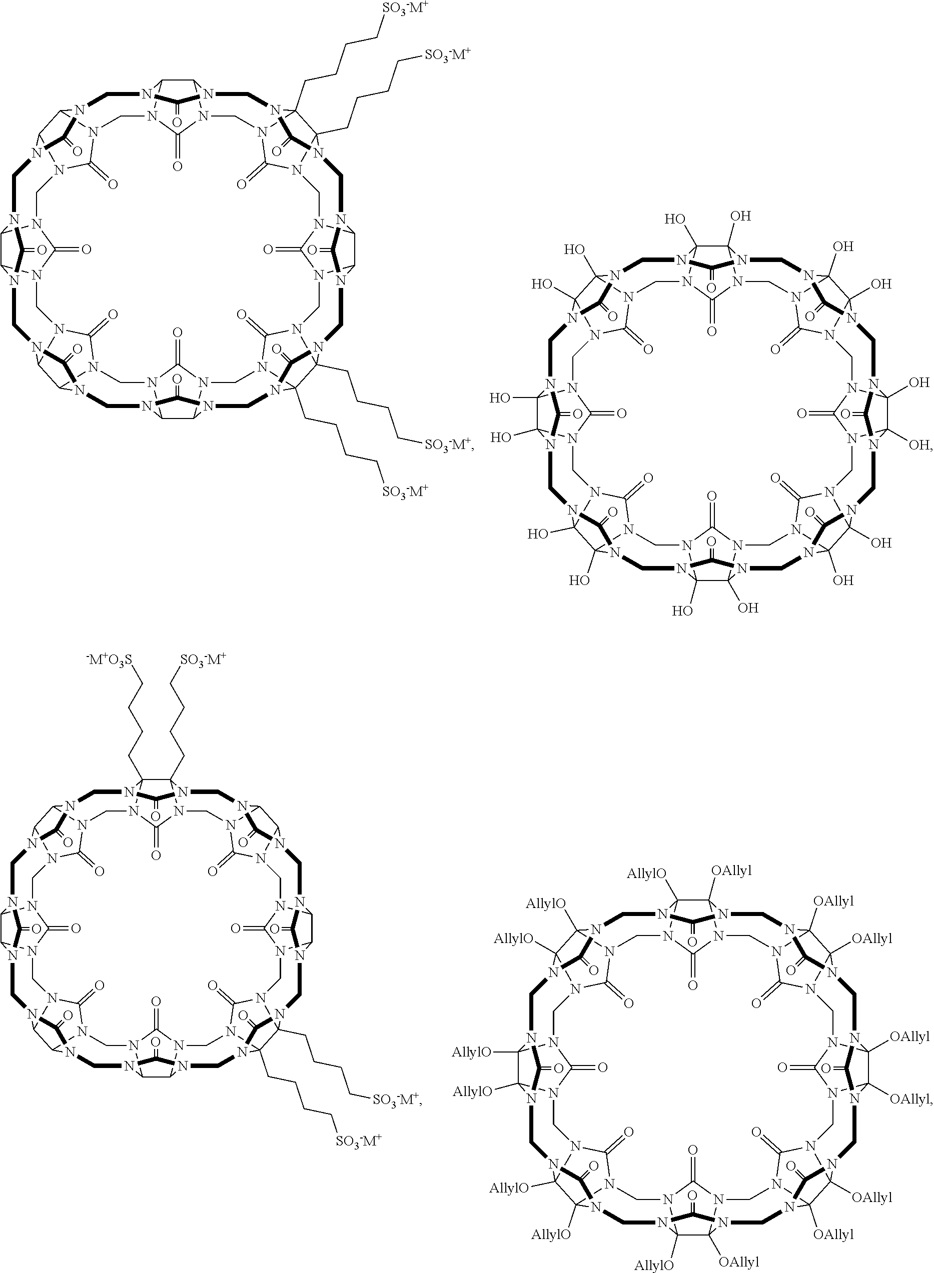

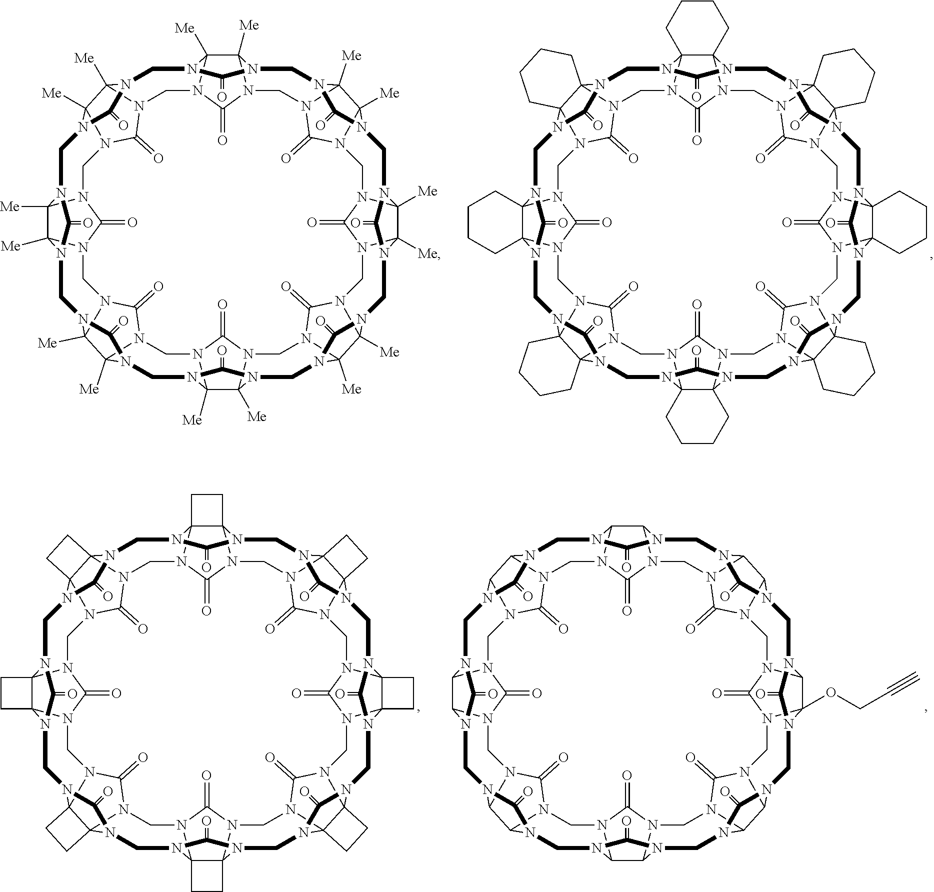

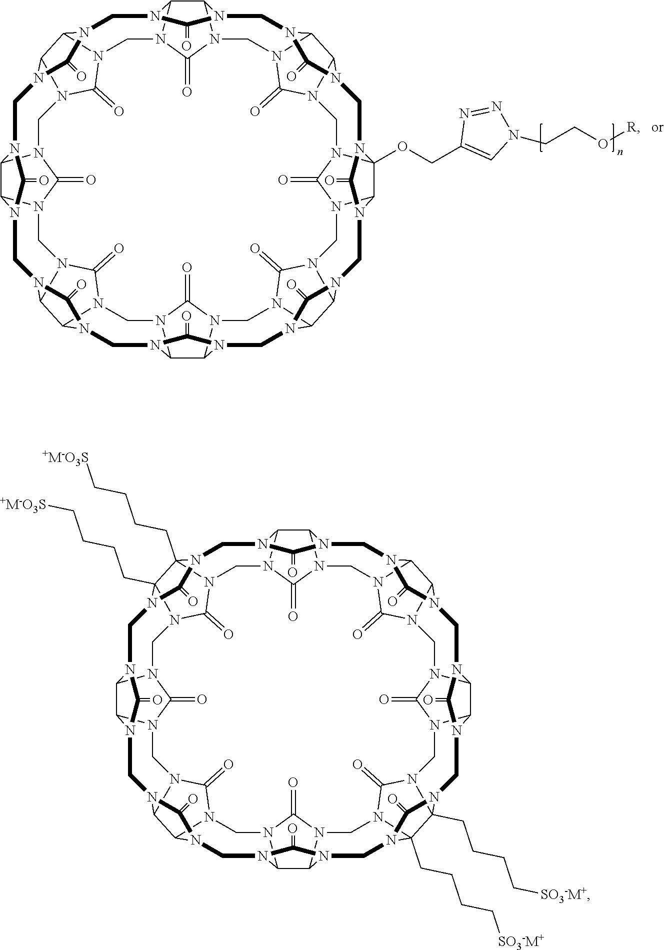

where n is 3 to 1000, including all values and ranges therebetween.
Statement 12. A method according to any one of Statements 9-11, where the compound has the following structure:

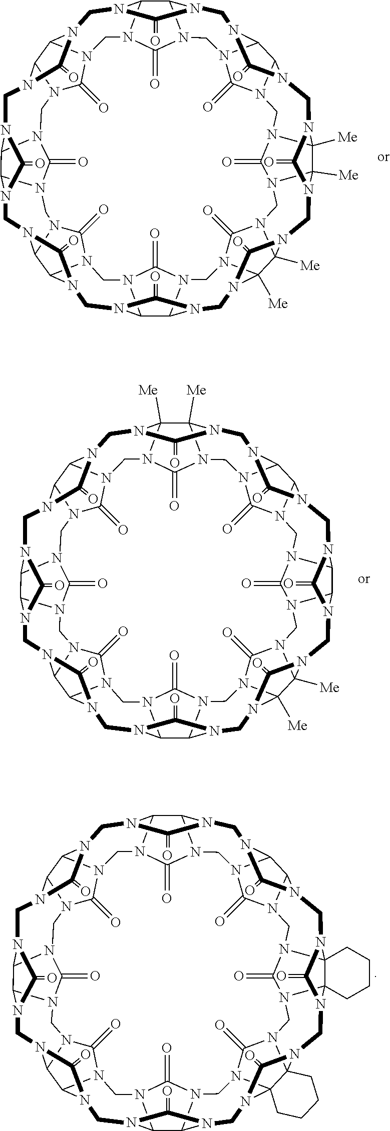

Statement 13. A method according to any one of Statements 9-12, where the individual is in need of reversal of drug-induced neuromuscular block and/or reversal of anesthesia.
Statement 14. A method according to any one of Statements 9-13, where the individual is in need of reversal of the effects of one or more drug(s) of abuse, one or more pesticide(s), one or more chemical warfare agent(s), one or more nerve agent(s), one or more hallucinogen(s), one or more toxin(s), one or more metabolite(s), or combinations thereof.
Statement 15. A method for prophylaxis and/or therapy of a condition in an individual comprising administering to an individual in need of the prophylaxis and/or the therapy a composition comprising a compound having the following structure:

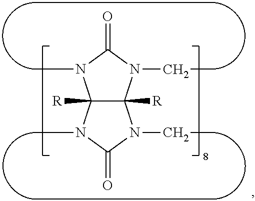

where each R is independently chosen from hydrogen, alkyl groups (e.g., methyl, ethyl, propyl, or isopropyl), hydroxyl groups, alkoxy groups, aryl groups, heteroaryl groups, amino groups, amide groups (e.g., N-and C-linked, primary secondary tertiary), carboxylic acid/carboxylate groups, ester groups (e.g., O— and C═O linked ester groups), imide groups, thiol groups, ether groups, polyether groups, phosphate groups, phosphonate groups, sulfonate groups (e.g., protonated and deprotonated sulfonate groups, where the counterion for the deprotonated sulfonated group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfonate groups (e.g., methyl sulfonate, ethyl sulfonate, propyl sulfonate, butyl sulfonate, and the like) (e.g., protonated and deprotonated alkyl sulfonate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), sulfate groups ((e.g., protonated and deprotonated sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfate groups (e.g., protonated and deprotonated alkyl sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), and the like, and combinations thereof, and optionally, one or more pharmaceutical agents, where the one or more compounds and the one or more pharmaceutical agents are a complex, and subsequent to the administration the therapy and/or the prophylaxis of the condition in the individual occurs.
Statement 16. A method according to Statement 15, where the one or more compounds have the following structure:

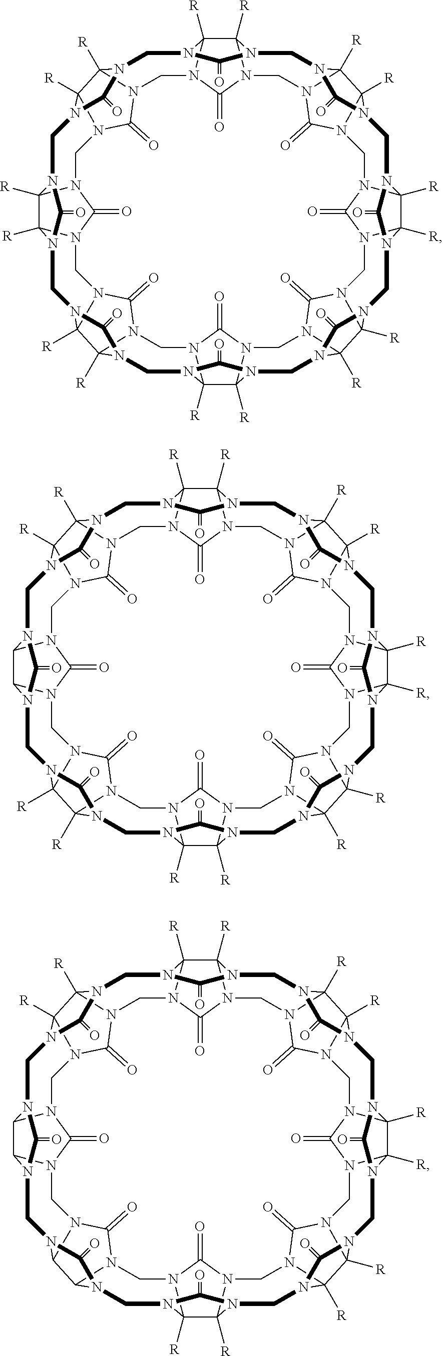

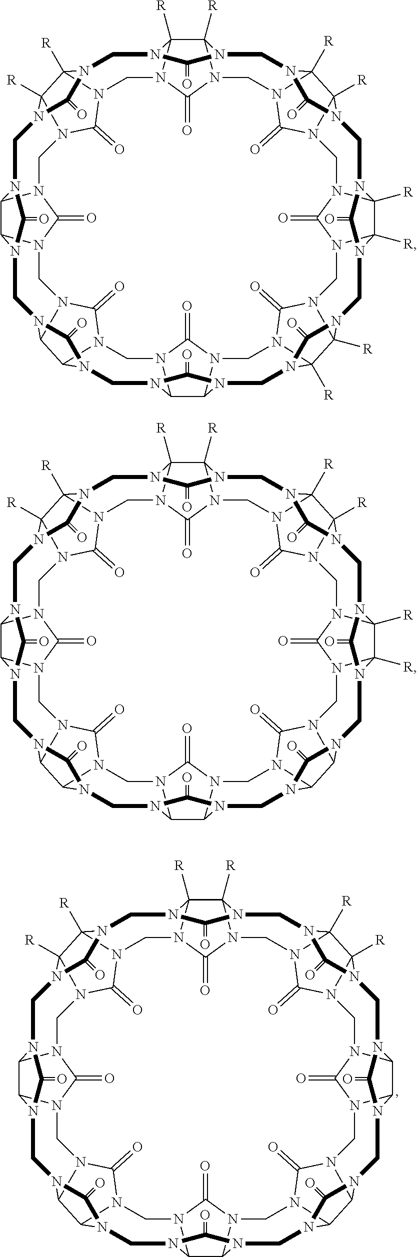

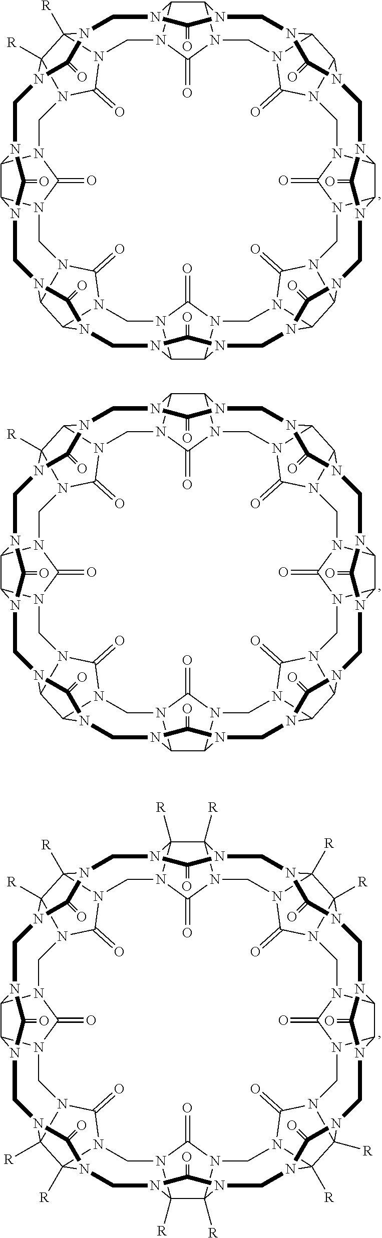

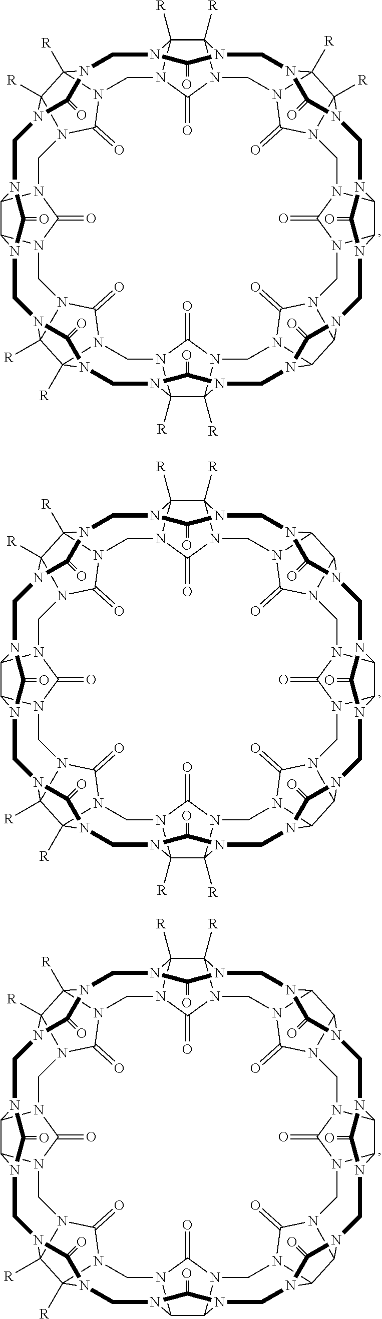

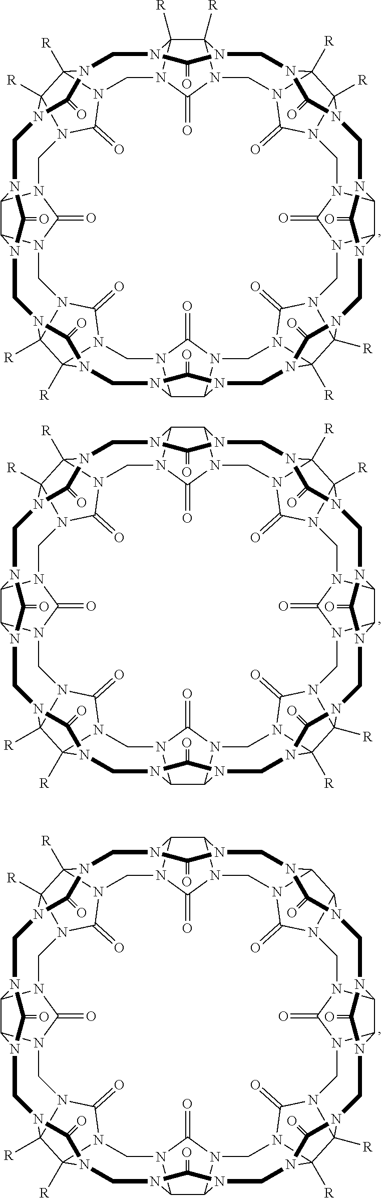

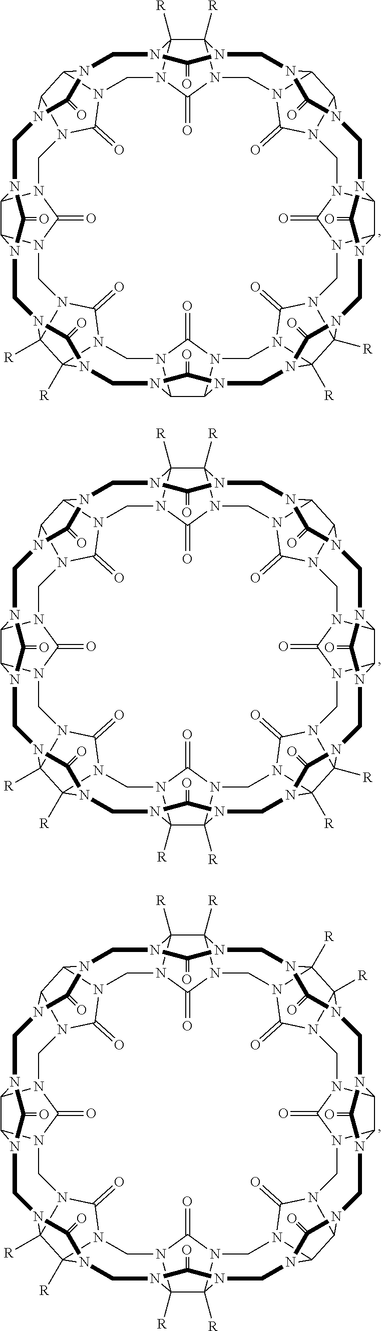

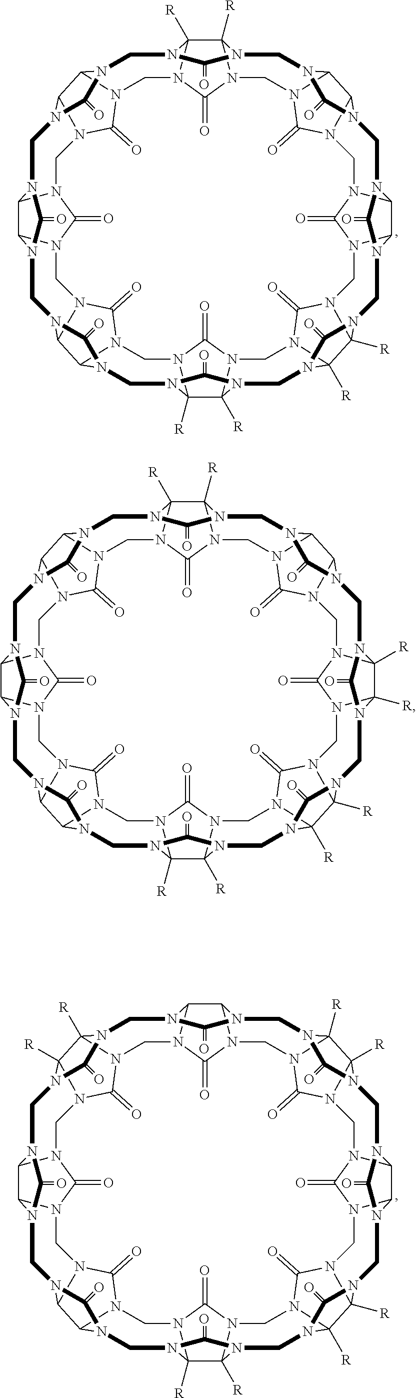

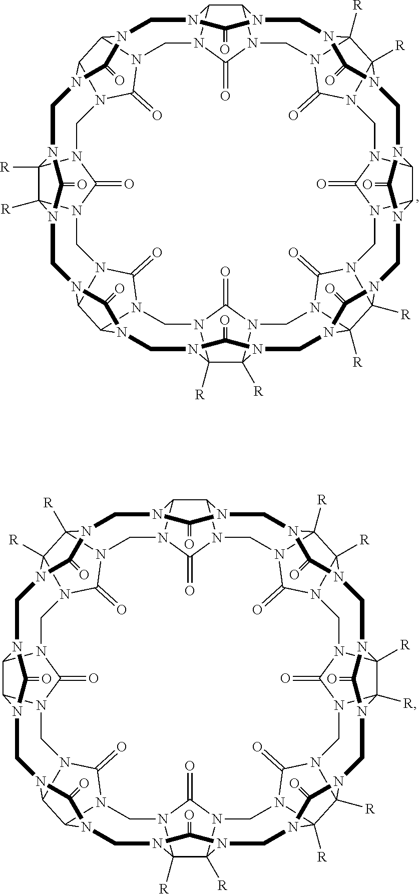

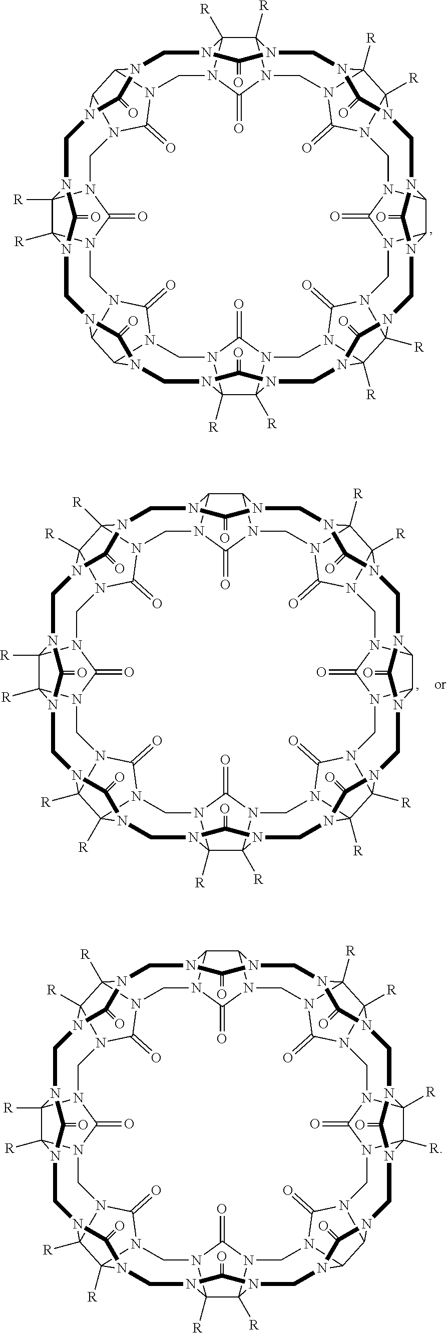

Statement 17. A method according to Statements 15 or 16, where the one or more compounds has the following structure:

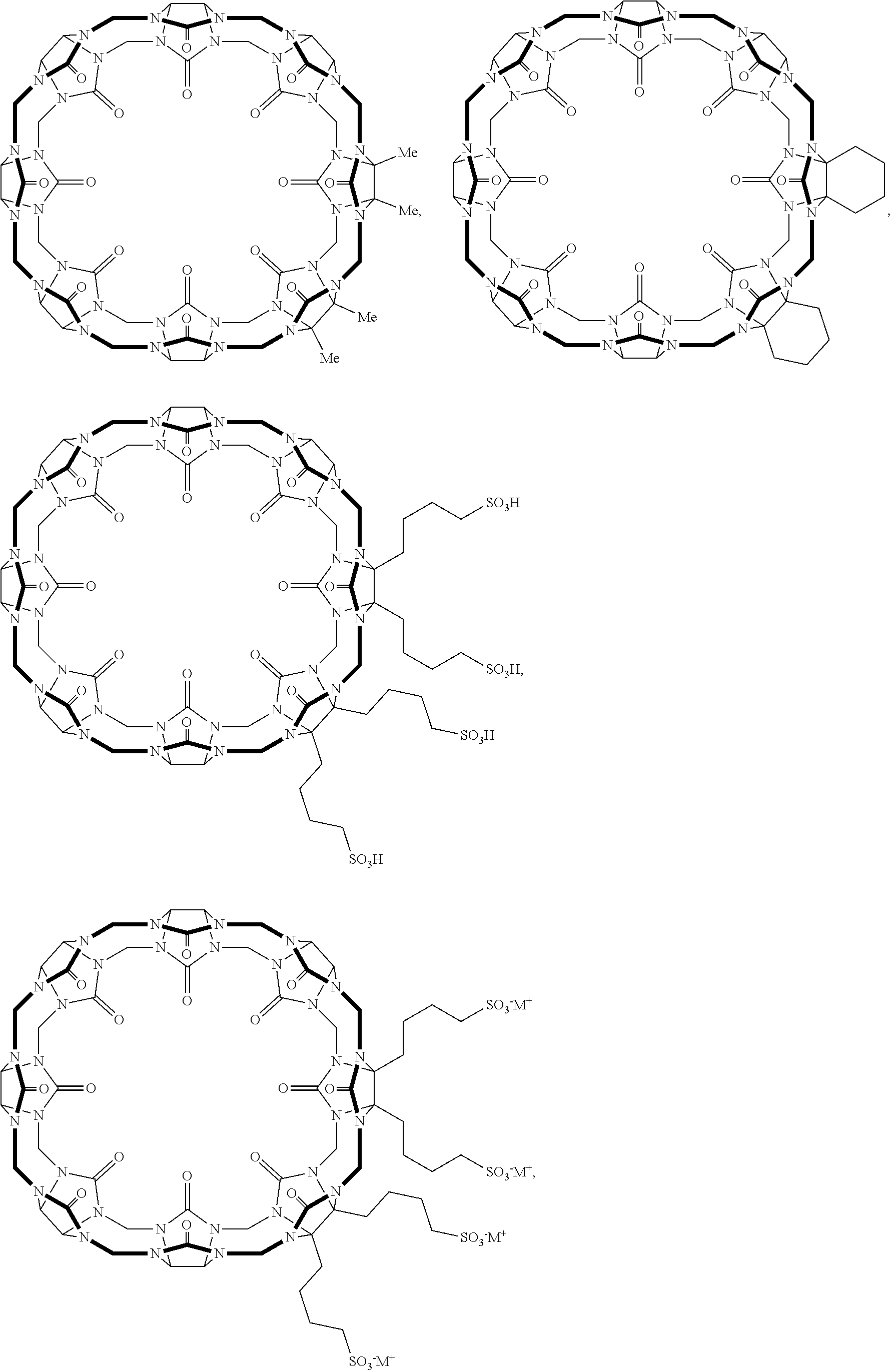

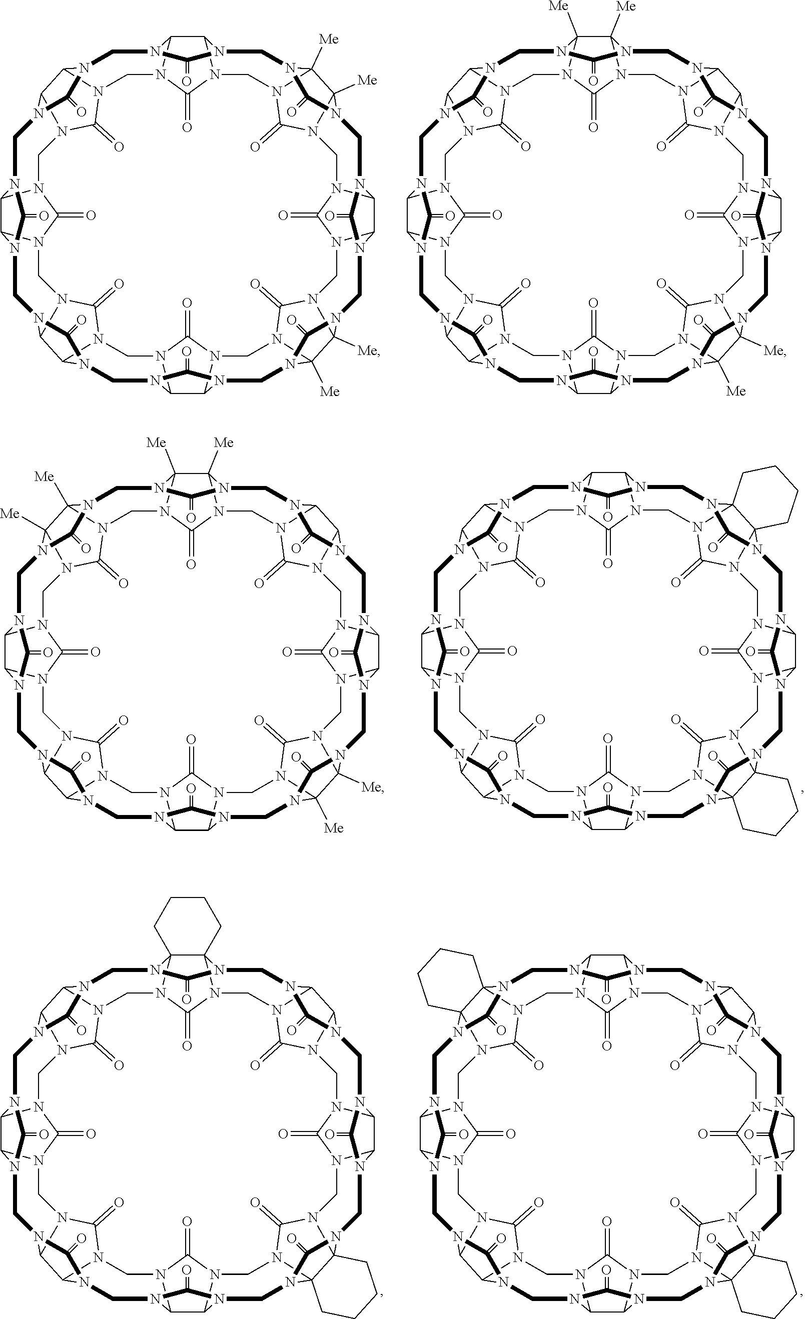

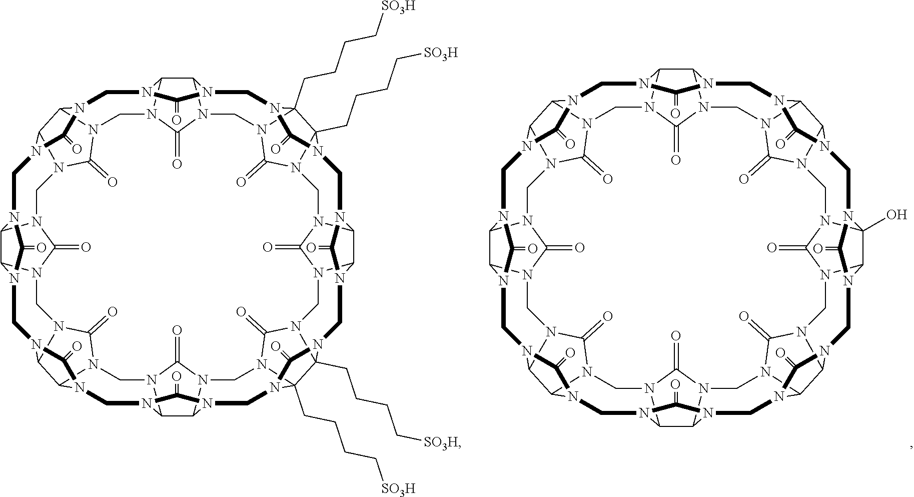

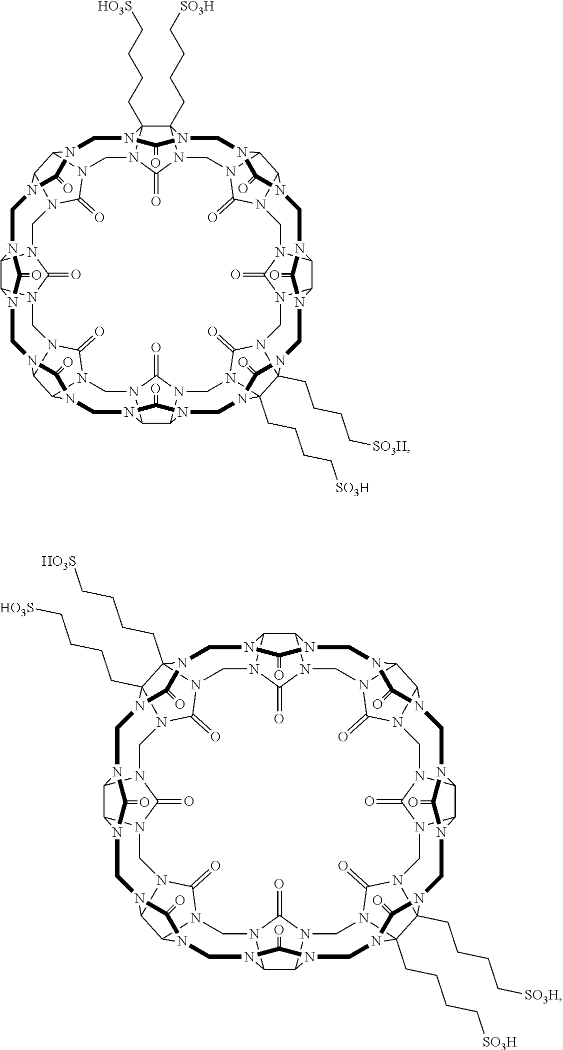

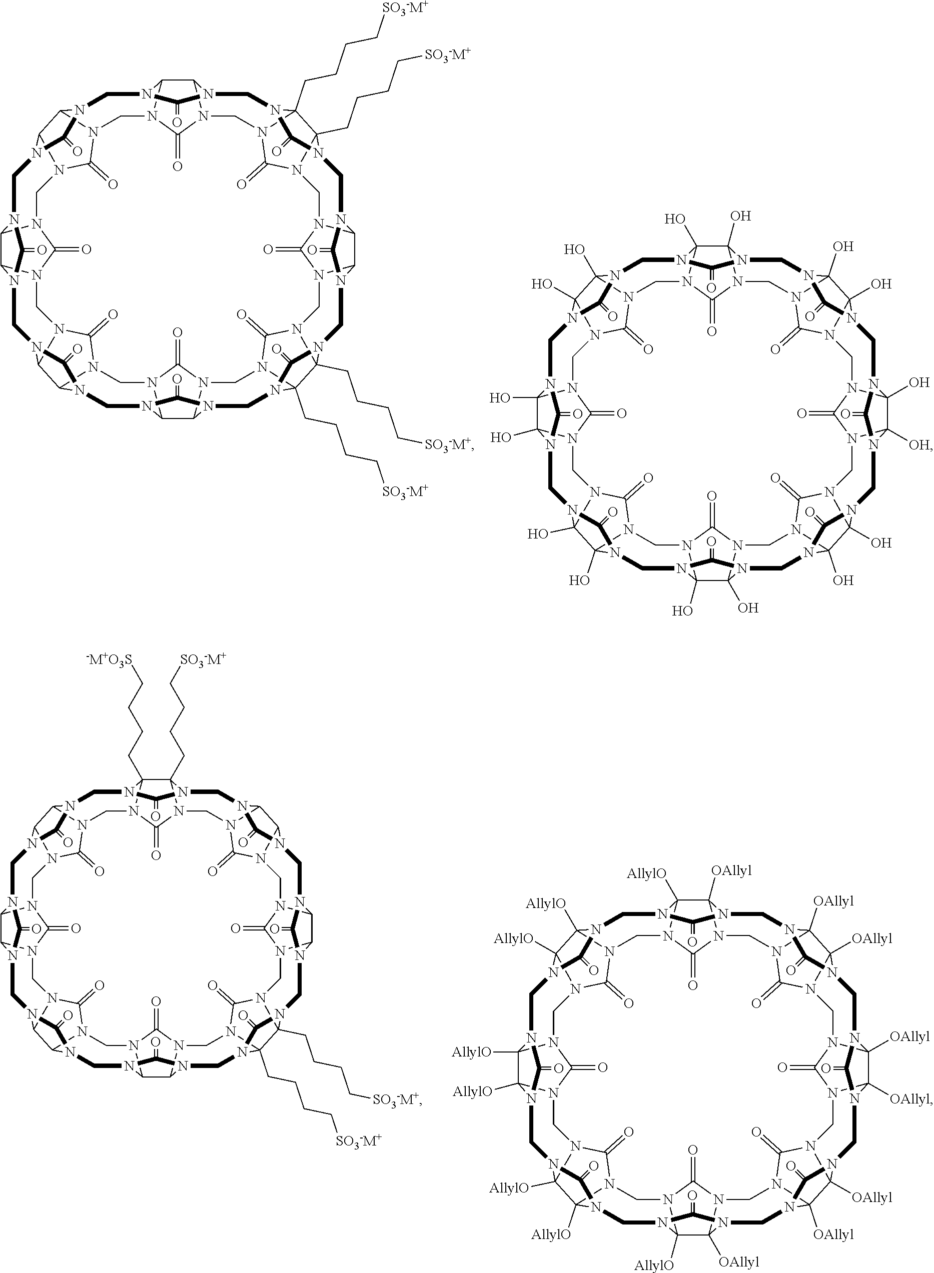

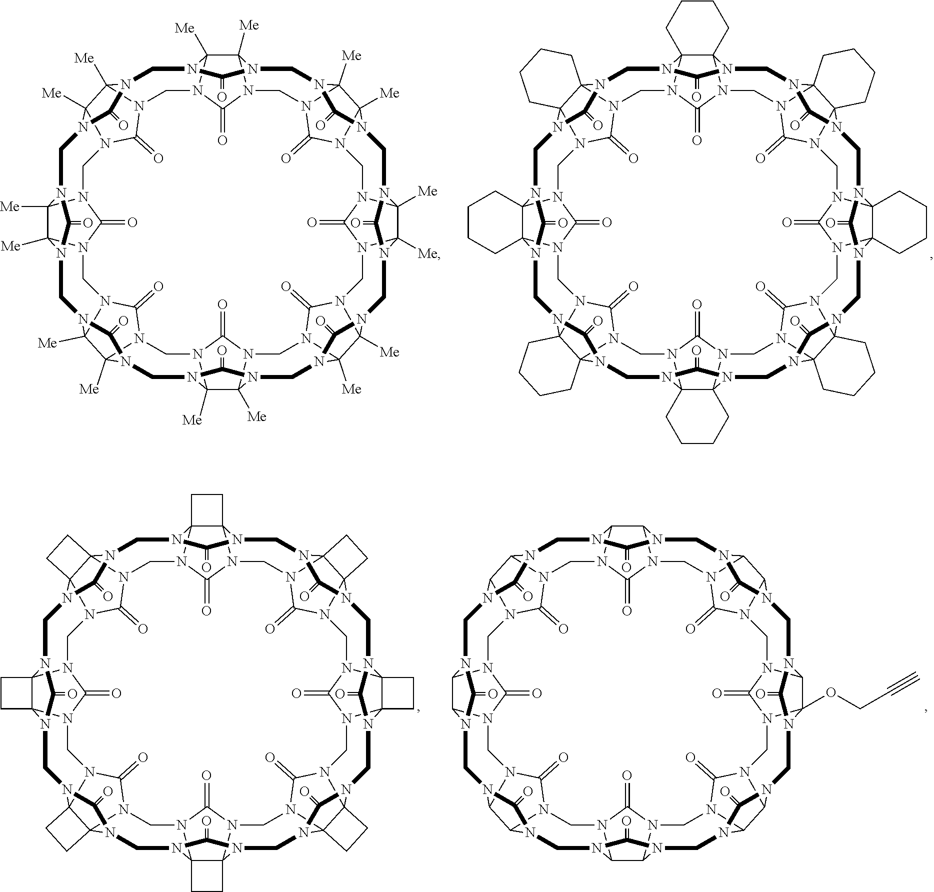

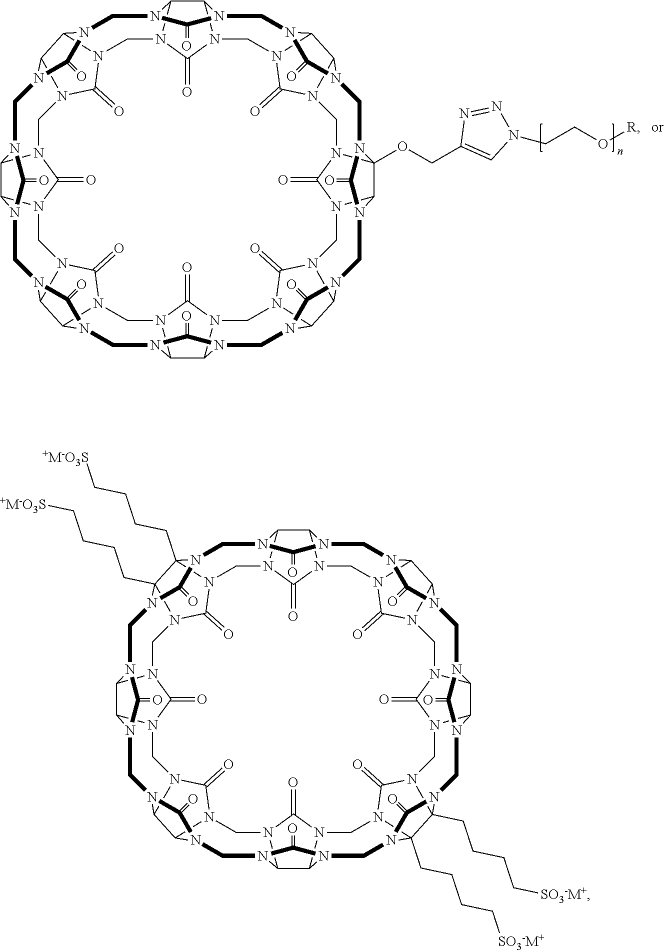

where n is 3 to 1000, including all values and ranges therebetween.
Statement 18. A method according to any one of Statements 15-17, where the one or more compounds have the following structure:

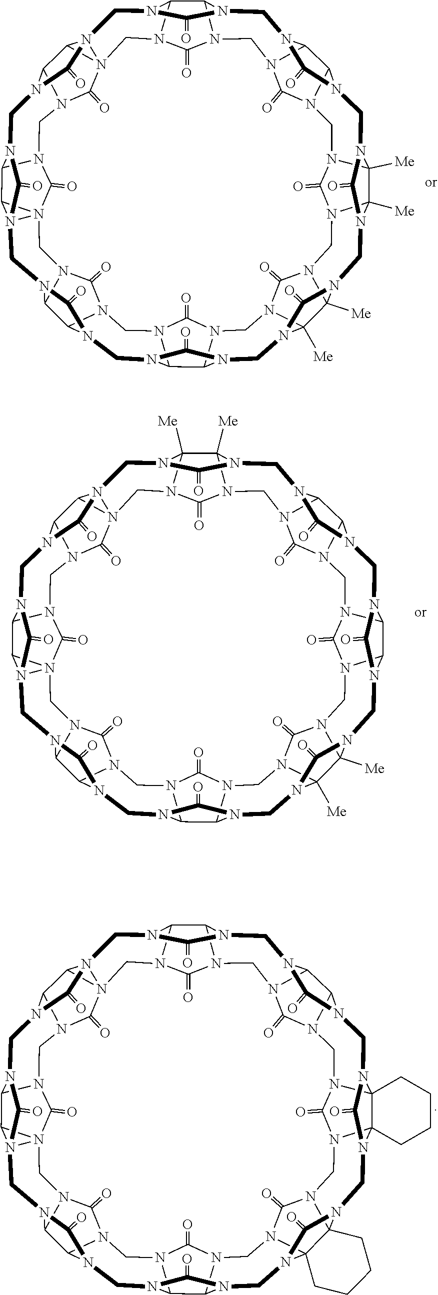

Statement 19. A method according to any one of Statements 15-18, where one or more of the pharmaceutical agent(s) has/have a solubility of less than 100 μM in an aqueous solvent.
Statement 20. A composition comprising a pharmaceutical carrier, one or more compounds having the following structure:

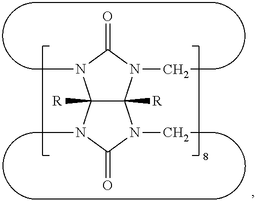

where each R is independently chosen from hydrogen, alkyl groups (e.g., methyl, ethyl, propyl, or isopropyl), hydroxyl groups, alkoxy groups, aryl groups, heteroaryl groups, amino groups, amide groups (e.g., N-and C-linked, primary secondary tertiary), carboxylic acid/carboxylate groups, ester groups (e.g., O— and C═O linked ester groups), imide groups, thiol groups, ether groups, polyether groups, phosphate groups, phosphonate groups, sulfonate groups (e.g., protonated and deprotonated sulfonate groups, where the counterion for the deprotonated sulfonated group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfonate groups (e.g., methyl sulfonate, ethyl sulfonate, propyl sulfonate, butyl sulfonate, and the like) (e.g., protonated and deprotonated alkyl sulfonate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), sulfate groups ((e.g., protonated and deprotonated sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), alkyl sulfate groups (e.g., protonated and deprotonated alkyl sulfate groups, where the counterion for the deprotonated sulfate group is M⁺, where M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS)), and, optionally, one or more pharmaceutical agents.
Statement 21. A composition according to Statement 20, where the one or more compounds have the following structure:

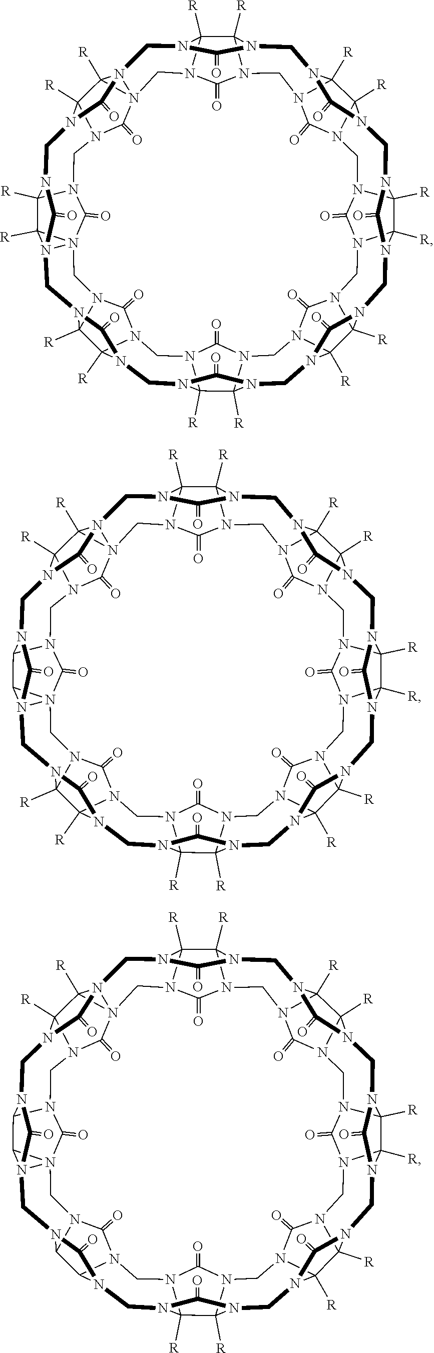

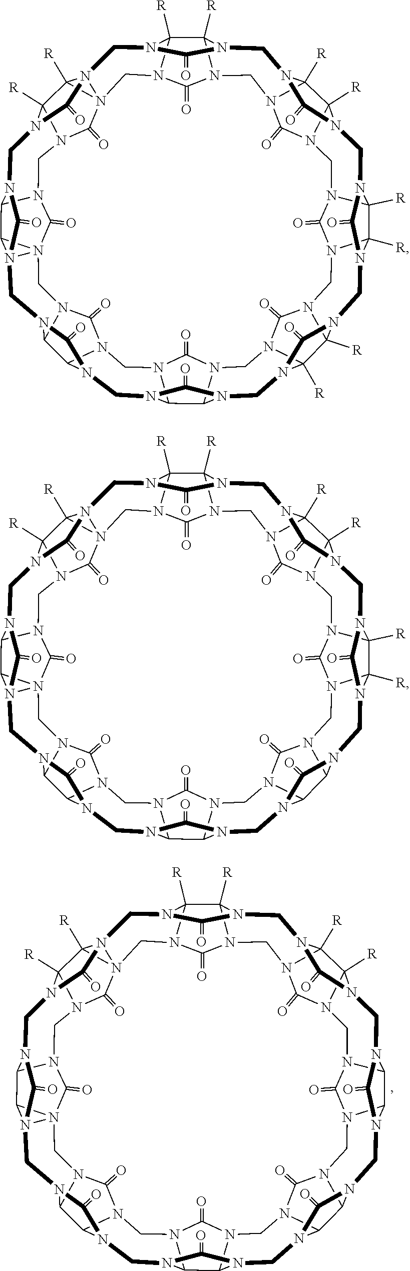

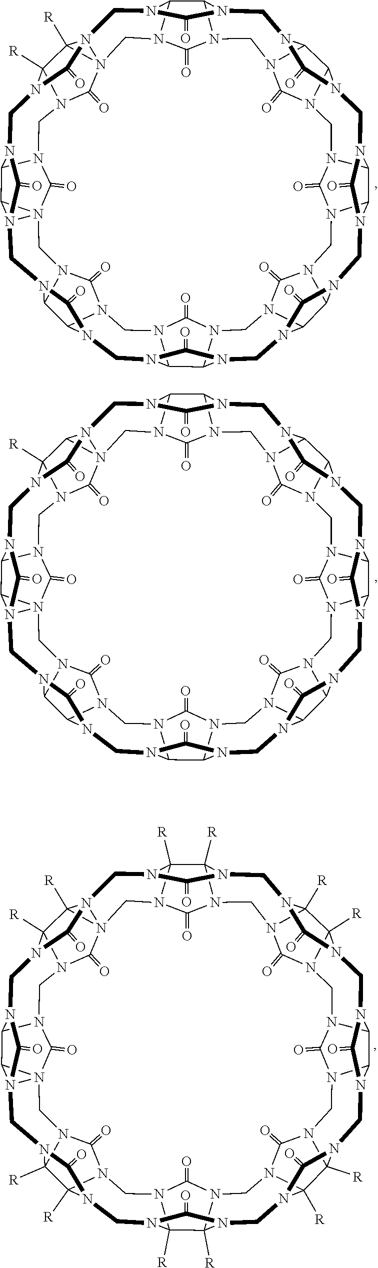

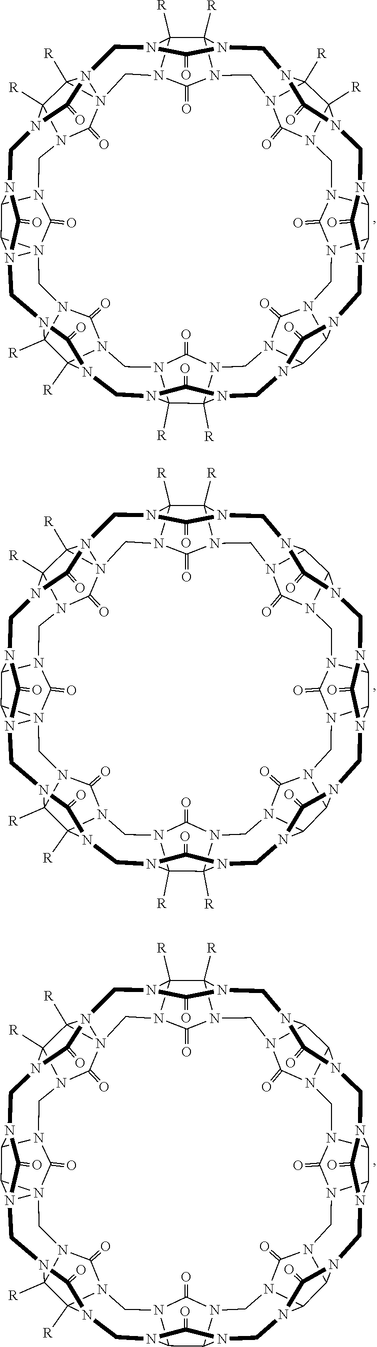

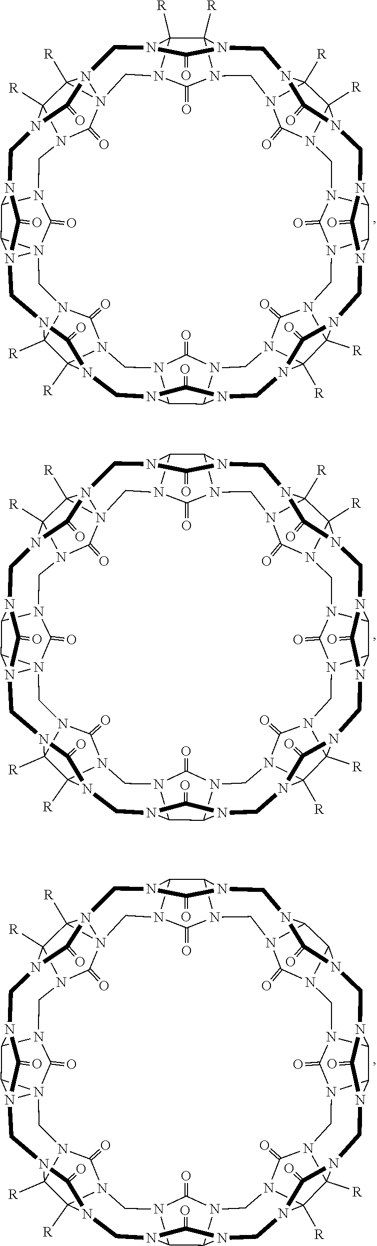

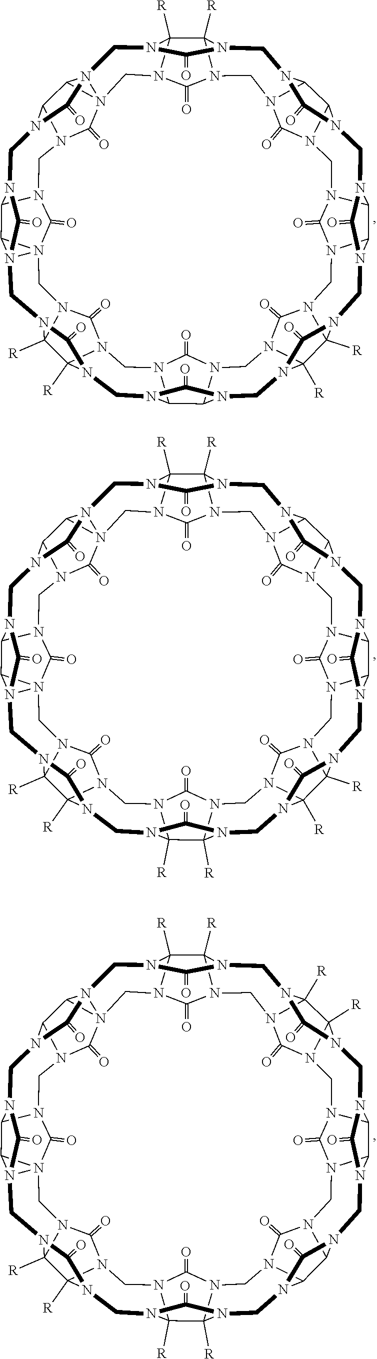

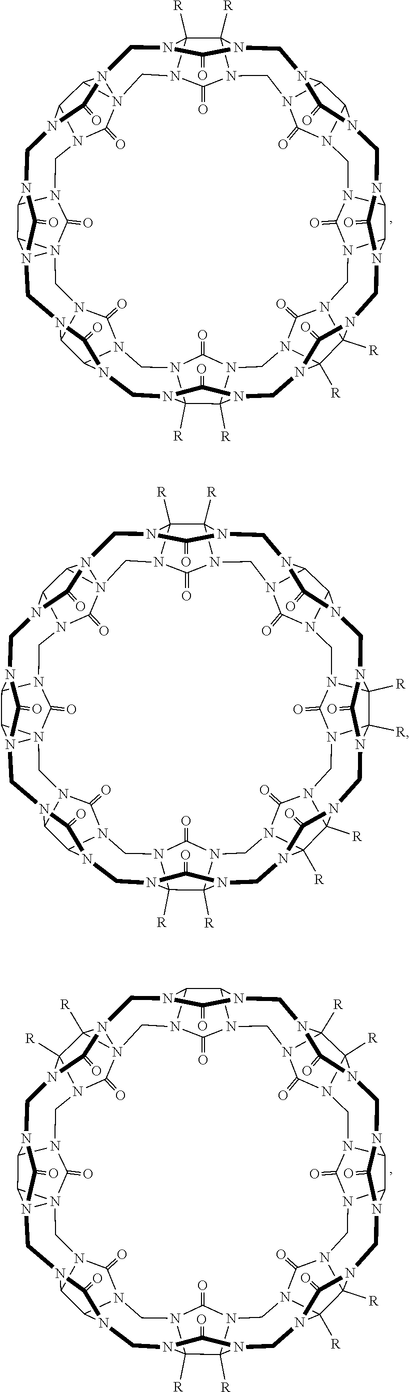

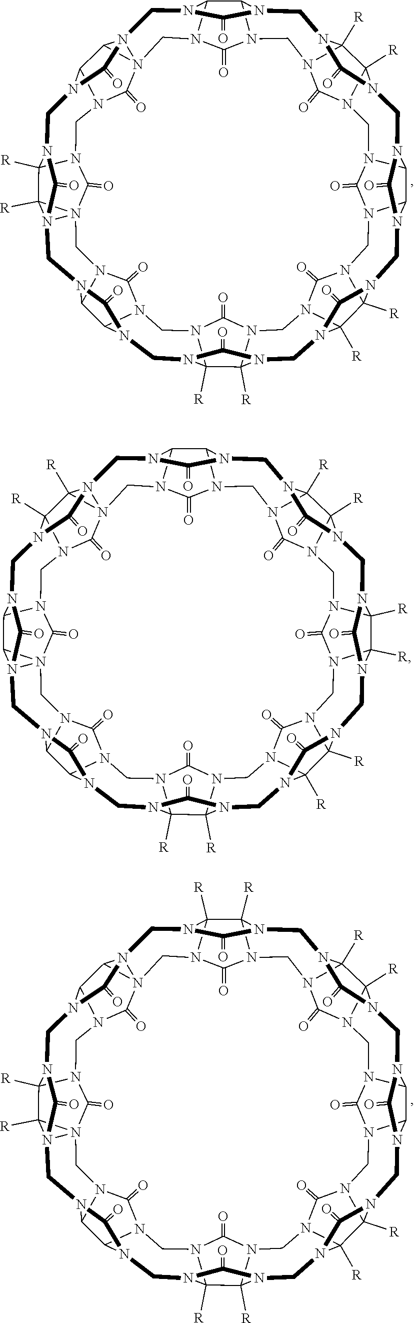

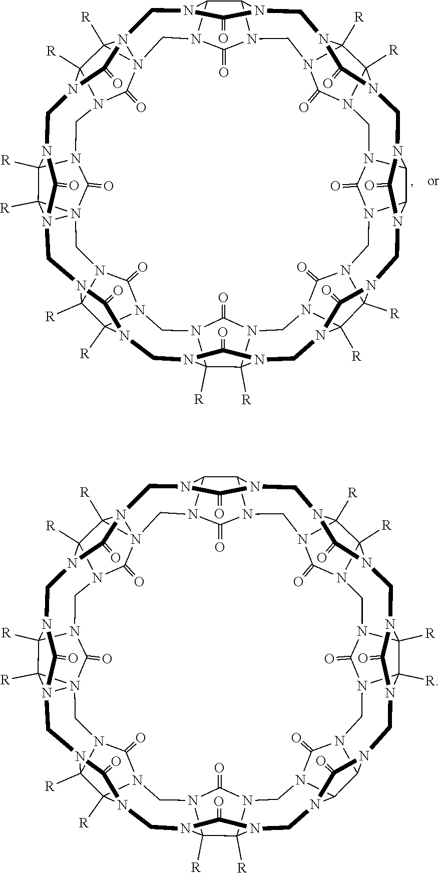

Statement 22. A composition according to Statements 20 or 21, where the one or more compounds have the following structure:

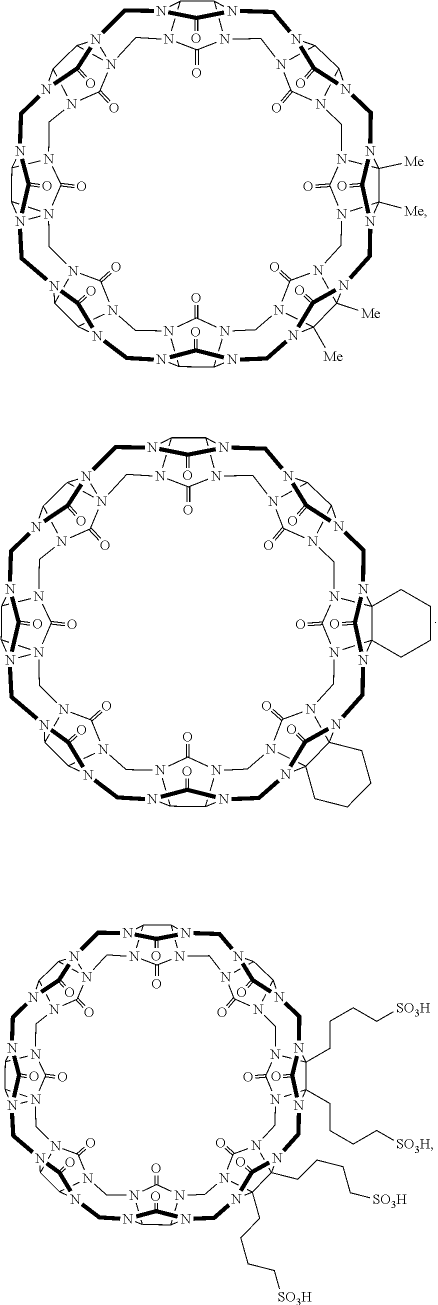

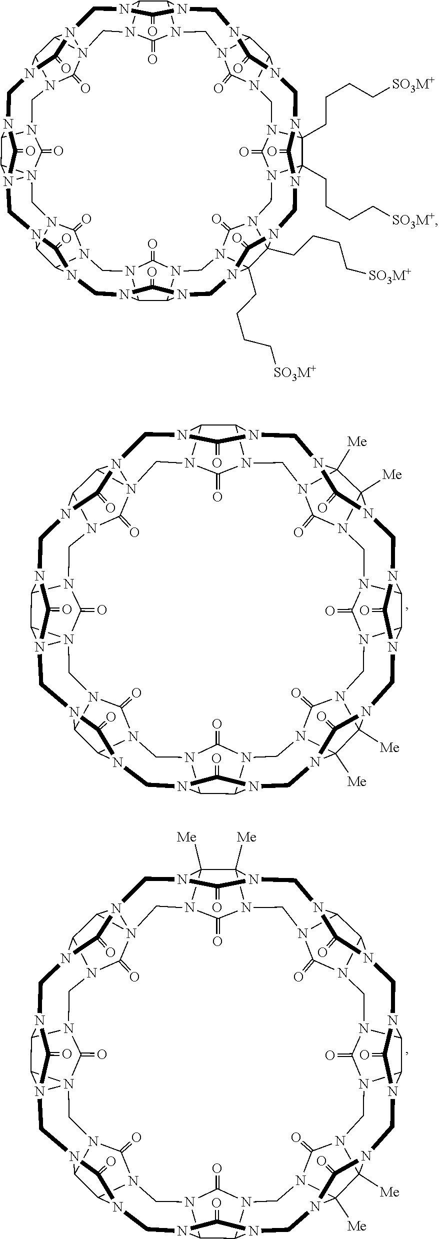

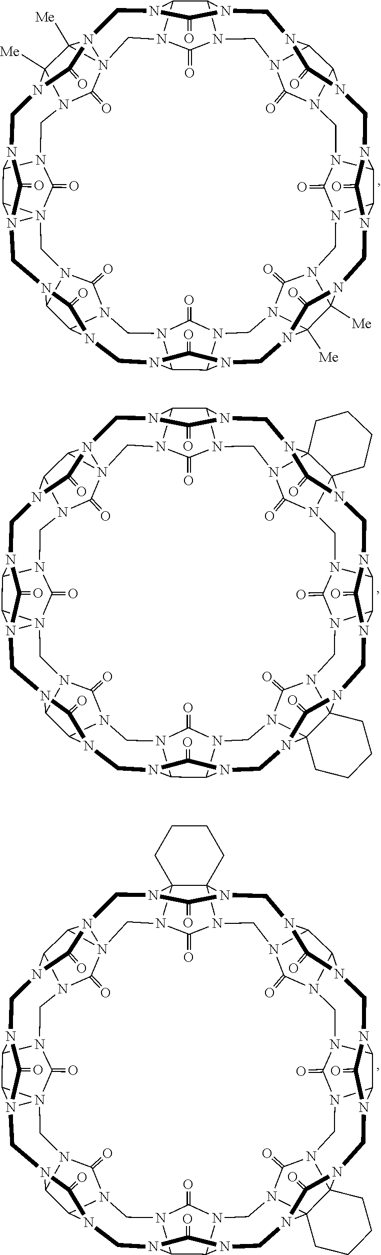

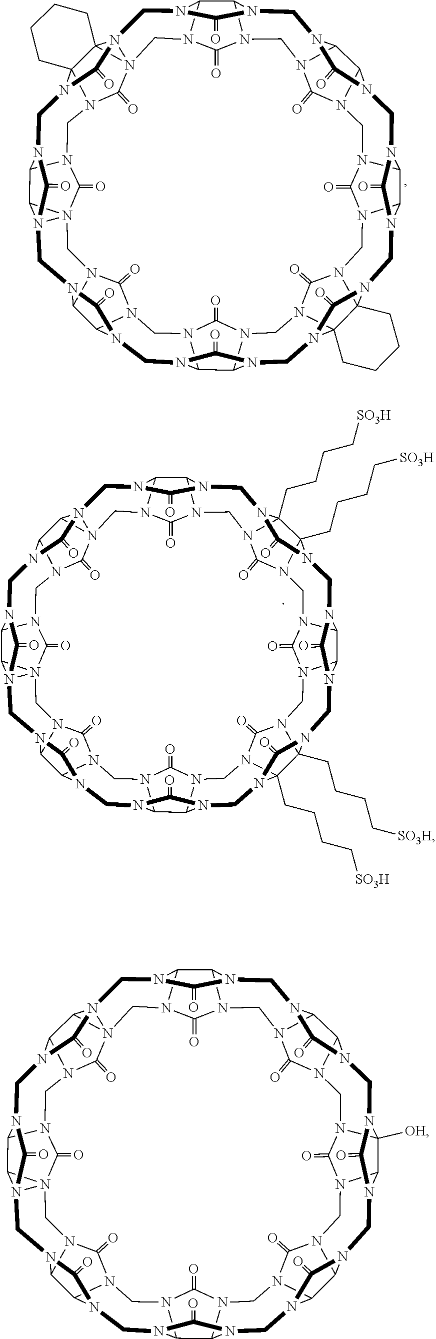

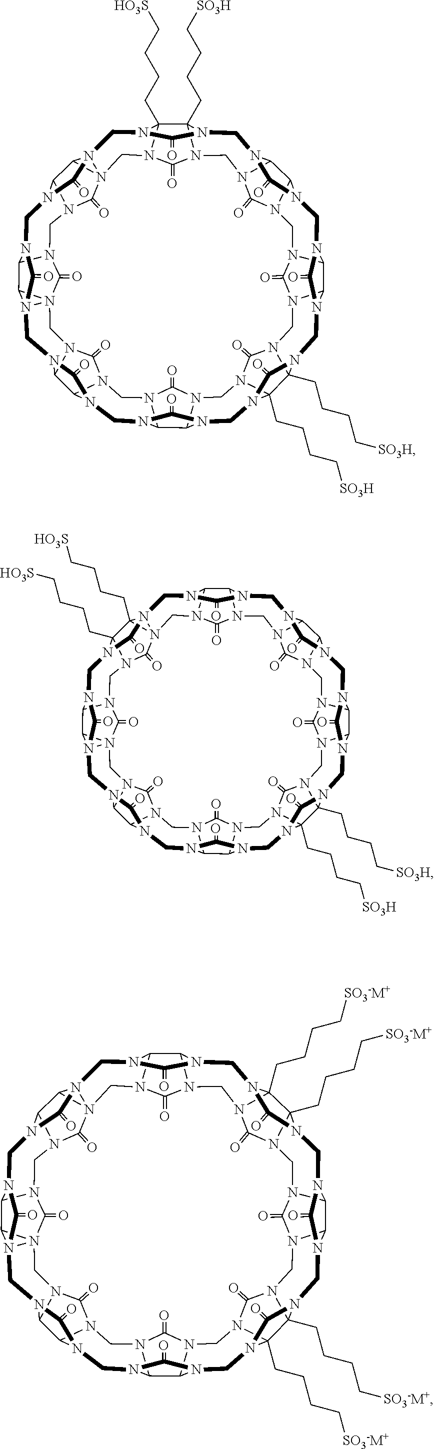

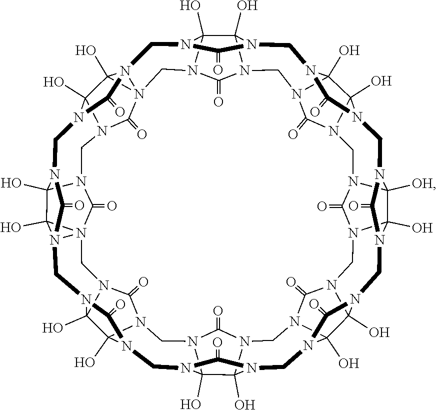

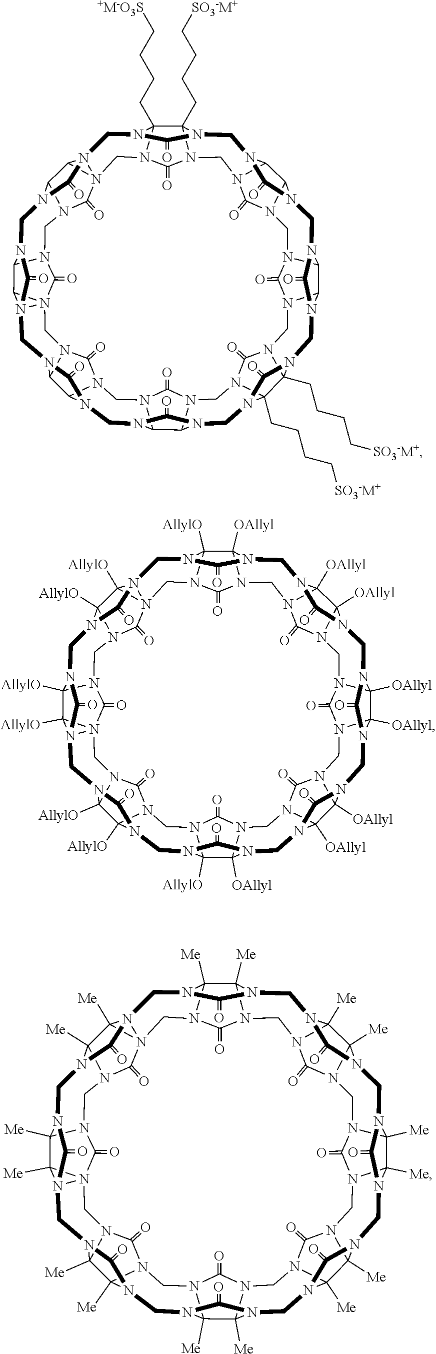

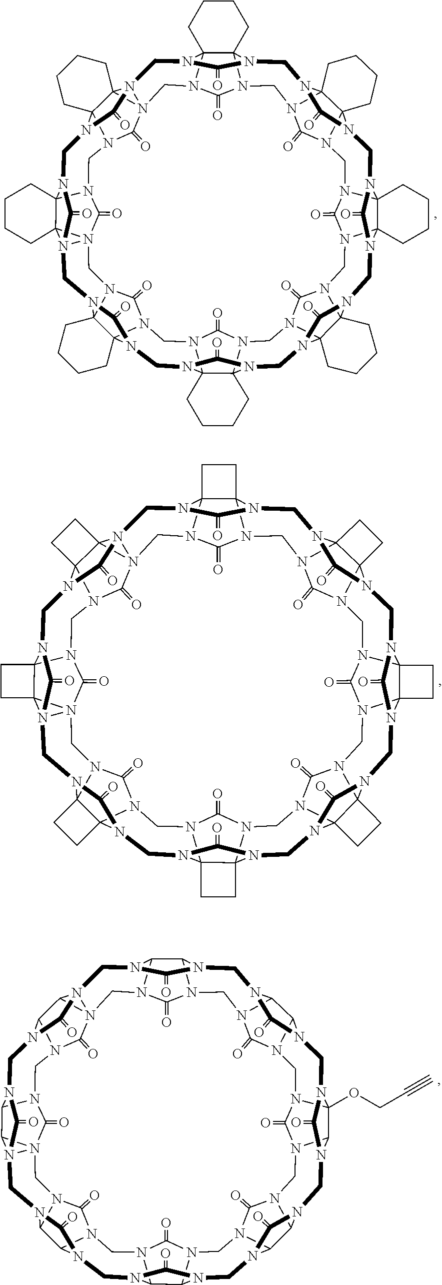

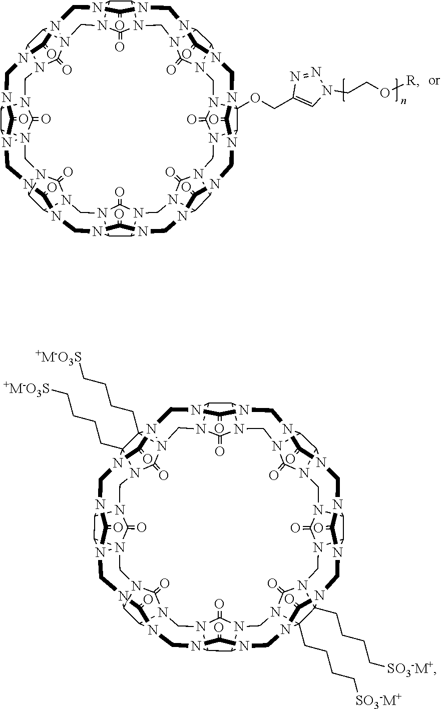

where n is 3 to 1000, including all values and ranges therebetween.
Statement 23. A composition according to any one of Statements 20-22, where the one or more compounds have the following structure:

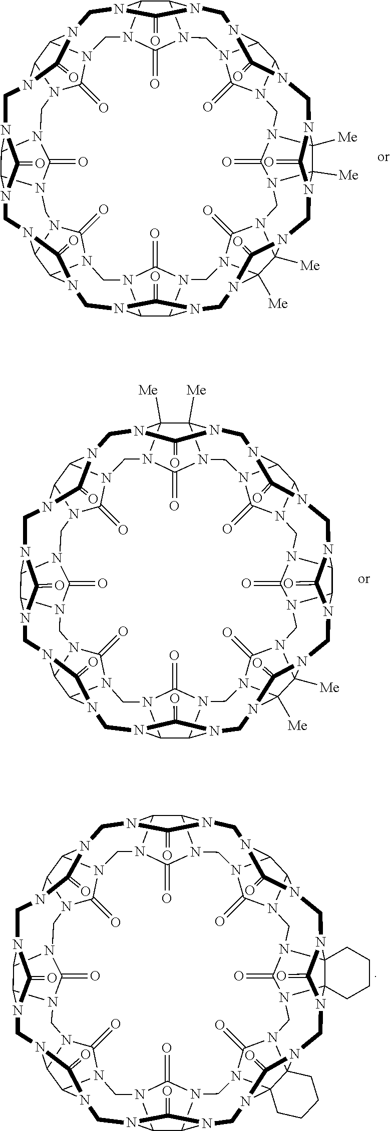

Statement 24. A composition according to any one of Statements 20-23, where the composition is suitable for use in a method according to any one of Statements 1-19.

The following example is presented to illustrate the present disclosure. It is not intended to be limiting in any matter.

### EXAMPLE

This example provides a description compounds and methods of the present disclosure.

Described herein is the use of cucurbit[8]uril (CB[8]) macrocycles as an antidote to counteract in vivo biological effects of phencyclidine. The binding of CB[8] and its derivative Me₄CB[8] was investigated toward ten drugs of abuse (3-9, 12-14) by a combination of ¹H NMR spectroscopy and isothermal titration calorimetry in phosphate buffered water. It was found that the cavity of CB[8] and Me₄CB[8] are able to encapsulate the 1-amino-1-aryl-cyclohexane ring system of phencyclidine (PCP) and ketamine, as well as the morphinan skeleton of morphine and hydromorphone with K_{d} values ≤50 nM. In vitro cytotoxicity (MTS metabolic and adenylate kinase cell death assays in HEK293 and HEPG2 cells) and in vivo maximum tolerated dose studies (Swiss Webster mice) which were performed for Me₄CB[8] indicated good tolerability. The tightest host•guest pair (Me₄CB[8]•PCP; K_{d}=2 nM) was advanced to in vivo efficacy studies. The results of open field tests demonstrate that pretreatment of mice with Me₄CB[8] prevents subsequent hyperlocomotion induction by PCP and also that treatment of animals previously dosed with PCP with Me₄CB[8] significantly reduces the locomotion levels.

A water soluble derivative of macrocyclic CB[8] (Me₄CB[8]) displayed enhanced binding affinity toward sets of drugs that are less efficiently sequestered by the smaller CB[n] homologues CB[6], CB[7], and acyclic CB[n] M1 and M2. Described herein is the in vitro binding affinity of CB[8] and its water soluble derivative Me₄CB[8] toward a panel of drugs of abuse and demonstrate that Me₄CB[8] acts as an in vivo sequestration agent to reverse the hyperlocomotion observed in mice treated with PCP.

Results and Discussion

This results and discussion section is subdivided into sections as follows. First, the binding properties of CB[8] and Me₄CB[8] toward a panel of commonly used and abused drugs (FIG. 3) were determined by a combination of ¹H NMR spectroscopy and isothermal titration calorimetry (ITC). Subsequently, the results of in vitro cytotoxicity and in vivo maximum tolerated dose studies conducted for Me₄CB[8] are detailed. Finally, the ability of Me₄CB[8] to sequester PCP in vivo and thereby reduce the hyperlocomotion observed for mice that had been treated with PCP was demonstrated.

Host Selection. It was previously found that the water soluble hosts M1, M2, CB[7], SC4A, and HP-β-CD exhibit poor binding affinity Kₐ≈10²−1×10⁶ M⁻¹ toward several members of the drug panel (morphine, hydromorphone, ketamine, PCP, cocaine). The non-opioids cocaine, PCP, and ketamine cannot be reversed by naloxone and no other specific therapeutics are used clinically to rescue patients in overdose cases. As mentioned above, CB[8] possesses such poor water solubility (<10 μM) that it would not be possible to administer sufficient doses of CB[8] (e.g., limited by CB[8] concentration and maximum volume) to act as an in vivo sequestration agent toward drugs of abuse like PCP. Accordingly, CB[8] and the previously reported water-soluble host Me₄CB[8] (3.1 mM) were synthesized by the literature procedures.

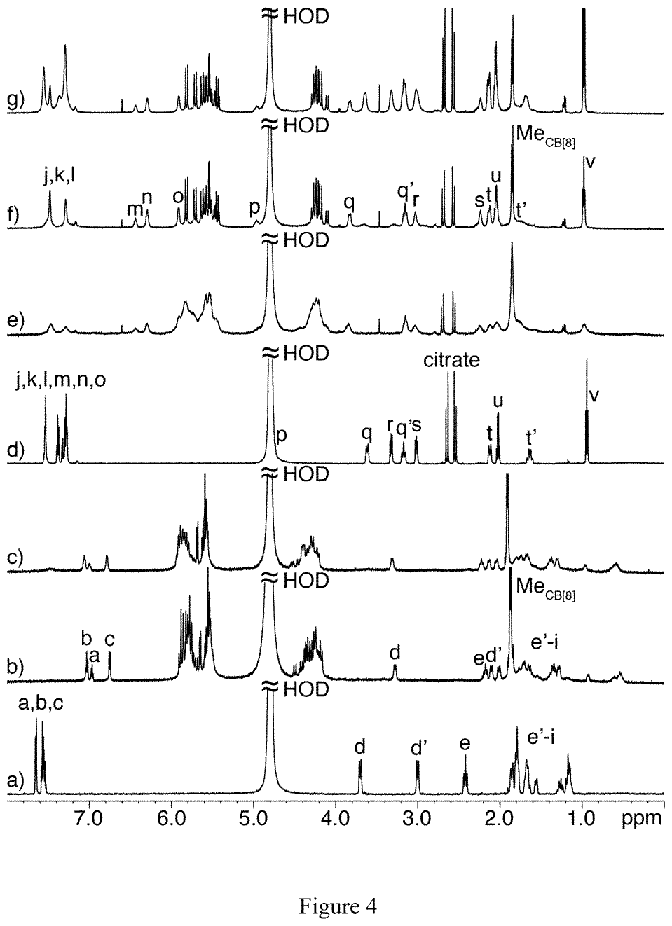

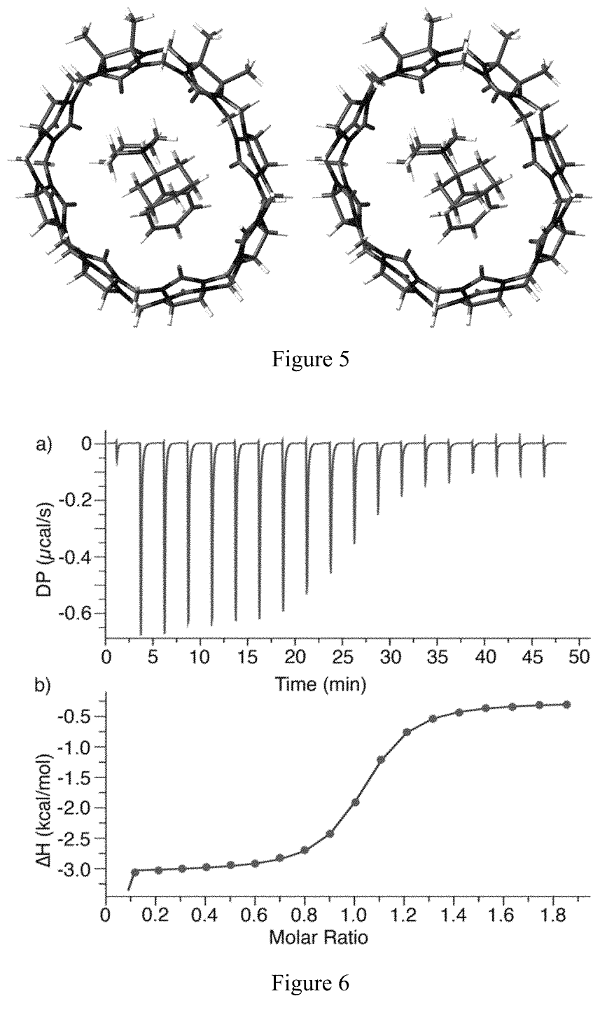

Qualitative ¹H NMR host guest recognition study. Initially, qualitative host•guest binding studies were performed between CB[8] and its water soluble derivative Me₄CB[8] and the drug panel (3-9, 12-14) by ¹H NMR spectroscopy to determine whether their cavities are large enough to bind drugs containing the morphinan ring system (5, 6, 14), the 1-aryl-1-amino cyclohexane moiety (7 and 8), and the aza-bicyclo[3.2.1]octane ring system of cocaine (9). For example, FIG. 4a-c shows the ¹H NMR spectra recorded for uncomplexed PCP (8) as well as 1:1 and 1:2 mixtures of Me₄CB[8] with 8. At a 1:1 ratio of Me₄CB[8]:8, significant complexation induced upfield shifting of the resonances was observed for the phenyl and piperidinium moieties of 8. These observed upfield shifts constitute good evidence for the inclusion of the phenyl and piperidinium moieties inside the cavity of Me₄CB[8] within the Me₄CB[8]•8 complex. In the spectrum recorded at 1:2 ratio of Me₄CB[8]:8 (FIG. 4c), the presence of separate but broadened resonances for free 8 indicates that the guest exchange process is in the slow to intermediate regime on the chemical shift timescale. Based on symmetry, the spectrum for unsubstituted CB[8]•8 displays two pairs of doublets for the diastereotopic CH2 groups on the top and bottom rim of CB[8] that become different in the complex. The inclusion of two 6-membered rings of 8 inside CB[8] and Me₄CB[8] and the slow to intermediate guest exchange rate indicated that high affinity binding of PCP was achievable. In contrast, CB[7] and acyclic CB[n]-type receptors M1 and M2 simply bind weakly to the aromatic residue of 8. FIG. 5 shows a cross-eyed stereoview of an MMFF minimized model of the Me₄CB[8]•8 complex which illustrates the simultaneous penetration of the phenyl and the piperidinium moieties into the cavity of Me₄CB[8] whereas the cyclohexyl ring remains at the ureidyl carbonyl portal of Me₄CB[8].

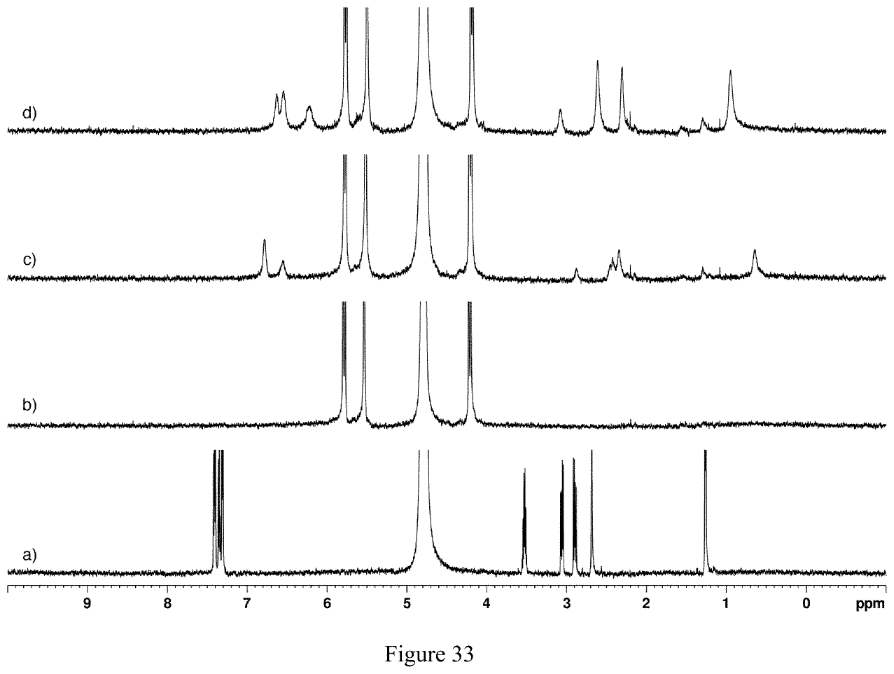

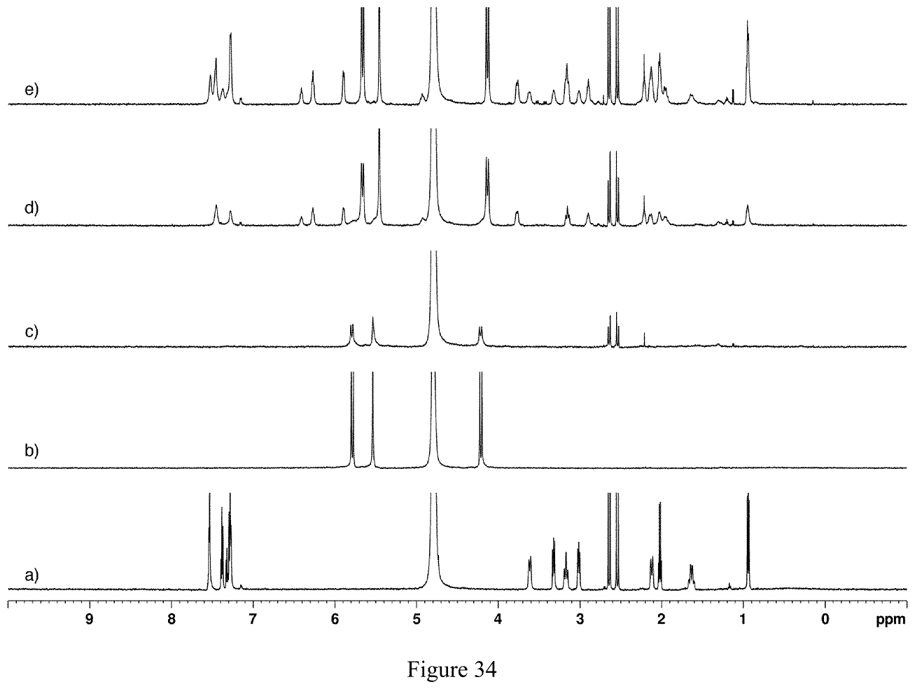

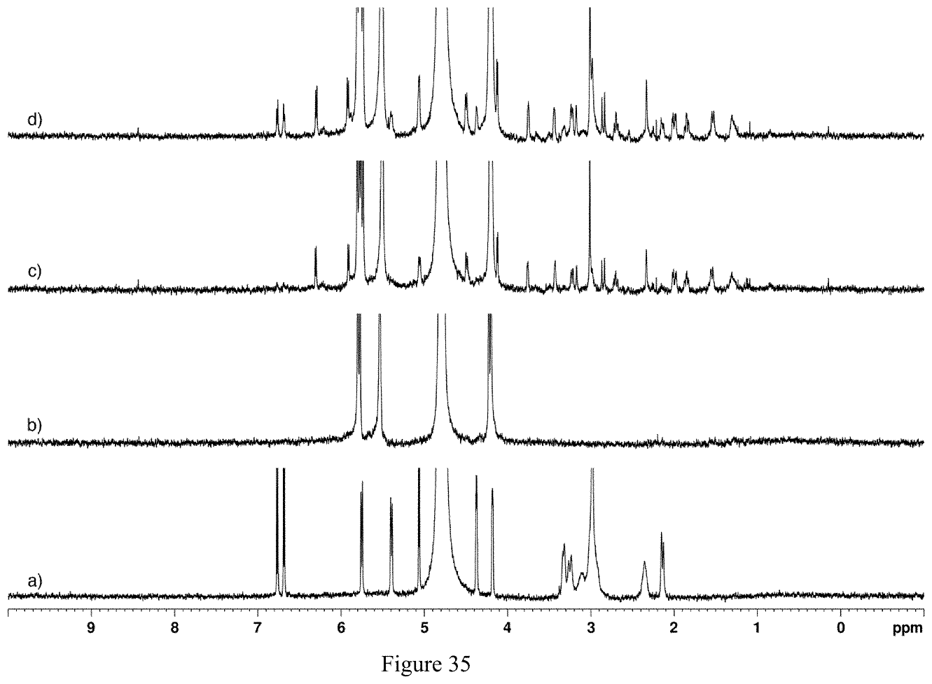

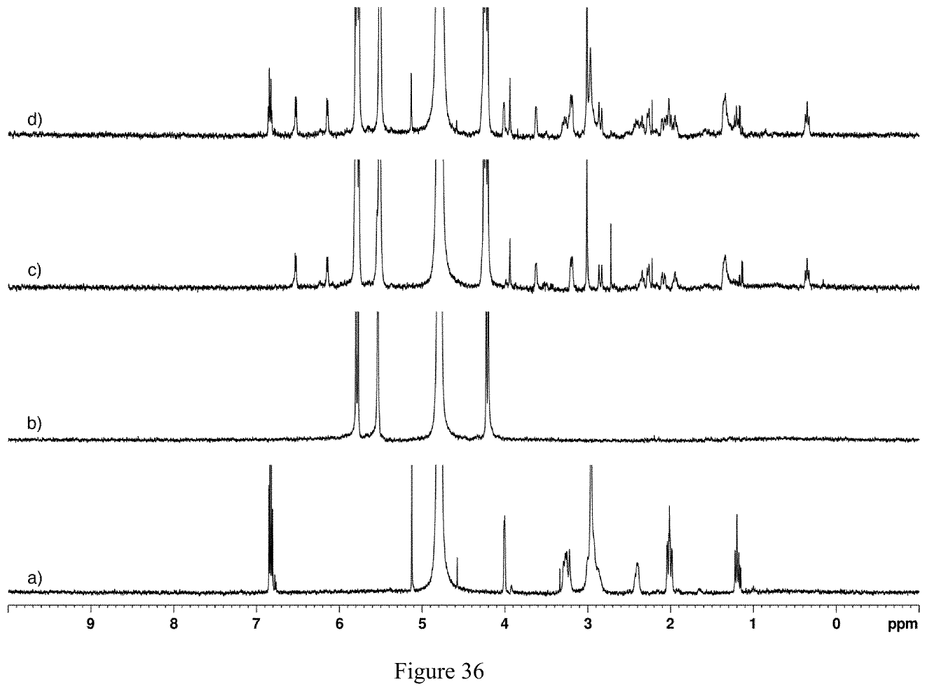

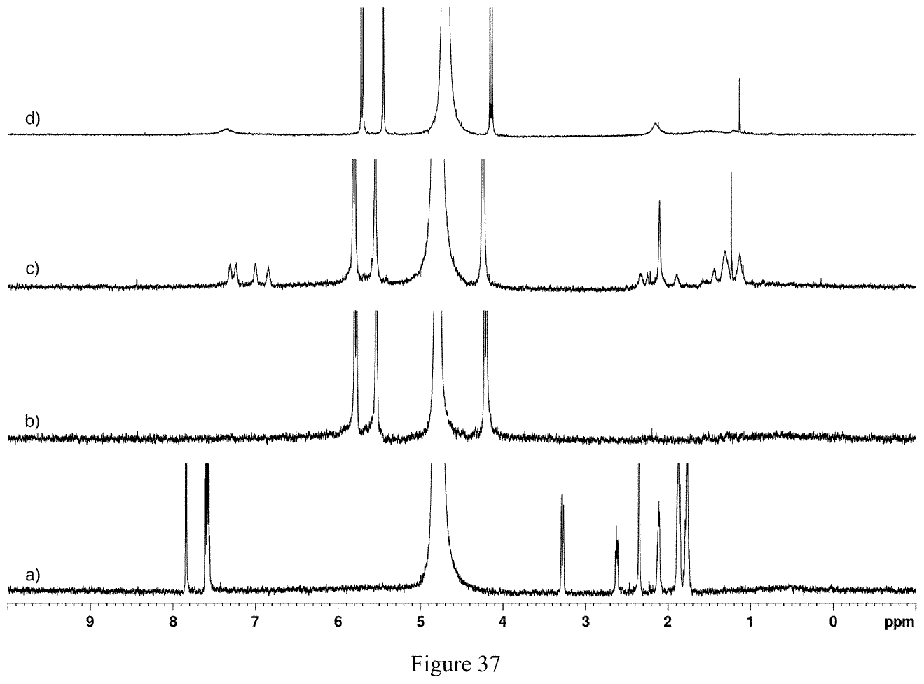

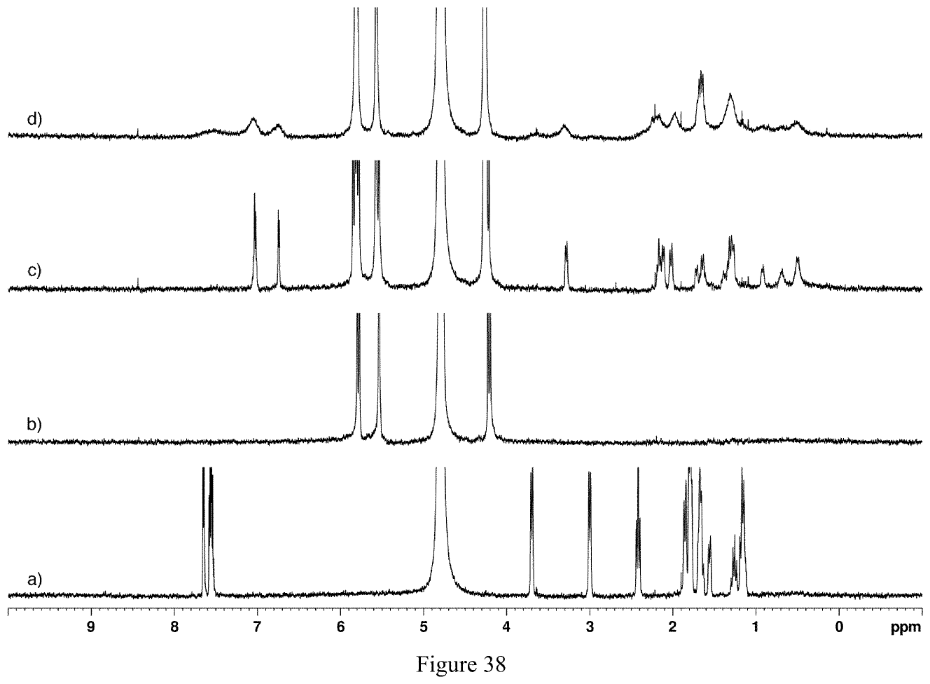

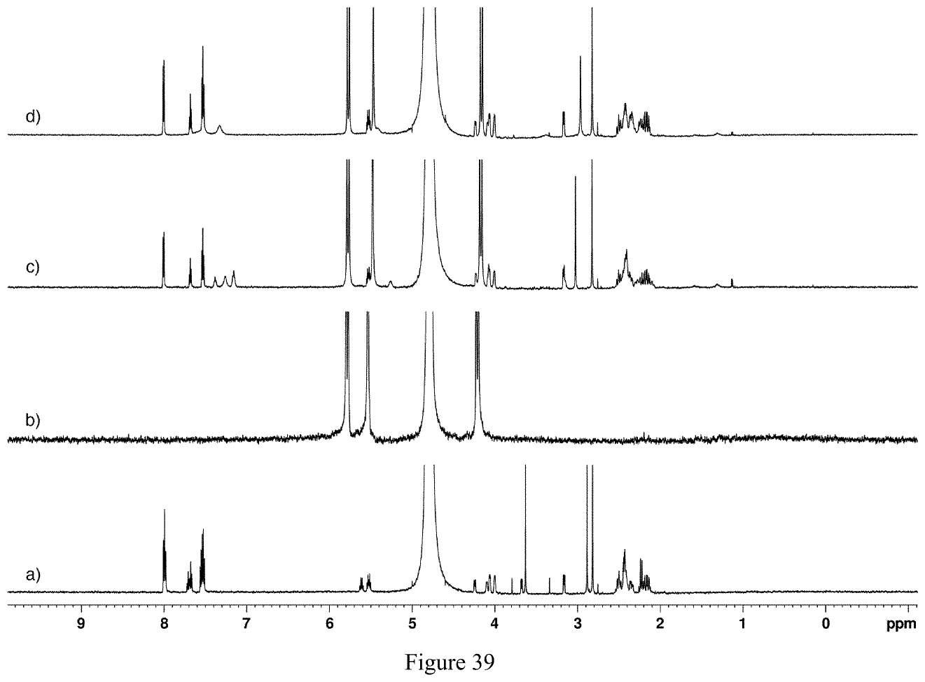

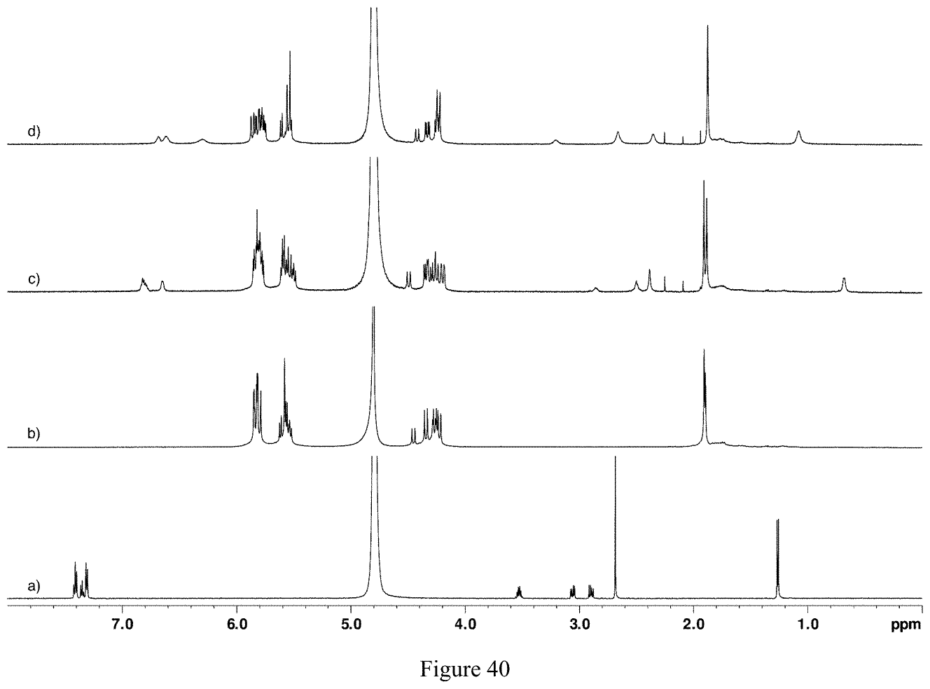

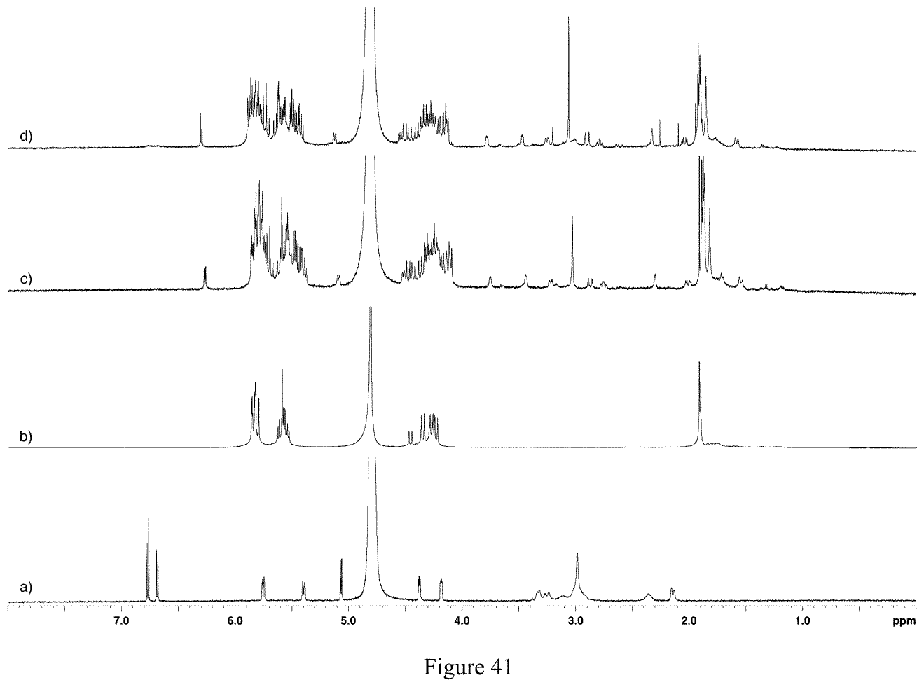

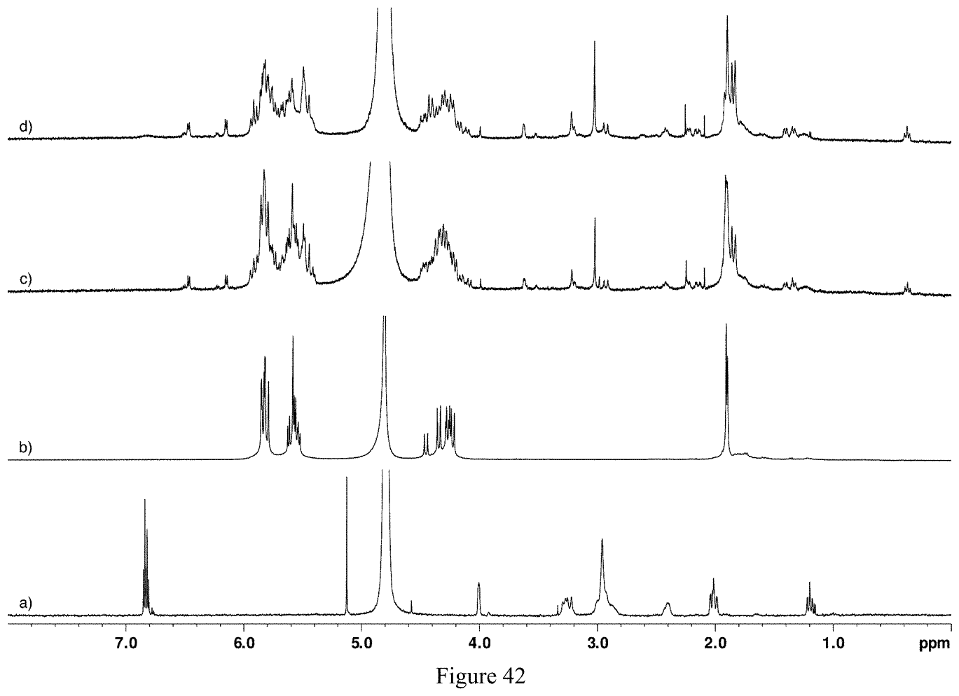

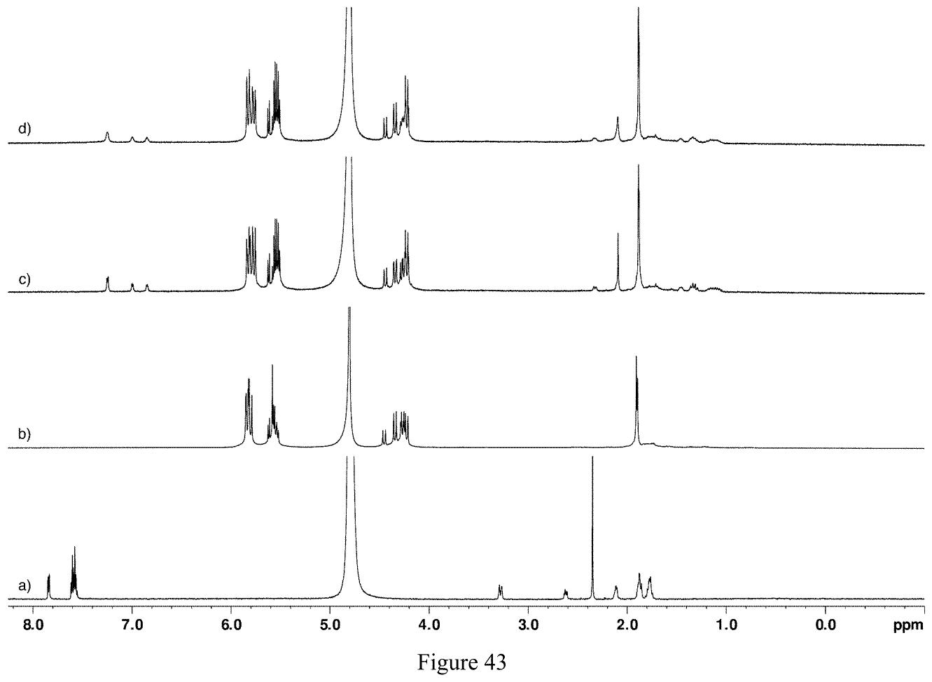

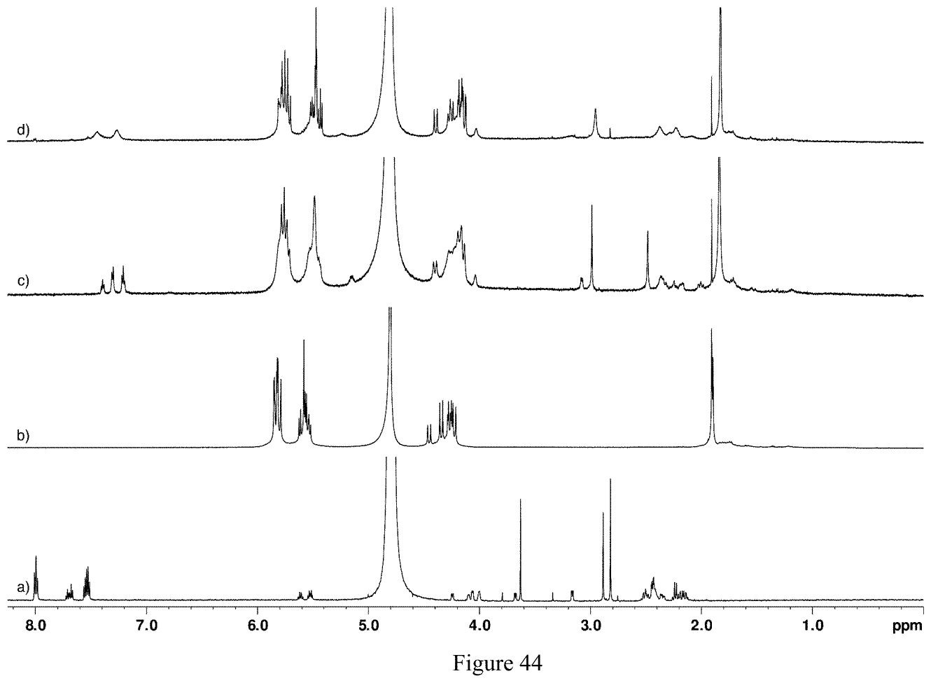

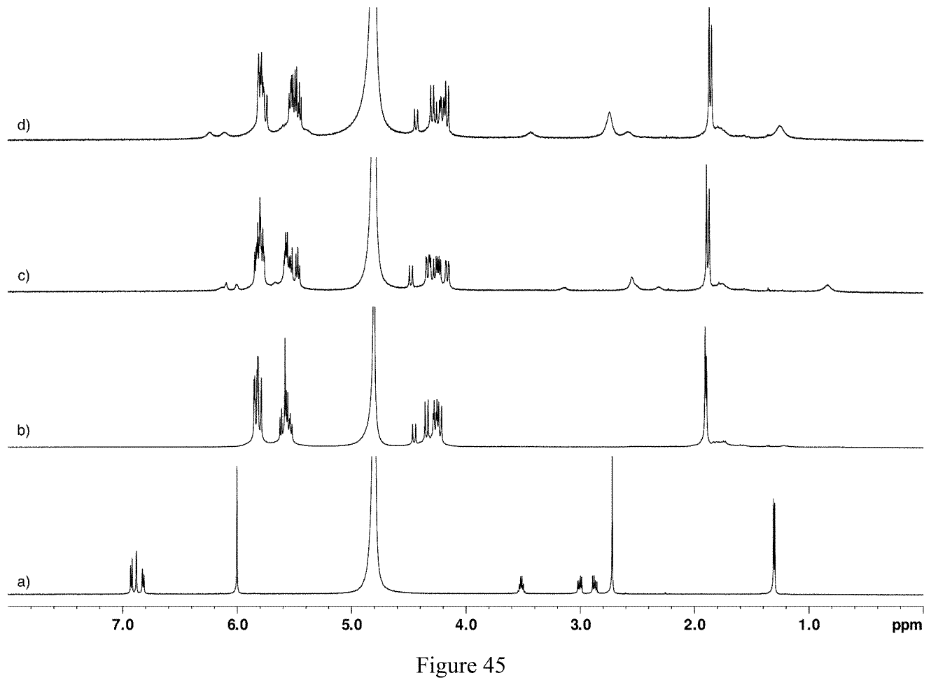

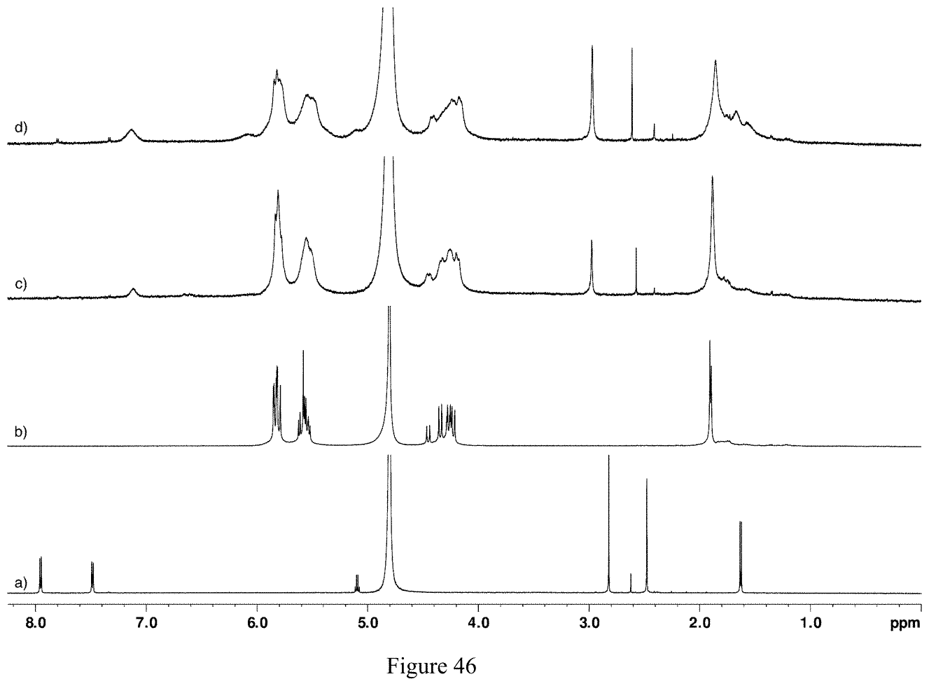

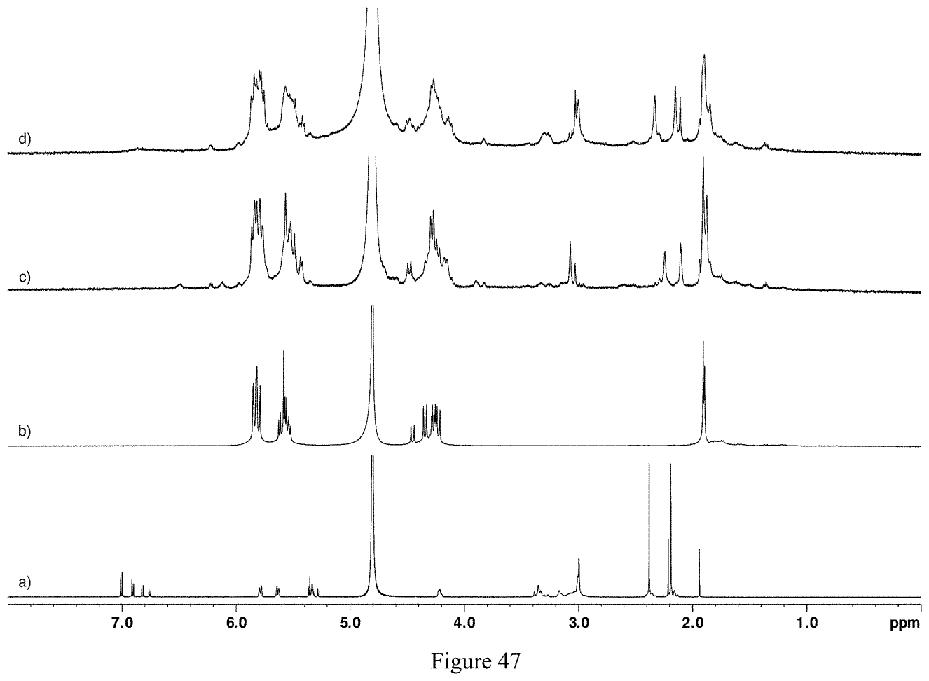

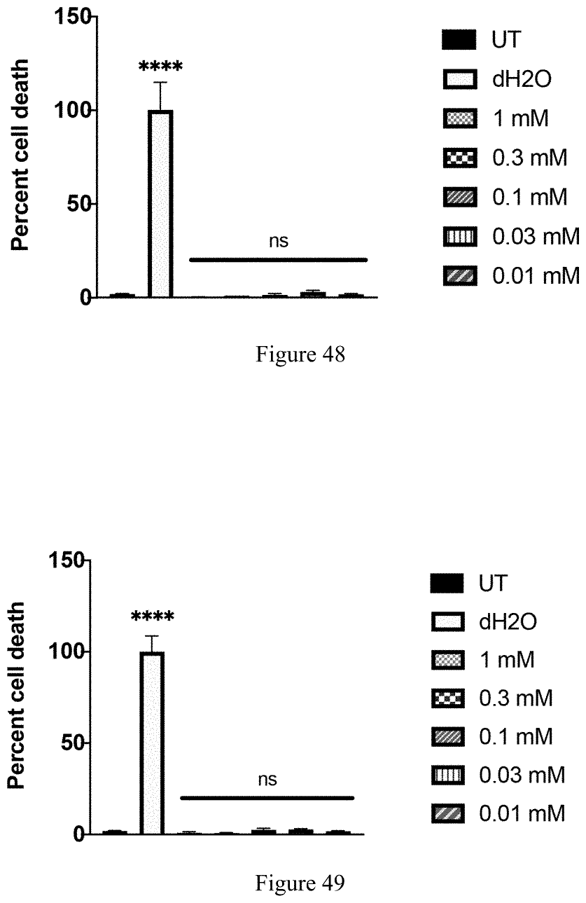

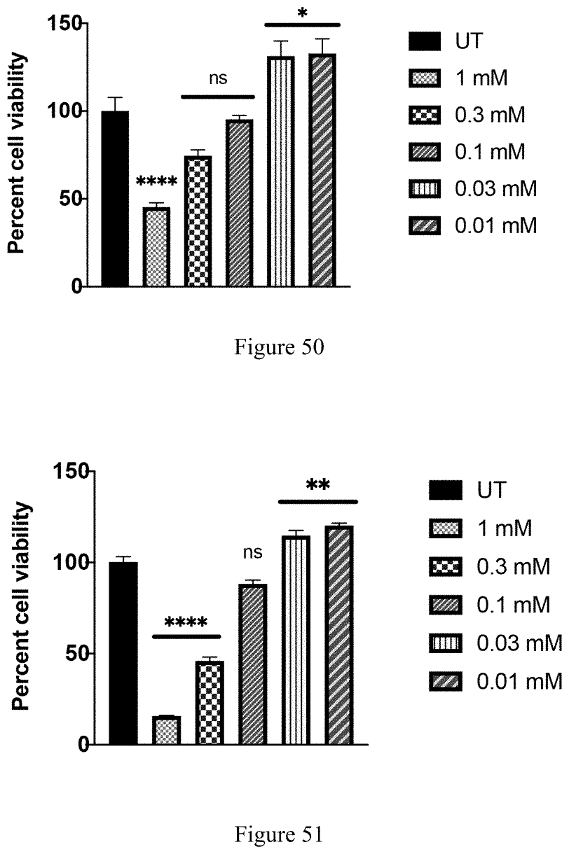

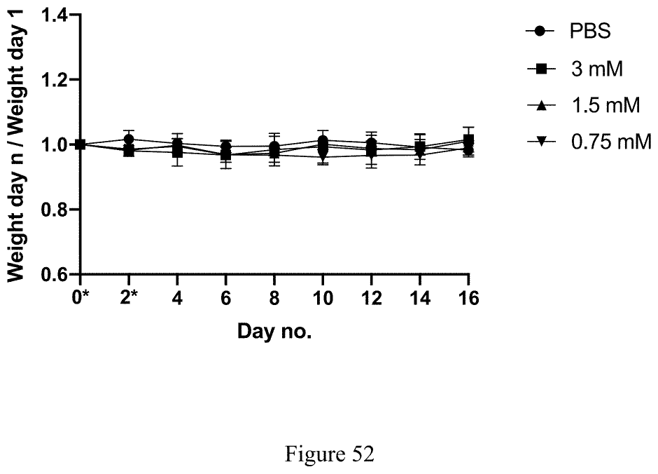

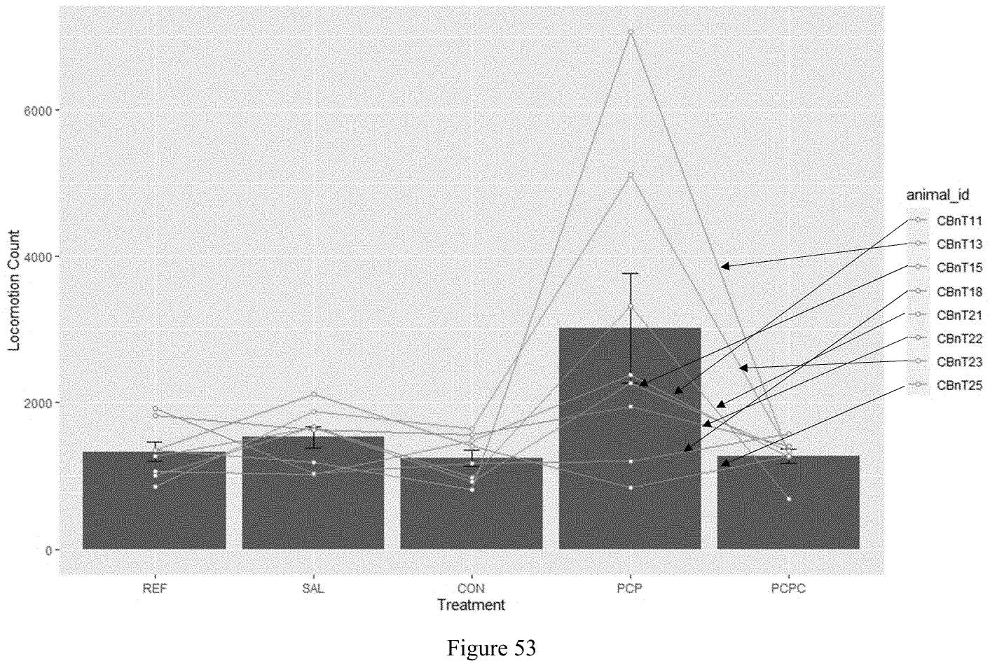

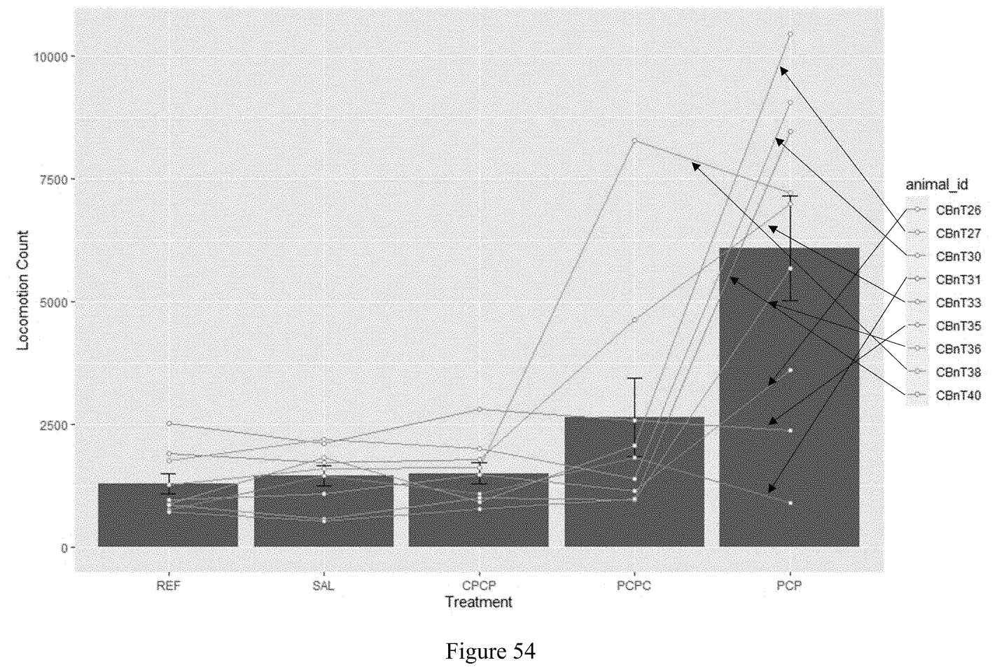

FIG. 4d-g shows the ¹H NMR spectra recorded for 4 (fentanyl) alone and 1:1, 1:2, and 1:4 mixtures of Me₄CB[8] and 4. At a 1:1 ratio of Me₄CB[8]:4 the resonances for most protons of 4 become broadened indicating complexation is occurring and that guest exchange is in the intermediate exchange regime on the chemical shift timescale. At a 1:2 ratio of Me₄CB[8]:4 (FIG. 4f), a single set of sharp resonances is observed for guest 4 which is consistent with the formation of a discrete Me₄CB[8]•4₂ complex. The three aryl resonances for the phenethylammonium ion moiety of 4 are shifted about 1 ppm upfield upon complexation whereas the resonances for the (C=O)NPh moiety do not shift. The observation of a single pair of diastereotopic CH₂ resonances for the CB[8] unit within the CB[8]•4₂ complex dictates a head-to-tail orientation of the cavity bound phenethylammonium ion groups which presumably exhibit π-π stacking interactions. The ability of CB[8] to promote homo and hetero-ternary complexation of aromatic resonances is well precedented. Finally, at a 1:4 ratio of Me₄CB[8]:4, separate resonances for complexed and uncomplexed guest 4 was observed, which indicates slow kinetics of guest exchange on the ¹H NMR timescale, which is typically observed for tight complexes. Related ¹H NMR experiments were performed for the remaining guests (3, 5, 6, 7, 9 and 12-14) with CB[8] and Me₄CB[8] and are presented in FIGS. 33-47. Complexes of CB[8] and Me₄CB[8] with 5 and 6 displayed upfield shifting of most of the protons of the morphinan ring system, which indicates that they fit in the cavity of CB[8] and Me₄CB[8]; they also displayed slow kinetics of guest exchange, which suggests strong binding. Similarly, the ¹H NMR spectrum of CB[8] and Me₄CB[8] with ketamine 7 displays upfield shifting for both the aromatic and cyclohexyl resonances of 7 which indicates the 1-aryl-1-amino cyclohexyl moiety is fully bound inside CB[8] and Me₄CB[8] (Supporting Information). Lastly, the ¹H NMR spectra of CB[8] and Me₄CB[8] with cocaine 9 shows small upfield shifts for the benzoyl group and little shifting for the remaining protons which shows that the benzoyl group is preferentially bound by CB[8].

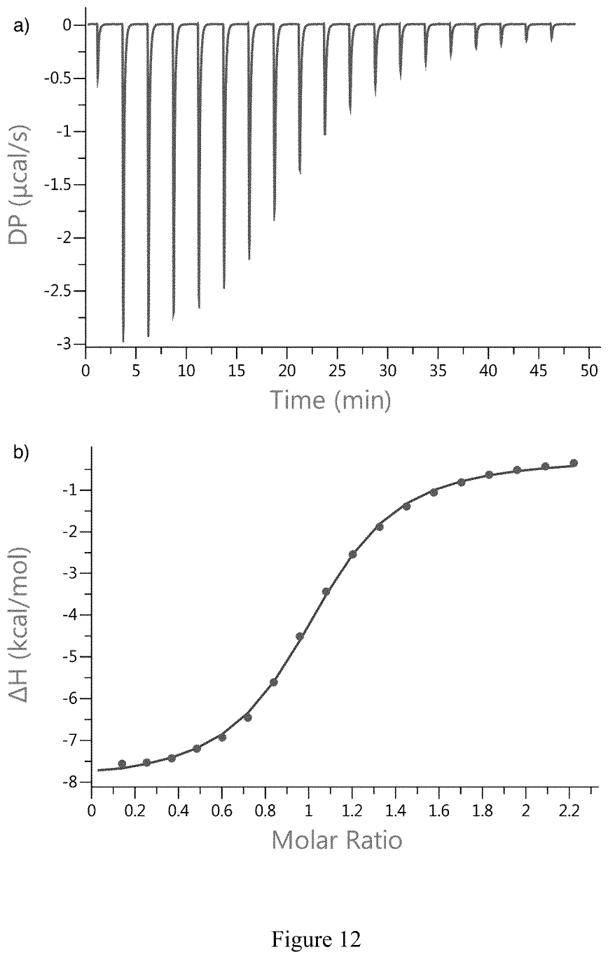

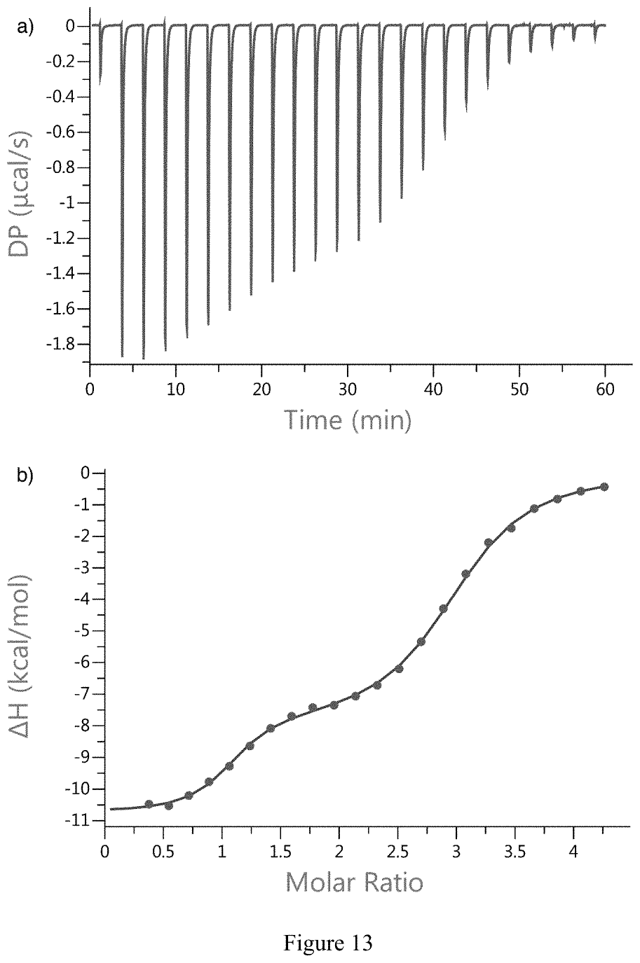

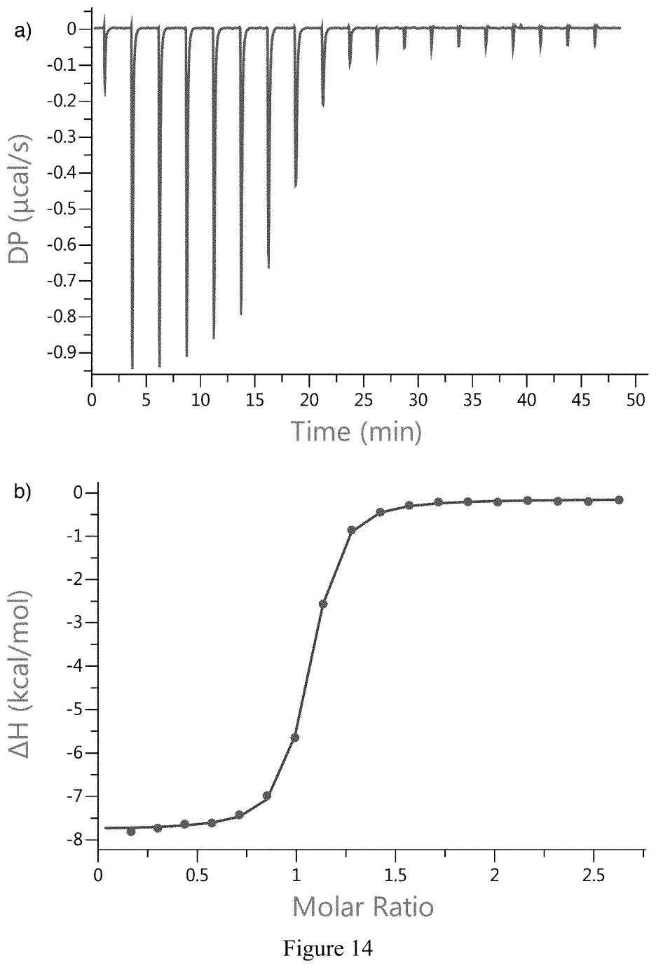

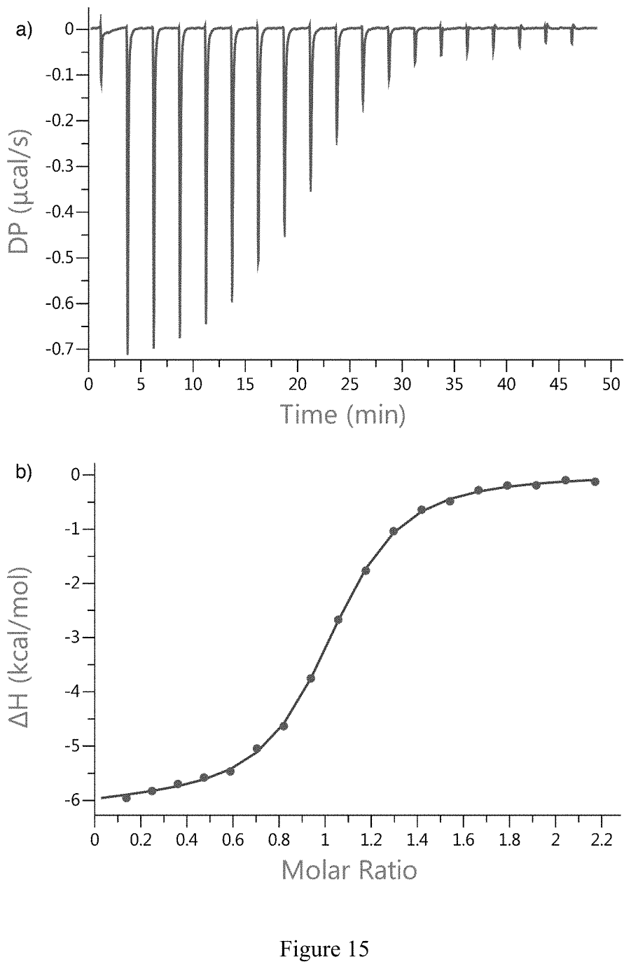

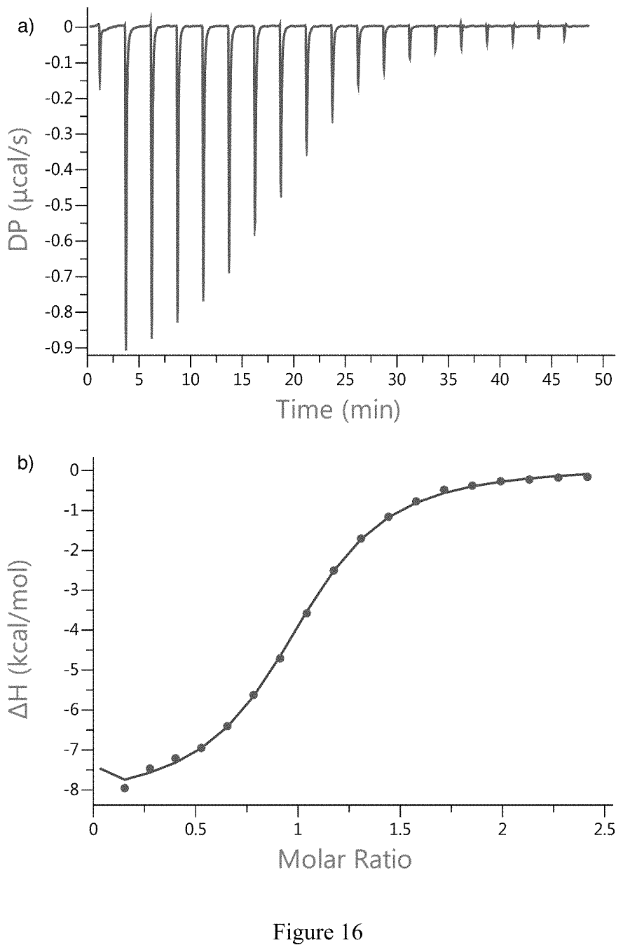

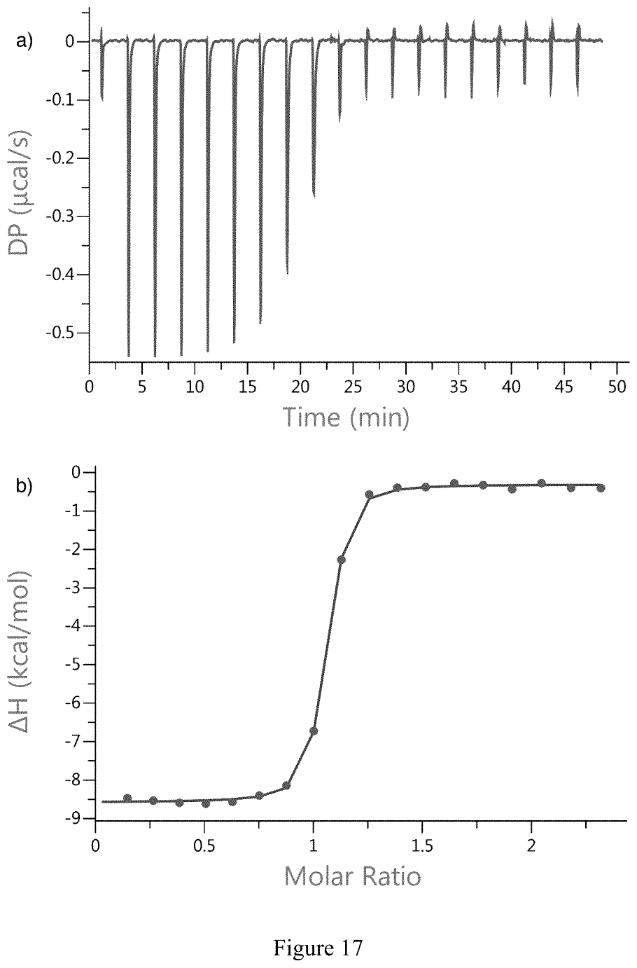

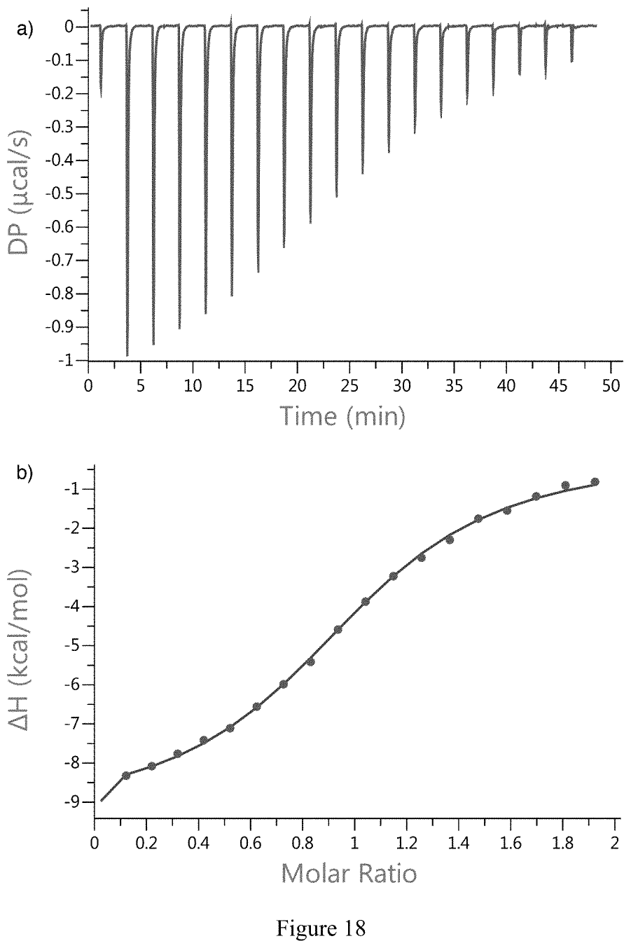

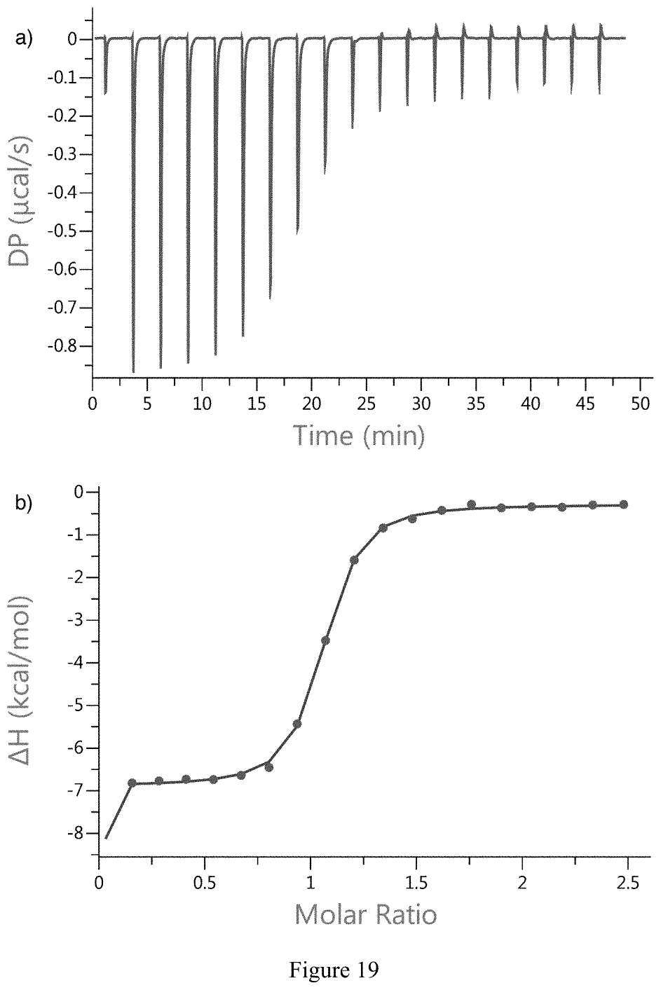

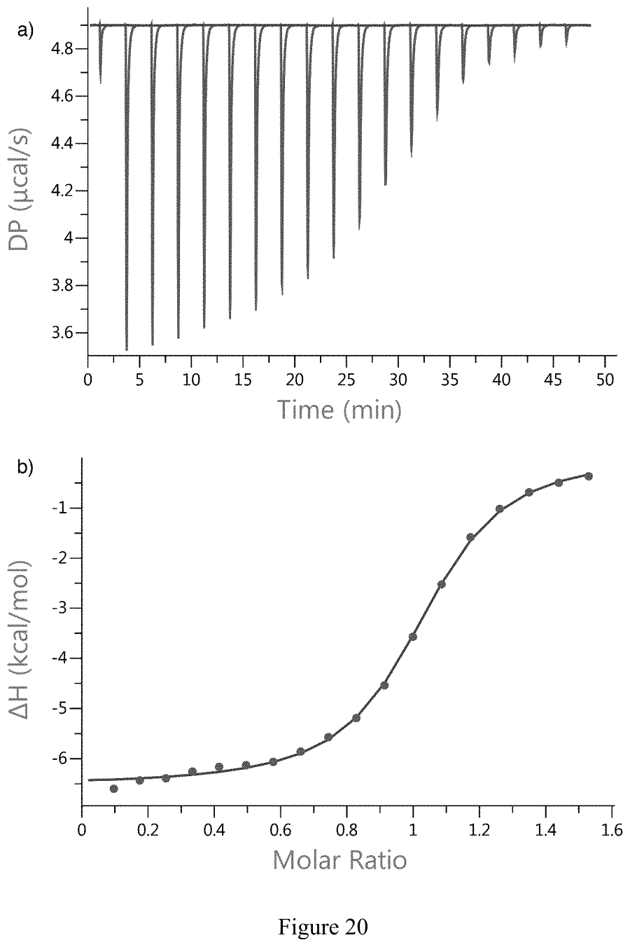

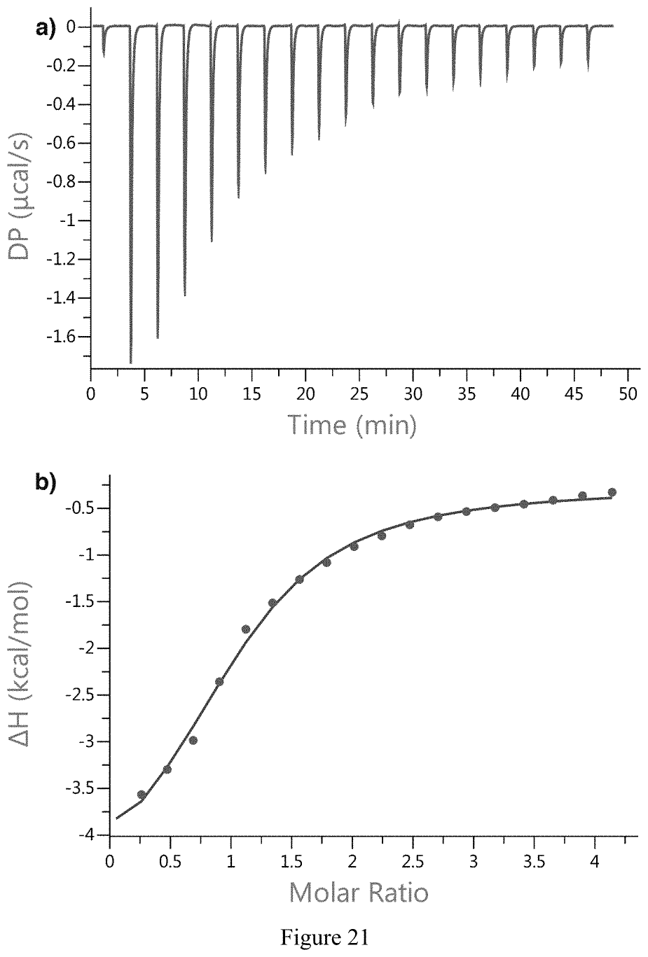

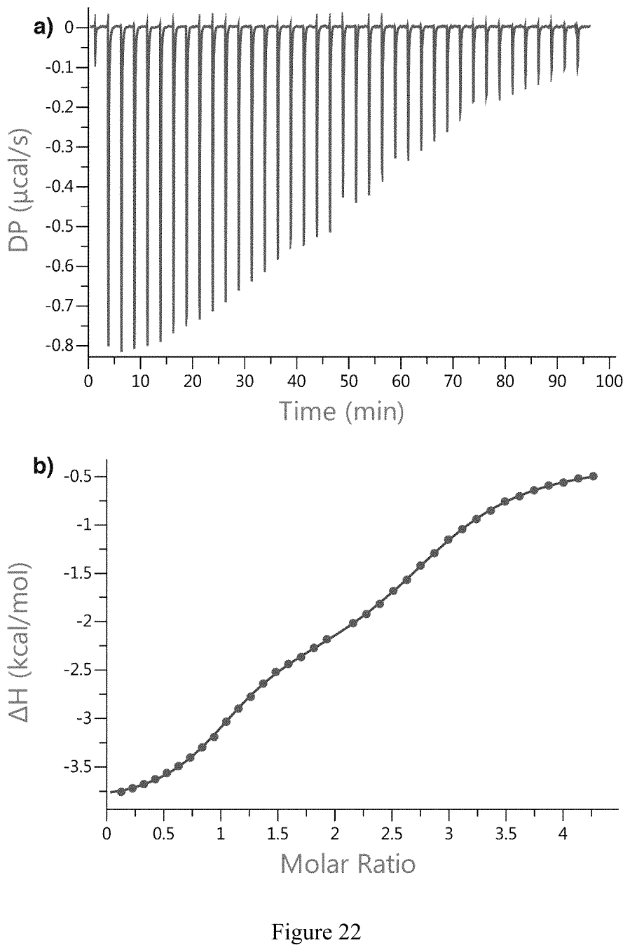

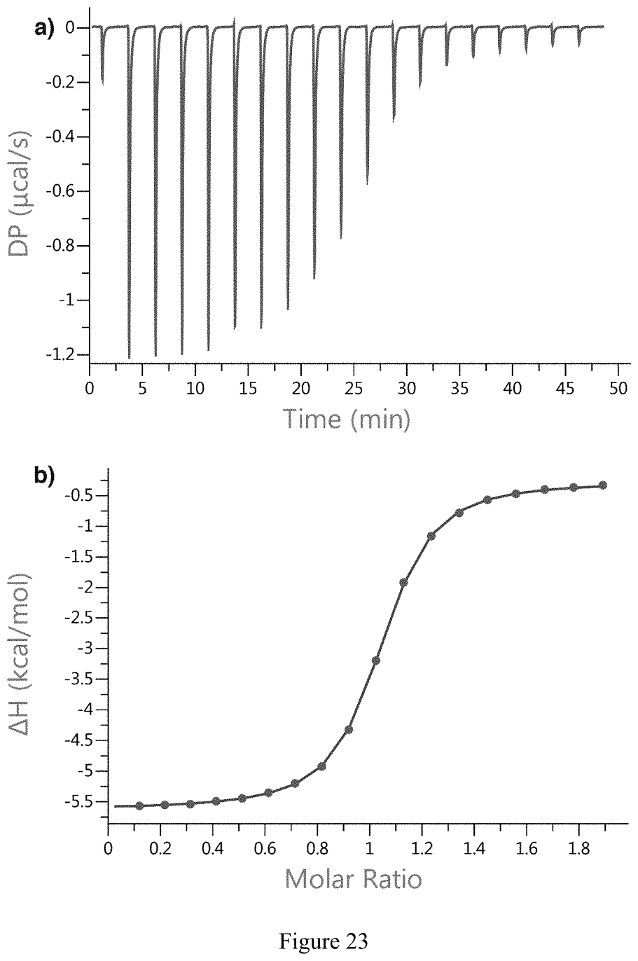

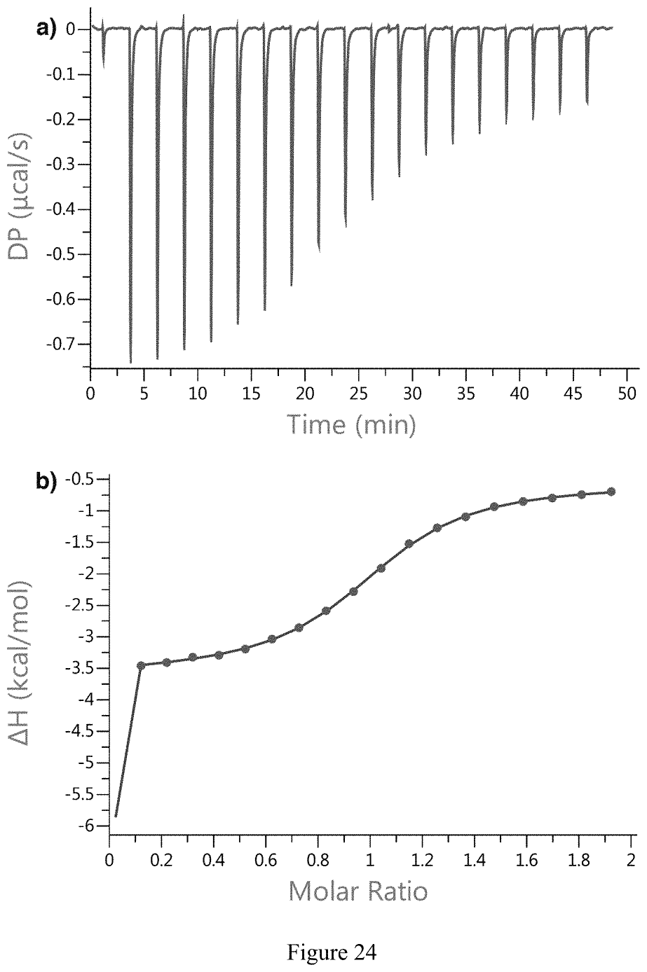

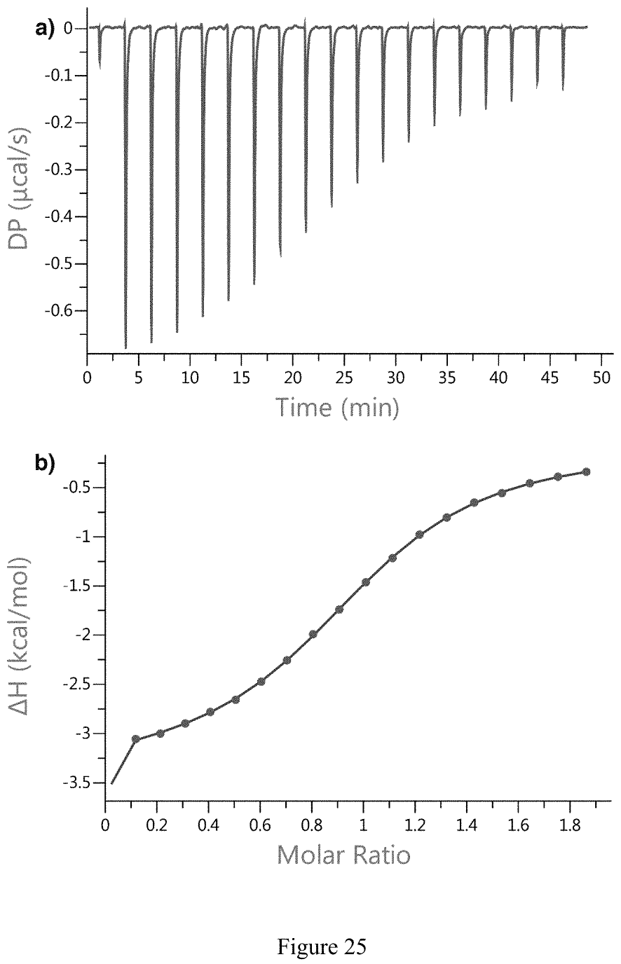

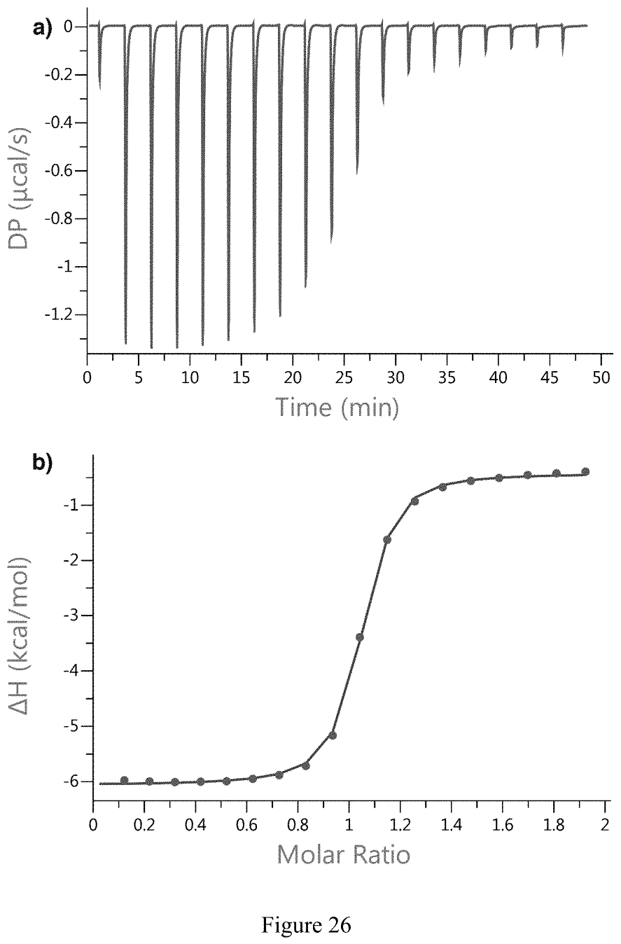

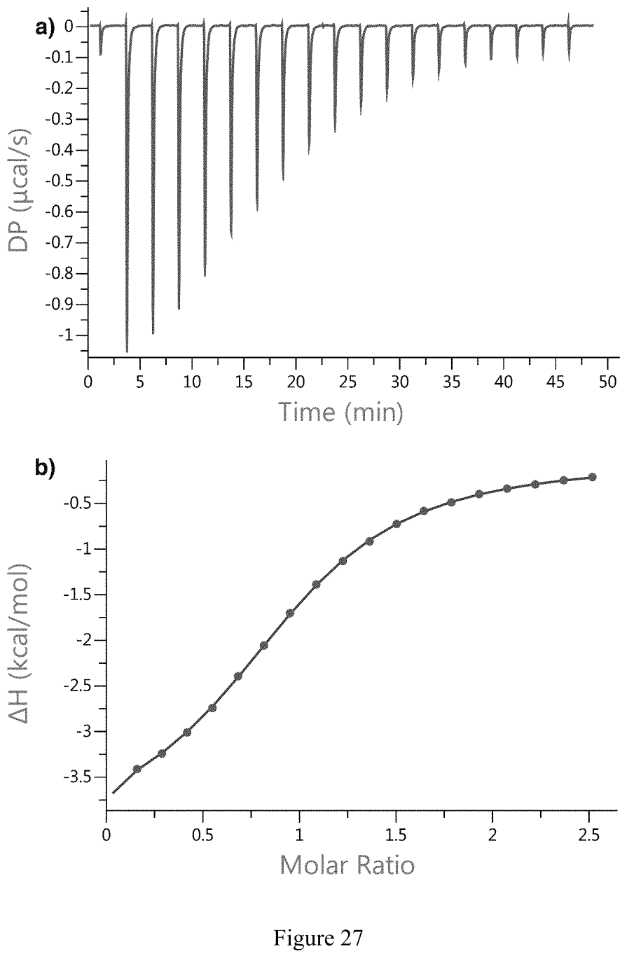

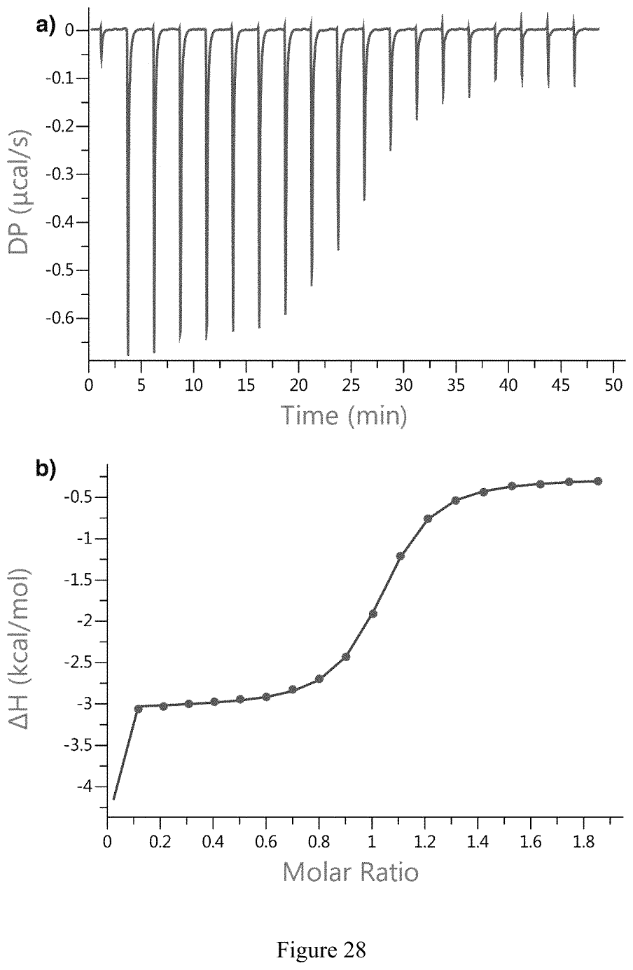

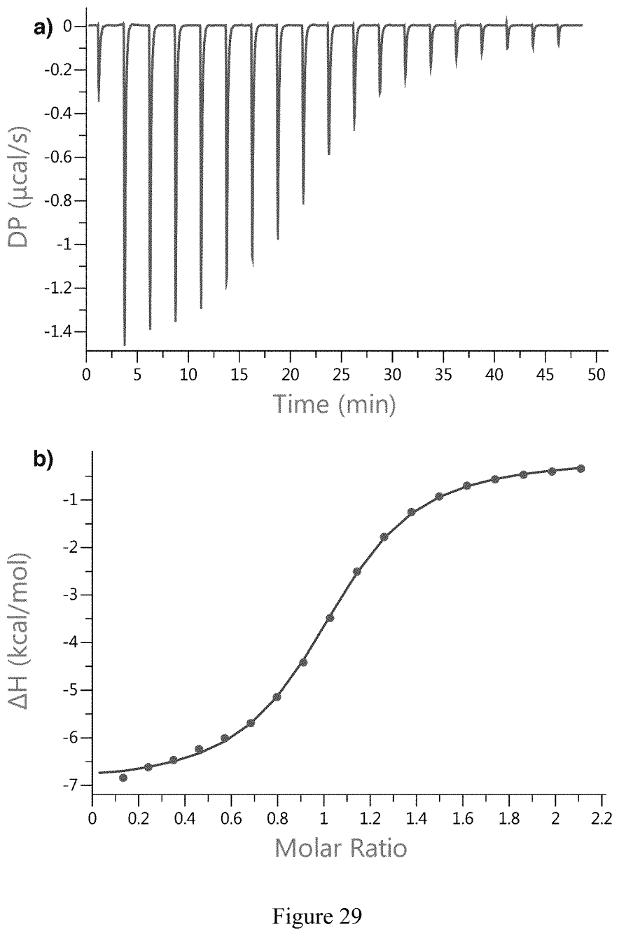

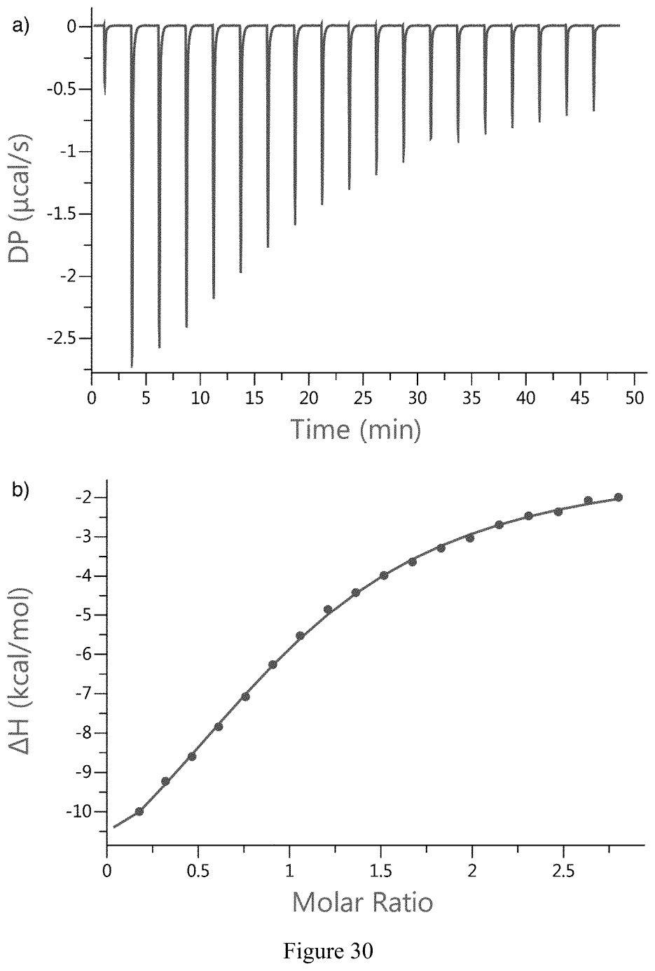

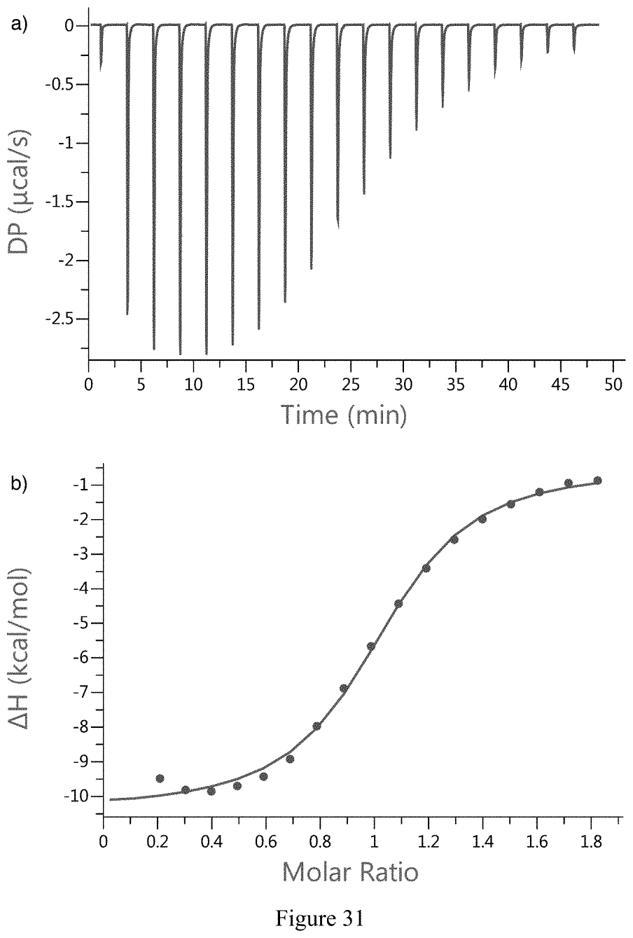

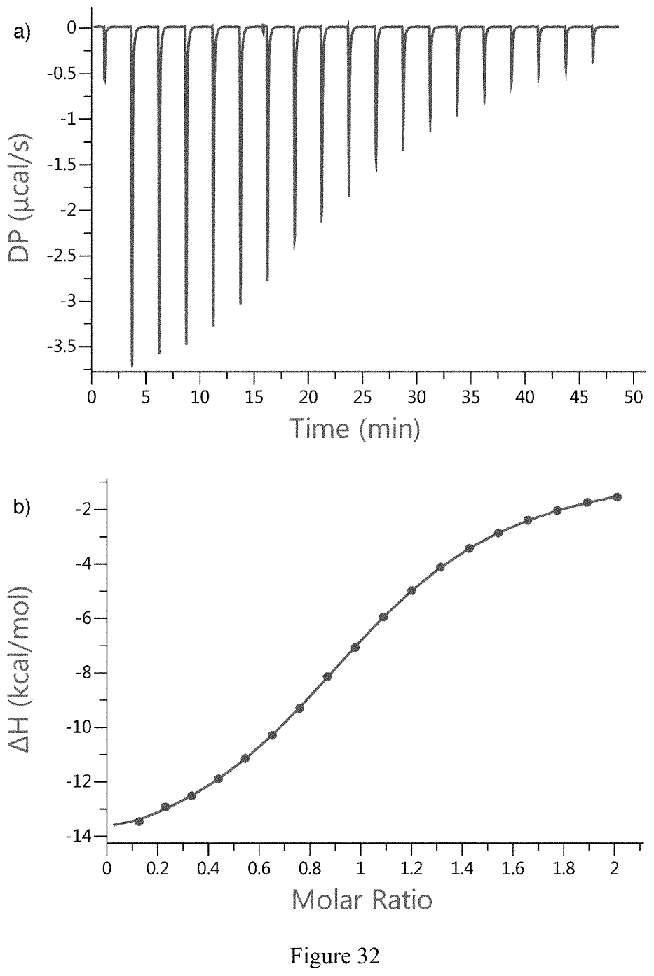

Measurement of the thermodynamic parameters of complex formation by ITC. Based on these qualitative results and the likelihood of tight binding complexes, the thermodynamic parameters of binding were measured by ITC (see FIGS. 12-32). ITC provides data such as Kₐ (M⁻¹), ΔH (kcal mol⁻¹), and stoichiometry of binding and has been previously used to study the complexation of guests 3-9 with CB[7] and acyclic CB[n]-type receptors M1 and M2. This data set provided a comparison to evaluate the potential of CB[8] and Me₄CB[8] as sequestering agents for drugs 3-9 and 12-14. Direct ITC titrations of 3, 4, 9, and 12-14 in the syringe into solutions of either CB[8] or Me₄CB[8] in the sample cell was performed and the resulting thermodynamic parameters are presented in Table 1. The Kₐ values for these complexes range from Kₐ=(2.98±0.47)×10⁴ M⁻¹ for Me₄CB[8]•3 to Kₐ=(1.9±0.09)×10⁷ M⁻¹ for CB[8]•4. The ITC results for guest 4 with CB[8] and Me₄CB[8] confirmed the overall 1:2 stoichiometry observed by NMR and showed a negative cooperativity in the formation of the ternary complexes. The remaining drugs displayed binding constants that exceed the range that can be measured accurately by direct titrations and therefore required competition ITC experiments. In competition ITC experiments, a solution of host and an excess of a weaker binding guest of known Kₐ and ΔH in the sample cell are titrated with a solution of the tighter binding guest in the syringe and the data is subsequently fit to a competition binding model by the PEAQ ITC data analysis software to extract the Kₐ and ΔH values of the tighter binding complex. As weaker binding competitors, we selected cycloalkylammonium ions 10 and 11 and first measured their Kₐ and ΔH values toward CB[8] and Me₄CB[8] by direct ITC titrations (Table 1). Subsequently, 10 and 11 were used as competitors to measure the thermodynamic parameters for the complexes between hosts CB[8] and Me₄CB[8] with drugs 5-8 by competition ITC measurements (Table 1). For example, FIG. 6a shows the thermogram recorded during the titration of a solution of Me₄CB[8] (0.104 mM) and 11 (0.2 mM) in the cell with 8 (1.0 mM) in the syringe. FIG. 6b shows the fitting of the integrated heat values to a competitive binding model to give the Kₐ=(5.35±0.19)×10⁸ M⁻¹ and ΔH=−8.39±0.01 kcal mol⁻¹ values for the Me₄CB[8]•8. The complexes are uniformly driven by favorable enthalpic contributions to free energy as expected based on the release of high energy water molecules from the cavity of CB[8] and Me₄CB[8] upon complexation. The measured values of Kₐ for CB[8] are consistently larger than those measured for Me₄CB[8] by factors of 2.3-fold for cocaine 9 to 39.3-fold for PCP (8). This effect is attributed to the ellipsoidal deformation previously observed for Me₄CB[8] by x-ray crystallography. Morphine 5 and hydromorphone 6 bind with high affinity (Kₐ>10⁷ M⁻¹), whereas the bulky diacetylated heroin 14 displays relative weak binding (Kₐ=7.94×10⁴ M⁻¹). Me₄CB[8] displays comparable affinity toward meth 4 and its methylenedioxy analog MDMA 12 which probably reflects counterbalancing effects of the larger but more hydrophilic ring system of MDMA 12. Conversely, mephedrone with its larger and more hydrophobic tolyl moiety binds 10-fold more tightly to Me₄CB[8] than meth 3 does. Very high binding constants were displayed by PCP and ketamine toward CB[8] and Me₄CB[8], which suggests they may function as in vivo sequestration agents for these drugs. We selected the Me₄CB[8] host and PCP (8) drug pair for advancement toward in vivo studies.

TABLE 1
Thermodynamic parameters (Kₐ (M⁻¹), ΔH° (kcal mol⁻¹)
determined for the complexes of CB[8] and Me₄CB[8] with
3-14 by ITC. Conditions: 20 mM NaH₂PO₄ buffer (pH = 7.4), 298K.

| | Kₐ (CB[8]•G) (M⁻¹) | Kₐ (Me₄CB[8]•G) (M⁻¹) | |
|---|---|---|---|
| Guest (G) | ΔH° (kcal/mol) | ΔH° (kcal/mol) | n |
| Meth 3^{[a]} | (1.47 ± 0.09) × 10⁵ | (2.98 ± 0.47) × 10⁴ | 1 |
| | (−7.84 ± 0.1) | (−4.61 ± 0.04) | |
| Fentanyl 4^{[a]} | (1.9 ± 0.09) × 10⁷ | (1.98 ± 0.02) × 10⁶ | 1 |
| | (−10.8 ± 0.06) | (−3.58 ± 0.01) | |
| | (3.7 ± 0.04) × 10⁵ | (9.52 ± 0.03) × 10⁴ | 2 |
| | (−7.67 ± 0.04) | (−1.94 ± 0.01) | |
| Morphine 5^{[b]} | (3.41 ± 0.15) × 10⁸ | (4.67 ± 0.17) × 10⁷ | 1 |
| | (−13.6 ± 0.04) | (−8.24 ± 0.02) | |
| Hydromorphone 6^{[b]} | (1.7 ± 0.11) × 10⁸ | (2.14 ± 0.05) × 10⁷ | 1 |
| | (−15.8 ± 0.1) | (−8.43 ± 0.02) | |
| Ketamine 7^{[c]} | (1.09 ± 0.07) × 10⁹ | (2.81 ± 0.67) × 10⁷ | 1 |
| | (−17.3 ± 0.16) | (−9.37 ± 0.02) | |
| PCP 8^{[c]} | (2.1 ± 0.2) × 10¹⁰ | (5.35 ± 0.19) × 10⁸ | 1 |
| | (−14.9 ± 0.04) | (−8.39 ± 0.01) | |
| Cocaine 9^{[a]} | (6.45 ± 0.43) × 10⁵ | (2.77 ± 0.15) × 10⁵ | 1 |
| | (−8.26 ± 0.15) | (−6.90 ± 0.07) | |
| 10^{[a]} | (5.3 ± 0.23) × 10⁶ | (1.04 ± 0.02) × 10⁶ | 1 |
| | (−7.62 ± 0.03) | (−5.35 ± 0.01) | |
| 11^{[a]} | (3.2 ± 0.26) × 10⁷ | (2.79 ± 0.17) × 10⁶ | 1 |
| | (−8.26 ± 0.04) | (−5.63 ± 0.03) | |
| MDMA 12^{[a]} | n.d. | (3.13 ± 0.32) × 10⁴ | 1 |
| | | (−13.8 ± 0.77) | |
| Mephedrone 13^{[a]} | n.d. | (3.07 ± 0.39) × 10⁵ | 1 |
| | | (−9.89 ± 0.29) | |
| Heroin 14^{[a]} | n.d. | (7.94 ± 0.07) × 10⁴ | 1 |
| | | (−15.1 ± 0.10) | |

Measured by ^{[a]}direct ITC titration, ^{[b]}competition ITC titration with 10, ^{[c]}competition ITC titration with 11, n.d. = not determined.

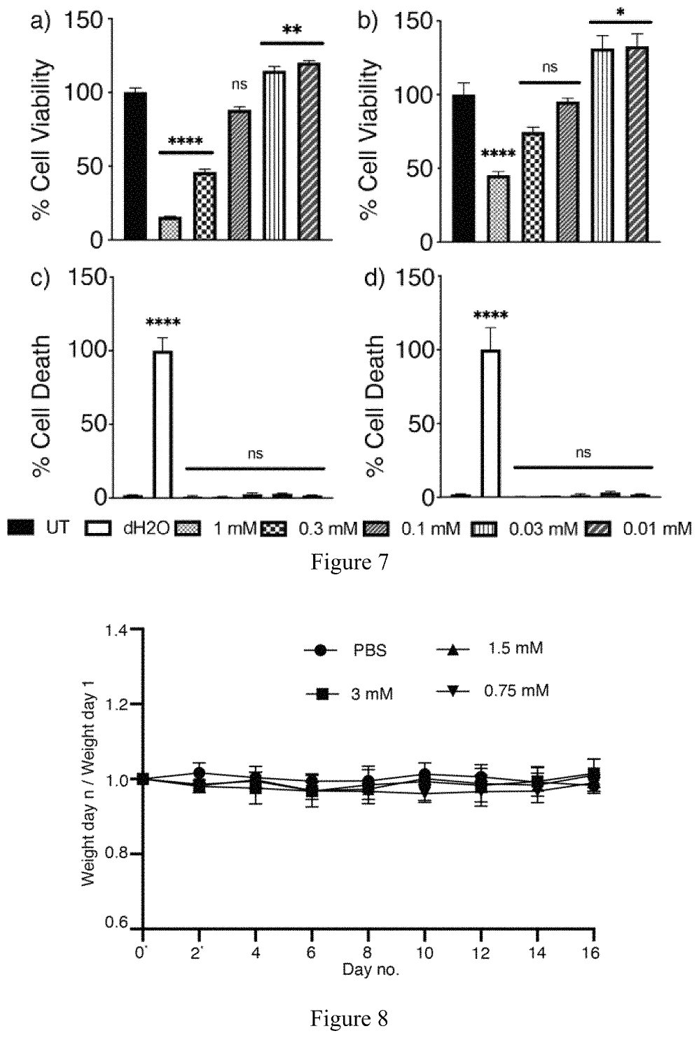

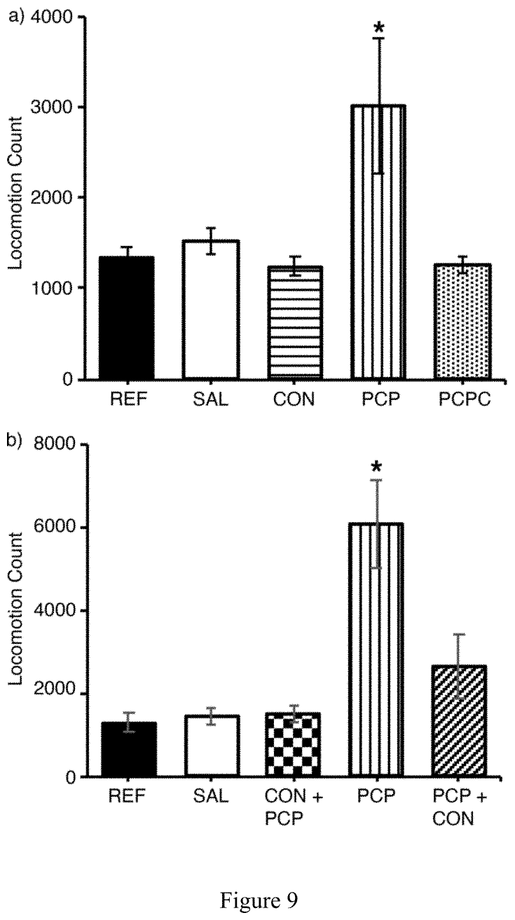

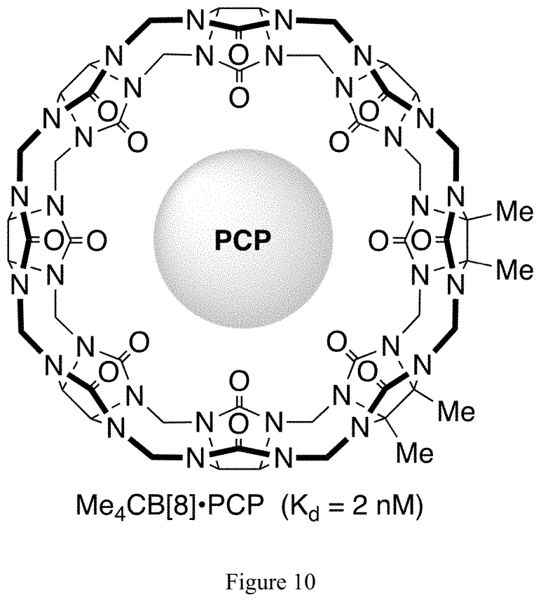

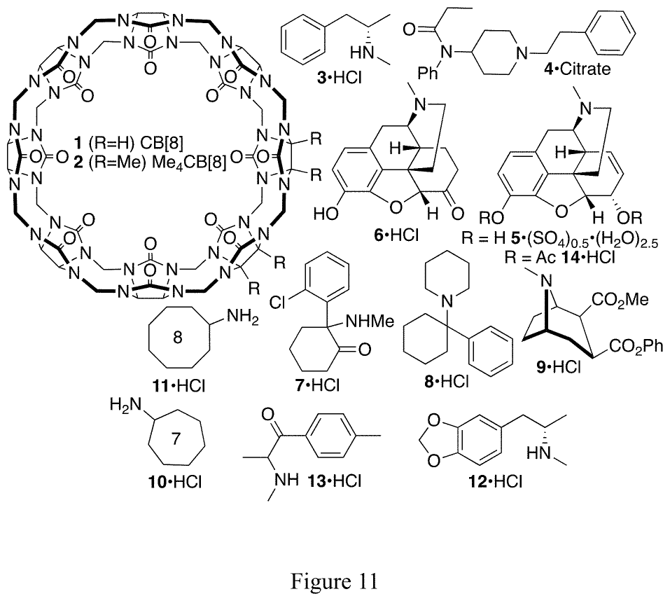

In Vitro Cytotoxicity and In Vivo Maximum Tolerated Dose Studies. Previous studies of macrocyclic unfunctionalized CB[n] have shown that they possess high biocompatibility across a wide range of in vitro cytotoxicity and in vivo tolerability studies. Before proceeding to in vivo efficacy studies, it was sought to confirm the expected high biocompatibility of the Me₄CB[8] host. First, in vitro cytotoxicity assays for Me₄CB[8] were performed using MTS metabolic and adenylate kinase (AK) release cell death assays (FIG. 7). Human kidney (HEK293) and liver (HEPG2) cell lines were selected because they are commonly used in drug toxicity studies to determine liver cell and renal cell toxicity, respectively, and because the kidney and liver are where drugs accumulate for processing and clearance by the body. Distilled water was used as a positive control for the AK assay (set to 100% release) and untreated (UT) cells were used as a reference for the MTS assay (100% cell viability). HEK293 and HEPG2 cells treated with Me₄CB[8] showed a dose-dependent response for cell viability. At the highest concentration tested (1 mM), the HEPG2 cells showed an ˜85% reduction in cell viability, and the HEK293 cells showed a ˜55% reduction (FIG. 7a,b respectively). This reduction of cell viability could be produced by cells that are dying but not yet lysed. The reduction in cell viability was absent at concentrations of Me₄CB[8] less than 0.1 mM. Interestingly, the lower doses yielded cell viability values above 100% for both cell types. The observed slight increase in the percentage of viability at low doses of Me₄CB[8] could be due to an increase in mitochondrial activity or to Me₄CB[8] induced interference in the colorimetric assay. Neither the HEK298 or HEPG2 cells show any significant amount of lysis (FIG. 7c,d) compared to the positive control.

With acceptable results from the cytotoxicity assays, in vivo compatibility of Me₄CB[8] was demonstrated by a maximal tolerated dose (MTD) study. Swiss Webster mice were dosed via tail vein injection (6 mL kg) of Me₄CB[8] (3 mM (maximal solubility), 1.5 mM, and 0.7 mM) on days 0 and 2 (denoted by *) along with PBS as a control (FIG. 8). The animals were weighed daily and monitored for a two-week period for signs of sickness or behavioral changes. Mice in all dosing groups showed no signs of sickness in terms of behavior or significant weight change over the course of the study (FIG. 8). As such, it was concluded that Me₄CB[8] can be used at its maximum solubility (3 mM) for the greatest potential of PCP reversal without any significant risks of associated toxicity.

In vivo reversal of PCP-induced hyperlocomotion. Next, it was determined whether the high in vitro binding affinity of Me₄CB[8] toward PCP could prevent or reverse the biological effects of PCP. As such, hyperlocomotive effects of PCP in mice was used as a way of monitoring its biological activity via open field tests. A preliminary study was conducted on 8 male Swiss Webster (CFW) mice (weight, mean:SD: 38.6:2.3 g) in a randomized controlled crossover manner. Over four consecutive days, mice were treated with a 0.2 mL infusion of either sterile saline (0.9%), Me₄CB[8] alone at (3 mM) in 1×PBS buffer, PCP (2 mg/kg) at (1.5 mM), or a premixed solution of Me₄CB[8] plus PCP (2 mg/kg) at a ratio of (Me₄CB[8]:PCP) (2:1). Treatments were counterbalanced across the four treatment days. A one-way repeated measures ANOVA revealed a significant effect of treatment (F(4,28)=4.331, p=0.0075). Pairwise-bonferroni corrected post-hoc comparisons of locomotion counts across treatments revealed that PCP treatment increased locomotion significantly more than treatments with saline (p=0.049), Me₄CB[8] alone (p=0.011), or treatment with the premixed solution of Me₄CB[8] and PCP (p=0.012), respectively. Treatment with the premixed solution of Me₄CB[8] and PCP did not significantly increase locomotion when compared to treatment with saline (p>0.05) or treatment with Me₄CB[8] alone (p>0.05), suggesting that PCP remained bound to Me₄CB[8] in vivo and prevented PCP-induced increases in locomotor behavior. Moreover, no effect of Me₄CB[8] alone was observed on locomotion (saline vs. Me₄CB[8]; p>0.05).

Next, it was determined whether the molecular recognition event of Me₄CB[8] toward PCP could occur in the biological setting instead of the syringe. This question was answered with a two-fold approach involving either prevention of PCP induced hyperlocomotion conducted by administering Me₄CB[8] before PCP, or treatment of PCP induced hyperlocomotion by administering Me₄CB[8] after PCP. The studies were performed using male Swiss Webster (CFW) mice (N=9; weight, mean:SD: 37.9:3.0 g) in a randomized controlled crossover manner. Over four consecutive days, mice were treated with either a 0.2 mL infusion of sterile saline (0.9%), PCP (1.95 mg/kg at 1.46 mM), a sequential infusion of Me₄CB[8] (2.93 mM) followed by PCP (1.95 mg/kg) at a 2:1 ratio of Me₄CB[8]:PCP, or a sequential infusion of PCP (1.95 mg/kg) followed by Me₄CB[8] (2.93 mM) at a Me₄CB[8]:PCP ratio of 2:1. For sequential infusions, the volume of both infusions totaled (0.2 mL), and were spaced 30 s apart (i.e., 30 s elapsed between the first and second infusions). Treatments were counterbalanced across the four treatment days. A one-way repeated measures ANOVA comparing locomotion counts across treatment conditions revealed a significant main effect of treatment (F(4,32)=11.44, p<0.0001). Pairwise bonferroni-corrected post-hoc comparisons revealed that PCP significantly increased locomotion counts compared to treatment with saline (p=0.0005), sequential infusion of Me₄CB[8] followed by PCP (p=0.0006) as well as sequential infusion of PCP followed by Me₄CB[8] (p=0.0034). However, no differences in locomotion counts were observed when comparing the two sequential infusion treatments (CON+PCP; PCP+CON) to saline, respectively (p's>0.05) or when comparing the two sequential infusion treatments against one another (p>0.05). This suggests that independent of the order in which the infusion is administered, Me₄CB[8] is able to bind PCP in vivo and prevent PCP-induced hyperlocomotion.

Conclusion

In summary, the binding affinities of two CB[n] hosts—CB[8] and its water soluble derivative Me₄CB[8]—were measured toward a panel of commonly used and abused drugs (3-9, 12-14) by isothermal titration calorimetry. Water soluble host Me₄CB[8] displays remarkable binding affinity toward both ketamine (7, K_{d}=36 nM) and PCP (8, K_{d}=2 nM). ¹H NMR spectroscopy shows that the large Me₄CB[8] cavity is capable of simultaneously encapsulating the phenyl and cyclohexyl rings of 7 whereas the phenyl and piperidium rings of 8 are hosted in the Me₄CB[8] cavity. Similarly, the morphinan ring system of morphine (5, K_{d}=21 nM) and hydromorphone (6, K_{d}=47 nM) are encapsulated inside the CB[8] cavity quite efficiently. The water soluble Me₄CB[8] host displays low in vitro cytotoxicity below 100 μM toward HEK293 and HEPG2 cells according to standard MTS metabolic and AK release cell death assays and no deleterious effects in maximum tolerated dose studies in mice up to 3 mM. Finally, in vivo efficacy studies showed that PCP induced hyperlocomotion can be effectively controlled by either the prevention or treatment approaches. Given that CB[8] and Me₄CB[8] also bind strongly toward ketamine, morphine, and hydromorphone suggests that interventions based on these hosts or other water soluble CB[8] derivatives holds promise as a new general purpose treatment of overdose with a variety of drugs of abuse.

TABLE 2
Binding constants determined for the various CB[8]•drug complexes

| | Kₐ (CB[8]•guest) | ΔH° | ΔG° | |
|---|---|---|---|---|
| Guest | (M⁻¹) | (kcal/mol) | (kcal/mol) | n |
| 3 Methamphetamine^{[a]} | (1.47 ± 0.09) × 10⁵ | (−7.84 ± 0.1) | −7.05 | 1 |
| 4 Fentanyl^{[a]} | (1.9 ± 0.09) × 10⁷ | (−10.8 ± 0.06) | −9.94 | 1 |
| | (3.7 ± 0.04) × 10⁵ | (−7.67 ± 0.04) | −7.60 | 2 |
| 5 Morphine^{[b]} | (3.41 ± 0.15) × 10⁸ | (−13.6 ± 0.04) | −11.6 | 1 |
| 6 Hydromorphone^{[b]} | (1.7 ± 0.11) × 10⁸ | (−15.8 ± 0.1) | −11.2 | 1 |
| 7 Ketamine^{[c]} | (1.09 ± 0.07) × 10⁹ | (−17.3 ± 0.16) | −12.3 | 1 |
| 8 Phencyclidine^{[c]} | (2.1 ± 0.2) × 10¹⁰ | (−14.9 ± 0.04) | −14.1 | 1 |
| 9 Cocaine^{[a]} | (6.45 ± 0.43) × 10⁵ | (−8.26 ± 0.15) | −7.79 | 1 |
| 10 Cy7NH₃^{[a]} | (5.3 ± 0.23) × 10⁶ | (−7.62 ± 0.03) | −9.18 | 1 |
| 11 Cy8NH₃^{[a]} | (3.2 ± 0.26) × 10⁷ | (−8.26 ± 0.04) | −10.2 | 1 |

Measured by ^{[a]}direct ITC titration, ^{[b]}competition ITC titration with 0.075 mM 10, or ^{[c]}competition ITC titration with 0.2 mM 11.

TABLE 3
Binding constants determined for the various Me₄CB[8]•drug complexes

| | Kₐ (Me₄CB[8]•guest) | ΔH° | ΔG° | |
|---|---|---|---|---|
| Guest | (M⁻¹) | (kcal/mol) | (kcal/mol) | n |
| 3 | (2.98 ± 0.47) × 10⁴ | (−4.61 ± 0.04) | −6.10 | 1 |
| Methamphetamine^{[a]} | | | | |
| 4 Fentanyl^{[a]} | (1.98 ± 0.02) × 10⁶ | (−3.58 ± 0.01) | — | 1 |
| | (9.52 ± 0.03) × 10⁴ | (−1.94 ± 0.01) | — | 2 |
| 5 Morphine^{[b]} | (4.67 ± 0.17) × 10⁷ | (−8.24 ± 0.02) | −10.5 | 1 |
| 6 Hydromorphone^{[b]} | (2.14 ± 0.05) × 10⁷ | (−8.43 ± 0.02) | −10.0 | 1 |
| 7 Ketamine^{[c]} | (2.81 ± 0.67) × 10⁷ | (−9.37 ± 0.02) | −10.2 | 1 |
| 8 Phencyclidine^{[c]} | (5.35 ± 0.19) × 10⁸ | (−8.39 ± 0.01) | −11.9 | 1 |
| 9 Cocaine^{[a]} | (2.77 ± 0.15) × 10⁵ | (−6.90 ± 0.07) | −7.43 | 1 |
| 10 Cy7NH₃^{[a]} | (1.04 ± 0.02) × 10⁶ | (−5.35 ± 0.01) | −8.21 | 1 |
| 11 Cy8NH₃^{[a]} | (2.79 ± 0.17) × 10⁶ | (−5.63 ± 0.03) | −8.79 | 1 |
| 12 MDMA^{[a]} | (3.13 ± 0.32) × 10⁴ | (−13.8 ± 0.77) | −6.13 | 1 |
| 13 Mephedrone^{[a]} | (3.07 ± 0.39) × 10⁵ | (−9.89 ± 0.29) | −7.49 | 1 |
| 14 Heroin^{[a]} | (7.94 ± 0.07) × 10⁴ | (−15.1 ± 0.10) | −6.70 | 1 |

Measured by ^{[a]}direct ITC titration, ^{[b]}competition ITC titration with 0.075 mM 10, or ^{[c]}competition ITC titration with 0.2 mM 11.

Cell Cytotoxicity Data for Me₄CB[8].To test the Cytotoxicity and Cell Viability of the above compounds, two different assays were used: an MTS (CellTiter 96 AQueous Kit®) assay that measures cellular metabolism, and the AK (Toxilight®BioAssay Kit) assay that measures cell death through release of the cytosolic enzyme adenylate kinase into the supernatant. Both assays were performed with two different cell lines. HEK293 and Hep G2cells, are frequently used in drug toxicity studies. HEK293, a human kidney cell line, is used to evaluate the effect of the drug on the renal system and Hep G2, a human hepatocyte cell line, is used to assess the response of liver cells where drugs are metabolized. The MTS and AK assays for both cell lines were conducted after 24 h of incubation with the compounds at concentrations of 0.01 mM, 0.03 mM, 0.1 mM, 0.3 mM, and 1 mM. Eight technical replicates were designated for untreated cells and four technical replicates were designated for the cells treated with each compound and staurosporine (apoptosis inducer).

The collected absorbance and relative luminescence data were normalized to percent cell viability (MTS) and percent cell death (AK) using equations 1 and 2:

% cell viability=(Abs sample/Average Abs UT)×100 1)

% cell death=(RLU samples/Average RLU Distilled water)×100 2)

In Vivo Maximum Tolerated Dose Study (MTD). Animals studies were performed at the University of Maryland, Microbiology Building under the supervision of Dr.

Volker Briken (IACUC #R-JAN-17-25). A total of 20 female Swiss Webster were used for this study. Three different concentrations of Me₄CB[8], 3 mM, 1.5 mM, and 7.5 mM were used. A PBS control group was included for each compound. Each concentration and control group contained 5 mice. The mice received the compound in 0.150 ml of PBS via tail vein injection, with 48 hours between injections. Mice were monitored for 2 weeks following last injection.

In Vivo Reversal of PCP-Induced Hyperlocomotion by Me₄CB[8]

Animals. Seventeen male Swiss Webster (CFW) mice were obtained from Charles River Laboratories that weighed ˜30 g upon arrival. Mice were individually housed in a temperature- and humidity-controlled room on a 12 h light/dark schedule with lights on at 6:00 am EST. For the duration of both experiments mice had ad libitum access to food and water. All behavioral testing occurred between 6:30 am and 2:00 pm EST, and all experimental procedures were approved by the University of Maryland Animal Care and Use Committee and conformed to the guidelines set forth by the National Research Council.

Surgical Procedures Mice were anesthetized with either isoflurane (Experiment 1; n=8) or an intraperitoneal (IP) injection of ketamine (100 mg/kg)/xylazine (10 mg/kg) (Experiment 2; n=9) and were implanted with jugular catheters with head-mounted ports. All surgical procedures were conducted using aseptic technique, with body temperature monitored and maintained throughout surgery. Catheters were placed in the right jugular vein with the port passed subcutaneously out towards the top of skull. Ports (5 MM Up Pedestal; P1 Technologies) were fixed to the skull with a combination of super glue (Loctite) and dental cement. Following surgery, mice received an immediate injection of Rimadyl (5 mg/kg) and 0.4 mL of warm sterile saline. Mice were treated post-operatively for two days with Rimadyl (5 mg/kg) and given a minimum of 5 days to recover before resuming training. Catheters were flushed daily with 0.1 mL sterile saline solution containing gentamycin (0.33 mg/mL) and 0.1 mL sterile saline solution containing heparin (20 IU/mL) in order to reduce clotting and maintain catheter patency. Catheter patency was assessed daily from the first day following surgery till the end of testing. Any mouse that's catheter exhibited significant flowback on a majority of days was excluded from analysis.

Behavioral Testing Mice were trained on a standard autoshaping task. All behavioral procedures were conducted in a Med Associates test chamber equipped with a food cup, a retractable lever, and 4 floor IR photobeams. Time stamps were generated from head entries into the food cup, downward deflections of the lever, or disruption of floor beams and recorded by the behavioral computer.

Mice were given one day of magazine training that consisted of the delivery of twenty-five 20 mg sucrose pellets (Bioserv) randomly delivered on a variable interval 30±15 schedule, in order to habituate mice to the box and pellet delivery. In order to minimize the impact of novelty-induced suppression of feeding, mice were given five to six 20 mg sucrose pellets each in their home cage for 2-3 days prior to the beginning of training.

Following magazine training, mice began Pavlovian training sessions which consisted of the presentation of the lever (CS) for 8 s, which was immediately followed by the delivery of a sucrose pellet and the retraction of the lever. The CS was presented on a random interval of 90±30 s schedule. Each Pavlovian session consisted of either 25 trials (experiment 1) or 30 trials (experiment 2). Pavlovian training continued for 4 days prior to surgery. Following surgery and recovery, mice underwent Pavlovian training for an additional 5 days while being exposed to various treatments.

Experimental Design. We assessed Me₄CB[8] efficacy using two separate experiments in which all mice received each possible experimental treatment in a counterbalanced manner, respectively. The purpose of the two experiments was to verify (1) that binding of PCP by Me₄CB[8] would not be compromised in vivo (experiment 1) (2) verify that Me₄CB[8] would not alter locomotor behavior (experiment 1), and (3) that Me₄CB[8] can sequester free floating PCP in vivo (experiment 2). Following surgery, all mice were trained for five additional days on the Pavlovian task. The first day, regardless of experiment, was a refresher session, where mice were allowed to complete the training free of treatment. On the following 4 sessions mice were treated with one of four possible treatments.

In experiment 1, mice (n=8) were treated with a 0.2 mL infusion of either sterile saline (0.9%), Me₄CB[8] alone, PCP (2 mg/kg) or a premixed solution of Me₄CB[8] plus PCP (2 mg/kg) (ratio of Me₄CB[8] to PCP was 2:1). Treatments were counterbalanced across the four treatment days. For the purposes of this manuscript only locomotion data will be presented.

In experiment 2, mice (n=9) were treated with a 0.2 mL infusion of either sterile saline (0.9%), PCP (1.95 mg/kg), a sequential infusion of PCP (1.95 mg/kg) followed by Me₄CB[8] (ratio of PCP to Me₄CB[8] was 1:2) or a sequential infusion of Me₄CB[8] followed by PCP (1.95 mg/kg) (ratio of Me₄CB[8] to PCP was 2:1). For sequential infusions the volume of both infusions totaled 0.2 mL, and sequential infusions were spaced 30 s apart (i.e., 30 s elapsed between the first and second infusions). As in experiment 1, for the purposes of this present disclosure only locomotion data will be presented.

For each experiment, total locomotion counts (i.e., the total number of beam breaks) were obtained for each mouse across the entirety of each training session. For each experiment, locomotion counts were then analyzed across treatments using one-way repeated measures ANOVAs with bonferroni-corrected pairwise post-hoc t-tests in R.

Although the present disclosure has been described with respect to one or more particular embodiments and/or examples, it will be understood that other embodiments and/or examples of the present disclosure may be made without departing from the scope of the present disclosure.

1. A method for sequestering one or more neuromuscular blocking agent(s), one or more anesthesia agent(s), one or more pharmaceutical agent(s), one or more drug(s) of abuse, one or more pesticide(s), one or more dyestuff(s), one or more malodorous compound(s), one or more chemical warfare agent(s), one or more hallucinogen(s), one or more toxin(s), one or more metabolite(s), or a combination thereof comprising:
contacting the neuromuscular blocking agent(s), the anesthesia agent(s), the pharmaceutical agent(s), one or more drug(s) of abuse, the pesticide(s), the dyestuff(s), the malodorous compound(s), the chemical warfare agent(s), one or more hallucinogen(s), one or more toxin(s), one or more metabolite(s), or a combination thereof with one or more compound(s) having the following structure: 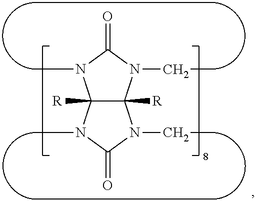
wherein each R is independently chosen from hydrogen, alkyl groups, hydroxyl groups, alkoxy groups, aryl groups, heteroaryl groups, amino groups, amide groups, carboxylic acid/carboxylate groups, ester groups, imide groups, thiol groups, ether groups, polyether groups, phosphate groups, phosphonate groups, sulfonate groups, alkyl sulfonate groups, sulfate groups, alkyl sulfate groups, and combinations thereof, and the neuromuscular blocking agent(s), the anesthesia agent(s), the pharmaceutical agent(s), the drug(s) of abuse, the pesticide(s), the dyestuff(s), the malodorous compound(s), the chemical warfare agent(s), one or more hallucinogen(s), one or more toxin(s), one or more metabolite(s), or a combination thereof are sequestered by the one or more compound(s).

2. The method of claim 1, wherein the one or more compounds have the following structure:

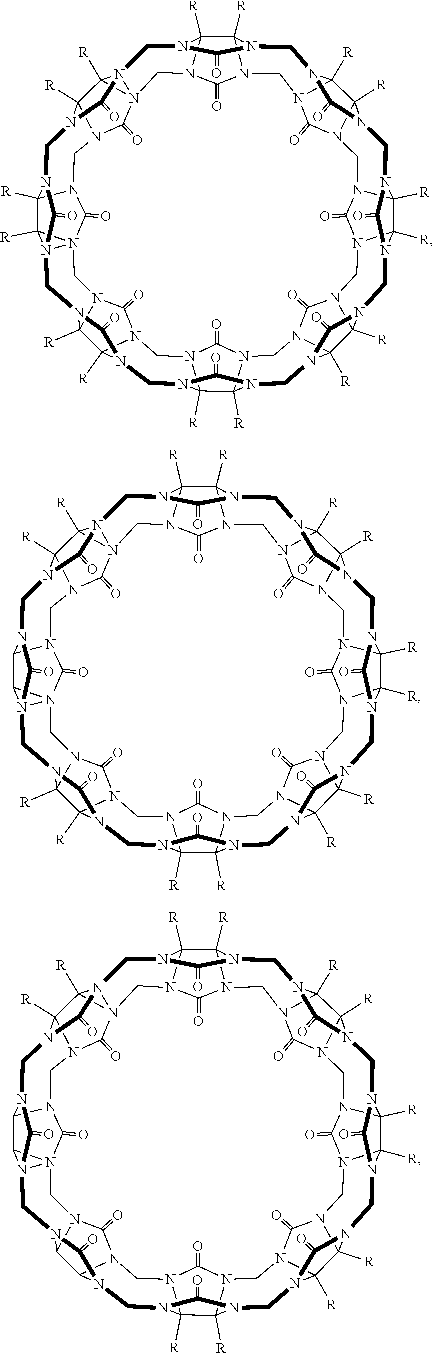

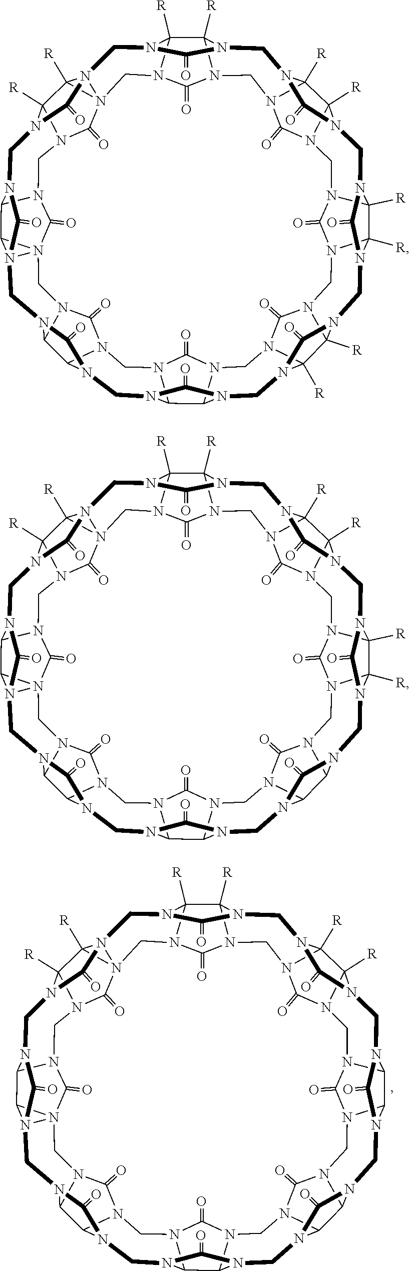

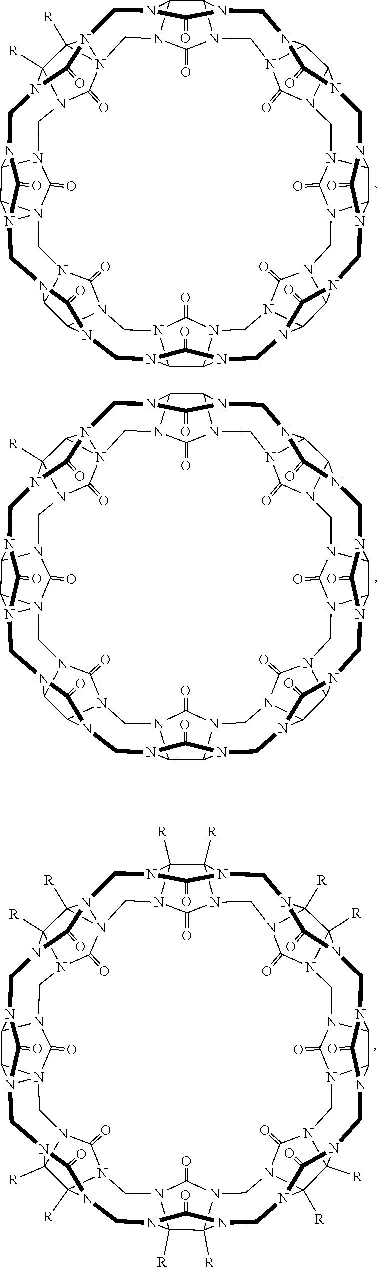

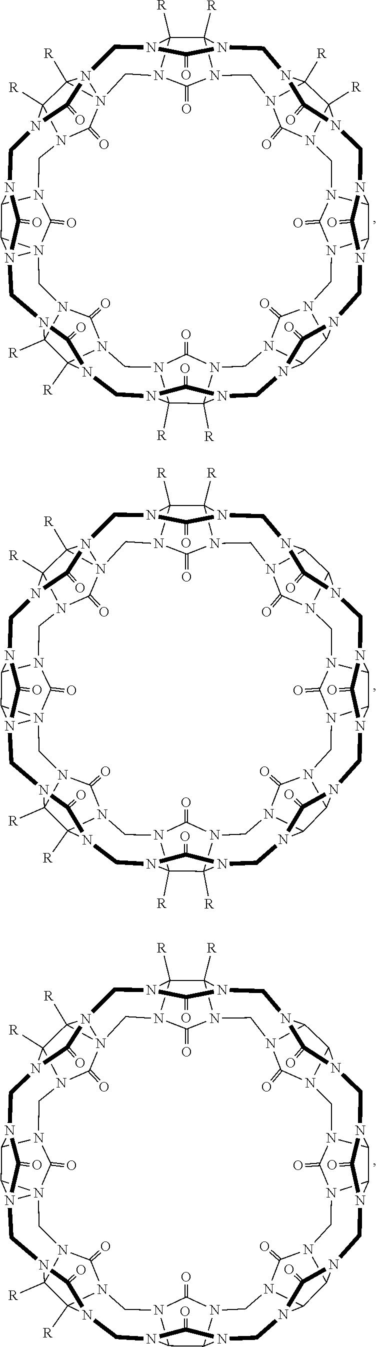

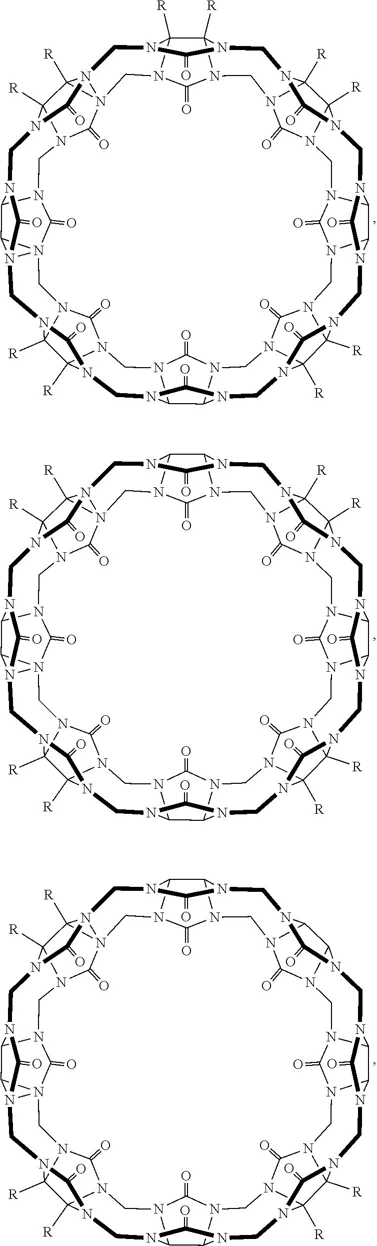

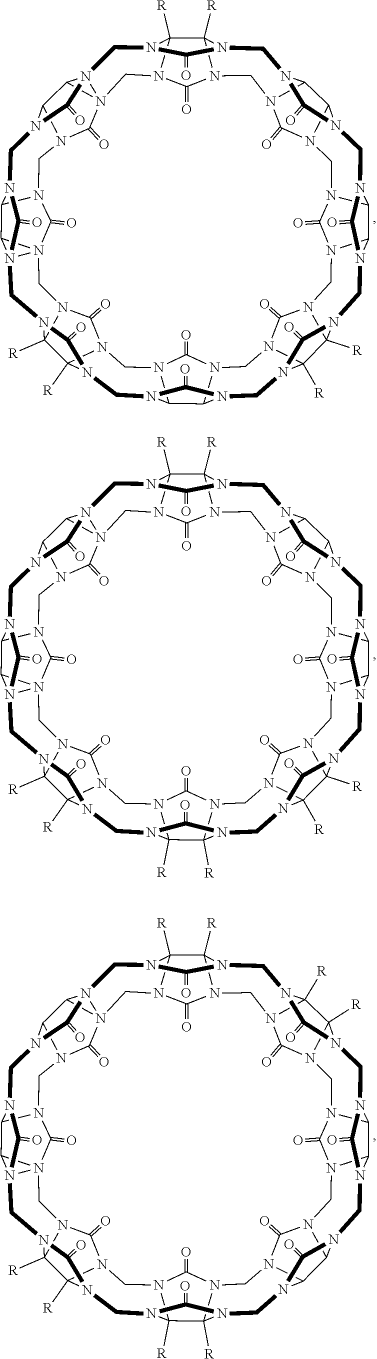

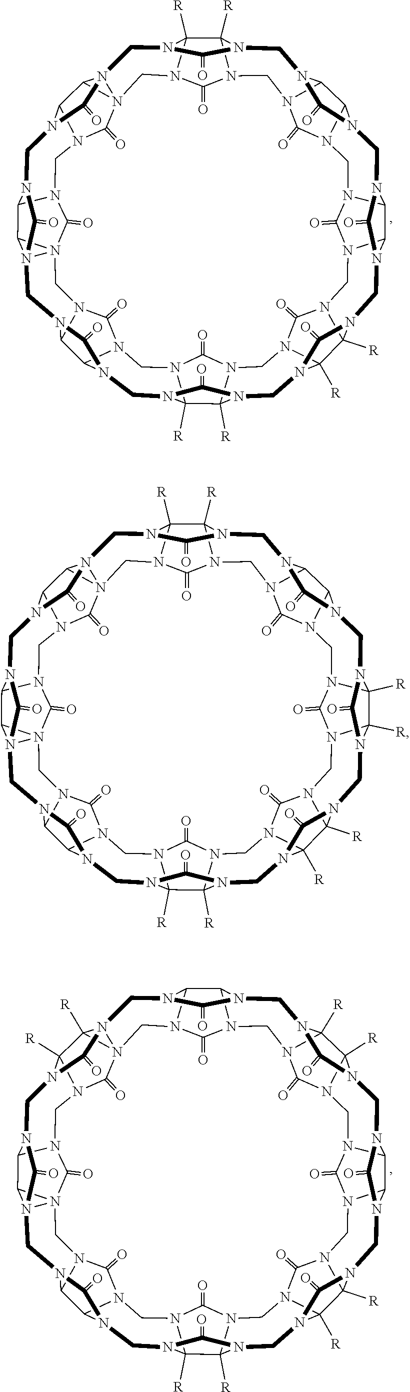

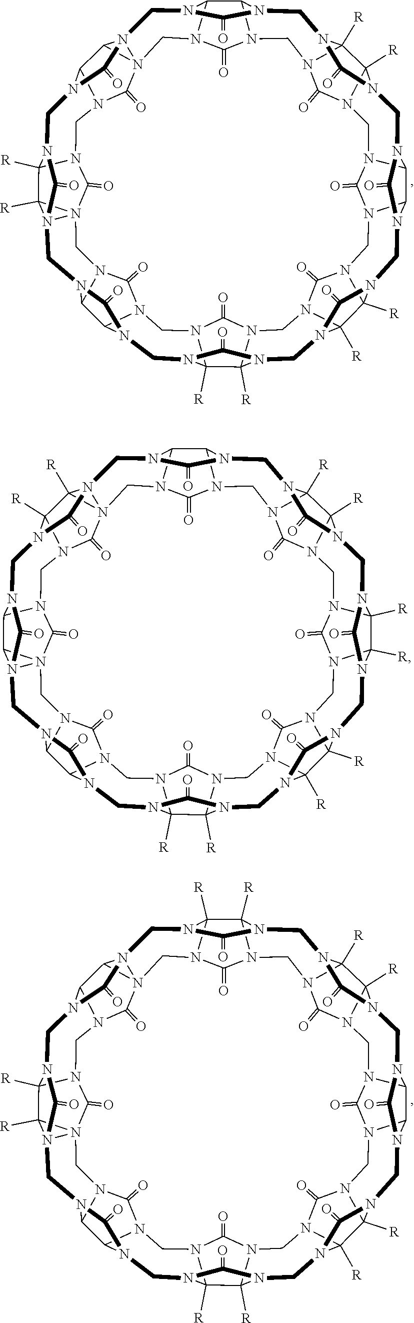

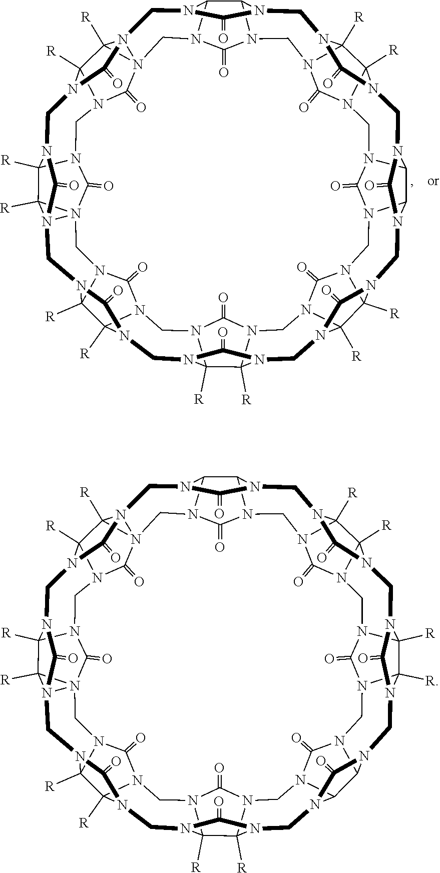

3. The method of claim 2, wherein the one or more compounds have the following structure: 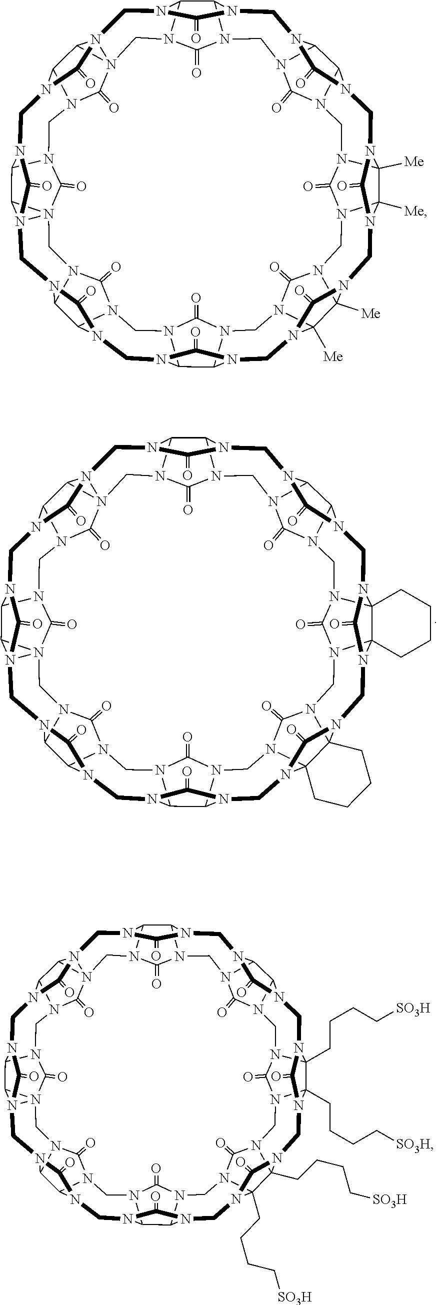 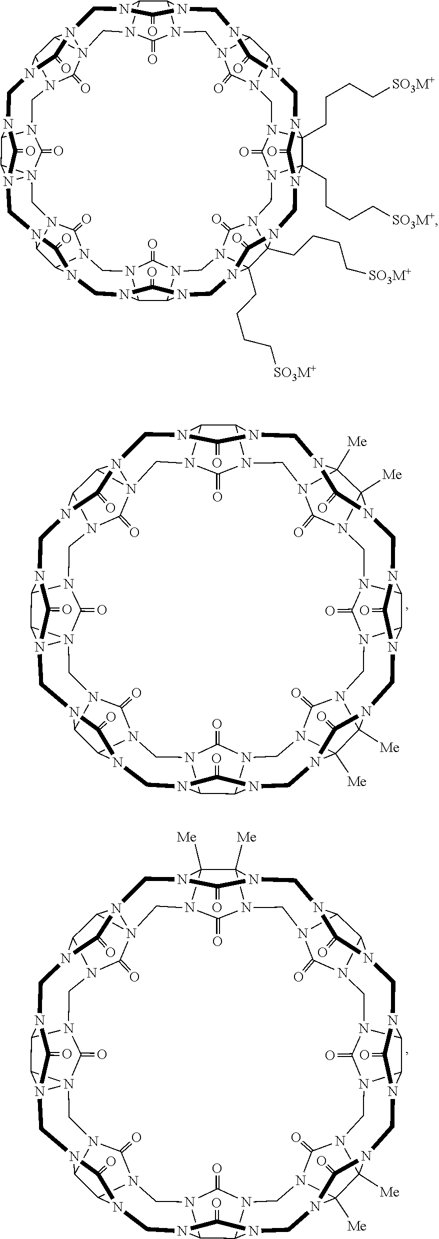 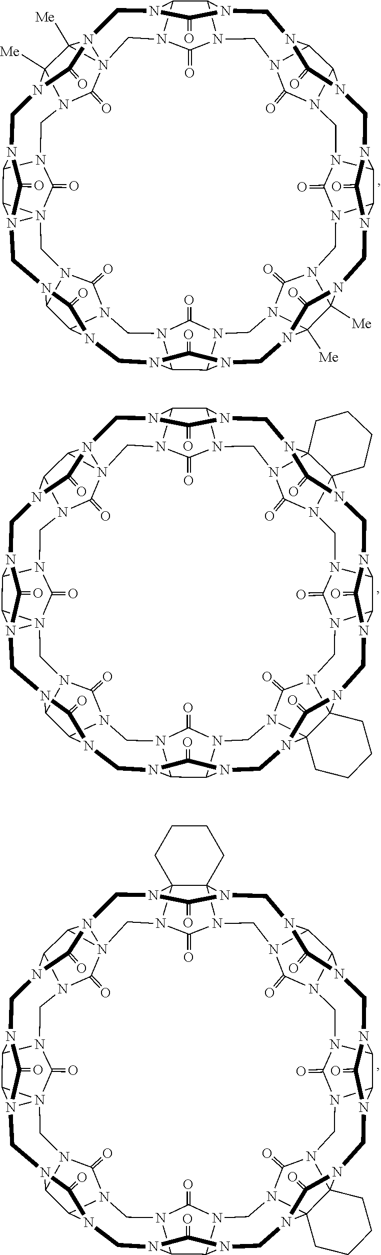 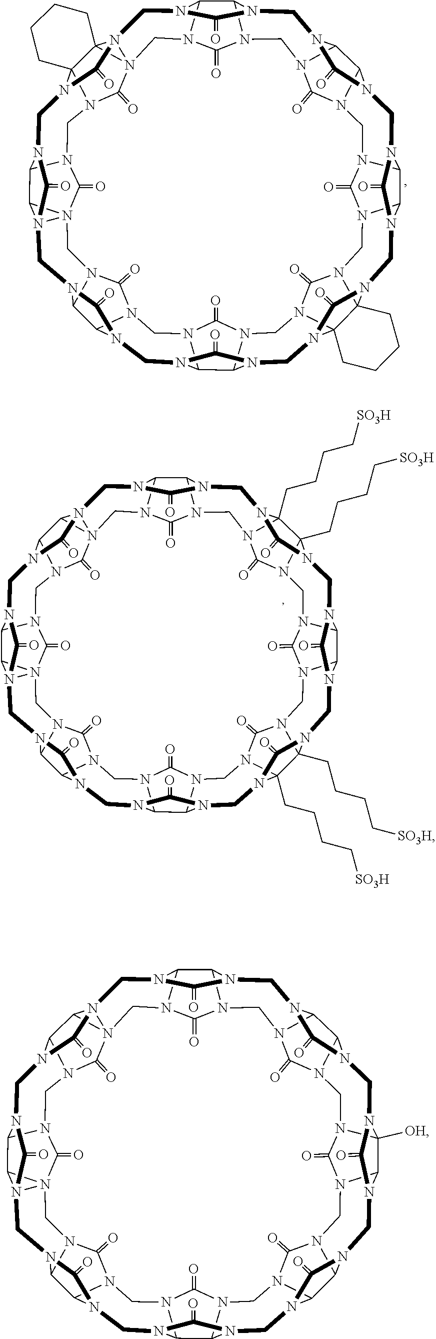 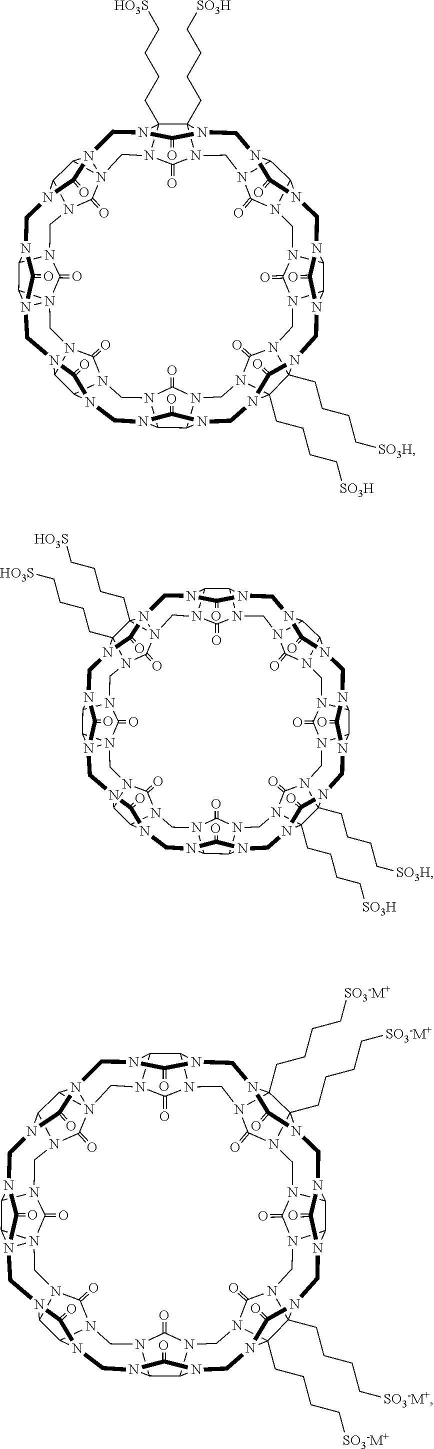 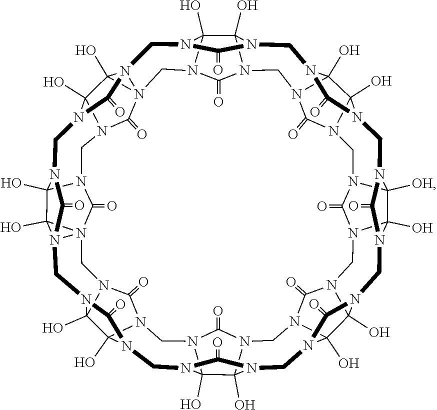 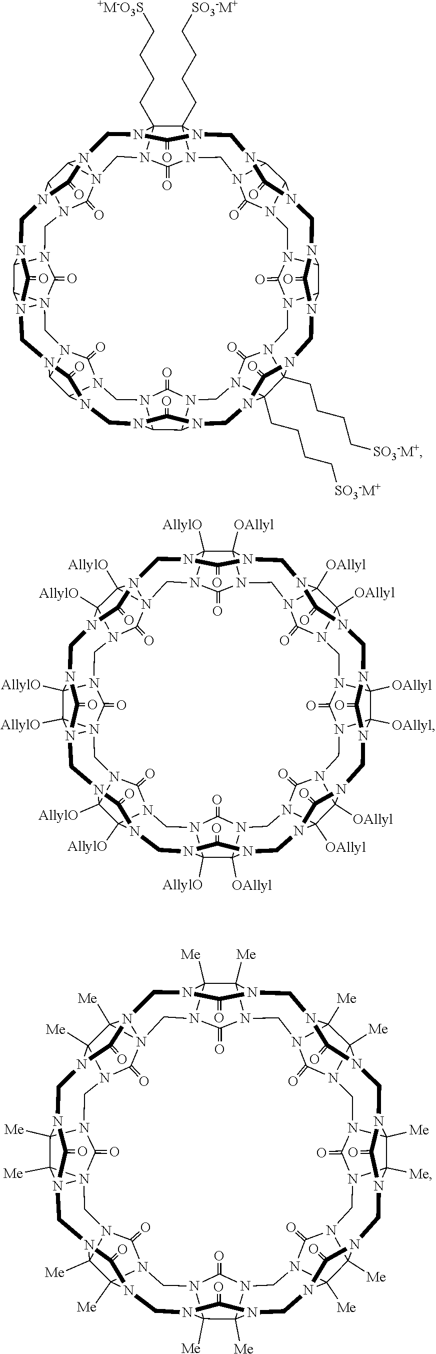 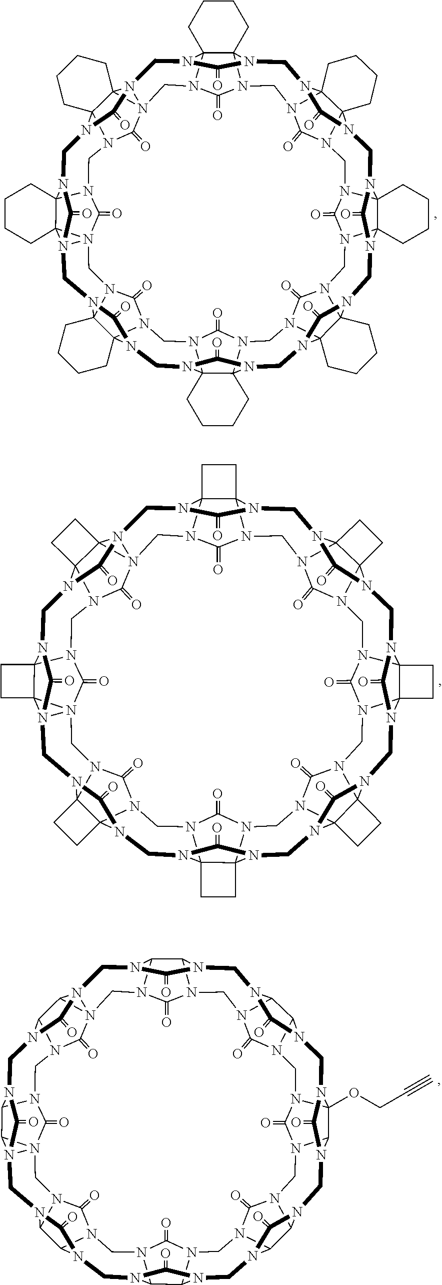 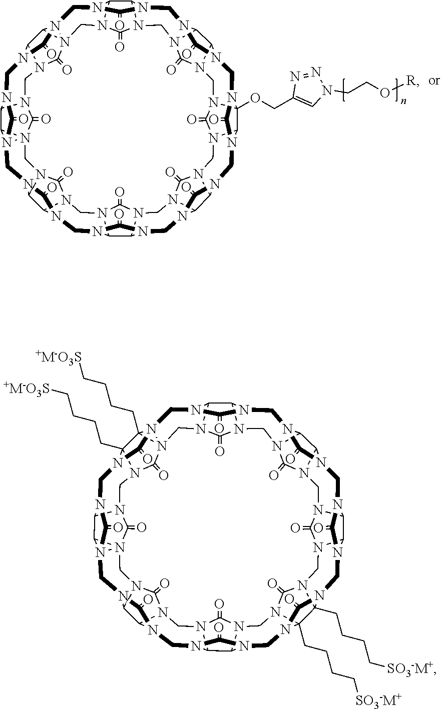
wherein n is 3 to 1000 and M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS).

4. The method of claim 3, wherein the one or more compounds have the following structure:

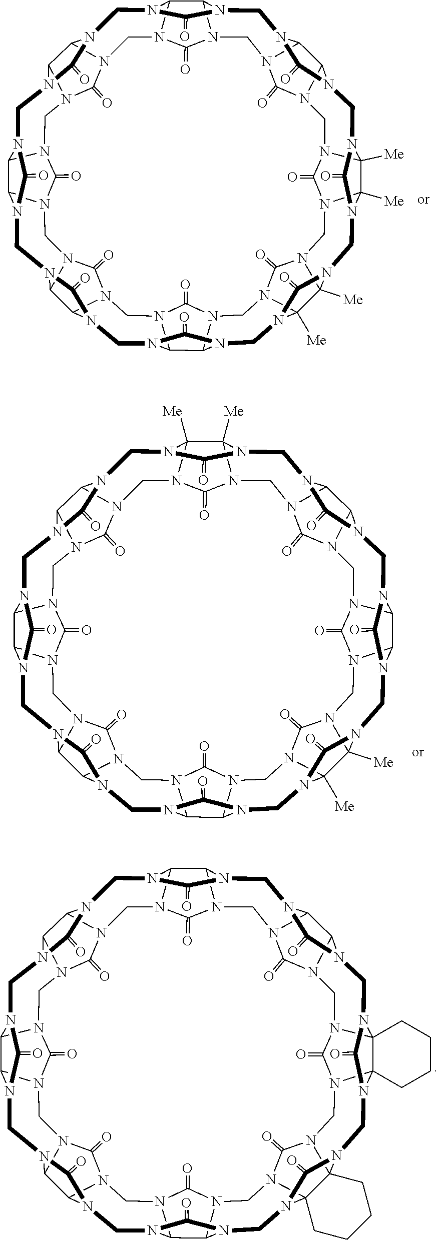

5. The method of claim 1, wherein the neuromuscular blocking agent(s), the anesthesia agent(s), the pharmaceutical agent(s), the drug(s) of abuse, the pesticide(s), the dyestuff(s), the malodorous compound(s), the chemical warfare agent(s), one or more hallucinogen(s), one or more toxin(s), one or more metabolite(s), or a combination thereof is present in an aqueous sample, in a solid sample, in a gas sample, or on a solid surface.

6. The method of claim 1, wherein a complex is formed from the one or more compound(s) and the neuromuscular blocking agent(s), the anesthesia agent(s), the pharmaceutical agent(s), the drug(s) of abuse, the pesticide(s), the dyestuff(s), the malodorous compound(s), the chemical warfare agent(s), one or more hallucinogen(s), one or more toxin(s), one or more metabolite(s), or a combination thereof.

7. The method of claim 1, wherein the complex is removed from the aqueous sample, the solid sample, or the gas sample.

8. The method of claim 1, wherein at least one R group is not H.

9. The method of claim 1, wherein the neuromuscular blocking agent(s), the anesthesia agent(s), the pharmaceutical agent(s), the drug(s) of abuse, the pesticide(s), the dyestuff(s), the malodorous compound(s), the chemical warfare agent(s), one or more hallucinogen(s), one or more toxin(s), one or more metabolite(s), or a combination thereof is present in and/or on an individual and the contacting comprises administration of the one or more compound(s).

10. A method for reversing drug-induced neuromuscular block and/or anesthesia and/or the effects of one or more pharmaceutical agent(s) and/or the effects of one or more drug(s) of abuse in an individual comprising administering to an individual in need of reversal of neuromuscular block and/or reversal of anesthesia and/or reversal of the effects of one or more pharmaceutical agent(s) and/or reversal of the effects of one or more drug(s) of abuse and/or the effects of one or more chemical warfare agents one or more compound(s) having the following structure: 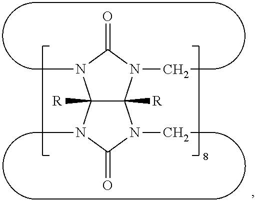
wherein each R is independently chosen from hydrogen, alkyl groups, hydroxyl groups, alkoxy groups, aryl groups, heteroaryl groups, amino groups, amide groups, carboxylic acid/carboxylate groups, ester groups, imide groups, thiol groups, ether groups, polyether groups, phosphate groups, phosphonate groups, sulfonate groups, alkyl sulfonate groups, sulfate groups, alkyl sulfate groups, and combinations thereof, wherein at least one R group is not H.

11. The method of claim 10, wherein the one or more compounds have the following structure:

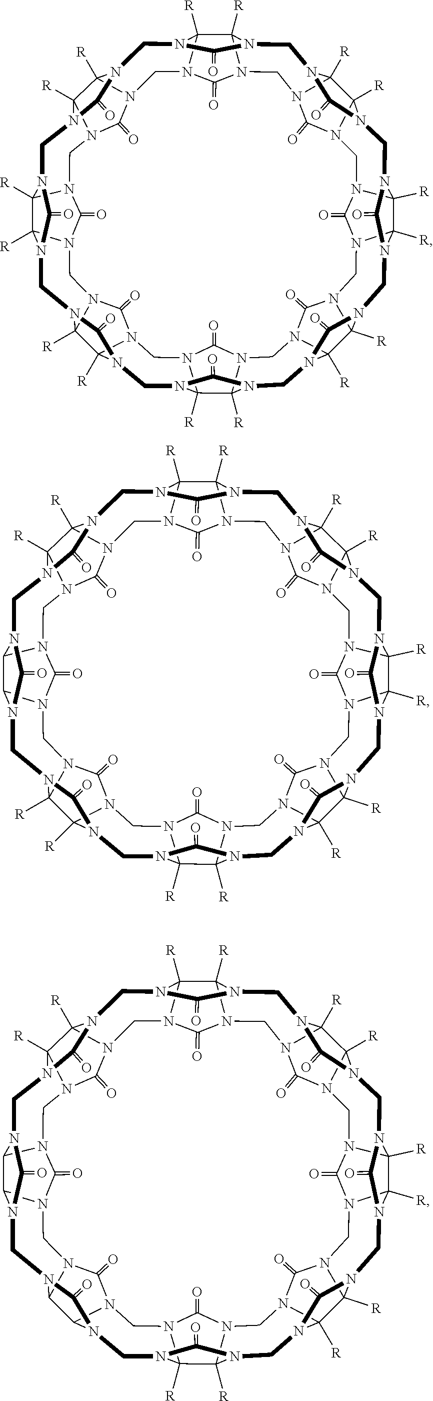

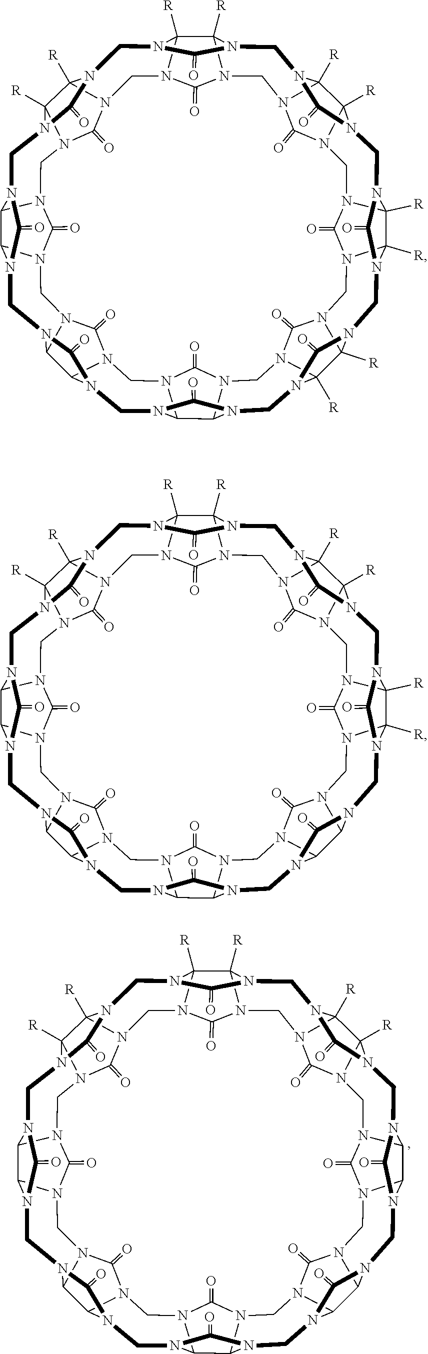

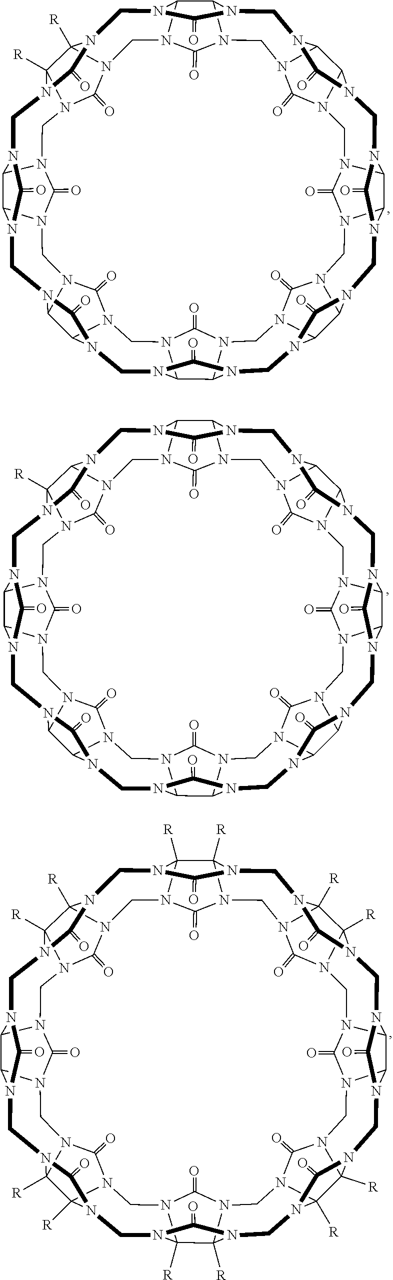

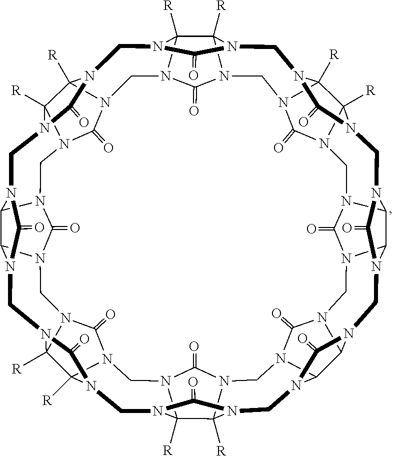

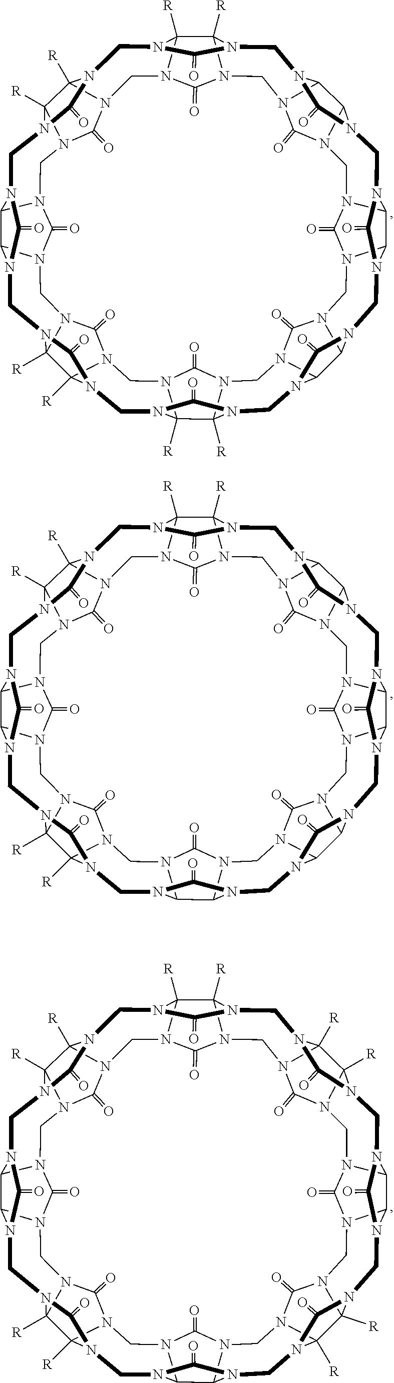

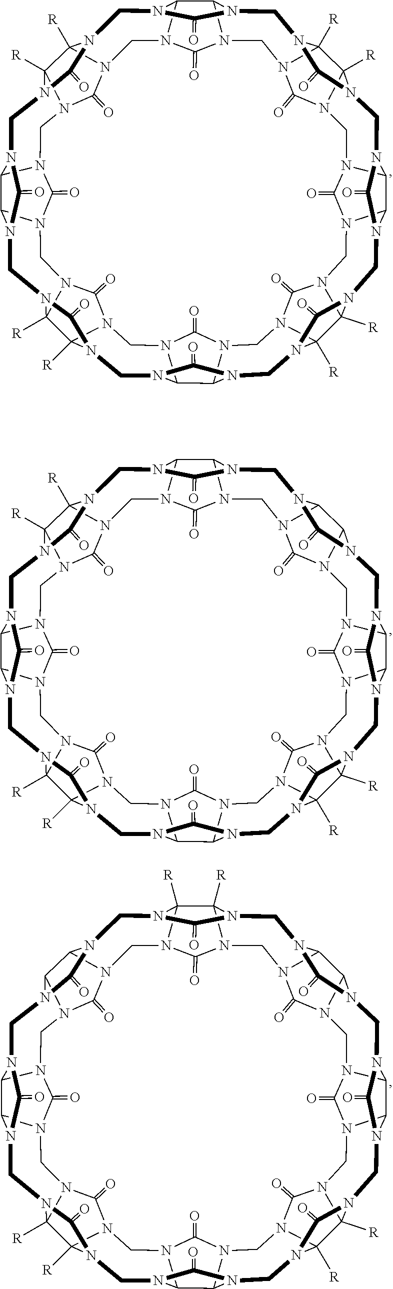

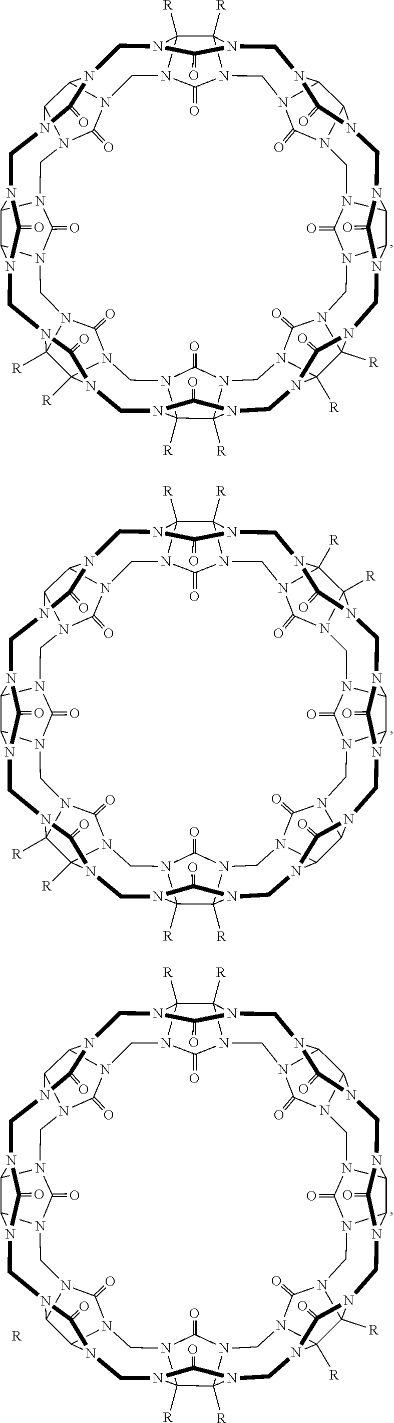

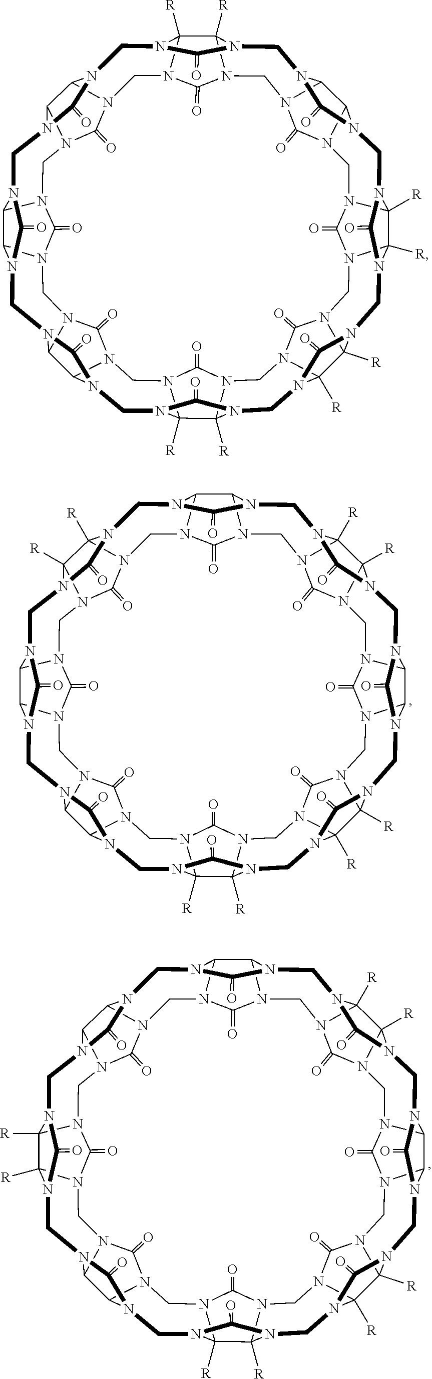

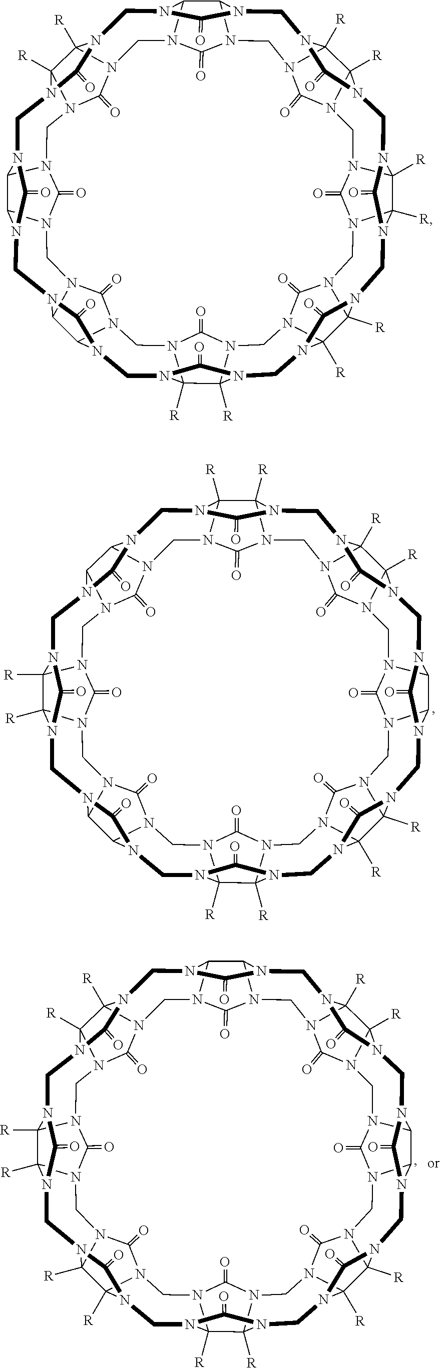

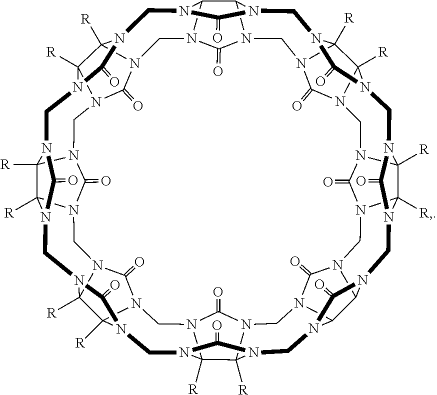

12. The method of claim 11, wherein the one or more compounds have the following structure: 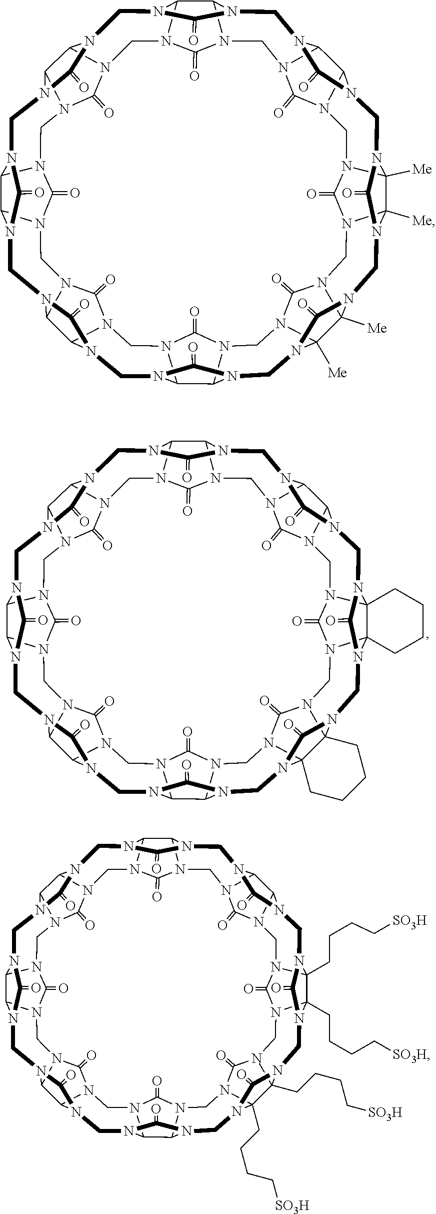 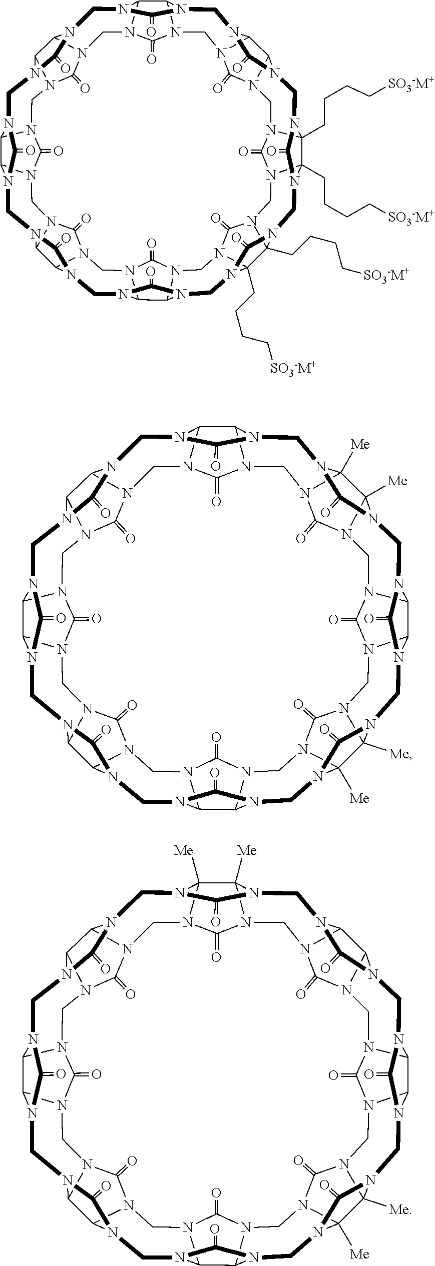 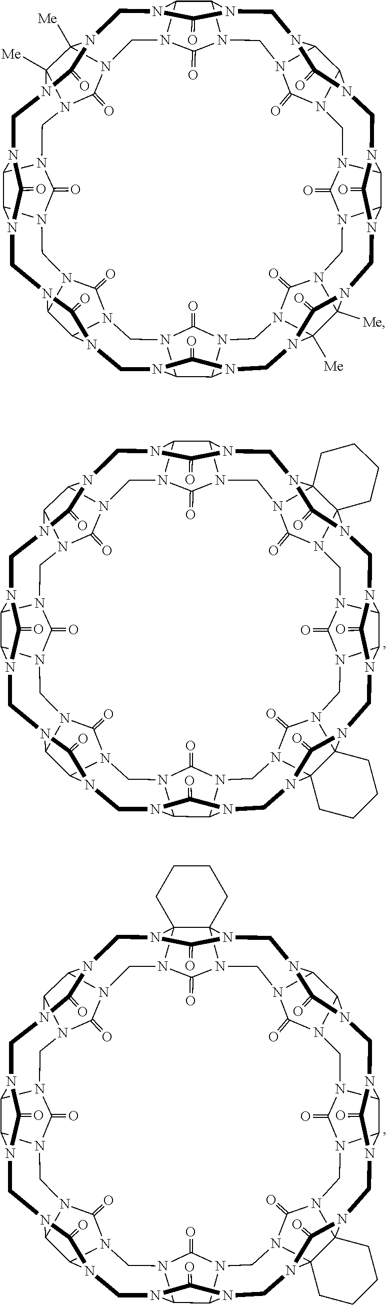 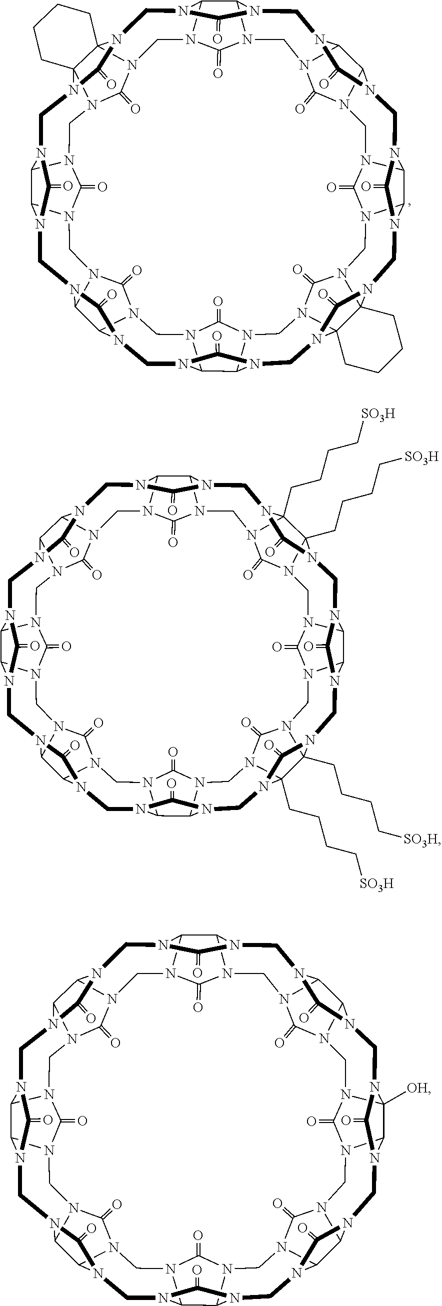 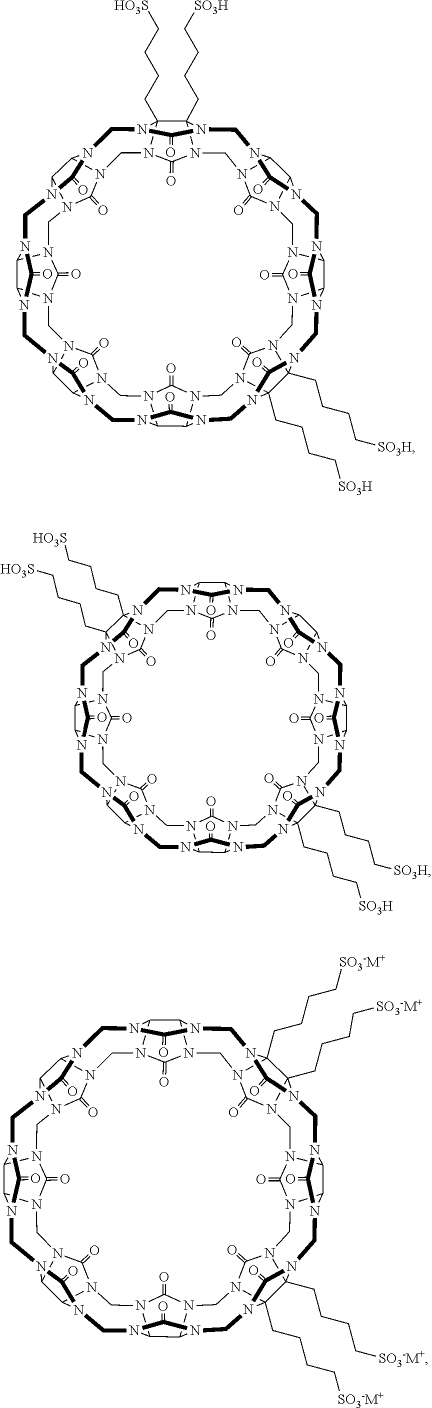 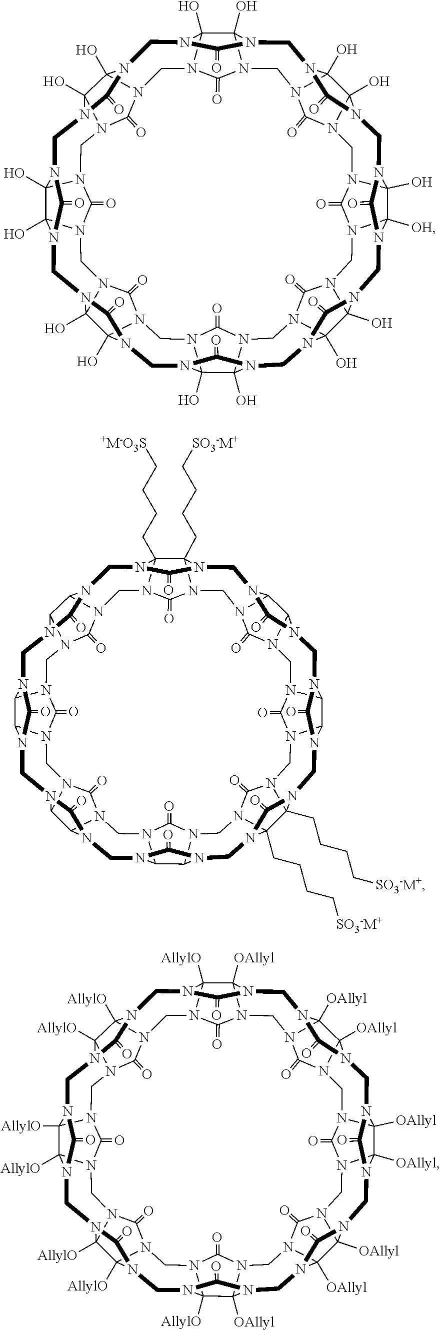 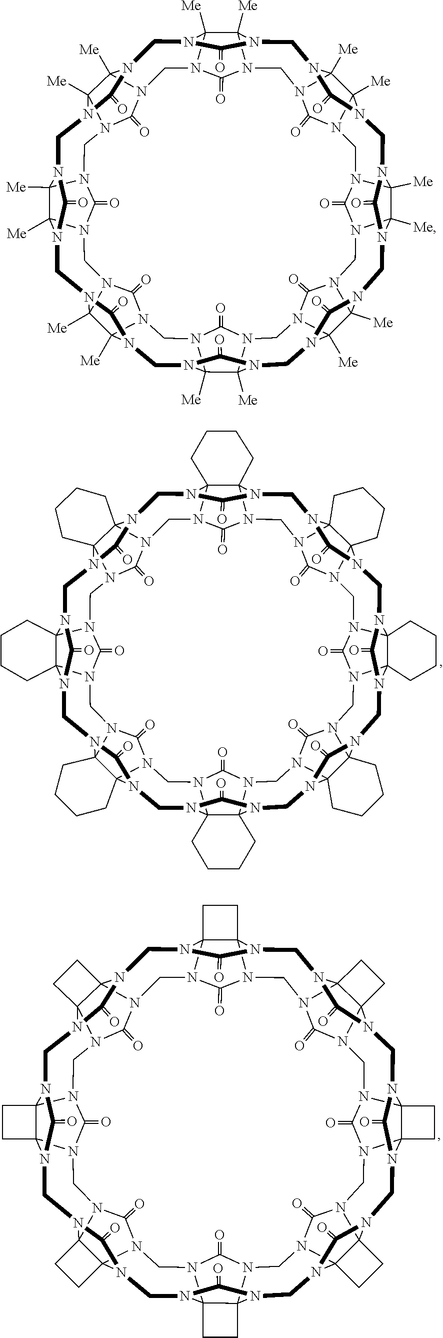 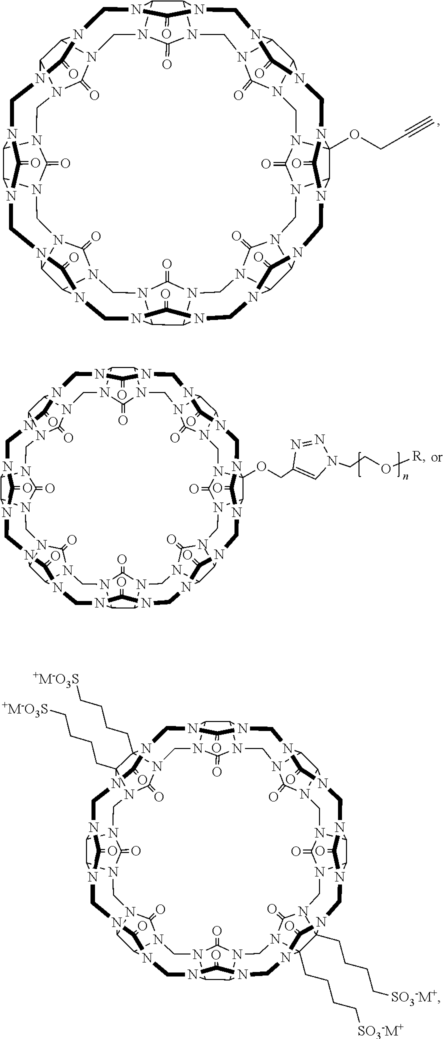
wherein n is 3 to 1000 and M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS).

13. The method of claim 12, wherein the one or more compounds have the following structure:

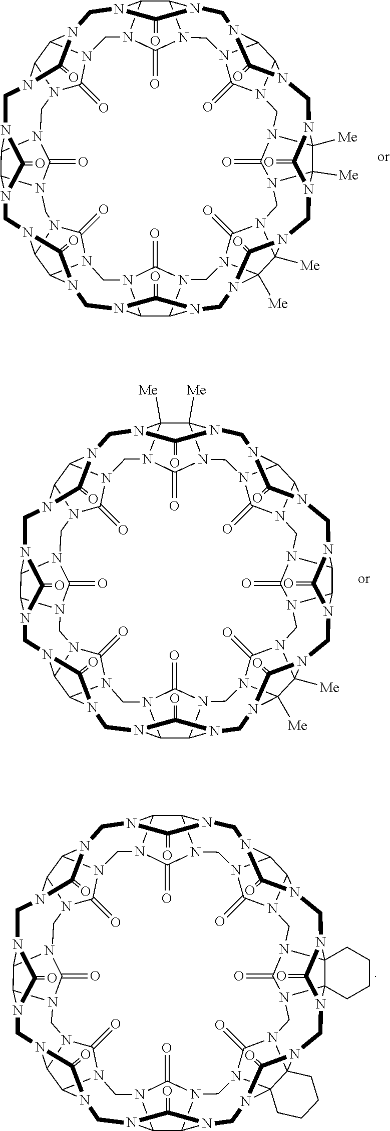

14. The method of claim 10, wherein the individual is in need of reversal of drug-induced neuromuscular block and/or reversal of anesthesia.

15. The method of claim 10, wherein the individual is in need of reversal of the effects one or more drug(s) of abuse.

16. A composition comprising a pharmaceutical carrier, one or more compounds having the following structure: 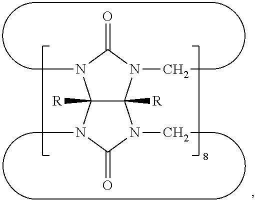
wherein each R is independently chosen from hydrogen, alkyl groups, hydroxyl groups, alkoxy groups, aryl groups, heteroaryl groups, amino groups, amide groups, carboxylic acid/carboxylate groups, ester groups, imide groups, thiol groups, ether groups, polyether groups, phosphate groups, phosphonate groups, sulfonate groups, alkyl sulfonate groups, sulfate groups, alkyl sulfate groups, and combinations thereof, and, optionally, one or more pharmaceutical agents.

17. The composition of claim 16, wherein the one or more compounds have the following structure:

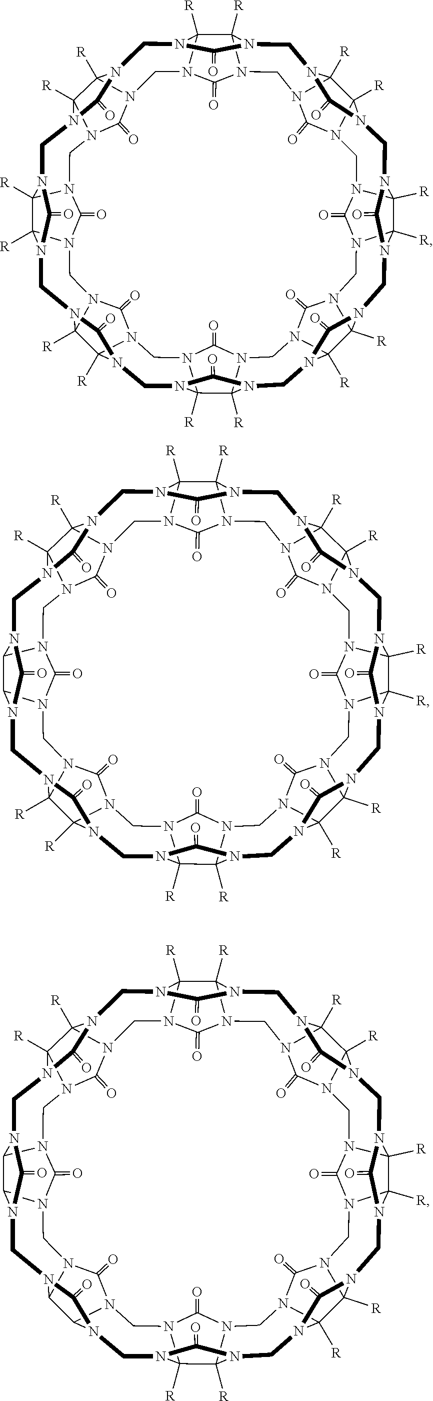

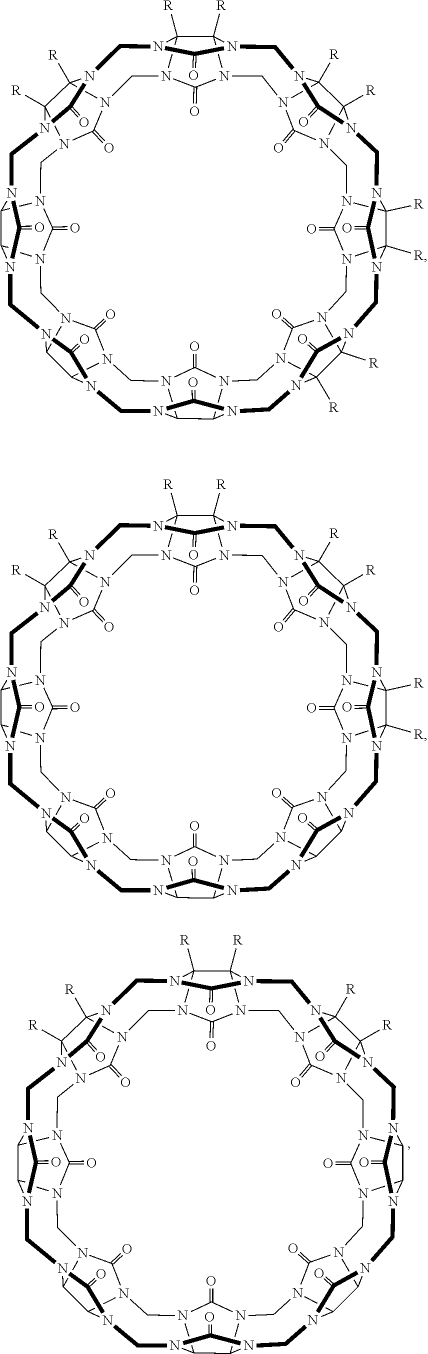

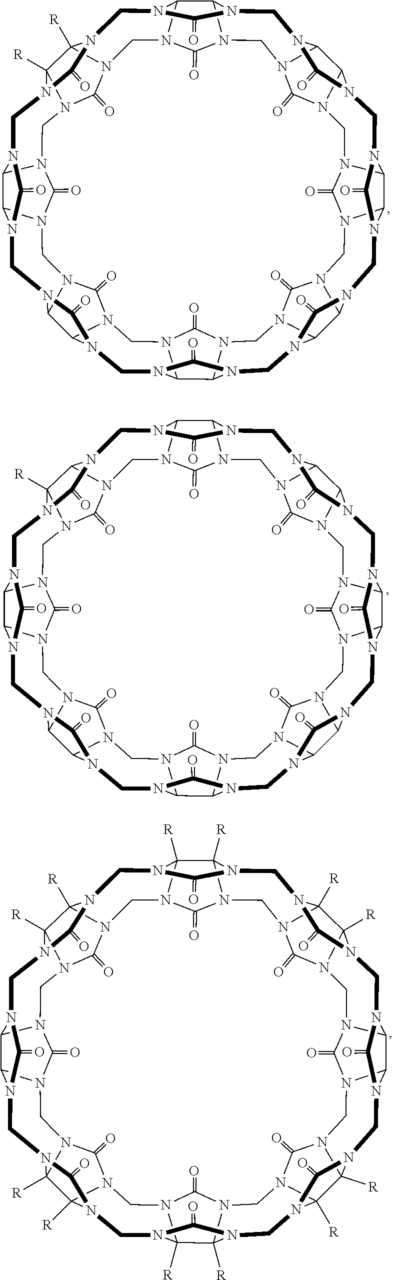

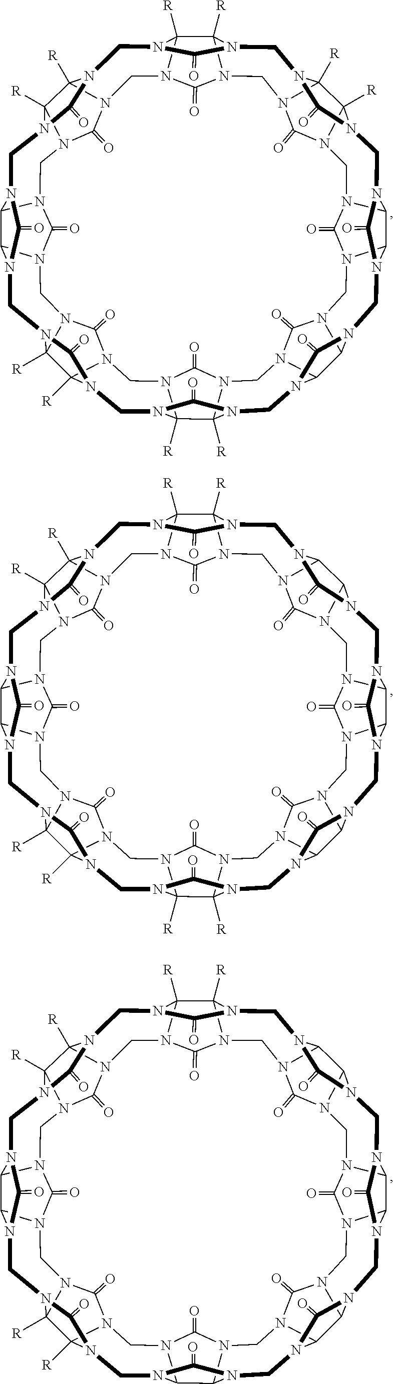

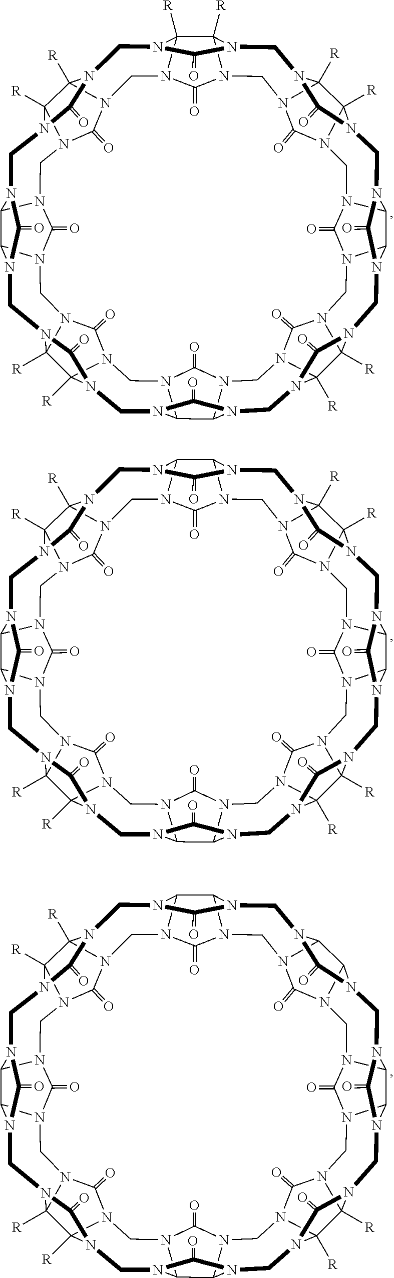

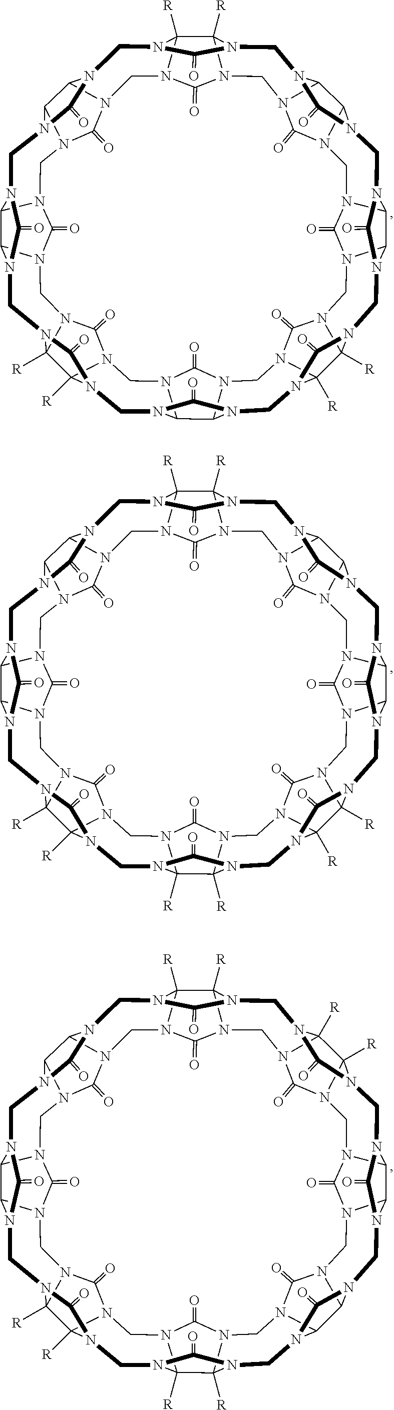

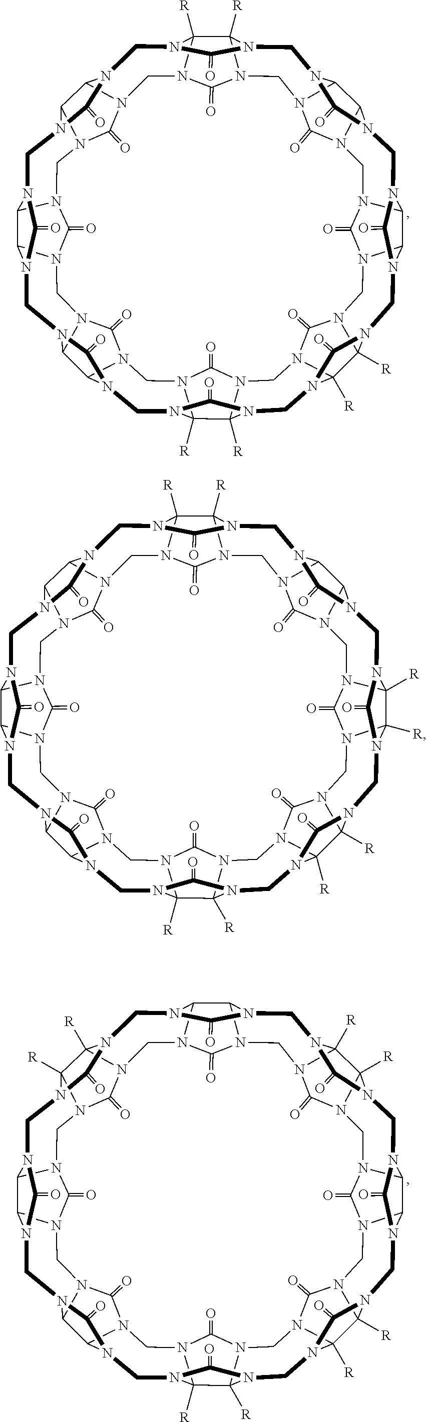

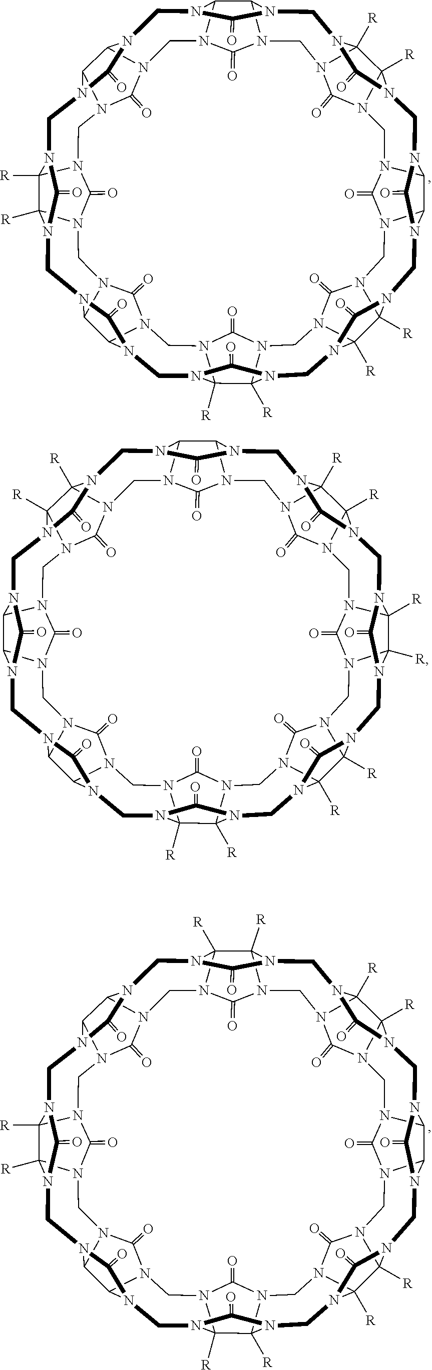

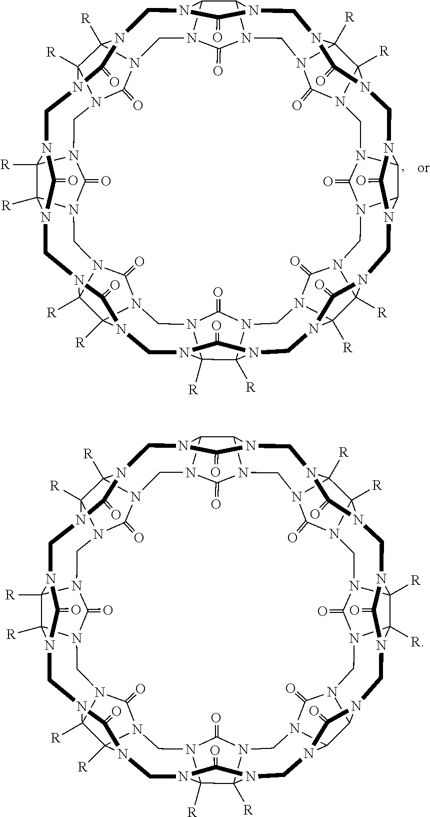

18. The composition of claim 17, wherein the one or more compounds have the following structure: 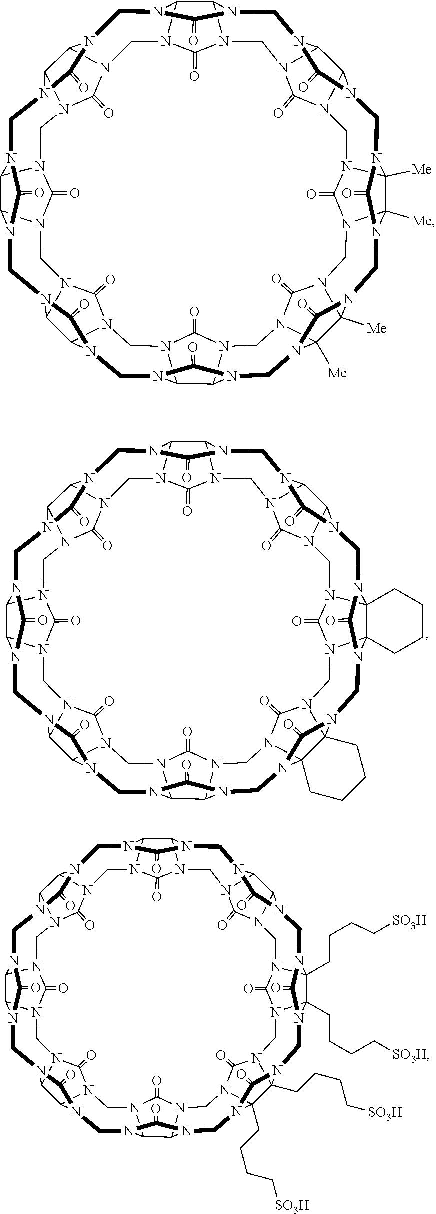 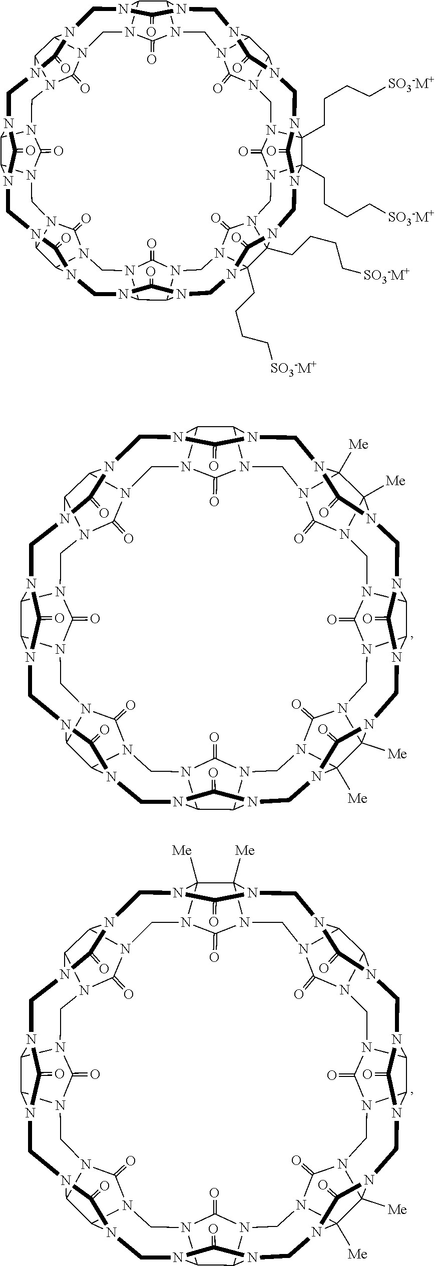 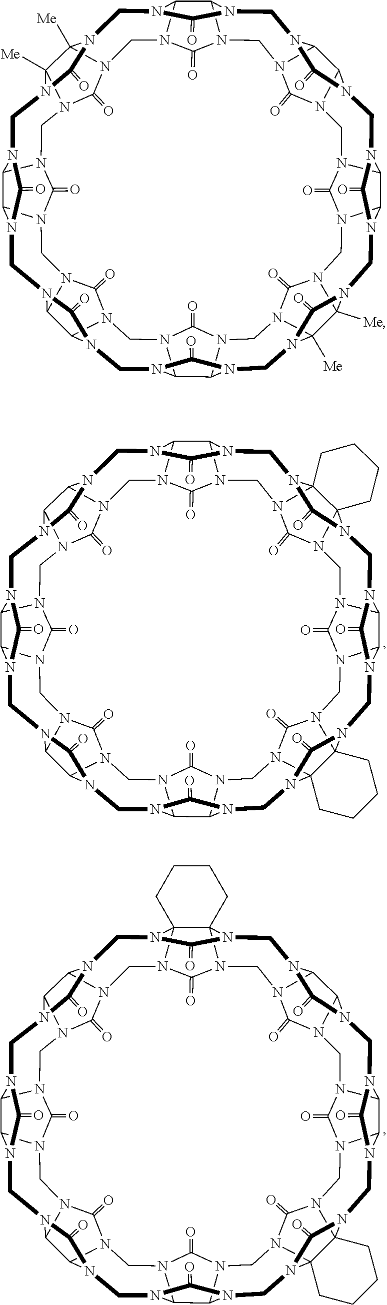 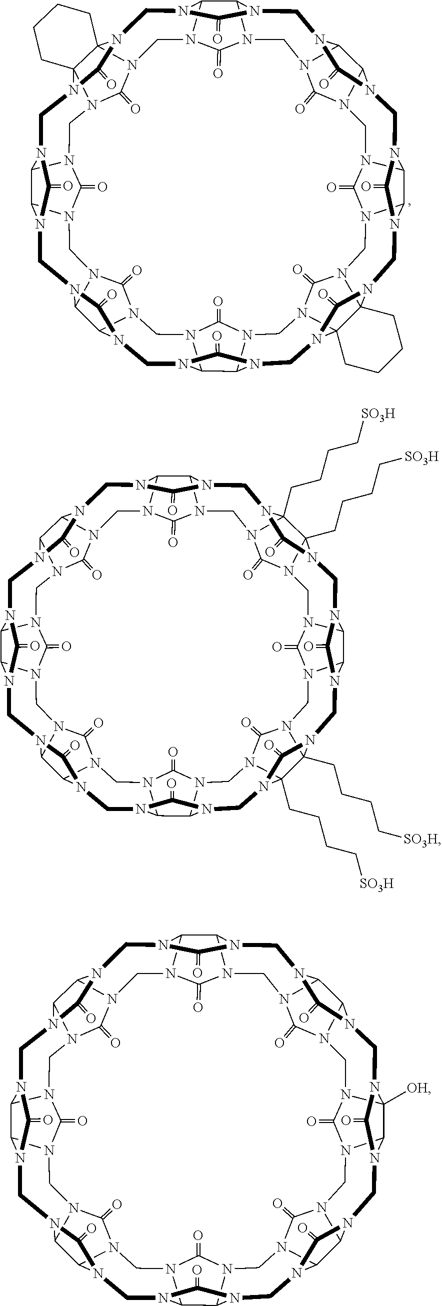 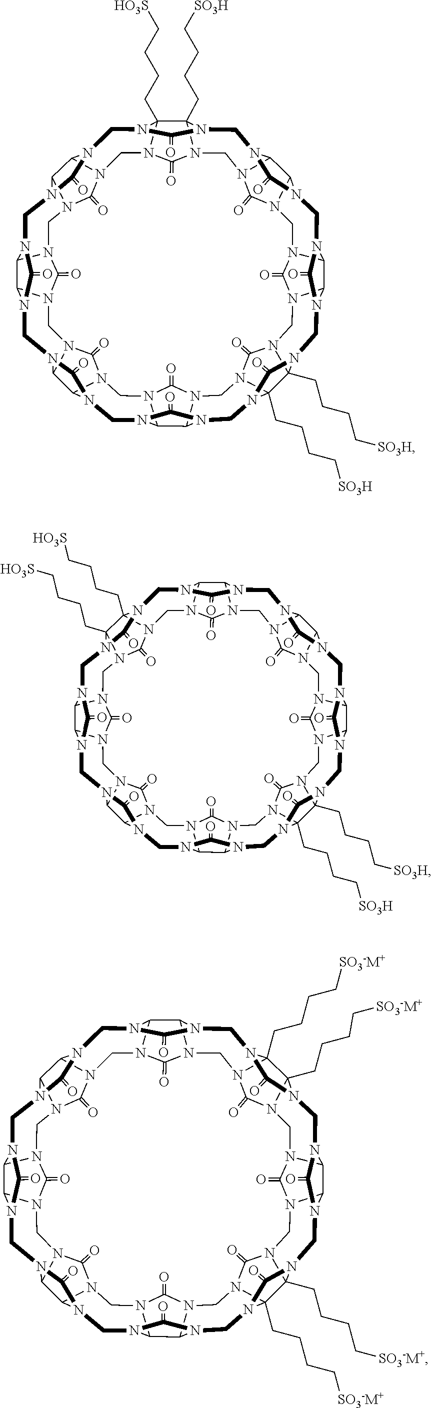 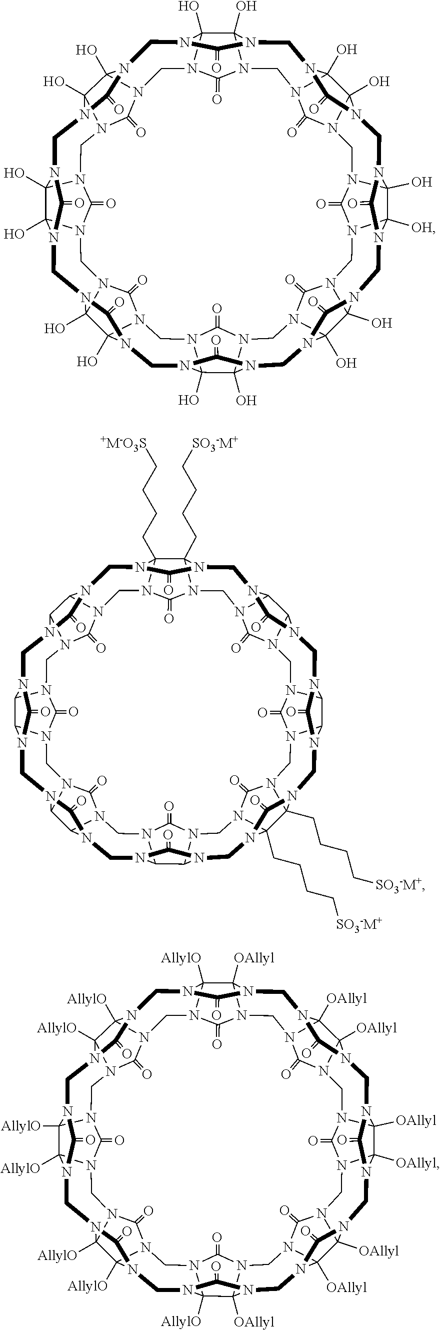 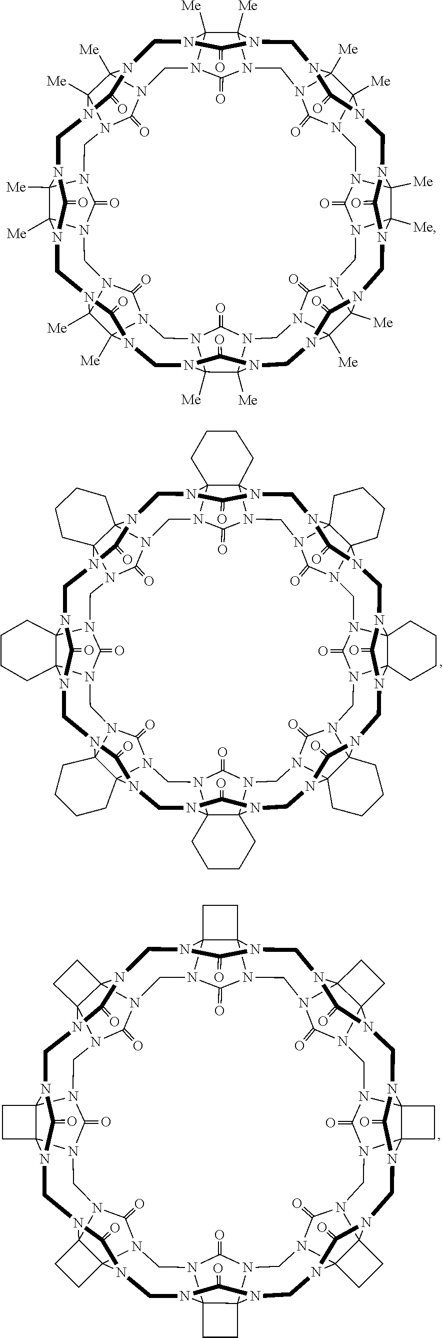 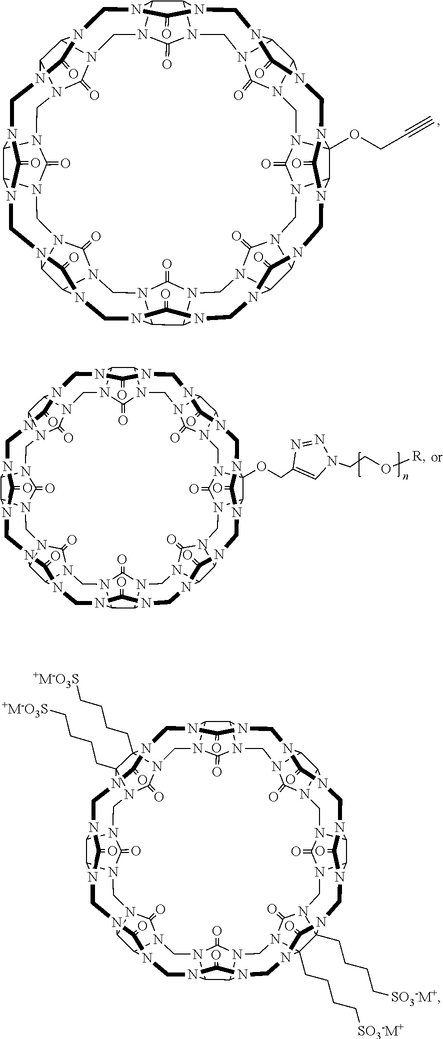
wherein n is 3 to 1000 and M⁺ is chosen from Na⁺, K⁺, Ca²⁺, Mg²⁺, Zn²⁺, H₄N⁺, Et₃NH⁺, Me₄N⁺, (HOCH₂CH₂)₃NH⁺, and cationic forms of ethylenediamine, piperazine, and trishydroxymethyl aminomethane (TRIS).

19. The composition of claim 18, wherein the one or more compounds have the following structure:

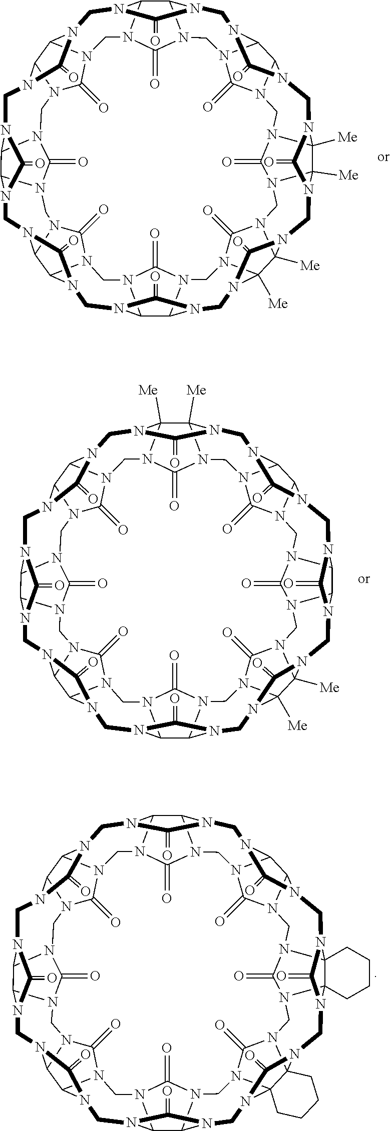

20. The composition of claim 16, wherein at least one R group is not hydrogen.
